(19) United States
(12) Patent Application Publication
(10) Pub. No.: US 2021/0301357 A1
(43) Pub. Date: Sep. 30, 2021

(54) COMPREHENSIVE AND COMPARATIVE FLOW CYTOMETRY-BASED METHODS FOR IDENTIFYING THE STATE OF A BIOLOGICAL SYSTEM

(71) Applicant: Enzo Biochem, Inc., New York, NY (US)

(72) Inventors: Elazar Rabbani, New York, NY (US); James J. Donegan, Amesbury, MA (US); Andrew Stewart L'Huillier, North Potomac, MD (US)

(73) Assignee: Enzo Biochem, Inc., New York, NY (US)

(21) Appl. No.: 17/307,202

(22) Filed: May 4, 2021

Related U.S. Application Data
(63) Continuation of application No. 15/474,271, filed on Mar. 30, 2017, now abandoned.
Application No. 15/474,271 is a continuation-in-part of application No. 14/611,702, filed on Feb. 2, 2015, now abandoned.
(60) Provisional application No. 62/317,007, filed on Apr. 1, 2016.
(60) Provisional application No. 62/095,540, filed on Dec. 22, 2014.

(51) Int. Cl. C12Q 1/70; C12Q 1/6841; C12Q 1/6886

(52) U.S. Cl. CPC C12Q 1/708; C12Q 2600/158; C12Q 1/6886; C12Q 1/6841

(57) ABSTRACT

The invention provides comprehensive and comparative flow cytometry-based methods for characterizing the state of a biological system by determining cell phenotypes and associated gene expression profiles.

### 1. CROSS-REFERENCE TO RELATED APPLICATIONS

This application is a continuation of U.S. application Ser. No. 15/474,271 filed Mar. 30, 2017, which claims the benefit of U.S. Provisional Application Ser. No. 62/317,007 filed Apr. 1, 2016 and is a continuation-in-part of U.S. application Ser. No. 14/611,702 filed Feb. 2, 2015 (now abandoned) which claims the benefit of U.S. Provisional Application Ser. No. 62/095,540 filed Dec. 22, 2014, each of which is hereby incorporated by reference in its entirety.

### 2. REFERENCE TO SEQUENCE LISTING

The instant application contains a Sequence Listing which has been submitted electronically in ASCII format and is hereby incorporated by reference in its entirety. Said ASCII copy, created on Mar. 30, 2017, is named ENZ-110-CIP1-CON-SL.txt and is 65,532 bytes in size.

### 3. BACKGROUND

Many diseases, including autoimmune disease, cancer and certain infections, progress largely due to imbalances in the immune system. Current therapies for treatment of autoimmune diseases, and similarly, organ transplantation, involve regimens of immunosuppression, which tend to be non-selective, leading to inhibition of not only the aberrant autoimmune response, but also of healthy responses to pathogens. Accordingly, immunosuppressive therapies can leave patients susceptible to opportunistic infections or drug toxicity. Furthermore, it would be ideal during treatment of cancer or infection to retrain the patient's immune system to recognize tumor cells or infected cells as non-self, leading to rejection of such diseased or infected cells and remission. Immune suppression and immune tolerance are complex processes which we are still learning to better control. In addition, many other diseases will require a better understanding of the role of the immune system in the etiology of disease in order to more effectively ameliorate or cure the diseases. The use of drugs or immunization with specific antigen epitopes to modulate patient immune responses has huge potential to treat a wide range of diseases. To better treat such patients, two things are needed: (i) methods for assessing patient response to epitopes for potential use in inducing immune tolerance or immune responsiveness, and (ii) methods for monitoring patient progress during both standard treatments and antigen challenge. These insights will allow tracking of the immune status of the patient, which will enable the development of effective therapy for the patient. Combined with the ability to monitor genes associated with lymphocyte memory, such techniques will allow for long-term immune modulation tailored to individual patients.

Current methods for tracking immune status or assaying gene activity (e.g., transcription) relate to lymphocyte memory, function or training and involve analyses of plasma cytokine levels and proliferation of peripheral blood mononuclear cells. However, information gained using these methods is not directly correlated with specific immune-mediated responses of cells that are involved in the etiology of disease, but include collective responses from circulating cells. A better understanding of disease progression and treatment efficacy requires methods for discovering what is lost or what is aberrantly gained in patients suffering from immune-mediated disorders or disease as compared to, for example, a healthy individual. Information gleaned from such methods should enable identification of treatments that impart a return of balance in the immune system that can then be monitored during clinical interventions.

Accordingly, there is a need for more rapid and sensitive methods for quantitative and/or qualitative multiplex phenotypic analyses, which are indicative of immune or other cell function using homogeneous systems that do not require excessive cell manipulation to insure accurate measurement of cellular targets. There is also a need for more rapid and accurate methods for detecting and/or characterizing abnormal cells or obtaining gene expression profiles of specific cell types present in heterogeneous cell populations. Such methods will allow for assessment of immune cells involved in the etiology or maintenance of autoimmunity, tumor growth, and other diseases, in addition to peripheral cells, providing a more complete picture of patient immune status, and can be used to glean information for the design and/or revision of targeted treatments for immune-mediated disorders, and other diseases.

### 4. SUMMARY

The present disclosure provides comprehensive and comparative flow cytometry-based methods for identifying the state of a biological system by identifying the phenotype of cells and correlating the phenotype with cell function. The present disclosure further relates to flow cytometry-based methods for identifying cell morphology and/or cell cycle progression and correlating these parameters with cell function. In particular embodiments, the cells are immune cells from a subject suffering from an immune-mediated disorder, including autoimmune disorders and immunodeficiency disorders that result in imbalance of the immune system and impair a subject's ability to recognize self-antigens or fight infection or disease. In certain aspects, cell phenotype is identified by detecting and/or quantifying one or more markers on the cell surface, which readily enables identification of specific sub-types of cells of interest, e.g., pro-inflammatory T-cells. In various embodiments, cell phenotype is identified using labeled antibodies specific for one or more markers on the cell surface.

Accordingly, in additional aspects, function of particular subsets of cells identified by cell surface markers or transcription factors is determined by detecting patterns of gene expression, e.g., cytokine expression, cytokine receptor expression, expression of microRNA or other RNA types, or other markers related to immune function, by detecting and/or quantifying transcription in the cell, by assaying DNA content, which is indicative of the rate of cell replication or cell death, by assaying cell receptors, and/or by detecting the number and/or state of organelles, such as one or more of Golgi apparatuses, nucleoli, nucleus, mitochondria, vesicles, lysosomes, phagophores, autophagosomes, autophagolysosomes, and/or transport systems such as calcium channels, endoplasmic reticulum, nuclear membranes, mitochondrial membranes, or other markers related to the functional state of the cell, such as mitochondrial potential, hypoxia, or reactive species (e.g., reactive oxygen species) generation. In certain embodiments, the number of organelles is compared (i.e., normalized) to an internal element such as the number of a cellular organelle that does not normally change, e.g., the nucleus. In other embodiments, the number and/or state of cellular organelles is normalized to the number and/or state of cellular organelles in external cells such as a cell line, or a cell from a healthy individual. In certain embodiments, gene expression is assayed using labeled probe systems for detecting single-stranded nucleic acids derived from homogeneous probe systems that enable the detection of cellular components of interest only when probes are bound to the target without the need for washing to remove unbound probes or excessive manipulation of the sample that may result in loss of cell contents. In other embodiments, cells and organelles are assayed using dye probes, such as cell staining dyes or organelle specific dyes. In preferred embodiments, the methods described herein include multiplex analyses that include two or more of cell transcription, DNA content, protein expression, organelle status, transport system status and the like. In various embodiments, the methods described herein further include a control element for an internal or external reference point, such as a housekeeping gene, control cells such as a cell line, primary cells from tissue, or cells of the same type that are from a healthy individual.

In particular embodiments, the flow cytometry methods described herein can be used to identify better tolerogenic or immunogenic epitopes for respectively treating subjects suffering from autoimmune disorders or immunodeficiency disorders. In these embodiments, the phenotype of particular cells of interest, e.g., responder T-cells or regulatory T-cells, are compared with the same subsets of cells from a healthy individual. In further embodiments, the cells of interest from a subject that has been treated for a disorder are compared to the same subset of cells taken from the same subject before treatment. In other embodiments, the flow cytometry methods described herein can be used to identify epitopes for immunization of healthy individuals.

One embodiment of the invention includes:

in a liquid medium such as a liquid aqueous medium, such as a nucleic acid hybridization buffer, a nucleic acid hybridization wash buffer or a flow cytometry medium,
a sample including isolated primary human cervical cells, the cells of said sample being fixed, permeabilized and at least predominantly suspended or suspendable as individual cells in the liquid medium; and
a plurality of different E6/E7 hybrid molecular probes (HMPs), each including two oligonucleotide hybridization probe portions with a FRET donor attached to one and a FRET acceptor attached to the other (thereby constituting a FRET donor acceptor pair), and each configured, by way of the nucleotide sequence of each of the two oligonucleotide hybridization probe portions thereof, to specifically hybridize to different, non-overlapping target sequences in E6/E7 RNA of a human papilloma virus, such as HPV-16 and/or HPV-18. The different hybrid molecular probes of the plurality may include the same FRET donor acceptor pair (allowing common simultaneous detection of hybridization) or may include different FRET donor-acceptor pairs among them. The primary human cervical cells may be ectocervical cells obtaining from the ectocervix of a human subject using, for example, conventional collection procedures.

The composition may further include one or both of:

at least one p16^{INK4A} hybrid molecular probe configured to specifically hybridize to human p16^{INK4A} RNA,
said p16^{INK4A} hybrid molecular probe comprising a FRET donor-acceptor pair that is compatible (selected) for distinguishable detection from the one or more FRET donor-acceptor pairs of the different E6/E7 hybrid molecular probes; and

an antibody that specifically binds human p16^{INK4A} protein, which antibody may, for example, be fluorescently labeled and which antibody may, for example, be a monoclonal antibody or a polyclonal antibody or a fragment of either that specifically binds human p16^{INKA} protein. The at least one p16^{INK4A} hybrid molecular probe may include a plurality of different p16^{INK4A} hybrid molecular probes configured to hybridize to different, non-overlapping target sequences in human p16^{INK4A} RNA. The different p16^{INK4A} hybrid molecular probes may include the same FRET donor-acceptor pairs.

In one variation of the composition, the plurality of different hybrid molecular probes configured to specifically hybridize to different, non-overlapping target sequences in E6/E7 RNA of a human papilloma virus includes:

(a) a plurality of different hybrid molecular probes configured to specifically hybridize to different target sequences in E6/E7 RNA of HPV-16; and

(b) a plurality of different hybrid molecular probes configured to specifically hybridize to different target sequences in E6/E7 RNA of HPV-18.

All the hybrid molecular probes of (a) and (b) may, for example, have the same FRET donor acceptor pair or pairs. All the hybrid molecular probes within (a) may have the same donor acceptor pair or pairs and all the hybrid molecular probes within (b) may have the same donor acceptor pair or pairs which are different from and distinguishably detectable from those in (a).

In the composition, at least some of the human cervical cells of the sample may include the E6/E7 RNA of the human papilloma virus, e.g., as a result of HPV viral infection of the cells, and at least some of the plurality of different E6/E7 hybrid molecular probes may be specifically hybridized to said E6/E7 RNA. Where the composition includes the at least one p16^{INK4A} hybrid molecular probe, at least some of the human cervical cells of the sample may include cellular p16^{INK4A} RNA and at least some of the at least one p16^{INK4A} hybrid molecular probe may be specifically hybridized to said cellular p16^{INK4A} RNA. Where the composition includes the antibody, that specifically binds human p16^{INK4A} protein, for at least some of the human cervical cells of the sample, said antibody may be specifically bound to cellular human p16^{INKA} protein.

Where a plurality of probes, such as hybrid molecular probes or molecular beacons, targeting different non-overlapping sequence portions of the same target nucleic acid strand (such as a target RNA, e.g., a target mRNA) are used, the plurality of said probes may, for example, consist of 2-10 of said probes, such as 3-6 or 3-5 of said probes, or any subrange or number of probes within said ranges. The sample of the composition may include, for example, at least 1,000 human cervical cells, at least 2,500 human cervical cells, at least 5,000 human cervical cells, or at least 10,000 human cervical cells.

It should be noted that the indefinite articles “a” and “an” and the definite article “the” are used in the present application to mean one or more unless the context clearly dictates otherwise. Further, the term “or” is used in the present application to mean the disjunctive “or” or the conjunctive “and.”

All publications mentioned in this specification are herein incorporated by reference. Any discussion of documents, acts, materials, devices, articles or the like that has been included in this specification is solely for the purpose of providing a context for the present disclosure. It is not to be taken as an admission that any or all of these matters form part of the prior art or were common general knowledge in the field relevant to the present disclosure as it existed anywhere before the priority date of this application.

The features and advantages of the disclosure will become further apparent from the following detailed description of embodiments thereof.

### 5. BRIEF DESCRIPTION OF THE DRAWINGS

FIGS. 1A-1E. FIG. 1A provides a binary probe system in which signal is generated when two labeled target-specific probes hybridize to the target such that the labels are in proximity and energy transfer occurs. FIG. 1B provides a yin-yang probe system in which signal is generated when the target-specific probe containing a donor moiety separates from a complementary probe comprising an acceptor moiety and binds to the target nucleic acid to produce a signal. FIG. 1C provides a binary yin-yang probe system which is a combination of the probe systems set forth in FIG. 1A and FIG. 1B. In this system, target-specific probes comprising a first donor and a second donor are individually quenched by the binding of complementary probes comprising an acceptor moiety. A signal is generated when each target-specific probe separates from its complementary probe comprising the quencher moiety and binds to adjacent areas on the target such that energy transfer occurs between each probe. FIG. 1D provides a lightup probe system in which a target-specific probe comprising a single label hybridizes to the target and generates a signal by interaction with unlabeled nucleotides that are present in the target. FIG. 1E schematically illustrates a hybrid molecular probe (HMP) that includes two oligonucleotide probe portions, one of which has a FRET donor attached thereto and the other of which has a FRET acceptor attached thereto, in which the two probe portions are connected by a polyethylene glycol linker. The sequences of the probe portions are selected to specifically hybridize to the same strand of a nucleic acid target, such as an RNA transcript, in sufficient proximity to each other that FRET can occur between the donor and acceptor when the probe portions are specifically hybridized to the target.

FIGS. 2A-2D. FIG. 2A provides molecular beacon probes having a stem-loop conformation where one end of the probe is labeled with a donor moiety, the other end of the probe is labeled with a quencher moiety and the loop portion is target-specific. When the probe is in the stem-loop formation, the donor signal is quenched, but when the probe is bound to the target, the donor and quencher are physically separated from each other and the donor signal is no longer quenched. FIG. 2B provides a stemless molecular beacon probe in which a random coil brings the ends of the linear strand, one of which is labeled with a donor and the other of which is labeled with a quencher, into proximity with each other such that a transition can be seen between bound and unbound beacons lacking secondary structure. FIG. 2C provides a binary molecular beacon system, which utilizes a pair of molecular beacons that hybridize to adjacent sites on a nucleic acid of interest such that when they bind to a target, the donor labeled ends of each molecule are no longer in proximity with the quenchers, but are in sufficient proximity to each other that they generate a signal through energy transfer from donor 1 to donor 2. FIG. 2D provides an extended molecular beacon in which not only the loop region, but also one of the stems and exterior sequences of the probe are complementary to the target of interest, which may be more efficient in binding to targets, since two separate regions (the loop and the extension) that are not involved in the secondary structure of these beacons are complementary to the target. As in other molecular beacon designs, signal is generated by target-dependent separation of the donor from the quencher.

FIGS. 3A-3C. FIG. 3A provides flow cytometry results using HPV-specific molecular beacon probes in an HPV-negative cell line (C33A). FIG. 3B provides flow cytometry results using the HPV specific molecular beacon probes in an HPV-positive cell line (HPV Ect E6/E7). Hybridization was also carried out with β-actin internal control molecular beacons in each cell line. In these diagrams, signals from the HPV-specific molecular beacons are recorded as FL1 and β-actin internal control molecular beacon signals are recorded as FL3. The percentage of cells registered as HPV positive signals is given in the upper left corner. In FIG. 3C, the data from FIG. 3A and FIG. 3B were reconfigured and presented together as the Mean Fluorescence Index for HPV-negative (C33A) cells (solid line peak) and HPV-positive cells (Ect1E6E7) (dotted line peak).

FIGS. 4A-4B. FIG. 4A provides flow cytometry results with an HPV-positive clinical sample using side scatter and forward scatter and gating to measure only ectodermal cells. FIG. 4B shows ectocervical cells selected from gating in FIG. 4A measured for hybridization with HPV-specific molecular beacons where FL1 represents E6/E7 probes and FL3 represents β-actin probes and 5.75% of the cells were positive for HPV. The results show a clear and distinct difference between the signals generated by the HPV-positive and HPV-negative cells as detected by the HPV E6/E7 molecular beacons.

FIG. 5 provides a comparison of flow cytometry results from HPV clinical samples numbered 1-18 using either the incellDX HPV OncoTect™ E6, E7 mRNA kit or HPV-specific molecular beacons. Numbers in the table are the percentages of cells identified as being positive for HPV E6/E7 in each sample. Test 1 and Test 2 are duplicate assays from the same specimen. Specimens having more than 2% of HPV-positive cells were considered to be positive for likelihood toward progression of infection to cancer.

FIG. 6 provides flow cytometry detection and quantification results using various molecular beacons specific for mRNA transcribed from GAPDH, 28S or β-actin sequences. Sequences for the beacons are provided in Example 1. Values were calculated as Mean Fluorescence Index.

FIG. 7 provides flow cytometry detection and quantification results using various molecular beacons specific for p16 mRNA derived from Ect1/E6E7 cells. Sequences for the beacons are provided in Example 1. Values were calculated as Mean Fluorescence Index.

FIG. 8 provides a comparison of results achieved with pathology evaluations and molecular beacons for HPV16/18 and p16. In addition, some of the clinical specimens were evaluated using the Roche HPV High Risk assay, Roche HPV 16 Assay, Roche HPV18 Assay, and the E-tect (incellDX HPV OncoTect™ E6, E7 mRNA) assay. Results are given as “P” for Positive and “N” for Negative results for the Roche and E-tect tests, while the molecular beacon assays give the percentage positive cells where HPV E6/E7 results higher than 2% are considered to be positive.

FIG. 9 provides an evaluation of the amount of signal (MFI) generated when molecular beacons for the E6/E7 gene of HPV16 and HPV 18 are assayed with artificial E6/E7 mRNA transcripts from a number of oncogenic HPV types.

FIG. 10 provides an evaluation of the amount of signal (MFI) generated when molecular beacons from the E6/E7 gene of HPV31 and HPV33 are assayed with artificial E6/E7 mRNA transcripts from a number of oncogenic HPV types.

FIGS. 11A-11D provide flow cytometry results from a HPV-positive clinical sample using side scatter and forward scatter and gating to measure only ectodermal cells. FIG. 11A provides flow cytometry results from cells prepared using the E-tect 2.0 protocol set forth in Example 13 in the absence of an E6/E7 specific molecular beacon probe, and FIG. 11B provides cytometry results from cells prepared using the E-tect 2.0 protocol in the presence of an E6/E7 specific molecular beacon probe. The amount of signal (MFI) generated by the probe in the cells of FIG. 11B was 29.8. FIG. 11C provides flow cytometry results from cells prepared using the E-tect 2.5 protocol described in Example 13 in the absence of an E6/E7 specific molecular beacon probe, and FIG. 11D provides cytometry results from cells prepared using the E-tect 2.5 protocol in the presence of the same E6/E7 specific molecular beacon probe used in FIG. 111B. The amount of signal (MFI) generated by the molecular beacon probe in the cells of FIG. 11D was 497.

FIGS. 12A-12C provide flow cytometry results from Jurkat cells activated by exposure to anti-CD3 using side scatter and forward scatter and molecular beacons specific for IFN-γ mRNA (FL1-H). FIG. 12A provides flow cytometry results from Jurkat cells that were not exposed to anti-CD3 in which a small percentage of cells are activated and producing INF-γ. FIG. 12B provides flow cytometry results from activated Jurkat cells 18 hours after activation and shows that a larger percentage of cells are producing INF-γ and therefore, are activated. FIG. 12C indicates that 36 hours after activation, an even larger number of Jurkat cells are activated and producing INF-γ.

FIGS. 13A-13D. FIG. 13A provides flow cytometry results in HPV-positive clinical samples using side scatter and forward scatter using NuclearID to gate on the G1, S and G2 phase populations based on DNA content. Each population was analyzed for E6/E7 mRNA separately. FIGS. 13B-13D provide flow cytometry results for E6/E7 mRNA (recorded as FL1-H) in G1 phase, S phase and G2 phase cells, respectively using molecular beacon probes specific for E6/E7. This example shows that resting G1 phase cells (FIG. 13B) have little to no viral DNA, while almost all of proliferating S phase cells (FIG. 13C) and G2 phase cells (FIG. 13D) have high virus content.

FIGS. 14A-14E. FIG. 14A provides flow cytometry results from biotin labeled beads for comparison of brightness (negative control). FIG. 14B provides flow cytometry results for SA-FITC at 18 nM (positive control). FIG. 14C provides flow cytometry results for 1 nM 1:1 bDNA. FIG. 14D provides flow cytometry results for 1 nM 1:3 bDNA. FIG. 4E provides flow cytometry results for 1 nM 1:4 bDNA. FIGS. 14C-14E show that 1 nM bDNA provides similar brightness to an 18-fold higher concentration of SA-FITC (14B).

FIGS. 15A-15H. FIG. 15A provides flow cytometry results from CD25+ cells that have not been stained (negative control). FIG. 15B provides flow cytometry results for T-cells stained with CD25 specific antibody and 18 nM SA-FITC. FIG. 15C provides flow cytometry results for T-cells stained with 1:1 bDNA in the absence of CD25 specific antibody, and FIG. 15D provides flow cytometry results for T-cells stained with 1:1 bDNA in the presence of CD25 specific antibody. FIG. 15E provides flow cytometry results for T-cells stained with 1:3 bDNA in the absence of CD25 specific antibody, and FIG. 15F provides flow cytometry results for T-cells stained with 1:3 bDNA in the presence of CD25 specific antibody. FIG. 15G provides flow cytometry results for T-cells stained with 1:4 bDNA in the absence of CD25 specific antibody, and FIG. 15H provides flow cytometry results for T-cells stained with 1:4 bDNA in the presence of CD25 specific antibody. FIGS. 15D, 15F and 15H show that bDNA gave much higher signal at a lower concentration than SA-FITC (FIG. 15B) or unstained cells (FIG. 15A). FIGS. 15C, 15E and 15G show that bDNA has background fluorescence when staining cells.

FIGS. 16A-16I. FIGS. 16A-16C provide flow cytometry results from cells stained with 1 nM 1:1 bDNA in phosphate buffered saline (PBS), sodium phosphate and heparin, respectively. FIGS. 16D-16F provide flow cytometry results from cells stained with 1 nM 1:3 bDNA in phosphate buffered saline (PBS), sodium phosphate and heparin, respectively. FIGS. 16G-16I provide flow cytometry results from cells stained with 1 nM 1:4 bDNA in phosphate buffered saline (PBS), sodium phosphate and heparin, respectively. As shown in FIGS. 16B, 16E and 16H, increased phosphate concentration (75 mM sodium phosphate) moderately reduced background as compared to PBS (FIGS. 16A, 16D and 16G, respectively). As shown in FIGS. 16C, 16F and 16I, addition of heparin (75 U/mL) completely removed background fluorescence as compared to PBS (FIGS. 16A, 16D and 16G, respectively).

FIGS. 17A-17D provide flow cytometry results from H9 T-cells or CaSki ectocervical cells using side scatter and forward scatter and molecular beacon probes specific for the GAPDH housekeeping gene. FIG. 17A provides flow cytometry results from H9 T-cell that were not exposed to GAPDH molecular beacons (negative control). FIG. 17B provides flow cytometry results for H9 T-cells that were exposed to a mixture of GAPDH molecular beacons having the sequences set forth in SEQ ID NO: 11 and SEQ ID NO: 12 as shown in Example 1. FIG. 17C provides flow cytometry results from CaSki cells that were not exposed to GAPDH molecular beacons (negative control). FIG. 17D provides flow cytometry results for CaSki cells that were exposed to a mixture of GAPDH molecular beacons having the sequences set forth in SEQ ID NO: 11 and SEQ ID NO: 12 as shown in Example 1. As shown in FIGS. 17B and 17D, respectively, H9 cells express less GAPDH than CaSki cells.

FIGS. 18A-18E. FIGS. 18A-18B provide flow cytometry results from SH-SY5Y neuronal cells using side scatter and forward scatter and a molecular beacon probe specific for poly-A regions of mRNAs (SEQ ID NO: 33). FIG. 18A provides flow cytometry results from SH-SY5Y cells that were not exposed to the poly-A specific molecular beacon (negative control). FIG. 18B provides flow cytometry results for SH-SY5Y cells that were exposed to the poly-A probe, which shows that the probe successfully labeled the neuronal cells and can be used as a control probe. FIGS. 18C-18E provide flow cytometry results for SH-SY5Y neuronal cells using side scatter and forward scatter using labeled probes for actin. FIG. 18C provides flow cytometry results from SH-SY5Y cells that were not exposed to the actin probes (negative control). FIG. 18D provides flow cytometry results for SH-SY5Y cells that were exposed to a SmartFlare™ probe (EMD Millipore), and FIG. 18E provides flow cytometry results for SH-SY5Y cells that were exposed to a cocktail of actin probes having the sequences of SEQ ID NOs: 7, 8, 9 and 10 as set forth in Example 1. FIGS. 18E and 18F show that the cocktail of actin probes provide a brighter signal (FIG. 18F) than the SmartFlare™ (FIG. 18E), indicating that different cells in the population express different amounts of actin mRNA.

FIGS. 19A-19D. FIGS. 19A-19B provide flow cytometry results from H9 cells, which are not cervical cancer cells, using side scatter and forward scatter and a cocktail of molecular beacon probes (SEQ ID NOs: 16, 17, 18, 19, 20, 21, 22, 23 of Example 1) specific for P16. FIG. 19A provides flow cytometry results from H9 cells that were not exposed to the P16 probes. FIG. 19B provides flow cytometry results for H9 cells that were exposed to the P16 molecular beacon cocktail, which shows that the H9 cells do not contain P16 mRNA. FIGS. 19C-19D provide flow cytometry results from CaSki cervical cancer cells using side scatter and forward scatter and a cocktail of molecular beacon probes (SEQ ID NOs: 16, 17, 18, 19, 20, 21, 22, 23 of Example 1) specific for P16. FIG. 19C provides flow cytometry results from CaSki cells that were not exposed to the P16 probes. FIG. 19D provides flow cytometry results from CaSki cells that were exposed to the P16 molecular beacon cocktail and shows that the cocktail of P16 specific molecular beacons efficiently detect P16 over-expression in infected cells.

FIG. 20 provides HPV E6/E7 coding sequences used for transcription cassettes including sequences of E6/E7 genes of HPV6, HPV11, HPV16, HPV18, HPV26, HPV31, HPV33, HPV35, HPV39, HPV42, HPV43, HPV44, HPV45, HPV51, HPV52, HPV53, HPV56, HPV58, HPV59, HPV66, HPV67, HPV68, HPV70, HPV73 and HPV82.

FIGS. 21A-21C. FIG. 21A provides flow cytometry results for streptavidin coated polystyrene beads without target in the presence of E6/E7 molecular beacons having SEQ ID NOs. 1-6 and shows baseline fluorescence. FIG. 21B provides flow cytometry results for streptavidin coated polystyrene beads bound to a sequence not found in HPV (“off-target” sequence) and shows fluorescence that is similar to the fluorescence from beads without target in FIG. 21A. FIG. 21C provides flow cytometry results for streptavidin coated beads bound to HPV E6/E7 target DNA as a positive control.

FIGS. 22A-22I. FIGS. 22A and 22B respectively provide flow cytometry results for expression of 28S and Gapdh housekeeping genes in E6/E7 negative primary cervical cells. Expression of 28S and Gapdh is robust in endocervical and PBMC cells (high SSC cells), but expression of 28S and GAPDH in ectocervical cells (low SSC cells) is low to negative. FIGS. 22C-22D show flow cytometry results for E6/E7 negative primary cervical cells from two patients in the absence of PolyA probes, and FIGS. 22E-22F show flow cytometry results for expression of PolyA in specimens from the same two patients in the presence of PolyA probes. FIGS. 22G-22I show 18S housekeeping gene expression in E6/E7 negative primary cervical samples. Endocervical cells (FIGS. 22H-22I) express high amounts of 18S, similar to other housekeeping genes. Compared to other housekeeping genes, significantly more ectocervical cells express 18S (FIG. 22G).

FIGS. 23A-23C provide flow cytometry results for expression of various cancer markers in cervical patient samples from patients verified to have high risk HPV infections. FIG. 23A and FIG. 23C respectively provide flow cytometry results for cancer marker KI67 and cancer marker Top2A, both of which are common markers for many cancers and are involved cell cycle progression. FIG. 23B provides flow cytometry results for cancer marker P16, which has been correlated with HPV induced cancer. Samples showed the presence of mRNA of all three cancer markers.

FIGS. 24A-24L provide flow cytometry results for expression of immune-related cytokines in PBMCs of healthy individuals and subjects suffering from immune-mediated disorders. FIGS. 24A-24D respectively provide flow cytometry results for expression of IFNγ, IL-17, Foxp3 and IL-10 in PBMCs of a healthy individual. FIGS. 24E-24H respectively provide flow cytometry results for expression of IFNγ, IL-17, Foxp3 and IL-10 in PBMCs of a subject suffering from systemic lupus erythematosus, and FIGS. 24I-24L provide flow cytometry results for expression of IFNγ, IL-17, Foxp3 and IL-10 in PBMCs of a subject suffering from diabetes mellitus.

FIGS. 25A-25F provide flow cytometry results for the effect of post-fixation treatment of cells with varying amounts of formaldehyde on false signal from no-probe controls or Negative Control probes in human ectocervical cells from patients with cervical cancer or from patients without cervical cancer. FIGS. 25A-25C respectively provide flow cytometry results in the absence of probes for samples from patients with high grade squamous intraepithelial lesions (HSIL patients) after treatment with 0% formaldehyde, 1% formaldehyde and 5% formaldehyde. FIGS. 25D-25F respectively provide flow cytometry results with the Negative Control probes with samples from the HSIL patients after treatment with 0% formaldehyde, 1% formaldehyde and 5% formaldehyde.

FIGS. 26A-26F provide flow cytometry results for the effect of increasing amounts of formaldehyde on false signal in human ectocervical cells from individuals who were double negative for high risk HPV infection and negative for cytology (N/N patients). FIGS. 26A-26C respectively provide flow cytometry results in the absence of probes for samples from N/N patients after treatment with 0% formaldehyde, 1% formaldehyde and 5% formaldehyde. FIGS. 26D-26F respectively provide flow cytometry results with Negative Control probes for samples from N/N patients after treatment with 0% formaldehyde, 1% formaldehyde and 5% formaldehyde. N/N samples exhibited higher false signal than HSIL samples.

FIGS. 27A-27D provide flow cytometry results for the effect of increasing SSC on false signal from PMBCs. FIG. 27A provides flow cytometry results for PMBCs in the presence of 1×SSC and in the absence of probes. FIG. 27B-27D provides flow cytometry results for PMBCs in the presence of 0.5 μL of the Negative Control probes and 1×SSC, 2.5×SSC and 5×SSC.

FIGS. 28A-28D provide flow cytometry results for the effects of heparin or NuclearID on false signal from PMBC cells. FIG. 28A shows background fluorescence detected by flow cytometry of PMBCs in the absence of probes. FIG. 28B shows background fluorescence detected by flow cytometry of PMBCs in the presence of Negative Control probes. FIG. 28C shows background fluorescence detected by flow cytometry of PMBCs in the presence of Negative Control probes and heparin and FIG. 28D shows background fluorescence detected by flow cytometry of PMBCs in the presence of Negative Control probes and NuclearID.

FIGS. 29A-29C provide flow cytometry results for the effects of Dextran Sulfate on false signals from PBMC cells. FIG. 29A shows background fluorescence detected by flow cytometry of PMBCs in the absence of probes. FIG. 29B shows background fluorescence detected by flow cytometry of PMBCs in the presence of Negative Control probes. FIG. 29C shows background fluorescence detected by flow cytometry of PMBCs in the presence of Negative Control probes and Dextran Sulfate

FIGS. 30A-30C provide flow cytometry results for the effects of Ethidium homodimer on false signals. FIG. 30A shows background fluorescence detected by flow cytometry of Caski cells in the absence of probes. FIG. 30B shows background fluorescence detected by flow cytometry of the Caski cells in the presence of Negative Control probes. FIG. 30C shows background fluorescence detected by flow cytometry of the Caski cells in the presence of Negative Control probes and Ethidium homodimer.

FIGS. 31A-31E provide flow cytometry results for the effects of increasing amounts of EDTA on false signal from PMBC cells using Negative Control probes. FIG. 31A shows background fluorescence detected by flow cytometry of PMBC cells in the absence of EDTA. FIG. 31B-31E shows background fluorescence detected by flow cytometry of PMBC cells with 31.25 mM EDTA, 62.6 mM EDTA, 125 mM EDTA and 250 mM EDTA.

FIGS. 32A-30F provide flow cytometry results for analysis of T-cells and identification of memory T-cells. FIG. 32A shows flow cytometry results indicating that less than 4% of lymphocytes in all lymphocytes in a T-cell sample were positive for IFNγ mRNA. FIG. 32B shows flow cytometry results that identify IL-2Rβ mRNA expression in all lymphocytes in the T-cell sample. FIG. 32C shows flow cytometry results indicating that the IL-2Rβ mRNA positive cells were over 40% positive for IFNγ mRNA, showing that these memory cells are primed for IFNγ production. FIG. 32D shows the gating used to select for either IL-2Rb Low (1.38%) or Il-2Rb High (0.48%). FIG. 32E shows that the IL-2R3 low population of cells had 12.5% of cells positive for IFNγ RNA, and FIG. 32F shows that the IL-2Rβ high population was over 80% positive for IFNγ mRNA, suggesting that the IL-2Rβ high population is the more strongly primed set of memory cells.

FIG. 33 shows the results for an experiment comparing the use of different intercalators with E6/E7 FAM probes.

FIG. 34 shows the results for an experiment comparing the use of different intercalators with 18s Cy3 probes.

### 6. DETAILED DESCRIPTION

### 6.1. Definitions

As used herein, the following terms are intended to have the following meanings.

As used herein, the terms “antibody” or “antibodies” include, but are not limited to, a human antibody, in which the entire sequence is a human sequence, a humanized antibody, which is an antibody from non-human species whose protein sequences have been modified to increase their similarity to antibody variants produced naturally in humans, and a chimeric antibody, which has certain domains from one organism (e.g., mouse) and other domains from a second organism (e.g., human) to yield, e.g., a partially mouse, partially human antibody. The antibody can include, but is not limited to, an antibody or antibody fragment such as Fab, Fab′, F(ab)₂, an Fv fragment, a diabody, a tribody, a linear antibody, a single chain antibody molecule (e.g. scFv) or a multi-specific antibody formed by fusions of antibody fragments. In particular embodiments, the antibody is a monoclonal antibody.

The phrase “a compound comprising an epitope” as used herein includes a compound, e.g., an antigen, comprising a contiguous region of monomers that elicits an immune response in a subject and/or in a healthy individual or that elicits immune tolerance in a subject and/or in a healthy individual, or a compound comprising monomers that adopts a three-dimensional structure such that specific non-contiguous monomers are brought together in a particular spatial configuration that is the epitope. See, e.g., U.S. patent application Ser. No. 09/254,825, filed Dec. 3, 1999, the contents of which are incorporated by reference herein in their entirety. In certain embodiments, the compound comprises an epitope (i.e., the epitope is a subset of contiguous monomers of the compound). In other embodiments, the compound consists of the epitope (i.e., the entire compound is the epitope). In come embodiments, the epitope is a portion of a larger molecule (e.g., a peptide derived from a whole protein). In various embodiments, the epitope is the entire compound (e.g., S-antigen protein). In various embodiments, the epitope is a self-epitope of the subject. In other embodiments, the epitope is a non-self epitope. In some embodiments, the epitope is from a protein that is responsible for the regulation of the immune system, such as a human leukocyte antigen (HLA), including but not limited to HLA A, HLA B, HLA C, HLA D, HLA E, and HLA F or any variants of HLA, such as HLA-B27. In various embodiments, HLA epitopes elicit immune tolerance in a subject. In certain embodiments, the epitope is organ specific. In other embodiments, the epitope is not organ specific. In various embodiments, the epitope is a human epitope. In other embodiments, the epitope is a non-human mammalian epitope. In still other embodiments, the epitope is from a pathogen, i.e., a bacterial, viral or parasite epitope. In some embodiments, the epitope is a mixture of epitopes from different organisms. In various embodiments, the compound comprising an epitope is a tumor antigen or a humanized antibody. In other embodiments, the compound is a self-epitope from an organ exhibiting autoimmune disease. In various embodiments, the compound includes one or more types of monomers, including but not limited to, naturally-occurring amino acids, non-naturally occurring amino acids, nucleotides, carbohydrates, and the like. In certain embodiments, the epitope consists of amino acids, which can be naturally occurring or non-naturally occurring. In particular embodiments, the epitope consists of at least 3, such as at least 4, at least 5, at least 6, at least 7, at least 8, at least 9, at least 10, at least 11, at least 12, at least 13, at least 14, at least 15, at least 16, at least 17, at least 18, at least 19, or at least 20 or more amino acids. In certain embodiments, the compound can be a single compound or an aggregate of compounds (e.g., cross-linked compounds). In other embodiments, compounds are chosen that have an amino acid sequence that adopts a three-dimensional structure that is an epitope that binds to a particular receptor and/or elicits a particular immune response. In various embodiments, the compound can be unmodified or can be directly or indirectly (i.e., through a linking moiety) linked to another moiety, e.g., a sugar, a fat, a label (e.g., a fluorescent or radioactive label) or an additional therapeutic agent.

The term “control element” as used herein refers to a composition that is used in the methods described herein to assess the validity of the method, e.g., as a gating parameter to exclude damaged cells or cells that have undesirable morphology (e.g., size, shape, cell surface marker) from an analysis or as a baseline for measuring nucleic acid targets of interest. In certain embodiments, the control element is selected from a probe (e.g., a nucleic acid probe), a cell, an antigen or combinations thereof. In certain embodiments, the control element is a probe (“control probe”) that is complementary to a gene that is naturally ubiquitously and constitutively expressed in a cell or that is artificially introduced into the cell. In various embodiments, the control probe is complementary to a gene, such as a “housekeeping gene”, that is required for maintenance of cellular function and is expressed in all eukaryotic tissues and cell types under normal or pathophysiological conditions. The skilled artisan will understand that, generally, expression of housekeeping genes does not change. Nevertheless, the skilled artisan will be able to identify one or more housekeeping genes that exhibit variable expression or stable expression in particular cell types. Accordingly, the skilled artisan will be able to identify particular housekeeping genes with stable expression that can be utilized as control elements in particular cell types and conditions. For an avoidance of doubt, a probe that is “complementary” to a target nucleic acid is at least about 50%, such as at least about 60%, at least about 70%, at least about 75%, at least about 80%, at least about 85%, at least about 90%, at least about 95%, or at least about 99% or more complementary to the target nucleic acid. In various embodiments, a probe is 100% complementary to a target nucleic acid. In certain embodiments, the control probe is not associated with a cell, e.g., the control probe is not in a cell or on the cell surface. In various embodiments, the cell-free control probe is attached to a bead. In certain embodiments, the probe is chemically attached to the bead either directly or through a linking group. In other embodiments, the probe is attached to the bead by way of a binding pair, such as biotin/streptavidin or other combinations of molecules with high binding affinity. See, e.g., U.S. patent publication no. 2014/0030787 for examples of other binding pairs. In various embodiments, the bead-bound probe that is complementary to a target of interest is used as a control for probe specificity and activity in the methods described herein. Such bead-bound control probes can be assayed by the methods described herein in parallel with samples of interest. In particular embodiments, the bead-bound probe is RNA. In other particular embodiments, the bead-bound probe is DNA. In still other embodiments, the bead-bound probe can include one or more nucleotide analogs. In various embodiments, bead-bound probes that are complementary to a target of interest can be used as positive controls. In other embodiments, bead-bound probes that are not complementary to the target or that include a scrambled sequence can be used as negative controls. In various embodiments, negative and/or positive control beads are used in lieu of negative and/or positive control cells, respectively, in the assays described herein. In various embodiments, bead-bound probes are run through the methods described herein alongside cells of interest using the exact same protocol. In other embodiments, the control element is a cell (“control cell”). In particular embodiments, the cell is a eukaryotic cell. The eukaryotic cell or cells analyzed/used may, for example, be plant cells or animal cells. The eukaryotic cell or cells may, for example, be mammalian cells, such as non-human mammalian cells or human cells. In various embodiments, the cell (or cell type) is selected from an active cell, an inactive cell, a healthy cell, a diseased cell, a cell with active disease, a cell with inactive disease, a cell that responds to an antigen, a cell that does not respond to an antigen, a cell line (e.g., a cell line that has a foreign genome, such as a viral genome, integrated into its genome, or a cell line that is known not to have such a foreign genome integrated into its genome, for positive and negative controls, respectively), non-genetically modified cells, genetically modified cells (e.g., with genomes edited, mutated or otherwise modified to contain genetic sequences that do not naturally occur in the cell (or were not previously present) and/or to delete genetic sequences that are otherwise naturally occurring in the cell), or an artificial cell. In certain embodiments, the control cell is a cell that has not been exposed to a treatment, e.g., a therapy, or a compound comprising an epitope that induces immune tolerance or immune responsiveness. In other embodiments, the control element is a cell-staining dye, such as CFSE and PKH26. In still other embodiments, the control element is a nucleic acid binding dye, such as an intercalating dye, a major grove binding dye or a minor groove binding dye. In various embodiments, the nucleic acid binding dye fluoresces only when bound to DNA or RNA. In particular embodiments, the nucleic acid binding dye is selected from, but is not limited to, Hoechst, DAPI, Propidium Iodide, Nuclear ID, SYBR Green, 7-AAD and combinations thereof. In yet other embodiments, the control element is a sub-cellular organelle tracing dye. In certain embodiments, the sub-cellular organelle tracing dye is specific for one or more of a nucleus, a nucleolus, a mitochondrion, a Golgi apparatus, a vesicle, a lysosome, a phagophore, an autophagosome, and an autophagolysosome. In other embodiments, the sub-cellular organelle specific dye detects reactive species in an organelle. In various embodiments, the sub-cellular organelle dye detects the state of transport systems such as calcium channels, endoplasmic reticulum, nuclear membranes, mitochondrial membranes, or membranes of other sub-cellular organelles. In yet other embodiments, the control element is a total cell stain. Examples of organelle-specific dyes and total cell stains can be found in co-owned U.S. publication no. 2010/0081159; U.S. publication no. 2010/0062429; U.S. publication no. 2010/0093004; and U.S. Pat. Nos. 8,715,944; 8,357,801; 8,674,102; 8,604,204; 8,357,801; and 8,362,250, the contents of all of which are incorporated by reference herein in their entirety. Without being bound by any particular theory, a control element is used to determine if cells are properly permeabilized and fixated, or for normalization or standardization of signal over a plurality of samples. For example, damaged cells can be identified by abnormally low levels of control elements (e.g., control probes) in the cells as compared to the majority of cells in the sample, which can also indicate abnormal levels of targets of interest, such as target nucleic acids, in these cells. Accordingly, in some embodiments, the amount of the control element in the cell is used as a gating parameter to exclude damaged cells from an analysis, thereby eliminating underestimation of targets in damaged cells. In other embodiments, the amount of control element provides a baseline for measuring targets of interest, i.e., using a ratio of target specific-probe:control probe in a cell.

The term “cytokine” as used herein refers to any proteins (e.g., lymphokines, interleukins, chemokines) that produce systemic or local immunomodulatory effects. Cytokines are generally classified into four structural families: the four α-helix bundle family, IL-1 family, IL-17 family and the cysteine-knot family.

The terms “inflammatory cytokine” and “pro-inflammatory cytokine” as used herein refer to a cytokine that is involved in inducing or amplifying an inflammatory reaction. Exemplary inflammatory cytokines include, but are not limited to, IFNγ, TNFα, IL-1, IL-6, IL-12, IL-17, IL-18, IL-15, IL-8, IL-21, and IL-25.

The term “anti-inflammatory cytokine” as used herein refers to a cytokine that is involved in the reduction of an inflammatory reaction. Exemplary anti-inflammatory cytokines include, but are not limited to, IL-4, IL-10, IL-11, IL-13, TGFβ, IL-35, LIF, CTLA-4, CD39, galectin 1, galectin 3, galectin 9, and PD-L1.

An “epitope binding agent” as used herein is a composition that binds to an antigen to facilitate binding of immune cells to the antigen. In some embodiments, an epitope binding agent is an antigen presenting cell (APC), a macrophage, a dendritic cell, a major histocompatibility complex (MHC) such as MHC class I and MHC class II, a labeled or unlabeled bead bound to multiple copies of MHCs or MHCs bound to a solid surface such as a plate.

The term “eukaryotic cell” as used herein refers to a cell from a human or non-human organism whose cells contain a nucleus and other structures (organelles) enclosed within membranes.

The term “gate” or “gating” refers to the imposition of a set of boundaries that serve to isolate a specific group of cytometric events from a large set, e.g., cytometric events from a particular sub-population of cells. A gate can isolate a group based on, e.g., cell morphology (cell shape or cell size), or display of a specific cell marker (e.g., a protein, an antigen, a nucleic acid) using an antibody or other probe capable of specific binding to the marker.

The term “genotype” as used herein refers to the cumulative nucleic acid content of a cell, including RNA and DNA, such that the unique expression of mRNA and other RNA in the cell provides a fingerprint of that cell, giving insight into how that cell will function. DNA and RNA content of the cell can be assessed by any method in the art. In specific embodiments, mRNA and/or other RNAs in the cell are measured using nucleic acid probes that bind to specific targets, producing fluorescence only when bound to the target, and are measured by flow cytometry to create a fingerprint of RNA expression in that particular cell type.

The term “homogeneous assay” as used herein refers to an assay in which all steps are performed in a single phase, for example, in solution.

The term “human cell” as used herein refers to a eukaryotic cell from a human.

The term “immune-mediated disorder” as used herein refers to any disorder that arises from an imbalance in the immune system, which results in inappropriate immune responses of a subject's body to self-antigens or foreign antigens. Immune-mediated disorders include, but are not limited to, disorders arising from an immune response of the body against substances and tissues normally present in the body (autoimmune disorders), or foreign tissues or antigens that are beneficial (e.g., an organ from a transplant), as well as disorders arising from a reduced or absent immune response to foreign or abnormally expressed antigens, e.g., antigens from infectious agents and cancer (immunodeficiency disorders). Immune-mediated disorders include, for example, uveitis and age-related macular degeneration. Particular immune-mediated disorders include, but are not limited to, those set forth in U.S. patent application Ser. No. 13/871,730, filed Apr. 26, 2013 and PCT Application no. PCT/US14/35549, filed Apr. 25, 2014, the contents of all of which are incorporated by reference herein in their entirety.

The terms “immune cell” and “immune cells” refer to any cell that is involved in modulation of an immune response in a subject. In various embodiments, an immune cell is a T-cell, such as a responder T-cell or a regulatory T-cell, an antigen-specific cytotoxic T-lymphocyte, a phagocyte, and any cell that produces and/or releases cytokines in response to an antigen, including, but not limited to, lymphocytes, leukocytes, dendritic cells, endothelial cells, epithelial cells, macrophages and NK cells.

The terms “responder T-cell” and “T-resp” as used herein refer to T-cells that mount an immune response to antigens, such as antigens presented on antigen presenting cells. In certain embodiments, T-resp cells referred to herein are cells that mount an immune response to foreign antigens or to self-antigens. T-resp cells include T-cells with certain phenotypes, including, but not limited to, CD8⁺ cells, CD4⁺ T-cells, naïve CD4⁺ CD25⁻ T-cells, CD4⁺ CD25⁻Foxp3⁻ T-cells, NK cells such as Vα24⁺ NKT cells, cytotoxic T lymphocytes (CTL), naïve T-cells or memory effector T-cells. T-resp cells can be non-homogeneous or they can be antigen-specific.

The terms “regulatory T-cell” and “T-reg” as used herein refer to T-cells that decrease or eliminate an immune response, e.g., the function of T-resp cells. In some embodiments, the T-reg cells inhibit T-resp cells that are not homogeneous or antigen-specific. In other embodiments, T-reg cells inhibit antigen-specific T-resp cells. In certain embodiments, T-reg cells have an anergic phenotype, i.e., they do not proliferate in response to T-cell receptor stimulation. In other embodiments, T-reg cells proliferate in response to antigen or cytokine stimulation. T-reg cells include T cells with particular phenotypes, including, but not limited to CD4⁺CD25⁺ T-cells, CD4⁺Foxp3⁺ T-cells, CD4⁺CD25⁺Foxp3⁺ T-cells, IL-10 producing CD4⁺ Tr1 cells, TGF-β producing Th3 cells, CD8⁺ NKT cells, CD4⁻CD8⁻ T-cells, γδ T-cells, thymic nT-reg cells, periphery induced i-Treg cells, CD4⁺ CD127^{lo/-} T-cells, CD4⁺CD127^{lo/-}CD25⁺ T-cells, and the CD45RA⁺ subset of CD4⁺CD127^{lo/-}CD25⁺ T-cells. In various embodiments, T-reg cells are negative for CD127 and positive for CD39. In various embodiments, T-reg cells are CD4⁺, CD25⁺ and CD127⁻. In certain embodiments, T-reg cells are induced from CD4⁺CD25⁻ cells by cytokines such as TGFβ1. In other embodiments, T-reg cells are induced from CD4⁺CD25⁻ cells by stimulation with irradiated allogenic stimulator PBMCs. In certain embodiments, the T-reg cells are naïve T-reg cells, such as CD45RA⁺ T-reg cells. In certain embodiments, T-reg cells are from T-resp cells that are induced to become T-reg cells either in vitro or in vivo.

As used herein, a “response” from at T-reg cell or a T-resp cell is an indication that a T-cell is activated or suppressed. In various embodiments, the response includes, but is not limited to, upregulation or downregulation of cell-surface markers, such as activation markers, increased or decreased levels of pro-inflammatory or anti-inflammatory cytokine synthesis and/or secretion (ELIspot or similar assay), or increased or decreased levels of pro-inflammatory cytokine receptors or anti-inflammatory cytokine receptors synthesis and/or appearance on the cell surface, and increased or decreased cell proliferation (expansion). T-cell proliferation can be measured by any method known in the art, including [³H]-thymidine uptake, or by flow cytometry based on the T-cell markers, increased DNA content measured by DNA intercalating dyes, or using cell tracking dyes such as CFSE to label T-resp, T-reg or T-resp and T-reg cells, and monitoring decreases in fluorescence associated with cell division.

The term “immune response” as used herein refers to a response that involves the production of antibodies, cytokines, cell-mediated cytotoxicity, or other responses that involve immune cells.

The term “normalization” as used herein refers to methods of removing technical (non-biological) variance in flow cytometry data from biologically equivalent populations of cells. In certain embodiments, normalization is single-marker normalization. Various methods for normalizing flow cytometry data will be known to the skilled artisan. See, e.g., Hahne et al. (2010) Cytometry A. 2010 February; 77(2): 121-131.

The term “nucleotide analogue” as used herein is a variant of a natural nucleotide, such as DNA or RNA nucleotides, by introduction of one or more modifications. In various embodiments, these modifications when incorporated into a nucleic acid will have a functional effect on the properties of the nucleic acid, for example, conferring higher or lower binding affinity for a target sequence, conferring detectability by inclusion of a label and/or conferring the property of degenerate binding to target nucleic acids.

The terms “probe system” and “homogeneous probe system” as used herein refer to an oligonucleotide hybridization probe that is complementary to a nucleic acid of interest and reports the presence of specific nucleic acids in homogeneous solutions and that generates signal only when bound to a complementary target nucleic acid in fixated and permeabilized cells. Various probe systems include, but are not limited to, those described in Section 5.3, below.

The phrases “prevention of,” “preventing”, and the like include the avoidance of the onset of a condition or a symptom thereof.

The terms “treatment” or “treating” and the like include the amelioration or cessation of a condition or a symptom thereof. In one embodiment, treating includes inhibiting, for example, decreasing the overall frequency of episodes of a condition or a symptom thereof.

### 6.2. Methods and Reagents for Fixating and Permeabilizing Cells

Previously, homogeneous probes were used with flow cytometry to measure the presence and expression of mRNA in living eukaryotic cells. However, the use of live cells was problematic with probe systems such as molecular beacons in which the integrity of probes was critical because the presence of active nucleases in living cells could result in the generation of signal in the absence of hybridization to a specific target by enzymatic separation of the quencher from the fluor of the beacon. Accordingly, a common practice of incorporating various nuclease-proof nucleotide analogues into the beacons to avoid high levels of background was adopted. In addition to the expense of using nucleotide analogues, their use could cause changes in Tm, affinity and especially with peptide nucleic acids, solubility. Accordingly, the use of a fixation/permeabilization step in the present methods reduces or even eliminates nuclease activity, thereby allowing the use of ordinary nucleotides for manufacturing homogeneous probes. Additionally, the fixation/permeabilization step allows for the simultaneous analysis of other cell characteristics in conjunction with mRNA detection, including intracellular and nuclear protein expression measured using antibodies, which are unable to enter living cells.

In various embodiments of the methods described herein, the cells are fixated. In other embodiments, the cells are permeabilized. In still other embodiments, the cells are fixated and permeabilized. In various embodiments, cells are permeabilized first and then fixated. In other embodiments, cells are fixated first and then permeabilized. In still other embodiments, cells are simultaneously fixated and permeabilized. Without being bound by any particular theory, fixation of cells provides a snapshot of the cells at the time of fixation by preserving cell morphology and cell contents. Accordingly, fixation allows preservation of the state of the cells just prior to fixation such that cell samples do not have to be assayed immediately after collection. The permeabilization methods described herein create small holes in the membranes of cells such that labeled probes, labeled antibodies, or dyes (e.g., cell-staining dyes) can enter the cells and bind to a target without significant loss of cell contents.

A variety of fixation and permeabilization methods and reagents can be used in the flow cytometry methods described herein. Reagents for fixation and/or permeabilization include, but are not limited to, commercially available reagents such as incellFP (incellDX), Fix & Perm® (Life Technologies), Cytofix/Cytoperm™ (Becton Dickinson), IntraPrep™ (Beckman Coulter), IntraStain (Dako), IntraStainCell (BenderMedSystems), IQ Starfiqs™ (IQ Products) and PermaCyte™ (Bioergonomics/Bio E), ThinPrep™ (Hologic), SurePath™ (Becton Dickinson). In various embodiments, the fixation and/or permeabilization reagent comprises one or more of ethanol, methanol, isopropanol and formaldehyde. In other embodiments, the fixation and/or permeabilization reagent comprises an amine. In yet further embodiments, the fixation and/or permeabilization reagent comprises a detergent. The skilled artisan will understand that the choice of fixation and/or permeabilization reagents and protocols can be influenced by factors such as the number and/or nature of the target or targets to be preserved in the cell and/or detected, the location of the target or targets in the cell, the type of cells to be assayed, the timeframe for assaying cells (e.g., immediately after fixation and/or permeabilization or after being stored for a period of time), the method of detection to be utilized, and the like. See, e.g., U.S. Pat. Nos. 5,422,277 and 7,326,577 for factors to consider when choosing fixation and/or permeabilization reagents and protocols.

In various embodiments, fixation and/or permeabilization conditions are appropriate for both cells that are assayed immediately and cells that are stored for a period of time before being assayed (e.g., positive and negative control cells, patient samples sent from physicians). In certain embodiments of the methods described herein, successive incubations and washes of cells are carried out in a fixation step with from about 0.5% to about 4.0% formaldehyde in phosphate buffered saline (PBS) followed by successive incubations with about 0.5% to about 2.0% Triton X-100 in PBS and a mixture of Tween 20 (0.01%-1.0%) and MgCl₂ (0.5-5.0 mM) in saline sodium citrate (SSC). In a second particular embodiment of the methods described herein, successive incubations and washes of cells are carried out in a fixation step using ThinPrep or SurePath with from about 0.0% to about 6.0% formaldehyde, followed by a single permeabilization step with about 0.5% to about 2.0% Triton X-100 in saline sodium citrate (SSC). SSC (1×) is 150 mM NaCl, 15 mM sodium citrate in distilled water.

In other particular embodiments, the fixated cells (about 500 μL) are permeabilized by adding 1 mL of Buffer A (1.5% formaldehyde in Dulbecco's phosphate-buffered saline (DPBS)) and are incubated for 1 hour at room temperature. Cells are washed and 1 mL of Buffer B (1% Triton X-100 in DPBS) is added to the washed cells, which are then mixed and incubated for 30 minutes at room temperature. Cells are then washed again and 1 mL of Buffer C (0.05% Tween and 2 mM MgCL₂ in 1×SSC) is added. The cells are mixed again and then incubated for 10 minutes at room temperature. After incubation, the cells are washed, and 1 mL of Buffer C with probes (1 μL probe/600 μL of buffer) is added. 300 μL of the probe mixture is added to each sample, the solution is mixed and the cells are incubated for 30 minutes at 65° C. in a light-protected environment. The samples are mixed and incubated at room temperature for 1 hour in a light-protected environment, after which the cells can be assayed by flow cytometry.

In yet other particular embodiments, fixated cells (about 500 μL) are washed, resuspended in 1 mL of hybridization buffer (1% Triton X-100 in 1×SSC) and washed again. About 150 μL of hybridization buffer with probes (1 μL of probe/600 μL of buffer) is added to the cells, and the sample is mixed and then incubated for 1 hour at 65° C. in a light-protected environment. Samples are mixed again and put on ice at 4° C. for 1 hour in a light-protected environment, after which the cells can be assayed by flow cytometry.

In various embodiments, fixated cells, permeabilized cells or fixated and permeabilized cells can be assayed alongside live cells of the same type, such as live cells from the same subject or, if the fixated and/or permeabilized cells are from a healthy individual, the live cells are from the same healthy individual.

### 6.3. Probe Systems for Detection or Quantification of Target Nucleic Acids

Although both secreted and internal proteins have been detected and/or quantified using flow cytometry, there are a number of advantages associated with using mRNA as the target for analyzing cells. First, when assaying secreted proteins, the proteins must be kept inside the cell by artificial means, which usually involves treating the cells with a pharmaceutical reagent such as Brefeldin A or monensin. Since protocols for retaining secreted proteins in the cell are not instantaneous, pharmaceutical treatment is carried out for an extended period of time, which may allow kinetic effects of either increasing or decreasing protein expression during the Brefeldin A treatment. In contrast, the use of mRNA as a target allows an instant snap-shot of the mRNA at a given time and does not require the use of agents that block secretion, which can affect the results being assayed. Accordingly, in the instant methods, measurement of protein levels is only carried out as an auxiliary measure in conjunction with detection and/or quantification of mRNA targets.

Probe systems for use in detecting single-stranded nucleic acids in the disclosed methods are derived from homogeneous probe systems that enable the detection of cellular components of interest only when probes are bound to the target without the need for washing to remove unbound probes or excessive manipulation of the sample that may result in loss of cell contents. The probe systems can take a variety of different forms in which signal is generated only upon binding of one or more probes to a complementary nucleic acid of interest, and have the advantage of reducing background fluorescence with less sample manipulation, which improves sensitivity and ease of use in a clinical setting. In various embodiments, the probes described herein are used in combination with markers that identify specific cell types such that assays are performed with heterogeneous mixtures of fixated and permeabilized cells. Accordingly, gene expression profiles can be determined in and/or linked to cells of a specific type.

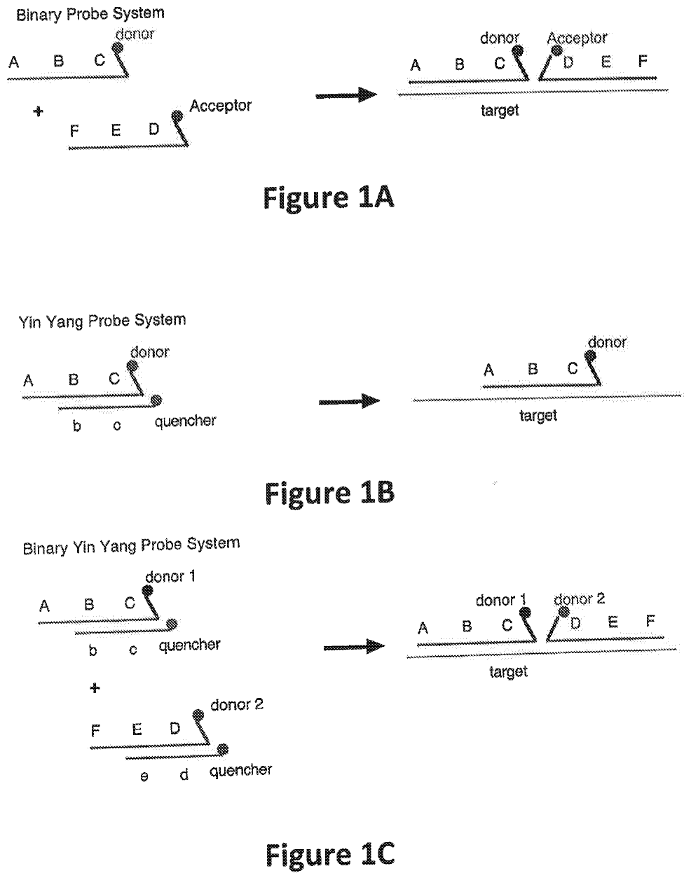

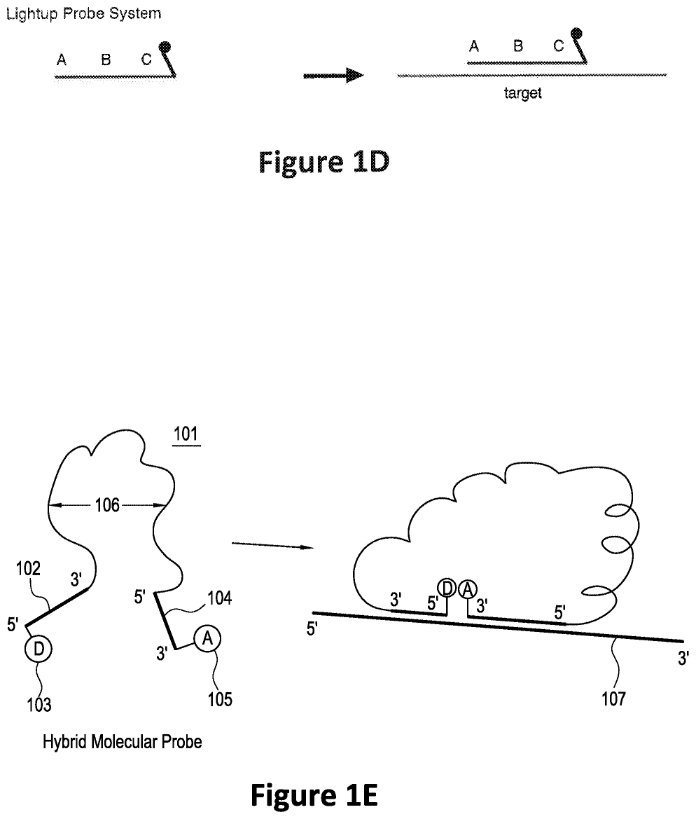

In some embodiments, the methods described herein utilize a binary probe system in which adjacent target-specific probes bind to a target such that their proximity allows for energy transfer to take place between a donor moiety on one probe and an acceptor moiety on the other probe. See Heller and Morrison (1985) in “Rapid Detection and Identification of Infectious Agents” pp 245-256, Academic Press, San Diego Calif. FIG. 1A illustrates this probe system.

In other embodiments, the methods described herein utilize a set of complementary nucleic acid probes in which a target-specific probe and a complementary competitor probe are respectively labeled with a donor and an acceptor. When hybridized with each other, the signal from the donor on the target-specific probe is quenched by the acceptor on the competitor probe. See FIG. 1B. When a target is present, the target-specific probe and the complementary competitor probe separate, and when the separated target-specific probe hybridizes to the target, signal is generated from the donor. See Morrison et al. (1989) Analyt Biochem 183: 231-244; Li et al. (2002) Nucl Acids Res 30:e5 (“yin yang probe”); Seferos et al. (2007) J Am Chem Soc 129:15477-15479 (disclosing a variant of this probe system known as SmartFlares™)

In certain embodiments, the methods described herein utilize a combination of a binary probe system and a yin yang system. In this embodiment, the compositions include a pair of target-specific signal probes designed as described for the binary probe system and two competitor probes labeled with acceptors that bind to and absorb the energy emitted from the donors on the two signal probes. See FIG. 1C. When target is present, signal probes and competitor probes separate, allowing the signal probes to bind to the target such that energy transfer takes place between the donors.

In various embodiments, the methods described herein utilize a lightup probe system. In these embodiments, a target-specific probe comprising a single label binds to a target and interacts with unlabeled nucleotides that are present in the target such that the probe produces signal when bound to the target. See FIG. 1D; Svanvik et al. (2000) Analyt Biochem 281; 26-36; see also Socher et al. (2008) Agnew Chem Int Ed 47:9555-9559 (use of a lightup probe system in conjunction with an acceptor in a linear peptide nucleic acid (PNA) molecule).

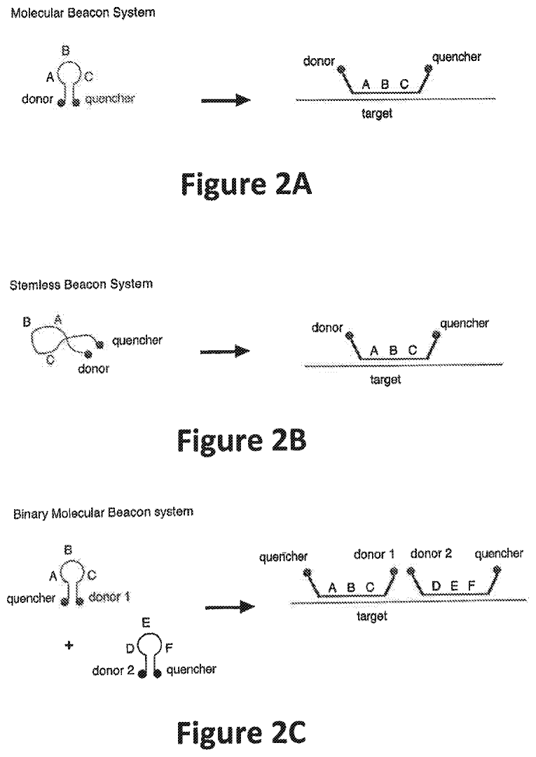

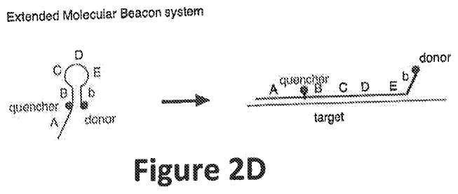

In particular embodiments, the methods described herein utilize a molecular beacon probe system in which oligonucleotide probes are labeled with a signal fluor at one terminus and a quencher at the other terminus. In the absence of analyte, molecular beacons adopt a stem-loop configuration in which the loop is complementary to the target. The stem loop configuration brings the signal fluor and quencher in proximity such that no signal is produced. When hybridized to a target analyte, the quencher is no longer in proximity to the signal fluor, allowing a signal to be generated from the signal fluor. See FIG. 2A, and Tyagi and Kramer (1996) Nature Biotechnology 14:303-308; see also FIG. 2B, and Seitz (2000) Agnew Chem Int Ed 39:3249-3252 (disclosing stemless beacons that lack self-complementarity).

In certain embodiments, the methods described herein utilize single probes known as hybeacons that include a single labeled nucleotide and are capable of generating different signals depending upon whether they are in single-stranded form or hybridized to a target. See French et al. (2001) Molec Cell probes 15:363-374.

In still other embodiments, the methods described herein utilize molecular beacons in various probe system designs. In some embodiments, the methods utilize a binary molecular beacon system in which a pair of molecular beacons hybridize to adjacent sites on a target nucleic acid such that when they bind to the target, the ends of each molecule are in proximity to each other and they generate a signal. See FIG. 2C; Santangelo et al. (2004) Nucl Acids Res 32:e57. In other embodiments, the probe system is an extended molecular beacon in which the loop region, one of the stems and exterior sequences are complementary to the target of interest. See FIG. 2D.

In still other embodiments, the methods described herein utilize a hybrid molecular probe (HMP) in which two nucleic acid hybridization probes, having sequences complementary to adjacent sequences of a target nucleic acid strand, are joined together by a flexible, polymeric linker, such as a non-nucleic acid, non-peptide linker, for example, a polyethylene glycol (PEG) linker, and upon hybridization to the target are adjacent to each other such that energy transfer occurs between a label attached, e.g., covalently, to one probe of the HMP and a label attached, e.g., covalently to the other probe of the HMP. See Yang et al. (2006) J Am Chem Soc 128:9985-9987, Martinez et al. (2008) Anal Bioanal Chem 391:983-991, and U.S. Publication No. 20090156416, each of which is incorporated by reference in its entirety herein. Hybrid molecular probes may be synthesized using phosphoramidite DNA oligonucleotide synthesis methods using phosphoramidite nucleosides and energy-transfer label-derivatized phosphoramidite nucleosides and phosphoramidite linker reagents, such as polyethylene glycol phosphoramidites, such as hexaethylene glycol phosphoramidite (e.g., “Spacer18” from TriLink Biotechnologies (San Diego, Calif.)) which has six PEG monomer units. Multiple PEG phosphoramidites and/or PEG phosphoramidites having different PEG lengths may be used in a synthesis to achieve a linker of any desired length in a hybrid molecular probe. Energy transfer labels capable of interacting with each other by Forster Resonance Energy Transfer (FRET) when in sufficient proximity to each other are disposed at (or near) the 5′ terminus of the HMP and at (or near) the 3′ terminus of the HMP. The sequence of each of the nucleic acid hybridization probe segments of the HMP is selected to have sufficient complementarity (such as but not limited to perfect complementarity) to a target nucleic acid, such as a particular RNA or mRNA of interest, so that in the presence of the target nucleic acid, the probe segments of a single HMP specifically hybridize to the target such that the 3′ and 5′ termini of the HMP (each presented by one of the probe segments thereof) become proximalized such that FRET can occur between the energy transfer labels. The sequences of the probe segments of the HMP may, for example, be selected such that when hybridized to the target nucleic acid the 3′ and 5′ nucleotides of the HMP are immediately adjacent each other (there is no gap of nucleotides between them with respect to the target sequence, or there may be a gap of 1, 2 or 3 nucleotide positions between them with respect to the target sequence. FRET donor/acceptor pairs that be used in a HMP include but are not limited to 6-FAM and Cy5 (Cyanine5; Enzo Life Sciences, Farmingdale, N.Y.).

FIG. 1E schematically illustrates (not drawn to scale) a hybrid molecular probe (HMP) 101 that includes two oligonucleotide probe portions 102 and 104, connected by a polyethylene glycol linker 106. Probe portion 102 has a FRET donor molecule (label) attached at its 5′ end and probe portion 104 has a FRET acceptor molecule (label) attached at its 3′ end, which ends are the two ends of the overall HMP 101. Various suitable FRET donor acceptor pairs known in the art and may be used. Generally, it does not matter at which end the donor or acceptor is attached and, thus, the positions may be reversed from that shown in FIG. 1E. As shown, the linear orientation with respect to 5′ to 3′ direction is the same for both oligonucleotide probe portions in the HMP. The sequences of the probe portions are selected to specifically hybridize to the same strand of a nucleic acid target 107, such as an RNA transcript, e.g., an mRNA, such that the 5′ terminus of 102 with its attached FRET donor and the 3′ terminus of 104 become adjacent in sufficient proximity to each other that FRET can occur between the donor and acceptor while the probe portions are specifically hybridized to the target.

In particular embodiments, multiple regions of a target nucleic acid of interest are bound to a plurality of target-specific probes or probe pairs to increase signal (multiplied in proportion to the number of probes) for detection of single mRNA molecules using linear probes. See, e.g., Narimitsu and Patterson (2005) Am J Clin Pathol 123:716-723 (hybridizing a cocktail of different fluorescein-labeled probes to HPV E6/E7 transcripts for detection by flow cytometry). In HMP-based embodiments of the invention, a plurality of HMPs, such as but not limited to 2, 3, 4, or 5 HMPs or 2-10 HMPs or any subrange therein or at least 2 or at least 3 HMPs, specific to different parts (sequences) of and/or splice variants of the same target nucleic acid molecule(s), such as HPV E6/E7 mRNA, may be provided and used together to increase the signal for target-specific detection. Where multiple HMPs targeting the same nucleic acid target are to be used, the probe portion sequences of the HMPs are selected such that the HMPs will not physicially overlap/interfere with each other when they are all hybridized to the target. Thus, the different multiple HMPs may be complementary to different, non-overlapping sequences of the target nucleic acid molecule. The plurality of HMP probes may be hybridized to the target in the same hybridization mixture. The same donor-acceptor label pairs may, for example, be used among the HMPs that bind different parts of the same target nucleic acid molecule (or to different variants, such as splice variants or preprocessed versions of the target nucleic acid molecule), or different donor-acceptor pair labels may be used among some or all of the different HMPs.

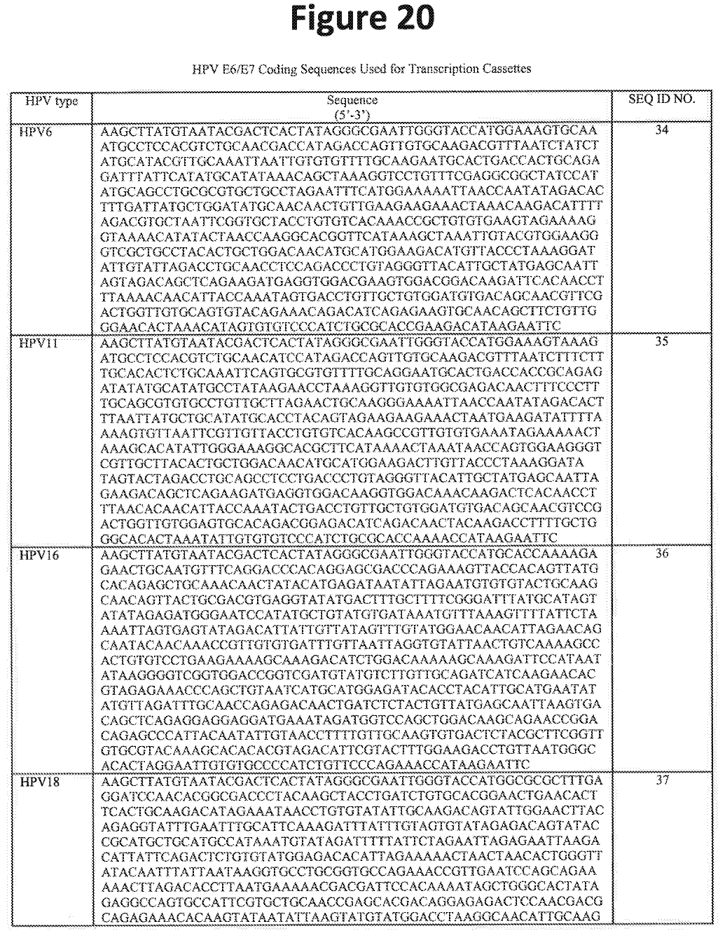

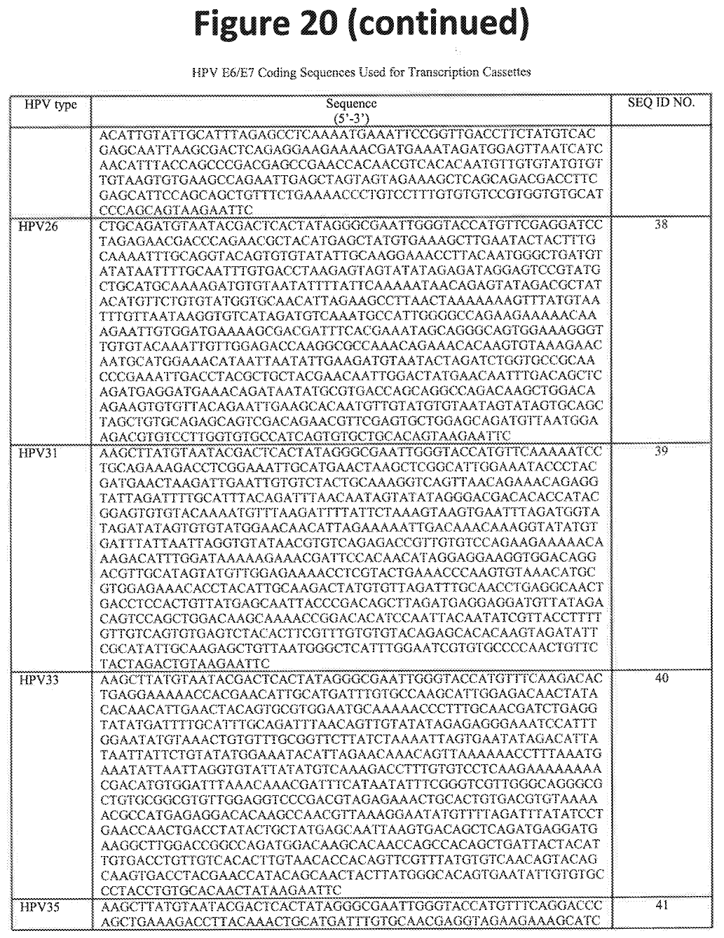

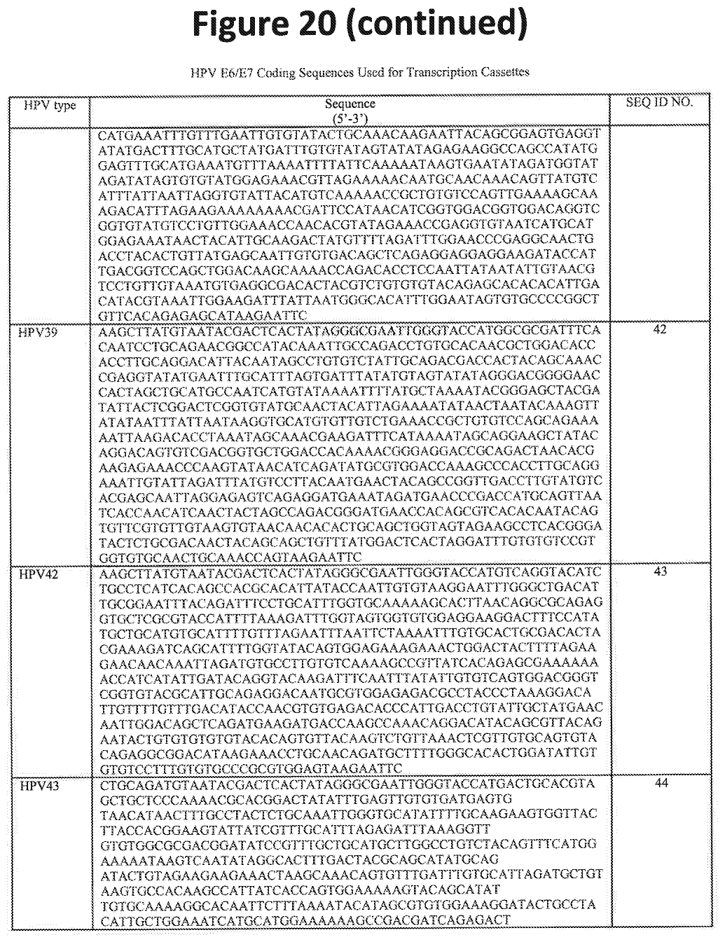

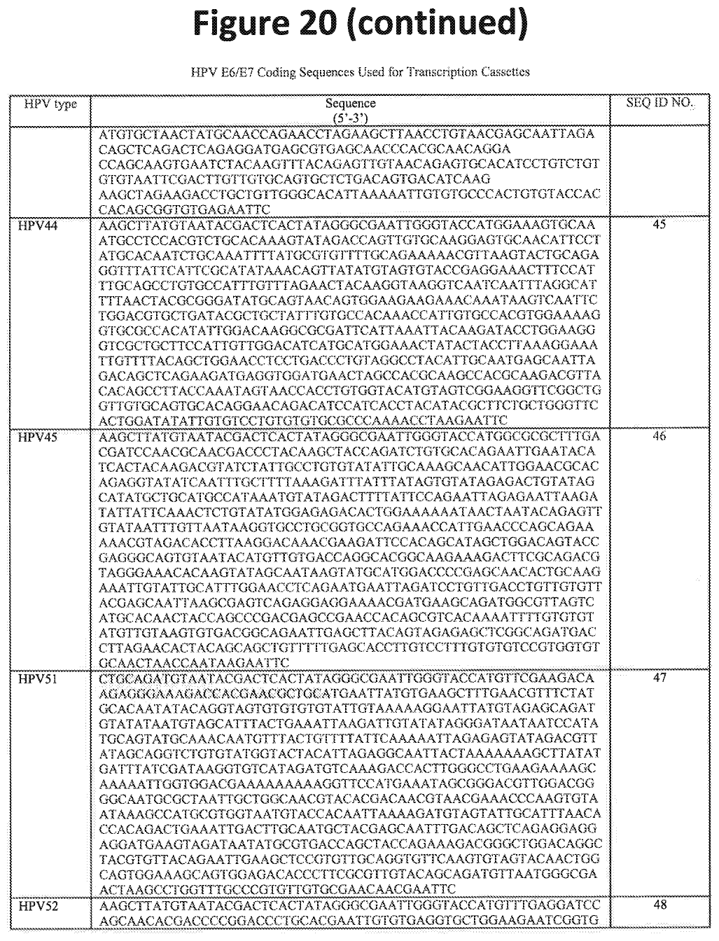

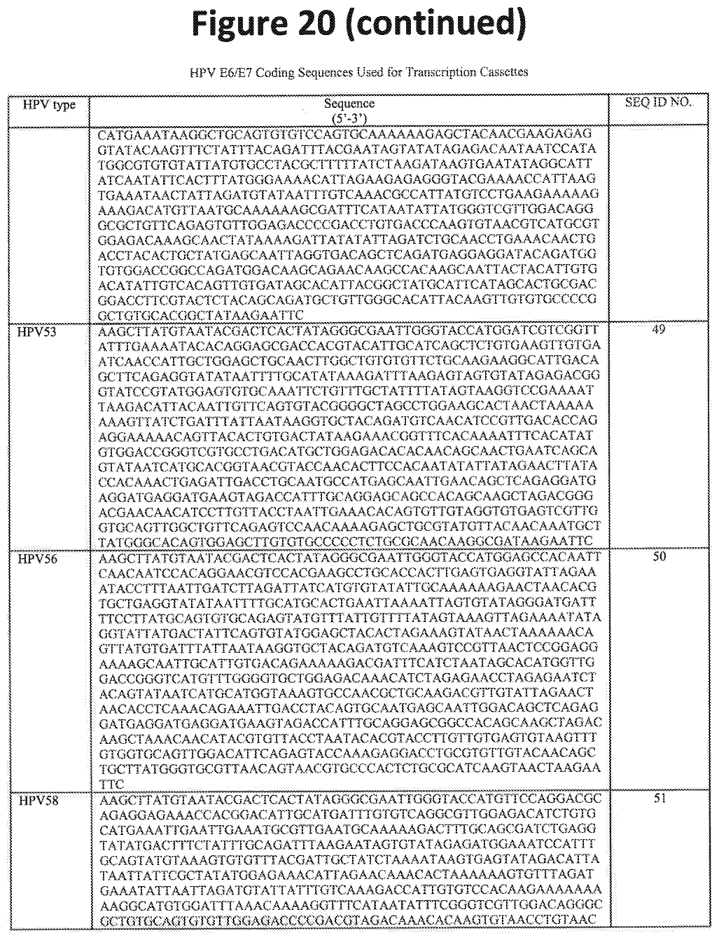

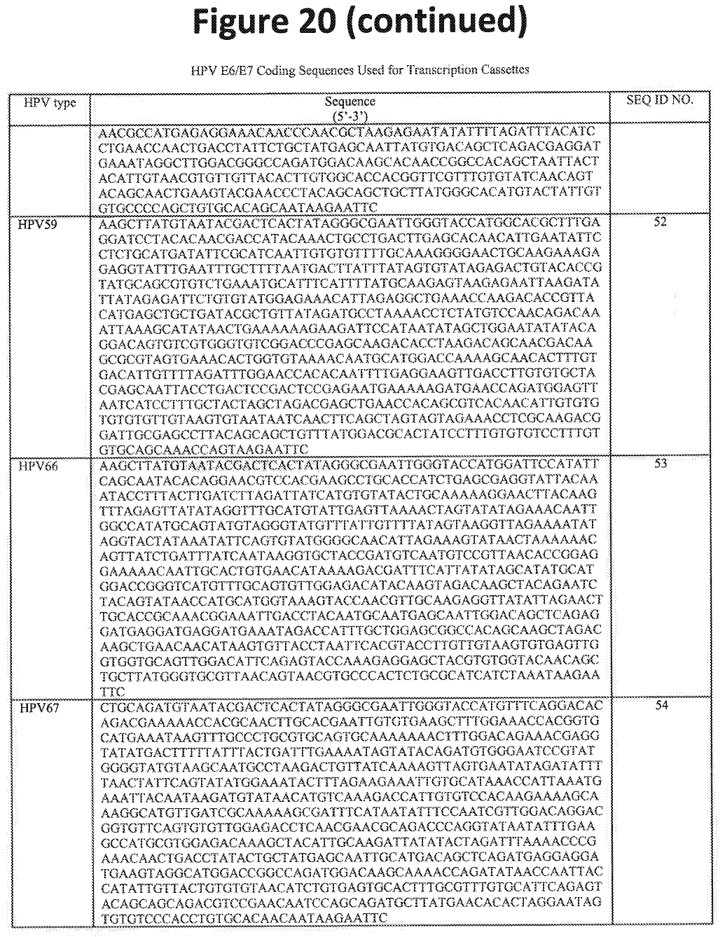

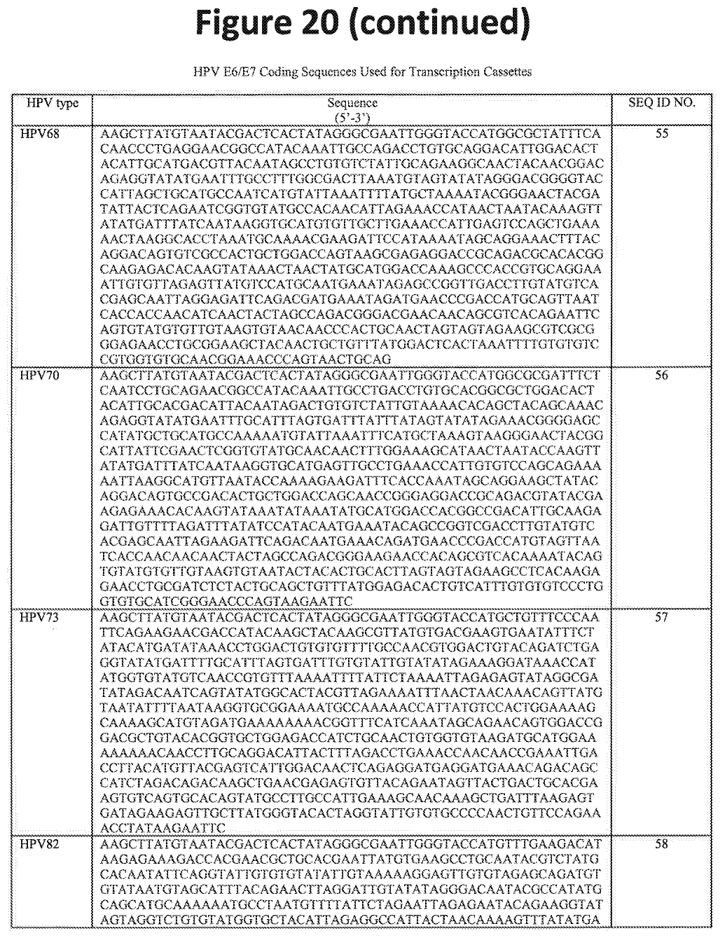

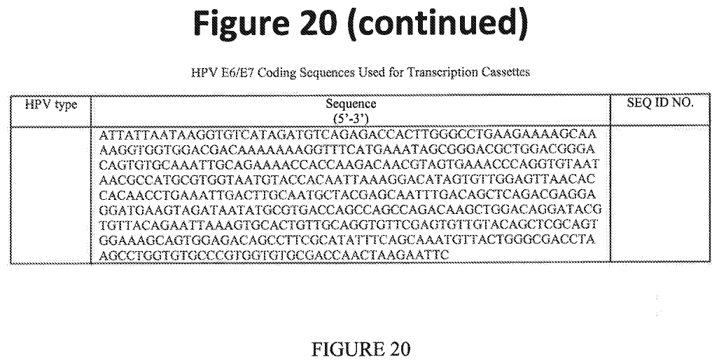

In general, sequences of the probe portions may be selected for HMPs that are sufficiently complementary to E6/E7 RNA of human papilloma viruses of interest to specifically hybridize to the RNA target. The coding sequences for E6/E7 of various human papilloma viruses shown in FIG. 20 may, for example, be used to select the probe portion sequences of HMPs. The probe portions of one or more HMPs may, for example, be designed to hybridize by way of sufficient complementarity to HPV-16 E6/E7 and/or HPV-18 E6/E7. The oligonucleotide probe portions of an HMP may contain non-complementary sequence that does not interfere with the function of the probe portion. For example, with respect to FIG. 1E, the 3′ terminal portion of oligonucleotide probe portion 102 could have sequence not complementary to the target nucleic acid and/or similarly, the 5′ portion of oligonucleotide probe portion 104 could have sequence not complementary to the target nucleic acid.

The following HMPs targeting HPV-16 E6/E7 RNA were synthesized and tested.

(SEQ ID NOS: 60 and 67)
{FAM}TCCAGATGTCTTT{Spacer18}₂GGAATCTTTGCTTT
{Cyanine5-C6-NH—};
(SEQ ID NOS: 61 and 68)
{FAM}TCCAGATGTCTTT{Spacer18}₁₄GGAATCTTTGCTTT
{Cyanine5-C6-NH—};
and
(SEQ ID NOS: 62 and 69)
{FAM}TCCAGATGTCTTT{Spacer18}₁₆GGAATCTTTGCTTT
{Cyanine5-C6-NH—}.

The oligonucleotide probe portions are DNA. Each Spacer18 linker unit provides a linear hexaethylene glycol linker portion. The HMP having only two Spacer18 units (equivalent to 12 ethylene glycols) did not perform as well as probes having longer linkers such as those having fourteen or sixteen Spacer18 units (equivalent to 84 and 96 ethylene glycols, respectively). Polyethylene glycol linker lengths used in HMPs according to the invention may, for example, be in the range of 65 to 150 ethylene glycol units, such as 80 to 130 ethylene glycol units. The nucleic acid monomer length of each of the probe portions of an HMP according to the invention may, for example, be in the range of 10-20 monomers, such as 12-18 monomers.

Hybridization conditions for HMPs on fixed, permeabilized cells such as a suspension of fixed, permeabilized isolated human cervical cells may, for example, include or be 2×PBS 0-10% ethylene carbonate, such as 2×PBS 5-10% ethylene carbonate, for example 2×PBS 5% ethylene carbonate. Hybridization temperature may, for example, be in the range of 35-50° C., such as 37-47° C., for example, 37° C. or 47° C. Hybridization duration may, for example, be in the range of 1 hour to 24 hours, such as 2 hours to 18 hours, such as 2 hours −12 hours. Following hybridization, the cells may, for example, be washed with clean hybridization buffer (with no added HMPs) and/or a more stringent, clean, dilution thereof. Detection and quantification of the target nucleic acids for the HMPs used in the sample of cells may then be analyzed by running the cells on a fluorescence analysis-equipped flow cytometer.

Various embodiments of the invention involve quantification of both HPV E6/E7 RNA, such as HPV-16 and/or HPV-18 RNA, in a sample of cervical cells and also quantification of one or more of p16^{INK4a} RNA expression and p16^{INK4a} protein expression in the sample of cervical cells. HMPs specifically hybridizing to p16^{INK4a} may be used to detect and quantify p16^{INK4a} RNA, such as mRNA. The hybridization of E6/E7 HMPs to a sample of cells and the hybridization of p16^{INK4a} HMP(s) to the sample of cells may be performed concurrently (e.g., within the same hybridization buffer mix) or sequentially and, similarly, the detection of signal from the hybridized E6/E7 HMPs and the p16^{INK4a} HMP(s) may be performed concurrently (e.g., in the same flow cytometry run or session) or sequentially. Alternatively, or in addition, an antibody such as a fluorescently labeled antibody, for example, monoclonal or polyclonal, that specifically binds p16^{INK4a} may be used to quantify p16^{INK4a} protein in the sample of cervical cells. Where an antibody is used, the antibody staining and detection may be performed concurrently with the HMPs hybridization or analysis or the antibody and HMP protocols may be performed sequentially in whole or part on the same sample of cells.

The sequences of the probe portions of the hybrid molecular probes specific for targets such as E6/E7 RNA or P16^{INK4a} RNA or any of the targets disclosed herein may, for example, be selected to specifically hybridize to the same transcript sequences or regions thereof targeted by the molecular beacon probe and other hybridization probe embodiments disclosed herein for said targets.

A strategy of using different HMPs having the same FRET label pairs to take combined measurements of gene expression or using different HMPs with different FRET label pairs that can be distinguishably detected, e.g., as a result of having distinguishable FRET absorption and/or emission spectra, to separately measure the target(s) is generalizable for any desired RNA(s) such as particular mRNAs. Different FRET label pairs used may, for example, have the same FRET donor such as fluorescein or FAM (peak emission nm (“Em.”) 520 for each) but different FRET acceptors having distinguishably different emission wavelengths such as but not limited to FRET donors selected from the group consisting of Roche LightCycler Red 610, 640, 670 and 705 (each having the recited Em.; Sigma-Aldrich Corp., St. Louis, Mo.), Texas Red (Em. 603), ROX (Em. 602), Cyanine5 (Em. 670) and Cyanine 5.5 (Em. 694). The fluorescein or FAM donor may, for example, be attached to the 3′ end of an HMP with the FRET acceptor attached to the 5′ end of the HMP. In one embodiment, a plurality of different HPV-16 E6/E7 HMPs all having the same FRET label pair, FAM-LightCycler Red 610, is provided and used and a plurality of different HPV-18 E6/E7 specific HMPs all having the same FRET label pair PAIR, FAM-LightCycler Red 705 is provided and used. Thus, with this embodiment, HPV-16 E6/E7 and HPV-18 E6/E7 RNA in a sample can be distinguishably detected. In a contrasting embodiment, a plurality of different HPV-16 E6/E7 HMPs and a plurality of different HPV-18 E6/E7 HMPs are provided and used that all have the same FRET label pair, such as FAM-LightCycler Red 610 or FAM-LightCycler Red 705.

In particular embodiments, the amount of signal produced by the probe systems described herein is increased by various modifications to the probes. In certain embodiments, the amount of signal to noise produced from an individual molecular beacon is increased by incorporating more than one quencher to the stem region of a molecular beacon to form a superquencher. See Yang et al. (2005) JACS 127:12772-12773. In other embodiments, molecular beacons with a single quencher are designed to include multiple pyrene donors that result in increased signal. See Conlon et al. (2008) JACS 130:336-342. In still other embodiments, wavelength shifting molecular beacons having superior fluorescent efficiency and increased specificity comprise a quencher, a fluor and a third harvester fluorophore that allows FRET to take place from the donor to the harvester when the probe is bound to a complementary target. See Tyagi et al. (2000) Nature Biotechnol 18:1191-1196; see also Marti et al. (2007) Tetrahedron 63:3591-3600 (applying the triple dye conformation of wavelength shifting molecular beacons to binary probe designs in which one probe is labeled with two different fluors and a second probe is labeled with a single dye resulting in probes that have increased signal to noise ratios). In certain embodiments, pooling is used to assay multiple markers as a single group, where an overall change in the group in individual cells is clinically significant.

In certain embodiments, the probes described herein are modified to, for example, enhance one or more of cellular uptake, probe stability, hybridization kinetics, nuclease resistance and/or provide other useful properties that will be recognized by the skilled artisan. Such modifications include, but are not limited to, introduction of peptide nucleic acids (PNAs), 2′-O methyl analogs, locked nucleic acids (LNA) and the like. See, e.g., Kimura et al. (2009) J Inf Disease 200:1078-1087; Molenaar et al. (2001) Nucl Acids Res 29:e89; Just et al. (1998) J Virol Methods 73:163-174; Xi et al. (2003) Appl Environ Micro 69:5673-5678; Robertson and Thach (2009) Analyt Biochem 390:109-114; Robertson et al. (2010) RNA 16:1679-1685. In various embodiments, a conjugate of TAT peptide and probes enhances entry into live cells without permeabilization. See, e.g., Nitin et al. (2004) Nucl Acids Res 32:e58; Sivaraman et al. (2013) Applied Environ Microbiology 79:696-700. In certain embodiments, the modifications result in chimeric nucleic acids, which include a combination of unmodified and modified nucleic acids. See Yang et al. (2007) Nucl Acids Res 35:4030-4041 (molecular beacons including unmodified nucleotides and LNAs to provide nuclease resistance).

### 6.4. Probe Systems for Detection of Double Stranded DNA

In various embodiments, homogeneous probe systems for use in the methods described herein are for detection and/or quantification of double stranded DNA in cells. In certain embodiments, the probe is capable of forming triple-stranded structures with dsDNA in which, in addition to canonical Watson-Crick binding of the two DNA strands, sequence-dependent third-strand binding is effected by Hoogsteen hydrogen bonding. Accordingly, the homogenous systems described in Section 5.3, above, and in FIG. 1 and FIG. 2 can be used for sequence-specific detection of DNA in cells for detection using flow cytometry. The skilled artisan will readily ascertain how to design sequence-specific probes for detection of dsDNA using well-known principles of triple-strand formation. See, e.g., Ghosh et al. (2006) Mol Biosyst 2:551-560; Mergny et al. (1994) Biochemistry 33:15321-15328; Yang et al. (1998) Analyt Biochem 259:272-274 (binary probes for detection of double stranded DNA); Antony et al. (2001) Biochemistry 40:9387-9395 (molecular beacon probes for detection of double stranded DNA).

Enhancement of probe binding to double-stranded nucleic acids can be increased by substituting nucleotides in the probe with various nucleotide analogues. See, e.g., Blommers et al. (1998) Biochemistry 37:17,714-17,725 (use of 2-aminoethoxy analogues); Bijapur et al. (1999) Nucl Acids Res 27:1802-1809 (use of propargylamino-dU); Karkar and Bhatnagar (2006) Appl Microbio Technol 71: 575-586; Hansen et al. (2009) Nucl Acids Res 37:4498-4507 (use of peptide nucleic acid or locked nucleic acid analogues).

In other embodiments, detection of DNA in the absence of denaturation includes induction of strand displacement and binding of a homogeneous probe. In certain embodiments, strand displacement is effected using a complementary nucleic acid that includes one or more modified nucleotides (e.g., peptide nucleic acid or locked nucleic acid analogues) and allow competitive displacement of one strand of DNA in a duplex followed by replacement with a labeled probe due to an enhanced ability for the probe to bind to its complement compared to the unmodified DNA strand. See, e.g., Kuhn et al. (2001) Antisense Nucleic Acid Drug Dev 11:265-270; Kuhn et al. (2002) J Am Chem Soc 124:1097-1103; Smolina et al. (2008) Bioorgan Med Chem 16:84-93 (using “opener” PNA oligonucleotides to increase efficiency of a strand displacement reaction); He et al. (2009) J Am Chem Soc. 131:12,088-12,090; Rapireddy et al. (2011) Biochemistry 50:3913-3918 (using preorganized D-PNAs to induce displacement and a subsequent binding in the absence of “openers”).

Amplification of signal for detection of DNA or RNA in the methods described herein can be effected by any method known to the skilled artisan, including, but not limited to, hybridizing labeled probes to multiple different sites on the target nucleic acid, or using probes that bind to repetitive sequences. See, e.g., Rufer et al. (1998) Nature Biotechnol 16:743-747; Baerlocher et al. (2006) Nature Protocols 1:2365-2376.

### 6.5. Flow Cytometry Methods of Identifying Phenotype of Cells of Interest

The flow cytometry methods described herein enable high-throughput phenotyping of cells to identify specific sub-types of cells of interest in a mixed cell population, and correlating the phenotype of the cells with their function. In various embodiments, phenotyping of cells is accomplished by detecting and/or quantifying one or more cell-surface markers, which is indicative of cell type and/or the tissue from which the cells originated, and/or by assaying two or more of cellular gene expression such as expression of genes encoding enzymes, cytokines, cytokine receptors or other proteins of interest, DNA content, cell receptor number and/or status, number and/or state of organelles and cell transport system status. In some embodiments, methods for elucidating phenotype (or genotype) of cells of interest described herein further comprises normalization with an internal or external control element such as a housekeeping gene, a cell line, primary cells from tissue, or cells of the same type from a healthy individual.

In various embodiments, the detection and/or quantification of two or more of nucleic acids, proteins, DNA, or other cellular components described herein can be used to detect increased or decreased function in a subset of cells such as diseased cells (e.g., cancer cells or virus infected cells), cells with active disease or cells that are responsive to a particular stimulus (e.g., an antigen), in comparison with control cells, respectively, healthy cells, cells that have inactive disease or cells that are not responsive to the stimulus. In various embodiments, the flow cytometry methods described herein can be used to scan large numbers of cells and identify a minority of cells in the sample with a specific phenotype, such as aberrant cells, that are difficult to identify by traditional detection methods such as imaging (e.g., ultrasound, MRI scans), endoscopy, or examination of biopsied tissue samples on slides using microscopy, especially during onset or early stages of disease or infection.

In various aspects, the present disclosure is directed to methods of phenotyping cells by, e.g., detecting and/or quantifying target nucleic acids in a cell. In certain embodiments, the cell is a eukaryotic cell. In various embodiments, the methods described herein are for detecting and/or quantifying more than one target nucleic acid in a cell. In other embodiments, the target nucleic acid is not indigenous to the cell, i.e., the target nucleic acid is from an agent that infects the cell. In various embodiments, the target nucleic acid is RNA or DNA, or both RNA and DNA. In particular embodiments, the target nucleic acid is selected from, but not limited to, dsDNA, mRNA, hnRNA, microRNA, snRNA, rRNA, snoRNA, SmY RNA, scaRNA, gRNA, tRNA, tmRNA, crRNA, IncRNA, miRNA, piRNA, siRNA, tasiRNA, rasiRNA, 7SK and combinations thereof. In particular embodiments, the target nucleic acid is mRNA. In other embodiments, the target nucleic acid is micro RNA (miRNA). An advantage of the present methods for detecting RNA in lieu of the translated protein product of mRNA is that the presence and/or amount of certain RNAs that are not translated, such as miRNAs, are significant as biomarkers of disease. See Wang et al. (2014) Proc Nat'l Acad Sci 111(11):4262-67 (micro RNA in HPV infection).

Accordingly, in some embodiments, the methods of analyzing cells by detecting and/or quantifying gene expression in a eukaryotic cell comprises the steps of (a) fixating and permeabilizing a eukaryotic cell; (b) contacting the fixated and permeabilized cells with a nucleic acid probe that is complementary to a nucleic acid of interest under conditions in which the nucleic acid probe hybridizes to a complementary nucleic acid sequence in the cell, wherein the nucleic acid probe is part of a homogeneous probe system that generates a signal when bound to a complementary sequence in the nucleic acid of interest; (c) detecting an amount of signal generated by the probe that is hybridized to the nucleic acid of interest in the eukaryotic cell using flow cytometry; and (d) detecting or quantifying the nucleic acid of interest in the eukaryotic cell by detecting or quantifying an amount of signal generated from the probe of step (b), wherein the presence or quantity of the target nucleic acid is associated with the presence of or susceptibility to a disease.

In particular embodiments, the method further comprises a step of contacting the fixated and permeabilized cells in step (b) with a control element, such as a control nucleic acid probe, under conditions in which the probe hybridizes to a complementary nucleic acid sequence in the cell, wherein the control nucleic acid probe is part of a homogeneous probe system that generates a signal when bound to a complementary sequence in a nucleic acid. In particular embodiments, simultaneous detection of signal from the target RNA probe and signal from the control RNA probe in individual cells is effected using fluorescent dyes of distinct colors for the target RNA and the control RNA. In further embodiments, detection of a signal from the target RNA probe and signal from a DNA intercalating dye in individual cells is effected using fluorescent dyes of distinct colors for the target RNA and the DNA of the cell.

In various embodiments where the control element is a control probe, the control probe is complementary to a housekeeping gene that encodes, for example, a transcription factor gene, a repressor gene, an RNA splicing gene, a translation factor, a tRNA synthetase gene, an RNA binding protein gene, a ribosomal protein gene, an RNA polymerase gene, a heat shock gene, a carbohydrate metabolism gene, a protein processing gene, or a channel or transporter gene. Various housekeeping genes that can be used as controls will be readily ascertainable by the skilled artisan and include, but are not limited to, GUSB, LDHA, NONO, PGK1, PPIH, RPLP0, and TFRC. In particular embodiments, the control probe is complementary to a gene selected from GAPDH, β-actin, 18S ribosomal RNA, 28S ribosomal RNA, HSP90, and poly-A tails of total cell mRNA. In other embodiments, the housekeeping gene is selected from PBDG, ALDOA, G6PD, GAPD, B2M, PFKP, PGK1, PGAM1, Tuba1, HPRT1, VIM, RPS27A, RPL19, HSPCA, RPL11, RP529, NONO, AAMP, RP53, ARHGDIA, PO, CYC, GUS, HPRT, TBP, TfR, B2M, HPRT1, LMNB1, SDHA and the like. See, e.g., Lupberger et al. (2002) Molecular and Cellular Probes 16:25-30; Ruan and Lai (2007) Clinica Chimica Acta 385:1-5; Tricarico et al. (2002) Analytical Biochemistry 309:293-300; Haller et al. (2004) Analytical Biochemistry 335:1-9; and Lee et al. (2001) Genome Research 12:292-297 for further examples of housekeeping genes that can be used in the methods described herein. In various embodiments, the control probe is complementary to a nucleic acid that is indigenous to the cell. In other embodiments, the control probe is complementary to a nucleic acid that is artificially introduced into the cell or that is transcribed from a nucleic acid that is artificially introduced into the cell. In various embodiments, the control probe binds to a cell-specific transcript present in a particular population of cells of interest. In further embodiments, the control probe is a randomized sequence that is not specific to any transcript in the target cell and is intended to show background fluorescence. In various embodiments in which infection of eukaryotic cells is detected and/or quantified, the control probe is a nucleic acid probe that is complementary to a gene of the infectious agent, such as a virus or parasite that shows stable expression during infection. In other embodiments, the control element is a control cell, as defined above. In still other embodiments, the control element includes both a control probe and a control cell. In certain embodiments, the control element is a positive control element, such as, e.g., a cell that is infected with a specific pathogen, such as a stable cell line. In other embodiments, the control element is a negative control element, such as, e.g., a cell that is known not to be infected with the pathogen.

In particular embodiments, the method further comprises a step of contacting the fixated and permeabilized cells in step (b) with a control nucleic acid probe under conditions in which the probe hybridizes to a complementary nucleic acid sequence in the cell, wherein the control nucleic acid probe is part of a homogeneous probe system that generates a signal when bound to a complementary sequence in a nucleic acid. In particular embodiments, simultaneous detection of signal from the target RNA probe and signal from the control RNA probe in individual cells is effected using different fluorescent dyes (i.e., distinct colors) for the target RNA and the control RNA.

In other embodiments, the present disclosure relates to a method for detecting or quantifying an amount of a target RNA in a eukaryotic cell comprising (a) fixating and permeabilizing (i) a eukaryotic cell and (ii) a control element, e.g., a eukaryotic control cell; (b) contacting the eukaryotic cell and the eukaryotic control cell with a nucleic acid probe that is complementary to a nucleic acid of interest; (c) detecting using flow cytometry (i) an amount of signal generated by the probe hybridized to the target nucleic acid in the eukaryotic cell, and (ii) an amount of signal generated by the probe hybridized to the target nucleic acid in the eukaryotic control cell; and (d) detecting or measuring the nucleic acid of interest in the eukaryotic cell by detecting or measuring the amount of signal generated from the probe of step (b), (e) detecting or measuring the nucleic acid of interest in the eukaryotic control cell by detecting or measuring the amount of signal generated from the probe of step (b); and (f) comparing the results of step (d) and step (e). In certain embodiments, in which the presence, absence or amount of a target RNA is indicative of a disease, the control cell is a cell that does not have the disease. In various embodiments in which the presence, absence or amount of an intracellular target, e.g., RNA, is indicative of active disease, the control cell is a cell that does not have active disease. In particular embodiments in which the presence, absence or amount of an intracellular target is indicative of inactive disease, the control cell is a cell that has active disease. In still other embodiments in which the presence, absence or amount of an intracellular target is indicative of the presence of an infection, the control cell is a cell that is not infected. In yet other embodiments in which the presence, absence or amount of an intracellular target is indicative of an inactive cell, the control cell is an active cell. The skilled artisan will appreciate that identification of a control element to be used in the flow cytometry methods described herein depends on the nature of the cells of interest in the specimen being assayed.

In various embodiments, the methods described herein are for detection and/or quantification of eukaryotic cells that are infected. In certain embodiments, the infection is a viral infection. In particular embodiments, the viral infection is a human immunodeficiency virus (HIV) infection, a hepatitis B virus (HBV) infection, a hepatitis C virus (HCV) infection, a human papilloma virus (HPV) infection or a herpes virus infection. In other embodiments, the infection is a parasitic infection such as malaria, toxoplasmosis, leishmaniosis, or schistosomiasis.

In certain embodiments, the flow cytometry methods include detecting and/or quantifying one or more target nucleic acids in cells of interest. In various embodiments, cells of interest are identified by cell-surface markers. In some embodiments, the methods described herein include detection and/or quantification of expression of multiple different proteins in identified cells of interest by detecting mRNA encoding the proteins, detecting non-coding RNA in the cell, detecting proteins in the cell, or detecting both nucleic acids and proteins in the cell. Accordingly, the methods described herein can be used to elucidate a pattern of gene expression in cells of interest in lieu of traditional nucleic acid or protein arrays without the problems associated with traditional DNA or protein arrays. In certain embodiments, the target nucleic acids include nucleic acids indigenous to the cell, target nucleic acids not indigenous to the cell, or a combination of both indigenous and foreign nucleic acids.

Detection and/or quantification of proteins can be effected using a labeled antibody that specifically binds to a cell-surface or intracellular protein to identify cell phenotype or lineage. Antibodies can be labeled by any method known in the art. In certain embodiments, a label, e.g., a fluorescent dye, a chemiluminescent compound, or a compound that can be detected by binding to a labeled binding partner, such as biotin, avidin or streptavidin, is covalently attached, either directly or through a linker arm (e.g., maleimide), to the antibody. In various embodiments, the antibody is labeled with a quantum dot, an electron dense component, a magnetic component, a hormone component, a chelating group, a chelated compound, an antigen or a combination thereof. In particular embodiments, exposure of cell targets to antibodies is followed by a washing step that removes unbound antibodies. In other embodiments, antibodies are used with protocols that do not require a washing step. See, e.g., Nolan and Young (2007) Briefings in Functional Genomics and Proteomics 6:81-90. In certain embodiments, transcripts of a protein e.g., a cytokine, can be detected and/or quantified using nucleic acid probes that bind to mRNA encoding the target, or labeled antibodies that specifically bind to the protein in the cell. The skilled artisan will readily ascertain when it is advantageous to detect and/or quantify nucleic acids, proteins or both nucleic acids and proteins in a single experiment. See, e.g., Phanuphak et al. (2013) PLOS ONE 8: e78291 (detecting E6/E7 mRNA by flow cytometry and p16 protein by in situ hybridization to detect and predict the severity of HPV lesions in anal specimens).

In particular embodiments, the methods described herein include a step of detecting and/or quantifying an HPV nucleic acid, e.g., mRNA, in an infected cell and detecting and/or quantifying one or more additional markers that elucidate the phenotype of infected cells. In certain embodiments, the target of interest is E6/E7, a gene whose increased expression is indicative of progression of HPV infection to cancer. In other embodiments, the relative level of E6/E7 (e.g., mRNA encoding E6/E7) in a cell is derived using another HPV gene as an additional maker that can be used for normalization. Accordingly, in some embodiments where HPV progression is assayed, an HPV marker whose expression is relatively stable, e.g., L1, is compared with expression of E6/E7 such that a ratio of E6/E7:L1, which can be assayed by detecting or quantifying mRNA encoding these proteins, shows an increase that is reflective of the extent of progression of the infection. In other embodiments, an HPV marker whose expression decreases when viral DNA is integrated into a host chromosome, e.g., E2, is compared with expression of E6/E7 such that a ratio of E6/E7:E2 shows an increase that is correlated with the state of progression. In this embodiment, the E6/E7:E2 ratio, measured by mRNA encoding these proteins, shows a pronounced change, since the E2 gene, which codes for a suppressor of E6/E7 expression, no longer produces the E6/E7 suppressor upon integration into the host genome. In certain embodiments, actively proliferating cells (e.g., S phase or G2 phase cells) or non-proliferating cells (G1 phase cells) are identified based on DNA content using DNA staining dyes such as Hoechst or Nuclear ID and are then assayed for levels of E6/E7 RNA, which may be correlated with progression of infection to cervical cancer. See, e.g., Perticarari et al. (1989) Path. Res. Pract. 185:686-688. In particular embodiments, cells that have elevated amounts of DNA as compared to amounts of DNA in the same type of cells that are not infected are identified as being more likely to have a higher degree of progression. See Example 13. In addition, recent studies have shown that the level of expression of E5 may serve as a progression marker similar to the E6/E7 marker. See, e.g., Liao et al. 2013) Oncology Reports 29:95-102.

In other embodiments, the methods described herein include a step of detecting and/or quantifying a host gene product that has altered expression levels associated with progression to cancer, and a step of detecting and/or quantifying HPV expression. In these embodiments, mRNA from host genes that can be detected or quantified include, but are not limited to, mRNA transcribed from genes coding for p53 or Rb proteins, TOP2a, MCM2, CK13, CK14, MCM5, CDC6, survivin, CEA, p63, pRb, p21WAF1, MYC cellular oncogene, CDK4, cyclin proteins such as cyclin A, Cyclin B, cyclin D and cyclin E, telomerase, minichromosome maintenance proteins such as minichromosome maintenance protein 2, minichromosome maintenance protein 4 and minichromosome maintenance protein 5, heat shock protein 40, heat shock protein 60, heat shock protein 70, and CA9/MN protein. See, e.g., Wentzensen and von Knebel Doeberitz (2007) Disease Markers 23:315-330; Sahasrabuddhe (2011) Future Microbiology 6:1083-1089. In various embodiments, other host gene mRNAs useful for detection and/or quantification in conjunction with HPV infection and/or progression include P16^{INK4a} and Ki67 mRNAs. See Sano et al. (1998) Am J Path 153:1741-1748; Klaes et al. (2001) J Cancer 92:276-284 (overexpression of P16^{INK4a} correlated with HPV directed oncogenesis); Zappracosta et al. Biomed Res Int. Vol. 2013, Article ID 453606 (dual assay that measures both the P16^{INK4a} and Ki67 avoids false positives identified by P16^{INK4a} alone). The skilled artisan will understand that, although in the majority of cases increased levels of expression of genes from the host or HPV are signposts of progression, in some cases, under expression or even no change in expression of particular markers can have significance with regard to the presence or stage of oncogenic progression.

In particular embodiments, the methods described herein include methods of determining malignant transformation of cells. In some embodiments, the cells are not infected by a virus. In other embodiments, the cells, e.g., cervical cells, anal cells or head or neck cells are infected with a virus such as HPV. Accordingly, in certain embodiments, the disclosure is directed to a method of determining malignant transformation of cells comprising (i) contacting a specimen of fixated and permeabilized cells with a virus-specific, e.g., HPV E6/E7 mRNA-specific, probe and a eukaryotic cell mRNA-specific probe and (ii) quantifying using flow cytometry a number of cells in the specimen having viral mRNA copies, such as E6/E7 mRNA, above a positive determined cutoff point, e.g., at least about 100, such as at least about 200 copies of HPV E6/E7 per cell, which is indicative of progression toward malignant transformation of said cells. In various embodiments, the probe is a molecular beacon. In particular embodiments, the HPV specific probe binds to HPV16 sequences. In other embodiments, the HPV specific probe binds to HPV18 sequences. In additional embodiments, the HPV specific probe binds to one or more other HPV types other than HPV16 and HPV18 that have also been liked to progression to cancer. In still other embodiments, the HPV specific probe binds to both HPV16 and HPV18. In various embodiments, the methods described herein further include a step of determining the percentage of cervical cells in the specimen having E6/E7 mRNA copies above a positive predetermined cutoff point, wherein the percentage is indicative of malignant transformation. See, e.g., U.S. Pat. Nos. 7,524,631 and 7,888,032. In additional embodiments, the methods of determining malignant transformation of cells further comprise the steps of (iii) contacting a fixated and permeabilized control cell with an HPV E6/E7 mRNA-specific probe and a host cell mRNA-specific probe; (iv) quantifying using flow cytometry a number of eukaryotic control cells in the sample having E6/E7 mRNA copies above a positive determined cutoff point, and comparing the amount of cells in step (ii) with the amount of cells in step (iv). In particular embodiments, the probes utilized in these methods are molecular beacons.

In certain embodiments, the methods described herein include a control cell which is from a cell line containing an integrated copy of the HPV genome. In various embodiments, the eukaryotic cell mRNA-specific probe is complementary to a housekeeping gene. In other embodiments, the eukaryotic cell mRNA-specific probe is complementary to a gene associated with the development of cancer. In particular embodiments, the gene associated with the development of cancer is selected from p53 or Rb proteins, TOP2a, MCM2, CK13, CK14, MCM5, CDCl₆, survivin, CEA, p63, pRb, p21WAF1, MYC cellular oncogene, CDK4, cyclin proteins such as cyclin A, Cyclin B, cyclin D and cyclin E, telomerase, minichromosome maintenance proteins such as minichromosome maintenance protein 2, minichromosome maintenance protein 4 and minichromosome maintenance protein 5, heat shock protein 40, heat shock protein 60, heat shock protein 70, CA9/MN protein and a combination of any of the foregoing.

In particular embodiments, the phenotyping methods and compositions described herein, which eliminate the need for potentially damaging washing steps that could destroy delicate cells, can be utilized for analyzing individual cells in the central nervous system. Accordingly, in some embodiments, the present disclosure relates to methods for analyzing gene expression and/or infection in specific nerve cells using flow cytometry, which distinguishes cell types in mixed populations of cells. In particular embodiments, neurons can be assayed by detecting mRNA coding for markers of neuronal function. See, e.g., Turac et al. (2013) PloS One 8. In various embodiments, the detection and/or quantification of nerve cell-specific markers, such as neuron-specific, astrocyte-specific, glia-specific, and other nerve cell-specific markers, using flow cytometry allows for fast and accurate assessment of changes in the central nervous system. In some embodiments, the methods described herein are used for analysis of nerve cells in tissue culture, analysis of nerve cells from in vivo animal models, or samples from a human subject. In addition, gene expression in multiple cell types from different parts of the brain can be rapidly assayed and compared. In other embodiments, the flow cytometry methods described herein facilitate the characterization and sorting of cells in the central nervous system that are differentiating into neurons in highly heterogeneous populations of nerve cells. In addition, differentiating nerve cells are identified using the homogeneous probes described herein by detecting patterns of mRNA expression (mRNA profiles) in the cells well in advance of the emergence of cell-surface protein markers or cell morphology changes that are indicative of differentiation. In various embodiments, the flow cytometry methods described herein are used to identify neurons for transplantation into subjects suffering from neurological diseases. See Chiu et al. (1998) Methods 16(3):260-267 (isolation of neurons for transplantation). In other embodiments, the detection and/or quantification of patterns of mRNA expression and/or the presence or absence of cell-surface markers in nerve cells is used to more accurately identify neurons and neuronal progenitor cells that are best suited for use in subjects suffering from neurological diseases.

### 6.6. Methods for Prognosticating, Diagnosing, or Monitoring Disease, and Monitoring Efficacy of Therapy

In particular embodiments, the methods described herein are used to detect and/or quantify a change in the presence or amount of cellular components using flow cytometry, wherein a change in the presence or amount of one or more cellular components is correlated with predicting disease in a subject, the onset of or progression of a disease in a subject, or amelioration of a disease in response to therapy. In various embodiments, the change in the presence or amount of one or more cellular components is indicative of future onset of a disease. In certain embodiments, the change in the presence or amount of one or more cellular components is indicative of the presence of a disease. In other embodiments, the change in the presence or amount of one or more cellular components is indicative of the increasing or decreasing severity of a disease. In still other embodiments, the change in the presence or amount of one or more cellular components is indicative of the efficacy of a therapeutic regimen. In particular embodiments, the cellular component is a nucleic acid. In certain embodiments, the nucleic acid is RNA, such as mRNA that encodes a cellular protein. In other embodiments, the cellular component is a non-coding RNA, such as miRNA. In yet other embodiments, the cellular component is a protein, such as an enzyme, a cell-surface protein and the like. In various embodiments, where certain diseases are correlated with overexpression of particular genes (e.g., survivin in cancer cells) or under expression of certain genes (e.g., genes that are turned off in or by cancer cells), the methods described herein are utilized to ascertain the presence and/or number of cells that have aberrant levels of expression. In particular embodiments, where certain diseases are correlated with the size or morphology of particular cells, the methods described herein are used in conjunction with means to ascertain the presence and/or number of cells that have aberrant size or morphology as compared to healthy cells of the same type or cells from a healthy individual. In yet other embodiments, certain diseases, such as cancer or infection, can be correlated with abnormalities in cell structure, such as with abnormalities of the organelles. Accordingly, in certain embodiments, the methods described herein are used in conjunction with an organelle staining dye to assay the state of the organelles, such as an abnormally low or abnormally high number of particular organelles, or an abnormally small number or abnormally large number of intact organelles as compared to organelles in the same type of cells that are not diseased. In still other embodiments, the methods described herein are used to quantitate the number of cells in a sample that, e.g., are infected with a virus or parasite, that exhibit over-expression or under-expression of various cell markers that are indicative of disease, and/or that exhibit over-expression or under-expression of various cell markers that indicate amelioration of a disease.

In certain embodiments, the present disclosure relates to a method of predicting or diagnosing a disease in a subject comprising the steps of. (a) providing cells from the subject suspected of having a disease and eukaryotic cells from a healthy individual; (b) fixating and permeabilizing the cells of step (a); (c) contacting the cells of step (b) with a nucleic acid probe that is complementary to a nucleic acid sequence of interest present in one or more cells under conditions in which the nucleic acid probe hybridizes to a complementary nucleic acid sequence in the cells; (d) detecting or quantifying an amount of signal generated by the probe of step (c) hybridized to a complementary target nucleic acid sequence in cells from the subject using flow cytometry; (e) detecting or quantifying an amount of signal generated by the probe of step (c) hybridized to a complementary target nucleic acid sequence in the cells from the healthy individual using flow cytometry, wherein a deviation in the level of the nucleic acid of interest measured in step (d) from the level of the nucleic acid of interest in step (e) indicates a disease in the subject or is predictive of a disease in the subject. In particular embodiments, the nucleic acid probe comprises at least one probe that is part of a homogeneous probe system that generates a signal when bound to a complementary sequence in the target nucleic acid.

In various embodiments, the flow cytometry methods described herein are used to detect and/or quantify small numbers of individual cells in a patient sample exhibiting levels of gene expression that vary from the norm in the sample or in a healthy individual. Without being bound by any particular therapy, the methods described herein enable accurate detection and/or quantification of a small minority of aberrant cells in a sample from a subject, which had not been possible using prior methods that assessed levels of expression collectively in the sample. In various embodiments, the detection of a small minority of aberrant cells in a sample identifies a disease state in a subject before there are physical manifestations in the subject. In certain embodiments, the detection of a small minority of aberrant cells in a sample is predictive of a disease in the subject. In yet other embodiments, the detection of specific cell surface markers on aberrant cells indicates the tissue of origin of the aberrant cells. In various embodiments, the detection and/or quantification of upregulation of transcripts, downregulation of transcripts and/or co-expression of various cell targets in or on the cells can be used to monitor physiological changes in the cells, such as a transition from healthy cells to diseased cells or infected cells. In certain embodiments, these changes can be identified before the subject has symptoms of the disease or infection. Accordingly, enabling prediction of disease or diagnosis of disease at an early stage could allow greater efficacy of traditional therapeutic regiments or result in the development and/or use of alternative therapies that are less toxic than therapies currently used in diseases that are detected only after physical symptoms appear in the subject. In certain embodiments, the present invention is used to monitor disease progression in a subject and/or the efficacy of a therapeutic regimen by measuring changes in the number of cells that have inappropriate expression levels at various times during the course of therapy, or the degree of over- or under-expression.

Thus, in various embodiments, the present disclosure relates to a method of monitoring a disease in a subject comprising the steps of. (a) fixating and permeabilizing cells from a first cell sample from the patient; (b) contacting the first cell sample with a nucleic acid probe that is complementary to a nucleic acid of interest under conditions that allow the probe to bind to the nucleic acid of interest; (c) measuring an amount of signal generated by the probe hybridized to the nucleic acid of interest in the first cell sample using flow cytometry; (d) fixating and permeabilizing cells from a second cell sample from the subject; (e) contacting the second cell sample with the nucleic acid probe of step (b); (f) measuring an amount of signal generated by the probe hybridized to the nucleic acid of interest in the second cell sample using flow cytometry; and (g) comparing the amount of signal measured in step (c) with the amount of signal measured in step (f), wherein the level of the target nucleic acid measured in step (c) compared to the level of the target nucleic acid measured in step (f) is correlated with the presence or severity of the disease. In particular embodiments, the probe of steps (b) and (e) comprise at least one probe that is part of a homogeneous probe system that generates a signal when bound to a complementary sequence in the target nucleic acid. In particular embodiments, the method further comprises a step of contacting the fixated and permeabilized cells in steps (b) and (e) with a control nucleic acid probe under conditions in which the probe hybridizes to a complementary nucleic acid sequence in the cell, wherein the control nucleic acid probe is part of a homogeneous probe system that generates a signal when bound to a complementary sequence in a nucleic acid.

In certain embodiments, the second cell sample is collected at least about 1 day, at least about 1 week, at least about 2 weeks, at least about 3 weeks, at least about 1 month, at least about 6 months, or at least about 1 year or more after collection of the first cell sample.

In other embodiments, the present disclosure relates to a method of determining the efficacy of treatment in a patient suffering from a disease comprising the steps of: (a) fixating and permeabilizing cells from a first cell sample from the patient before treatment; (b) contacting the first cell sample with a nucleic acid probe that is complementary to a nucleic acid of interest under conditions that allow the probe to bind to the nucleic acid of interest; (c) measuring an amount of signal generated by the probe hybridized to the nucleic acid of interest in the first cell sample using flow cytometry; (d) fixating and permeabilizing cells from a second cell sample from the subject after treatment; (e) contacting the second cell sample with the nucleic acid probe of step (b); (f) measuring an amount of signal generated by the probe hybridized to the nucleic acid of interest in the second cell sample using flow cytometry; and (g) comparing the amount of signal measured in step (c) with the amount of signal measured in step (f), wherein the level of the target nucleic acid measured in step (c) compared to the level of the target nucleic acid measured in step (f) is correlated with the presence or severity of the disease. In various embodiments, the level of the target nucleic acid measured in step (c) compared to the level of the target nucleic acid measured in step (f) is correlated with the efficacy of the treatment. In particular embodiments, the probe of steps (b) and (e) comprise at least one probe that is part of a homogeneous probe system that generates a signal when bound to a complementary sequence in the target nucleic acid. In particular embodiments, the method further comprises a step of contacting the fixated and permeabilized cells in steps (b) and (e) with a control nucleic acid probe under conditions in which the probe hybridizes to a complementary nucleic acid sequence in the cell, wherein the control nucleic acid probe is part of a homogeneous probe system that generates a signal when bound to a complementary sequence in a nucleic acid.

In certain embodiments, the second cell sample is collected at least about 1 day, at least about 1 week, at least about 2 weeks, at least about 3 weeks, at least about 1 month, at least about 6 months, or at least about 1 year or more after collection of the first cell sample. In various embodiments, the second cell sample is collected during therapy. In other embodiments, the second cell sample is collected after therapy has been completed, such as at least about 1 day, at least about 1 week, at least about 2 weeks, at least about 3 weeks, at least about 1 month, at least about 3 months, at least about 6 months, or at least about 1 year or more after therapy has been completed.

In particular embodiments of the methods described herein, the disease is an infection, such as a parasitical infection or a viral infection. In other embodiments, the disease is cancer. In still other embodiments, the disease is an immune-mediated disorder.

In various embodiments, cells can be assayed by the methods described herein for patterns of overexpression, patterns of under expression, or a combination of overexpression and under expression of multiple (e.g., two or more) genes that together provide a “signature” indicative of a susceptibility to, presence or severity of, or stage of, a particular disease or cancer. In particular embodiments, cells can be assayed by the methods described herein for patterns of overexpression and/or under expression of multiple genes that together provide a “signature” indicative of a susceptibility to, presence of, or severity of age-related macular degeneration (Liu et al. (2011) J Transl Med 9:1-12, disclosing that levels of C5a were elevated, which resulted in increased levels of IL17, and Wei et al. (2012) Cell Rep 2:1151-1158 showing that hypomethylation of the IL17C receptor gene led to increased levels of expression of the receptor).

### 6.7. Flow Cytometry Methods for Identifying an Epitope that Induces Immune Modulation in a Subject, Assaying Immune Cell Phenotype, Responses, Function, Memory, and Immune Profiling of a Subject

Immune-mediated disorders result from an imbalance of the immune system. Immune-mediated disorders include conditions in which the body fails to distinguish “self” from “non-self” and, as a result, mounts an inappropriate immune response to a subject's healthy tissues, as well as conditions in which the immune system is unable to mount a response to foreign agents such as infectious agents (e.g., bacteria or virus) or to aberrant cells such as cancer cells to restore health to the subject. Traditional assays for immune responses have included measuring production of cytokines, e.g., pro-inflammatory or anti-inflammatory cytokines that are secreted from a subject's immune cells, such as peripheral blood mononuclear cells (PBMC), and have a systemic role in immune modulation. However, cytokines secreted into a subject's blood are necessarily separated from their cells of origin, and consequently, assays for detection of cytokines collected in the blood provide no information about the subject's immune status at the cellular level.

In contrast, the compositions and flow cytometry methods described herein allow for individually assaying cells with particular phenotypes, e.g., T-reg or T-resp cells, identified by the presence of one or more protein markers, e.g., cell-surface protein markers, using a multiplex format that provides comprehensive and comparative characterization of specific cells. Such multiplex analyses include, but are not limited to, identifying the tissue of origin of cells of interest, establishing correlations between cell function, degree of responsiveness or non-responsiveness and cell phenotype, and profiling the state of a cell by, for example, identifying patterns of gene expression or non-expression or changes in such gene expression or non-expression, elucidating the degree of cell activity or inactivity, identifying compounds comprising epitopes that activate or deactivate particular immune cells, and detecting the presence or absence of cell memory for specific epitopes. In various embodiments, assays are carried out with cells from a subject compared to cells of the same type from a healthy individual. In other embodiments, the assayed cells of the subject are compared to cells of the same type that have not been exposed to, e.g., a specific epitope. T-cells of interest and subsets thereof can be identified by phenotype, as determined by differential expression of cell surface markers or internal markers. In certain embodiments, the presence of one or more cell markers is detected using fluorescently labeled antibodies, and phenotypes are identified using flow cytometry. In particular embodiments, differential expression of cell surface markers such as CD3, CD4, CD8, CD25, CD127 and CD196 are used to identify specific T-cell phenotypes. In further embodiments, differential expression of one or more additional markers such as CD197, CD62L, CD69, CD45RO and Foxp3 is used in conjunction with one or more of antibodies to CD3, CD4, CD8, CD25, CD127 and CD196 in order to identify particular T-cell subsets of interest. In particular embodiments, a pattern of expression of cell surface markers such as those described in this paragraph can be used to assay memory in T-cells and/or activation status of particular types of T-cells. In particular embodiments, flow cytometry can be used to analyze activation of CD4+ T-cells, which can differentiate into Th1, Th2, Th9, Th17, Tfh or T-reg cells. In specific embodiments, T-cell differentiation can be detected or monitored by detecting or quantifying by flow cytometry expression of cytokines, transcription factors and phosphorylated proteins. In various embodiments, cytokine and transcription factor expression can be assayed by detecting or quantifying mRNA that encodes particular cytokines or transcription factors. In some embodiments, cytokine and transcription factor expression and/or phosphorylated proteins can be assayed by detecting or quantifying proteins in the cell. In yet other embodiments, the flow cytometry methods described herein can be used to T-cell fate, such as T-reg/Th17 plasticity, which can be influenced by the local milieu of the cells, e.g., levels of TGF-β, levels of pro-inflammatory cytokines such as IL-1 and IL-6. Various antibodies and reagents for assaying immune cells using flow cytometry are available from BD Biosciences.

The compositions and methods described herein allow for high-throughput, comprehensive immune profiling of a subject suffering from an immune-mediated disorder, or a subject susceptible to an immune-mediated disorder as compared to, e.g., a healthy individual. In particular embodiments, the compositions and methods described herein are used for assaying a subject's particular response to an epitope, either in vivo or in vitro, and/or a subject's particular response to an antigen versus a control. In various embodiments, the compositions and methods described herein are used for assaying memory of T-cells for specific epitopes that the cells had previously encountered and to which they previously responded. In certain embodiments, memory of T-cells is assayed by detecting a receptor for the epitope on the T-cell surface. In other embodiments, detection of markers associated with cell memory is used in conjunction with detecting receptors of the specific epitope.

Accordingly, in certain embodiments, the present disclosure is directed to compositions and methods for identifying nucleic acids coding for cytokine and/or cytokine receptor proteins, and/or cell-surface markers and/or internal markers in individual cells, thereby facilitating identification of particular cell types involved in producing various cytokines, cytokine receptors or other immune factors. In some embodiments, the methods further include identifying proteins such as cell-surface markers or intra-cellular proteins in addition to identifying nucleic acid expression. The compositions and methods described herein can also be used to identify and test various compounds comprising an epitope that induce immune modulation—immune tolerance or immune responsiveness—in a subject and/or in a healthy individual or the presence or lack of cell memory for specific epitopes. In various embodiments, the flow cytometry methods described herein can be used for, e.g., high throughput in vitro screening of cells from a tissue under attack by the immune system of a subject suffering from an immune-mediated disorder against libraries of compounds comprising epitopes to identify compounds that induce immune tolerance in the subject's cells. In various embodiments, changes in gene expression are monitored to identify compounds that will expand regulatory cells and decrease autoimmunity. In other embodiments, the flow cytometry methods described herein can be used to identify compounds that induce specific responsiveness of responder T-cells to an infection or cancer in a subject. In these embodiments, changes in gene expression are monitored to identify compounds that will expand effector cells to enhance, e.g., the subject's response to the aberrant cells. In yet other embodiments, differences in gene expression assayed in vitro can be monitored to identify specific epitopes that induce long-term memory of lymphocytes, thereby conferring upon the subject the benefit of a treatment that has lasting effects.

In various embodiments, the present disclosure relates to compositions comprising a fixated immune cell comprising two or more labeled probes that bind to (i) a target; and (ii) a control element, wherein the probes are part of a homogeneous probe system that generates a signal when bound to a target, and wherein the target is associated with an immune-related function. In particular embodiments, the fixated immune cell is from a subject suffering from an immune-mediated disorder. In other embodiments, the fixated immune cell is from a healthy individual. In some embodiments, the control element is a control probe. In other embodiments, the control element is a control cell.

In certain aspects, the present disclosure relates to flow cytometry methods for identifying a compound comprising an epitope that induces immune tolerance in a subject. In other aspects, the present disclosure relates to methods for identifying a compound comprising an epitope that induces immune tolerance in a subject suffering from an immune-mediated disorder. In yet other embodiments, the present disclosure relates to flow cytometry methods for identifying a compound comprising an epitope that induces immune responsiveness in a subject suffering from a disease including, but not limited to, an infection or cancer. In still other embodiments, the present disclosure relates to flow cytometry methods for identifying specific memory of immune cells for an epitope that induces an immune response or an epitope that induces immune tolerance.

In various embodiments, the compound comprising an epitope that induces immune tolerance or immune responsiveness in a subject is identified from a library or collection of compounds. In some embodiments, the library is a library of biological epitopes, e.g., a library of organ specific epitopes. In these embodiments, the epitopes are from a particular organ of the body, e.g., the eye, and the library of organ specific epitopes is, for example, a library of S-antigen epitopes. In other embodiments, the library is a library of epitopes that are not organ specific, e.g., that are found throughout the body. An example of this embodiment is a library of HLA epitopes, such as a library of variant HLA epitopes (e.g., a library of HLA-B27 epitopes). In some embodiments, the library can be a library of epitopes from the subject (a library of self epitopes) or a library of epitopes that are not from the subject (a library of non-self epitopes). In various embodiments, the compound comprising an epitope that induces immune tolerance or immune responsiveness in a subject is identified from tissues of the subject or from tissues of a healthy individual, such as from a colon tissue extract, a lung tissue extract, a cervical tissue extract, a breast tissue extract or from combinations of extracts from various tissues. In certain embodiments, the compound comprising an epitope that induces immune tolerance or immune responsiveness in a subject is identified from tissues at the site of the disorder, disease or infection. In some embodiments, the compound comprising an epitope that induces immune responsiveness in a subject is identified from a cancerous tumor tissue extract, and/or a benign tumor extract from the subject or from a healthy individual. In particular embodiments, the compound that induces immune tolerance or immune responsiveness in a subject is a mixture of epitopes, such as a mixture of epitopes from cancerous tissue or tumor and from benign tissue, or a mixture of different organ-specific epitopes.

In particular embodiments, the library is a library of peptides. In certain embodiments, the library comprises synthetic peptides. In certain embodiments, the compound comprising an epitope that induces immune tolerance or that induces a pro-inflammatory immune response in a subject is identified from a collection of compounds. A collection of peptides can be tested for induction of immune tolerance or immune responsiveness. In certain embodiments, optimal lengths of such peptides vary from 8-15 amino acids. See Ekeruche-Makinde et al. (2013) Blood 121(7):1112-23; Percus et al. (1993) Proc. Natl. Acad. Sci. USA 90:1691-95. In some embodiments, peptides are synthesized with a given length and a predetermined overlapping sequence so that the library encompasses a particular protein. See, e.g., deSmet et al. (2001) Investigative Ophthalmology & Visual Science (2001) 42(13):3233-38; Gershoni et al. (2007) BioDrugs 21(3):145-56. In other embodiments, peptide libraries are created using mass spectrometry, such as by Solid Phase Epitope Recovery (SPHERE). See Lawendowski et al. (2002) J. Immunol. 169:2414-21. In certain embodiments, a library for use in the methods described herein includes, but is not limited to a phage display library, a bacterial or yeast display library, an mRNA display library, a ribosomal display library, a polysomal display library and a peptide matrix. See e.g., U.S. Patent publication no. 2013/0004513 (Osterroth et al.). In various embodiments, identification of a compound comprising an epitope that induces immune tolerance or a pro-inflammatory immune response can be carried out using a synthetic random library of peptides. In these embodiments, the protein that is associated with aberrant immune reactivity need not be identified. Accordingly, although most peptides used for screening are octamers or longer, penta-peptides, tetra-peptides and even tri-peptides can be recognized by CD4+ T-cells. See Hemmer et al. (2000) International Immunology 12(3):375-383.

In yet other embodiments, combinatorial epitope collections are utilized. Accordingly, in certain embodiments, the collection comprises at least substantially all permutations of a compound having 4 monomers. In certain embodiments, the compound is a peptide and the collection comprises at least substantially all permutations of a tetrameric peptide with all 20 amino acids at each position such that the collection includes 204 peptide tetramers. In other embodiments, the compound is a peptide and the collection comprises at least substantially all permutations of a pentameric peptide with all 20 amino acids at each position such that the collection includes 20′ peptide pentamers. In other embodiments, the compound is a peptide and the collection comprises at least substantially all permutations of a hexameric peptide, a septameric peptide, an octameric peptide, a nonameric peptide, or a decameric peptide.

In still other embodiments, the compound identified by a method described herein is used as a reference sequence to search a library for additional compounds that have homology to the reference sequence. In certain embodiments, the reference sequence is the entire protein target sequence. In various embodiments, the reference sequence and identified compounds are compared using a comparison window, a contiguous specific segment of the polypeptide sequence, which can have gaps compared to the reference sequence, for optimal alignment of peptides. In certain embodiments, the comparison sequence is at least about 10 amino acids, at least about 15 amino acids, at least about 20 amino acids, or at least about 25 or more amino acids. Tools for aligning sequences for comparison are well known in the art and include, but are not limited to, CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif, USA). In certain embodiments, compounds are chosen that have at least about 30%, such as at least about 35%, at least about 40%, at least about 45%, at least about 50%, at least about 55%, at least about 60%, at least about 65%, at least about 70%, at least about 75%, at least about 80%, at least about 85%, or at least about 90% or more homology or identity to the reference sequence. In other embodiments, compounds are chosen that have an amino acid sequence that adopts a three-dimensional structure that is a recognized epitope that binds to a particular receptor and/or elicits a particular immune response. See, e.g., U.S. patent application Ser. No. 09/254,825. The identified compound can then be assayed by a method described herein.

In various embodiments, the T-resp cells or T-reg cells have been primed. In particular embodiments, the T-resp cells or T-reg cells are primed in culture. In other embodiments, the T-resp cells or T-reg cells are primed in the host. In some embodiments, the T-resp cells or the T-reg cells are not primed. In particular embodiments, unprimed T-resp cells are primed by pharmacologic means (calcium ionophore and phorbol ester stimulation), by direct cross-linking of a T-cell receptor on a large percentage of responder cells, by cross-linking the receptors on certain T-resp sub-populations with monoclonal antibodies specific for V regions of R chains of T-cell receptor or with superantigens specific for certain VO-chain regions, or by indirectly cross-linking the T-cell receptor to non-T-cell receptor antigens. Various agents that can be used to activate unprimed T-resp cells in proliferative assays can be found in Kruisbeek, A. M., Shevach, E. and Thornton, A. M. (2004) Proliferative Assays for T Cell Function. Current Protocols in Immunology. 60:III:3.12:3.12.1-3.12.20 at Table 3.12.1. In particular embodiments, unprimed T-resp cells can be activated with plate-bound antibodies, such as monoclonal antibodies to the CD3/TCR complex. In still other embodiments, stimulation and expansion of T-resp cells is carried out by exposure to beads coated with anti-CD3 and anti-CD28. See Trickett and Kwan (2003) J Immunol Methods 275(1-2):251-255.

In various embodiments, “geographic” considerations may be taken into account when identifying a specimen to be assayed in the flow cytometry methods described herein. Specifically, blood samples are commonly used to test the presence and amount of certain diseases or disorders, such as HBV or HCV infection. This is essentially a matter of convenience since neither of these viruses replicates in blood cells, but rather, viral particles shed after viral growth takes place in the liver and can be detected in the blood. Similarly, the cytokines secreted by T-resp cells and T-reg cells, or antibodies from B-cells can be measured in blood or serum regardless of the particular location of an immune-mediated disorder. An example of this is macular degeneration, where an inflammatory response takes place in the eye, and yet, changes in methylation patterns detected in peripheral blood cells (Wei et al., 2012, Cell Reprtr 2; 1151-1158.) and serum levels of IL-17 (Liu et al., 2011, J. Transpl. Med. 9, 1-12) are correlated with the disorder. Accordingly, although such global effects can be measured outside of the physical location where a pathogenic immune reaction is taking place, there are advantages to also obtaining clinical specimens that are in proximity to the affected organ or location. For instance, it has been previously shown that the immune profile of T-cells in synovial fluid from a subject differs from the majority of T-cells at large.

Accordingly, in some embodiments, the present disclosure relates to a method for identifying a compound comprising an epitope from a library or collection of epitopes that induces immune tolerance in a subject suffering from an immune-mediated disorder comprising the steps of (a) exposing live T-reg cells from the subject to the compound in the presence of an epitope binding agent; (b) incubating (i) the T-reg cells of step (a) and (ii) control T-reg cells that have not been exposed to the compound for a period of time to allow cell activation; (c) permeabilizing and fixating (i) the T-reg cells of step (b)(i) and (ii) the control T-reg cells of step (b)(ii); (d) contacting (i) the T-reg cells of step (c)(i) and (ii) the control T-reg cells of step (c)(ii) with a nucleic acid probe that is complementary to a nucleic acid sequence associated with T-cell activation or T-reg activity under conditions in which the nucleic acid probe hybridizes to a complementary nucleic acid sequence in the T-cells; (e) detecting using flow cytometry (i) an amount of signal (S₁) generated by the probe that is hybridized to the complementary target nucleic acid sequence in the T-reg cells of step (d)(i), and (ii) an amount of signal (S₀) generated by the probe that is hybridized to the control T-reg cells of step (d)(ii); and (f) detecting or measuring (i) the amount of signal (S₁) generated in step (e)(i), and (ii) the amount of signal (S₀) generated in step (e)(ii), wherein the compound that induces a S₁/S₀≥1 or a S₀/S₁<1 is identified as a candidate compound for inducing immune tolerance in the subject. In certain embodiments, the live control T-reg cells are from the subject. In other embodiments, the live T-reg cells are from a healthy individual. In various embodiments, nucleic acid probes utilized in the methods disclosed herein are part of a homogeneous probe system that generates a signal when bound to a complementary sequence in the target nucleic acid.

In various embodiments, a target nucleic acid sequence associated with an anti-inflammatory response is a nucleic acid that encodes an anti-inflammatory cytokine or a receptor for an anti-inflammatory cytokine present in one or more T-cells. In various embodiments, the anti-inflammatory cytokine is selected from IL-4, IL-10, IL-11, IL-13, TGFβ, IL-35, LIF, CTLA-4, CD39, galectin 1, galectin 3, galectin 9, PD-L1 and combinations thereof. In other embodiments, the receptor for an anti-inflammatory cytokine is selected from an IL-4 receptor, an IL-10 receptor, an IL-11 receptor, an IL-13 receptor, a TGFβ receptor, an IL-35 receptor, a LIF receptor, a CTLA-4 receptor, a CD39 receptor, a galectin 1 receptor, a galectin 3 receptor, a galectin 9 receptor, a PD-1 receptor and combinations thereof. Other targets that are associated with pro-inflammatory responses can be found in McHugh et al. (2002) Immunity 16:311-323.

In certain embodiments, the immune-mediated disorder is selected from age-related macular degeneration or uveitis.

In various embodiments, the present disclosure relates to methods of identifying a compound comprising an epitope from a library or collection of epitopes that induces immune tolerance in a subject suffering from an immune-mediated disorder comprising the steps of (a) exposing (i) live T-resp cells from the subject, and (ii) live T-resp cells from a healthy individual to the compound in the presence of an epitope binding agent; (b) incubating the cells for a period of time to allow cell activation; (c) permeabilizing and fixating the cells of step (b); (d) contacting the cells of step (c) with a nucleic acid probe that is complementary to a nucleic acid sequence that is part of a gene associated with a pro-inflammatory response under conditions in which the nucleic acid probe hybridizes to a complementary nucleic acid sequence in the T-resp cells; (e) detecting using flow cytometry (i) an amount of signal generated by the probe that is hybridized to the complementary target nucleic acid sequence associated with a pro-inflammatory response under conditions in which the nucleic acid probe hybridizes to a complementary nucleic acid sequence in the T-resp cells of the subject (RespP), and (ii) an amount of signal generated by the probe that is hybridized to the complementary target nucleic acid sequence associated with a pro-inflammatory response under conditions in which the nucleic acid probe hybridizes to a complementary nucleic acid sequence in the T-resp cells of the healthy individual (RespH); and (f) detecting or measuring (i) the amount of signal (RespP) measured in step (e)(i), and (ii) the amount of signal (RespH) measured in step (e)(ii), wherein the compound that induces a RespH/RespP≥1 or a RespP<RespH is identified as a candidate compound for inducing immune tolerance. In various embodiments, the method further comprises the steps of (g) determining the total number of T-cells (P1) from the healthy individual that provides an amount of regulatory T-cells that induces 50% suppression of responder T-cell activity in the presence of the compound; and (h) determining the total number of T-cells (P2) from the subject that provides an amount of regulatory T-cells that induces 50% suppression of responder T-cell activity in the presence of the compound, wherein the compound that induces a RespH/RespP≥1, a P1/P2>1 or RespH/RespP≥1 and a P1/P2>1 is identified as a candidate compound for inducing immune tolerance in the subject. In various embodiments, a target nucleic acid sequence associated with a pro-inflammatory response is a nucleic acid that encodes a pro-inflammatory cytokine or a receptor for a pro-inflammatory cytokine present in one or more T-cells. In various embodiments, the pro-inflammatory cytokine is selected from IFNγ, TNFα, I, IL-6, IL-12, IL-17, IL-18, IL-15, IL-8, IL-21, IL-25 and combinations thereof. In other embodiments, the receptor for a pro-inflammatory cytokine is selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof.

In particular embodiments, the present disclosure relates to a method of identifying a compound comprising an epitope that induces immune tolerance in a subject suffering from an immune-mediated disorder comprising the steps of (a) exposing (i) live T-resp cells from the subject and live T-resp cells from a healthy individual; and (ii) live T-reg cells from the subject and live T-reg cells from a healthy individual to the compound in the presence of an epitope binding agent; (b) incubating the cells for a period of time to allow cell activation; (c) fixating and permeabilizing the cells of step (b); (d) contacting (i) the T-resp cells of step (c) with a nucleic acid probe that is complementary to a nucleic acid that is part of a gene associated with a pro-inflammatory response in one or more T-resp cells under conditions in which the nucleic acid probe hybridizes to a complementary nucleic acid sequence in the T-resp cells; and (ii) the T-reg cells of step (c) with a nucleic acid probe that is complementary to a nucleic acid that is part of a gene associated with an anti-inflammatory response under conditions in which the nucleic acid probe hybridizes to a complementary nucleic acid sequence in the T-reg cells; (e) detecting using flow cytometry (i) an amount of signal generated by the probe of step (d)(i) hybridized to the T-resp cells of the healthy individual (RespH), (ii) an amount of signal generated by the probe of step (d)(i) hybridized to the T-resp cells of the subject (RespP), (iii) an amount of signal generated by the probe of step (d)(ii) hybridized to the T-reg cells of the healthy individual (RegH), and (iv) an amount of signal generated by the probe of step (d)(ii) hybridized to the T-reg cells of the subject (RegP); and (f) detecting or measuring (i) the amount of signal generated in step (e)(i) (RespH), (ii) the amount of signal generated in step (e)(ii) (RespP), (iii) the amount of signal generated in step (e)(iii) (RegH), and (iv) the amount of signal generated in step (e)(iv) (RegP), wherein the compound that induces a RespH/RespP<1, a RegH/RegP≥1 or a RespH/RespP<1 and a RegH/RegP≥1 is identified as a candidate compound for inducing immune tolerance.

In particular embodiments, the methods described herein include an in vitro method of identifying from a library or collection of compounds a compound comprising an epitope that induces immune tolerance in a human subject suffering from an immune-mediated disorder comprising the steps of (a) contacting one or more live immune cells of the subject with (i) a labeled compound comprising the epitope in the presence of an epitope binding agent under conditions in which the compound binds to a T-cell receptor on a regulatory T-cell; and (ii) a labeled probe specific for a regulatory T-cell marker; (b) identifying or isolating regulatory T-cells bound to the probe of step (a)(iii) by flow cytometry; and (c) detecting or measuring an amount of the labeled compound of step (a)(i) bound to the T-cell receptors of the regulatory T-cells of step (b) by flow cytometry, wherein the presence or quantity of the labeled compound of step (a)(i) bound to the subject's regulatory T-cells identifies an epitope that elicits immune tolerance in the subject. In certain embodiments, the library is a library of biological epitopes, such as HLA epitopes, HLA variant epitopes, S-antigen epitopes, self biological epitopes, non-self biological epitopes or a mixture thereof, all permutations of epitope pentamers or all permutations of epitope tetramers. In certain embodiments, the probe of step (a)(ii) is an antibody. In particular embodiments, the antibody binds to a marker selected from CD25, CD39, Foxp3, CTLA-4, HLA-DR, CD45RA, CD73, GITR, TGFβ, GARP and LAP, or a combination thereof.

In some embodiments, the methods described herein include an in vitro method of identifying from a library or collection of compounds a compound comprising an epitope that induces immune tolerance in a human subject suffering from an immune-mediated disorder comprising the steps of (a) contacting one or more live immune cells of the subject with (i) a labeled compound comprising an epitope in the presence of an epitope binding agent under conditions in which the compound binds to a T-cell receptor on a regulatory T-cell; and (ii) a labeled probe specific for a regulatory T-cell-specific surface marker; (b) identifying or isolating regulatory T-cells bound to the probe of step (a)(ii) by flow cytometry; (c) detecting or measuring an amount of the labeled compound of step (a)(i) bound to the T-cell receptors of the regulatory T-cells of step (b) by flow cytometry; (d) fixating and permeabilizing the cells of step (c); (e) contacting the regulatory T-cells of step (d) with (i) a labeled nucleic acid probe that is complementary to a target nucleic acid that is part of a gene that codes for an anti-inflammatory cytokine or for a receptor for an anti-inflammatory cytokine; and (ii) an antibody to a intracellular target; (f) detecting or measuring an amount of signal generated by the probe of step (e)(i) and the antibody of step (e)(ii) that is hybridized to the target nucleic acid in the cell using flow cytometry, wherein the presence or quantity of the target nucleic acid, the intracellular target or both the target nucleic acid and the intracellular target in the cell identifies an epitope that induces immune tolerance in the subject. In various embodiments, the nucleic acid of step (e)(i) is mRNA. In various embodiments, the mRNA encodes an anti-inflammatory cytokine selected from IL-4, IL-10, IL-11, IL-13, TGFβ, IL-35, LIF, CTLA-4, CD39, galectin 1, galectin 3, galectin 9, PD-L1 and combinations thereof. In other embodiments, the target nucleic acid is mRNA that encodes a receptor for an anti-inflammatory cytokine selected from an IL-4 receptor, an IL-10 receptor, an IL-11 receptor, an IL-13 receptor, a TGFβ receptor, an IL-35 receptor, a LIF receptor, a CTLA-4 receptor, a CD39 receptor, a galectin 1 receptor, a galectin 3 receptor, a galectin 9 receptor, a PD-1 receptor and combinations thereof.

In particular embodiments, the disclosure relates to an in vitro method of identifying a compound from a library or collection of compounds comprising an epitope that induces immune tolerance in a human subject suffering from an immune-mediated disorder comprising the steps of (a) exposing (i) an amount of T-reg cells from a healthy individual; and (ii) an equal amount of T-reg cells from the subject to the compound; (b) fixating and permeabilizing the cells of step (a); (c) contacting the cells from step (a) with a nucleic acid probe that is complementary to a nucleic acid coding for a T-reg marker; (d) detecting using flow cytometry (i) an amount of signal generated by the probe of step (c) hybridized to T-reg cells from the healthy individual (RegH), and (ii) an amount of signal generated by the probe of step (c) hybridized to the T-reg cells from the subject (RegS); (e) detecting or measuring (i) the amount of signal generated in step (d)(i) (RegH); and (ii) the amount of signal generated in step (d)(ii) (RegS); wherein the compound that induces RegH/RegS<1 or a RegS/RegH>1 is identified as a candidate compound for inducing immune tolerance in the subject. In various embodiments the T-reg cells have the CD4+CD25+ phenotype or the CD4+CD25+Foxp3 phenotype. In particular embodiments, the T-reg cell marker is selected from IL-4, IL-10, IL-11, IL-13, TGFβ, IL-35, LIF, CTLA-4, CD39, galectin 1, galectin 3, galectin 9, PD-L1 and combinations thereof. In additional embodiments, the T-reg cell marker is selected from an IL-4 receptor, an IL-10 receptor, an IL-11 receptor, an IL-13 receptor, a TGFβ receptor, and IL-35 receptor, a LIF receptor, a CTLA-4 receptor, a CD39 receptor, a galectin 1 receptor, a galectin 3 receptor, a galectin 9 receptor, a PD-1 receptor, and combinations thereof. In yet other embodiments, the T-reg cell marker is selected from CD25, CD39, Foxp3, CTLA-4, HLA-DR, CD45RA, CD73, GITR, GARP and LAP and combinations thereof.

In various embodiments, the present disclosure relates to in vitro methods of identifying compounds that comprise an epitope that induces immune tolerance or immune responsiveness in a cell of interest using a cell proliferation assay. T-cell proliferation can be measured using fluorescent dyes (cell tracking dyes) to label T-cells and monitor decreases in fluorescence associated with cell division. In some embodiments in which a mixed-cell assay is used, different T-cells can be independently labeled with two readily distinguishable dyes in order to discriminate each T-cell population in co-cultures. See Brusko et al. (2007) Immunol. Investigations 36:607-628. See, e.g., Venken et al. (2007) J. Immunol. Methods 322:1-11. As used in the cell proliferation assays described herein, “an equal amount” of T-cells recited in the methods described in this section means that the amounts of T-cells being compared differ by no more than about 10%, such as by not more than 8%, such as no more than about 6%, no more than about 5%, no more than about 4%, no more than about 3%, no more than about 2%, no more than about 1%, no more than about 0.5%, no more than about 0.25%, or no more than about 0.1% or less. In some embodiments, the comparison is between equal numbers of T-cells. As used in the cell proliferation methods described herein, a “low fluorescent cell-staining dye signal” is any fluorescent signal that is weaker than the fluorescent dye signal measured in the cells before the cells have proliferated, such as a signal that is about 5% weaker, such as about 10% weaker, about 15% weaker, about 20% weaker, about 25% weaker, about 30% weaker, about 35% weaker, about 40% weaker, about 45% weaker, about 50% weaker, about 55% weaker or lower than the fluorescent cell-staining dye signal measured in the cells before they have proliferated.

In one embodiment, the present disclosure relates to an in vitro method of identifying from a library or collection of compounds a compound comprising an epitope that induces immune tolerance in a subject suffering from an immune-mediated disorder comprising the steps of (a) exposing (i) an amount of T-cells from the subject, and (ii) an equal amount of T-cells from the subject with T-reg cells, e.g., CD25+ cells, depleted to a fluorescent cell-staining dye to a fluorescent cell-staining dye; (b) exposing (i) the T-cells of step (a)(i) and (ii) the T-cells of step (a)(ii) to the compound in the presence of an epitope binding agent; (c) incubating the T-cells of (i) step (b)(i) and (ii) step (b)(ii) for a period of time to allow cell proliferation; (d) fixating and permeabilizing (i) the cells of step (c)(i) and (ii) the cells of step (c)(ii); (e) contacting (i) the T-cells from step (d)(i) with a CD4+ cell-surface probe and a labeled nucleic acid probe that is complementary to a target nucleic acid that is part of a gene that codes for a pro-inflammatory cytokine or for a receptor of a pro-inflammatory cytokine under conditions in which the nucleic acid probe hybridizes to a complementary nucleic acid sequence in the cells, and (ii) the T-cells with CD25+ cells depleted from the subject of step (d)(ii) with a CD4 cell-surface probe and a labeled nucleic acid probe that is complementary to a target nucleic acid that is part of a gene that codes for a pro-inflammatory cytokine or for a receptor of a pro-inflammatory cytokine under conditions in which the nucleic acid probe hybridizes to a complementary nucleic acid sequence in the cells; (f) detecting by flow cytometry (i) a CD4+ signal, a fluorescent cell-staining dye signal, and a signal from the labeled nucleic acid probe in the cells of step (e)(i), and (ii) a CD4+ signal, a fluorescent cell-staining dye signal, and a signal from the labeled nucleic acid probe in the cells of step (e)(ii); (g) detecting or measuring by flow cytometry (i) an amount of CD4+ T-cells in step (g)(i) having a low fluorescent cell-staining dye signal (D₁) and a signal from the labeled nucleic acid probe (T-Resp₁); and (ii) an amount of CD4+ T-cells in step (g)(ii) having a low fluorescent cell-staining dye signal (D₂) and a signal from the labeled probe (T-Resp₂), wherein the compound that induces a T-Resp₁≤T-Resp₂ or a T-Resp₂>T-Resp₁ or the compound induces a D₁≥D₂ or a D₂<D₁ or both a T-Resp₁≤T-Resp₂ or a T-Resp₂>T-Resp₁ and a D₁≥D₂ or a D₂<D₁ is identified as a candidate compound for inducing immune tolerance in the subject. Accordingly, in these embodiments, the T-cells of step (a)(i) include both T-resp cells and T-reg cells, and the T-cells of step (a)(ii) include T-resp cells depleted of T-reg cells, which can be effected by any method known to the skilled artisan. In some embodiments, T-reg cells are depleted from a T-cell sample using anti-CD25 antibody or beads. In other embodiments, T-reg cells are depleted from a T-cell sample using fluorescence activated cells sorting (FACS). In these embodiments, mean purity of sorted cells is at least about 94%, such as at least about 95%, at least about 96%, at least about 97% or at least about 98% or more. See Venken et al. (2007) J. Immunol. Methods 322:1-11.

In various embodiments, the disclosure relates to an in vitro method of identifying from a library or collection of compounds a compound comprising an epitope that induces immune tolerance in a subject suffering from an immune-mediated disorder comprising the steps of (a) exposing (i) an amount of T-reg cells from the subject, and (ii) an equal amount of T-resp cells from the subject to a fluorescent cell-staining dye; (b) exposing (i) the cells of step (a)(i) and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of T-reg cells of step (c)(i) having a low fluorescent cell-staining dye signal (RegS); and (ii) an amount of T-resp cells in step (c)(ii) having a low fluorescent cell-staining dye signal (RespS), wherein the compound that induces a RegS≥RespS or a RespS<RegS is identified as a candidate compound for inducing immune tolerance in the subject. In particular embodiments, the T-reg cells have the CD4+CD25+ phenotype and the T-resp cells have the CD4+CD25− phenotype. In certain embodiments, the method further comprises after step (c) a step of fixating and permeabilizing the cells of step (c) and a step of contacting the fixated T-reg cells and the fixated T-resp cells with a probe that is specific for Foxp3.

In particular embodiments, the disclosure relates to an in vitro method of identifying from a library or collection of compounds a compound comprising an epitope that induces immune tolerance in a subject suffering from an immune-mediated disorder comprising the steps of (a) exposing (i) an amount of T-reg cells from the subject (RegS) and an equal amount of T-reg cells from a healthy individual (RegH), and (ii) an amount of T-resp cells from the subject (RespS) and an equal amount of T-resp cells from a healthy individual (RespH) to a fluorescent cell-staining dye; (b) exposing (i) the cells of step (a)(i) and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of RegS cells of step (c)(i) having a low fluorescent cell-staining dye signal (RegS), (ii) an amount of RegH cells of step (c)(i) having a low fluorescent cell-staining dye signal (RegH), (iii) an amount of RespS cells of step (c)(ii) having a low fluorescent cell-staining dye signal (RespS), and (iv) an amount of RespH cells of step (c)(ii) having a low fluorescent cell-staining dye signal (RespH), wherein the compound that induces a RespH/RespS≥1, a RegH/RegS≤1 or a RespH/RespS≥1 and a RegH/RegS≤1 is identified as a candidate compound for inducing immune tolerance in the subject. In particular embodiments, the method further comprises after step (c) a step of fixating and permeabilizing the cells of step (c), and a step of contacting the fixated T-reg cells and the fixated T-resp cells with a probe that is specific for Foxp3.

In certain embodiments, the present disclosure relates to an in vitro method of identifying from a library or collection of compounds a compound comprising an epitope that induces immune tolerance in a human subject suffering from an immune-mediated disorder comprising the steps of (a) exposing (i) an amount of responsive T-cells from the subject in the presence of regulatory T-cells from the subject, and (ii) an equal amount of responsive T-cells from the subject in the absence of regulatory T-cells to a fluorescent cell-staining dye; (b) exposing (i) the cells of step (a)(i), and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of cells of step (c)(i) having a low fluorescent cell-staining dye signal (RespS₁), and (ii) an amount of cells of step (c)(ii) having a low fluorescent cell-staining dye signal (RespS₀), wherein the compound that induces a RespS₁<RespS₀ is identified as a candidate compound for inducing immune tolerance in the subject. In various embodiments, the responder T-cells of step (a) have the phenotype CD4+CD25−Foxp3−, and the regulatory T-cells of step (a) have the phenotype CD4+CD25+Foxp3+. In various embodiments, the CD4+CD25-Foxp3− are isolated from peripheral blood mononuclear cells.

In various embodiments, the disclosure relates to an in vitro method of identifying from a library or collection of compounds a compound comprising an epitope that induces immune tolerance in a human subject suffering from an immune-mediated disorder comprising the steps of (a) exposing (i) an amount of T-resp cells from the subject, and (ii) an equal amount of T-resp cells from the subject to a fluorescent cell staining dye; (b) exposing the cells of step (a)(i) to the compound in the presence of an epitope binding agent; (c) incubating (i) the cells of step (a)(ii) and (ii) the cells of step (b) for a period of time to allow cell proliferation; (d) detecting or measuring by flow cytometry (i) an amount of cells of step (c)(ii) having a low fluorescent cell-staining dye signal (RespS); and (ii) an amount of cells of step (c)(i) having a low fluorescent cell-staining dye signal (RespS₀), wherein the compound that induces a RespS/RespS₀≤1 is identified as a candidate compound for inducing immune tolerance. In various embodiments, the responder T-cells have the phenotype CD4+CD25−Foxp3−.

In certain embodiments, the disclosure relates to an in vitro method of identifying from a library or collection of compounds a compound comprising an epitope that induces immune tolerance in a human subject suffering from an immune-mediated disorder comprising the steps of (a) exposing (i) an amount of regulatory T-cells from a healthy individual to the compound in the presence of responder T-cells from the healthy individual, and (ii) an equal amount of regulatory T-cells (as provided in step (a)(i)) from the subject to the compound in the presence of an equal amount of responder T-cells (as provided in step (a)(i)) from the subject in the presence of a fluorescent cell-staining dye; (b) exposing (i) the cells of step (a)(i); and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of regulatory T-cells of step (c)(i) having a low fluorescent cell-staining dye signal (RegH); and (ii) an amount of regulatory T-cells of step (c)(ii) having a low fluorescent cell-staining dye (RegS), wherein the compound that induces a RegH/RegS≤1 or a RegS/RegH>1 is identified as a candidate compound for inducing immune tolerance in the subject. In certain embodiments, the regulatory T-cells have the phenotype CD4+CD25+ and the responder T-cells have the phenotype CD4+CD25-.

Additional methods for identifying a compound comprising an epitope that induces immune tolerance can be found in U.S. patent application Ser. No. 13/871,730 filed Apr. 26, 2013 and PCT application no. PCT/US14/35549 filed Apr. 25, 2014, the contents of all of which are incorporated by reference herein in their entirety.

In additional embodiments, the present disclosure relates to in vitro methods of identifying a compound comprising an epitope from a library or collection of compounds that elicits immune responsiveness in a subject suffering from a disease. Accordingly, in some embodiments, the subject suffers from a disease, such as cancer or an infection, e.g., a bacterial or viral infection, but is unable to mount an adequate immune response to the aberrant or affected cells in order to alleviate or cure the disease. The deficiencies of the subject's immune system can arise from a lack of recognition of affected cells as foreign due to the similarity between the diseased cells and the subject's normal cells, as a result of a weak immune response from the subject that is unable to destroy the diseased or infected cells, or because aberrant cells give off substances that keep the immune system in check. Accordingly, the methods described herein are directed to identification of compounds comprising an epitope that elicit immune responsiveness in a subject suffering from a disease or infection.

In certain embodiments, the present disclosure relates to an in vitro method of identifying a compound comprising an epitope from a library or collection of compounds that elicits immune responsiveness in a human subject suffering from a disease comprising the steps of (a) contacting one or more immune cells of the subject with (i) a labeled compound comprising the epitope in the presence of an epitope binding agent under conditions in which the compound binds to a T-cell receptor on a responder T-cell; and (ii) a labeled probe specific for a responder T-cell-specific surface marker; (b) identifying or isolating responder T-cells bound to the labeled probe of step (a)(ii) by flow cytometry; and (c) detecting or measuring an amount of the labeled compound of step (a)(i) bound to the T-cell receptors on the responder T-cells of step (b) by flow cytometry, wherein the presence or quantity of the labeled compound of step (a)(i) bound to the subject's responder T-cells identifies an epitope that elicits an immune response in the subject. In various embodiments, the library is a library of biological epitopes, such as a library of HLA epitopes, a library of HLA variant epitopes, a library of viral epitopes, a library of S-antigen epitopes, a library of self and/or non-self biological epitopes, or a collection that includes all permutations of epitope pentamers or that includes all of the permutations of epitope tetramers. In various embodiments, the labeled probe of step (ii) is an antibody that binds to a responder T-cell marker selected from CD8, CD16, CD56, CD4, CD3, CD69, CD45RO, Tbet, perforin, Granzyme B, Nk1.1, NKG2D and combinations thereof.

In various embodiments, the methods described herein are directed to an in vitro method of identifying from a library or collection of compounds a compound comprising an epitope that induces immune tolerance in a human subject suffering from a disease comprising the steps of (a) contacting one or more live immune cells of the subject with (i) a labeled compound comprising an epitope in the presence of an epitope binding agent under conditions in which the compound binds to a T-cell receptor on a responder T-cell; and (ii) a labeled probe specific for a responder T-cell-specific surface marker; (b) identifying or isolating responder T-cells bound to the probe of step (a)(ii) by flow cytometry; (c) detecting or measuring an amount of the labeled compound of step (a)(i) bound to the T-cell receptors of the responder T-cells of step (b) by flow cytometry; (d) fixating and permeabilizing the cells of step (c); (e) contacting the responder T-cells of step (d) with (i) a labeled nucleic acid probe that is complementary to a target nucleic acid that is part of a gene that codes for a pro-inflammatory cytokine or for a receptor of a pro-inflammatory cytokine; and (ii) an antibody to a protein target; and (f) detecting or measuring an amount of signal generated by the probe of step (e)(i) and the antibody of step (e)(ii) that is hybridized to the target nucleic acid in the cell using flow cytometry, wherein the presence or quantity of the target nucleic acid, the intracellular target or both the target nucleic acid and the intracellular target in the cell identifies an epitope that induces immune tolerance in the subject. In particular embodiments, the target nucleic acid of step (e)(i) is mRNA. In particular embodiments, the mRNA codes for a pro-inflammatory cytokine selected from IFNγ, TNFα, I, IL-6, IL-12, IL-17, IL-18, IL-15, IL-8, IL-21, IL-25 and combinations thereof. In further embodiments, the mRNA codes for a protein used to lyse target cells, including, but not limited to, perforin and granzyme B. In other embodiments, the mRNA codes for a receptor for a pro-inflammatory cytokine selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof. In various embodiments, the antibody of step (e)(ii) binds to a marker selected from CD8, CD16, CD16, CD56, CD4, CD3, CD69, CD45RO, Tbet, Nk1.1, NKG2D and combinations thereof.

In various embodiments, the disclosure relates to an in vitro method of identifying a compound from a library or collection of compounds comprising an epitope that induces immune responsiveness in a human subject suffering from a disease comprising the steps of (a) exposing (i) an amount of responder T-cells from a healthy individual, and (ii) an amount of responder T-cells from the subject to the compound in the presence of an epitope binding agent; (b) incubating the cells of step (a) for a period of time sufficient to allow cell activation; (c) fixating and permeabilizing the cells of step (b); (d) contacting the cells of step (c) with a nucleic acid probe that is complementary to a nucleic acid coding for a responder T-cell marker of cell activity; (e) detecting using flow cytometry (i) an amount of signal generated by the probe of step (d) hybridized to responder T-cells from the healthy individual (RespH); and (ii) an amount of signal generated by the probe of step (d) hybridized to responder T-cells from the subject (RespS); (f) detecting or measuring (i) the amount of signal generated in step (e)(i) (RespH); and (ii) the amount of signal generated in step (e)(ii)(RespS), wherein the compound that induces a RespS/RespH≤1 is identified as a candidate compound for inducing immune tolerance in the subject. In certain embodiments, the method further includes before step (b) a step of washing the cells of step (a) to remove unbound compound. In various embodiments, the responder T-cells have the CD4+CD25− phenotype. In particular embodiments, the responder T-cell marker is selected from IFNγ, TNFα, IL1, IL-6, IL-12, IL-17, IL-18, IL-15, IL-8, IL-21, IL-25 and combinations thereof. In other embodiments, the responder T-cell marker is selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof.

In particular embodiments, the present disclosure relates to a method of identifying a compound comprising an epitope from a library or collection of epitopes that induces immune responsiveness in a subject suffering from a disease comprising the steps of (a) exposing responder T-cells from the subject to the compound in the presence of an epitope binding agent; (b) incubating the cells of step (a) for a period of time sufficient to allow cell activation; (c) fixating and permeabilizing (i) the responder T-cells of step (b), and (ii) an equal amount of control responder T-cells that have not been exposed to the compound; (d) contacting (i) the responder T-cells of step (c)(i) and (ii) the control responder T-cells of step (c)(ii) with a nucleic acid probe that is complementary to a nucleic acid sequence associated with a pro-inflammatory response under conditions in which the nucleic acid probe hybridizes to a complementary nucleic acid sequence in the T-cells; (e) detecting using flow cytometry (i) an amount of signal (S₁) generated by the probe that is hybridized to the complementary target nucleic acid sequence in the responder T-cells of step (d)(i), and (ii) an amount of signal (S₀) generated by the probe that is hybridized to the control responder T-cells of step (d)(ii); (f) detecting or measuring (i) the amount of signal (S₁) generated in step (e)(i), and (ii) the amount of signal (S₀) generated in step (e)(ii),wherein the compound that induces a S₁/S₀>1 or a S₀/S₁≤1 is identified as a candidate compound for inducing immune responsiveness in the subject.

In particular embodiments, the disclosure further relates to method of identifying a compound comprising an epitope from a library or collection of epitopes that induces immune responsiveness in a subject suffering from a disease comprising the steps of (a) exposing (i) responder T-cells from the subject, and (ii) responder T-cells from a healthy individual to the compound in the presence of an epitope binding agent; (b) incubating the cells of step (a) for a period of time sufficient to allow cell activation; (c) fixating and permeabilizing the cells of step (b); (d) contacting the cells of step (c) with a nucleic acid probe that is complementary to a nucleic acid sequence that is part of a gene associated with a pro-inflammatory response under conditions in which the nucleic acid probe hybridizes to a complementary nucleic acid sequence in the T-cell; (e) detecting using flow cytometry (i) an amount of signal generated by the probe that is hybridized to the complementary target nucleic acid sequence associated with a pro-inflammatory response in the responder T-cells of the subject (RespP), and (ii) an amount of signal generated by the probe that is hybridized to the complementary target nucleic acid sequence associated with a pro-inflammatory response in the responder T-cells of the healthy individual (RespH); and (f) detecting or measuring (i) the amount of signal (RespP) measured in step (e)(i), and (ii) the amount of signal (RespH) measured in step (e)(ii), wherein the compound that induces a RespH/RespP<1 or a RespP≥RespH is identified as a candidate compound for inducing immune responsiveness. In particular embodiments, the responder T-cells from the subject in step (a)(i) are in the presence of regulatory T-cells from the subject, and the responder T-cells from the healthy individual of step (a)(ii) are in the presence of an equal amount of regulatory T-cells (as in step (a)(i)) from the healthy individual.

In particular embodiments, the present disclosure relates to a method of identifying from a library or collection of compounds a compound comprising an epitope that induces immune responsiveness in a human subject suffering from a disease comprising the steps of (a) exposing a portion of T-resp cells to the compound in the presence of an epitope binding agent; (b) incubating the cells of step (a) for a period of time sufficient to allow cell activation; (c) fixating and permeabilizing the cells of step (b) and an equal amount of T-resp cells that were not exposed to the compound; (d) contacting the cells of step (c) with a nucleic acid probe that is complementary to a nucleic acid sequence present in one or more responder cells that is associated with a pro-inflammatory response in the cells under conditions in which the nucleic acid probe hybridizes to a complementary nucleic acid sequence in the T-resp cells; (e) detecting by flow cytometry (i) an amount of signal generated by the probe that is hybridized to the complementary target nucleic acid sequence associated with a pro-inflammatory response in the responder T-cells that were exposed to the compound, and (ii) an amount of signal generated by the probe that is hybridized to the complementary target nucleic acid sequence associated with a pro-inflammatory response in the equal amount of responder T-cells that were not exposed to the compound; and (f) detecting or measuring (i) the amount of signal generated in step (e)(i) (S₁), and (ii) the amount of signal generated in step (e)(ii) (S₀),wherein the compound that induces S₁/S₀>1 is identified as a candidate compound for inducing immune responsiveness in the subject. In various embodiments, the T-resp cells have the phenotype CD4+CD25−.

In some embodiments, the in vitro methods for identifying from a library or collection of compounds a compound comprising an epitope that induces immune responsiveness in a subject suffering from a disease are effected by a cell proliferation assay. Accordingly, in some embodiments, the present disclosure is directed to an in vitro method of identifying from a library or collection of compounds a compound comprising an epitope that induces immune responsiveness in a subject suffering from a disease comprising the steps of (a) exposing (i) an amount of CD4+ T-cells from the subject to the compound, and (ii) an equal amount of T-cells with T-reg cells depleted from the subject to the compound in the presence of an epitope binding agent and a fluorescent cell-staining dye; (b) incubating (i) the cells of step (a)(i), and (ii) the cells of step (a)(ii) for a period of time sufficient to allow cell proliferation; (c) fixating and permeabilizing (i) the cells of step (b)(i); and (ii) the cells of step (b)(ii); (d) contacting (i) the cells of step (c)(i), and (ii) the cells of step (c)(ii) with a cell surface marker for CD4, and a nucleic acid probe that is complementary to a target nucleic acid sequence that is part of a gene associated with a pro-inflammatory response present in one or more cells; (e) detecting by flow cytometry (i) a CD4 signal, and a fluorescent cell-staining dye signal in the cells of step (d)(i); and (ii) a CD4 signal, and a fluorescent cell-staining dye signal in the cells of step (d)(ii); and (f) detecting or measuring by flow cytometry (i) an amount of T-resp cells in step (e)(i) having a low fluorescent cell-staining dye signal (T-Resp1); and (ii) an amount of T-resp cells in step (e)(ii) having a low fluorescent cell-staining dye signal (T-Resp2), wherein the compound that induces a T-Resp1≤T-Resp2 is identified as a candidate compound for inducing immune responsiveness in the subject. In various embodiments, the T-reg cells have a CD4+CD25+ phenotype, and the T-resp cells have a CD4+CD25− phenotype.

In various embodiments, the present disclosure relates to an in vitro method of identifying from a library or collection of compounds a compound comprising an epitope that induces immune responsiveness in a human subject suffering from a disease comprising the steps of (a) exposing (i) an amount of T-reg from the subject, and (ii) an equal amount of T-resp cells from the subject to the compound in the presence of an epitope binding agent and a fluorescent cell-staining dye; (b) incubating (i) the cells of step (a)(i), and (ii) the cells of step (a)(ii) for a period of time sufficient to allow cell proliferation; (c) detecting by flow cytometry (i) a CD4 signal, and a fluorescent cell-staining dye signal in the cells of step (b)(i); and (ii) a CD4 signal, and a fluorescent cell-staining dye signal in the cells of step (b)(ii); and (d) detecting or measuring by flow cytometry (i) an amount of T-reg cells in step (c)(i) having a low fluorescent cell-staining dye signal (RegS); and (ii) an amount of T-resp cells in step (c)(ii) having a low fluorescent cell-staining dye signal (RespS), wherein the compound that induces an RegS≤RespS or an RespS>RegS is identified as a candidate compound for inducing immune responsiveness in the subject. In certain embodiments, the T-reg cells have the CD4+CD25+Foxp3+ phenotype, and the T-resp cells have the CD4+CD25−Foxp3− phenotype.

In various embodiments, the present disclosure relates to an in vitro method of identifying from a library or collection of compounds a compound comprising an epitope that induces immune responsiveness in a human subject suffering from a disease (a) exposing (i) an amount of CD4+CD25+Foxp3+ T-cells from the subject (RegS), (ii) an equal amount of CD4+CD25+Foxp3+ T-cells in step (a)(i) from a healthy individual (RegH), (iii) an amount of CD4+CD25−Foxp3− T-cells from the subject (RespS), and (iv) an equal amount of CD4+CD25−Foxp3− T-cells in step (a)(iii) from a healthy individual (RespH) to the compound in the presence of an epitope binding agent and a fluorescent cell-staining dye; (b) separately incubating the cells of steps (a)(i), (a)(ii), (a)(iii) and (a)(iv) for a period of time sufficient to allow cell proliferation; (c) detecting by flow cytometry (i) a CD4 signal, and a fluorescent cell-staining dye signal in the cells of step (a)(i); (ii) a CD4 signal, and a fluorescent cell-staining dye signal in the cells of step (a)(ii); (iii) a CD4 signal, and a fluorescent cell-staining dye signal in the cells of step (a)(iii); and (iv) a CD4 signal, and a fluorescent cell-staining dye signal in the cells of step (a)(iv); and (d) detecting or measuring by flow cytometry (i) an amount of T-reg cells from the subject having a low fluorescent cell-staining dye signal (RegS); (ii) an amount of T-reg cells from the healthy individual having a low fluorescent cell-staining dye signal (RegH); (iii) an amount of RespS cells having a low fluorescent cell-staining dye signal (RespS); and (iv) an amount of RespH cells having a low fluorescent cell-staining dye signal (RespH), wherein the compound that induces a RespH/RespS<1, a RegH/RegS≥1 or a RespH/RespS<1 and a RegH/RegS≥1 is identified as a candidate compound for inducing immune responsiveness.

In particular embodiments, the present disclosure relates to an in vitro method of identifying from a library or collection of compounds a compound comprising an epitope that induces immune responsiveness in a human subject suffering from an immune-related disorder comprising the steps of (a) exposing (i) an amount of T-resp cells from the subject in the presence of T-reg cells from the subject; and (ii) an equal amount of T-resp cells from the subject in the absence of T-reg cells to the compound in the presence of an epitope binding agent and a fluorescent cell-staining dye; (b) incubating (i) the T-resp cells in the presence of T-reg cells of step (a)(i), and (ii) the T-resp cells of step (a)(ii) in the presence of the compound for a time sufficient to allow cell proliferation; (c) detecting by flow cytometry (i) an amount of T-resp cells of step (b)(i) having a low fluorescent cell-staining dye signal; and (ii) an amount of T-resp cells of step (b)(ii) having a low fluorescent cell-staining dye signal; (d) detecting or measuring by flow cytometry (i) an amount of T-resp cells of step (c)(i) having a low fluorescent cell-staining dye (RespS₁), and (ii) an amount of T-resp cells of step (c)(ii) having a low fluorescent cell-staining dye signal (RespS₀), wherein the compound that induces a RespS₀>RespS₁ is identified as a candidate compound for inducing immune responsiveness in the subject. In particular embodiments, the T-resp cells have the CD4+CD25−Foxp3− phenotype and the T-reg cells have the CD4+CD25+Foxp3+ phenotype.

In various embodiments, the present disclosure relates to an in vitro method of identifying a compound from a library or collection of compounds comprising an epitope that induces immune responsiveness in a subject suffering from an immune-mediated disorder comprising the steps of (a) exposing (i) an amount of T-resp cells from a healthy individual to the compound in the presence of T-reg cells from the healthy individual for a period of time to allow cell proliferation, and (ii) an equal amount of T-resp cells from the subject to the compound in the presence of an equal amount of T-reg cells from the subject for a period of time to allow cell proliferation in the presence of a fluorescent cell-staining dye; (b) incubating (i) the T-resp cells of step (a)(i), and (ii) the T-resp cells of step (a)(ii) in the presence of the compound for a time sufficient to allow cell proliferation; (c) detecting by flow cytometry (i) an amount of T-resp cells of step (b)(i) having a low fluorescent cell-staining dye signal; and (ii) an amount of T-resp cells of step (b)(ii) having a low fluorescent cell-staining dye signal; (d) detecting or measuring by flow cytometry an amount of T-resp cells of step (c)(i) having a low fluorescent cell-staining dye signal (RespH); and (ii) an amount T-resp cells of step (c)(ii) having a low fluorescent cell-staining dye signal (RespS), wherein the compound that induces a RespH/RespS≤1 or a RespS/RespH>1 is identified as a candidate compound for inducing immune responsiveness in the subject. In various embodiments, the T-resp cells have the CD4+CD25− phenotype and the T-reg cells have the CD4+CD25+ phenotype.

In various embodiments, the present disclosure relates to methods of detecting in a T-cell memory for a particular epitope. In particular embodiments, the T-cells are selected from responder T-cells or a regulatory T-cells and can have various phenotypes as discussed above in Section 5.1. In certain embodiments, cell memory can be detected by proliferation of T-cells in the presence of an epitope or a compound comprising an epitope. In other embodiments, cell memory can be detected by a change in expression of various T-cell markers, such as an increase in one or more T-cell markers, a decrease in one or more T-cell markers, or a pattern of expression (increase and/or decrease) of T-cell markers as compared to T-cells that do not have memory for the epitope. In certain embodiments, the T-cell has lost expression of CD45RA and gained expression of CD45RO. In certain embodiments, the T-cell is a responder T-cell and the T-cell marker is selected from a pro-inflammatory cytokine selected from INFγ, TNFα, IL-1, IL-6, IL-12, IL-17, IL-18, IL-15, IL-8, IL-21, IL-25 and combinations thereof. In other embodiments, the T-cell is a responder T-cell and the T-cell marker is selected from a receptor for a pro-inflammatory cytokine selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof. In other embodiments, the T-cell is a regulatory T-cell and the T-cell marker is selected from an anti-inflammatory cytokine selected from IL-4, IL-10, IL-11, IL-13, TGFβ, IL-35, LIF, CTLA-4, CD39, galectin 1, galectin 3, galectin 9, PD-L1 and combinations thereof. In various embodiments, the T-cell is a regulatory T-cell and the T-cell marker is selected from a receptor for an anti-inflammatory cytokine selected from an IL-4 receptor, an IL-10 receptor, an IL-11 receptor, an IL-13 receptor, a TGFβ receptor, an IL-35 receptor, a LIF receptor, a CTLA-4 receptor, a CD39 receptor, a galectin 1 receptor, a galectin 3 receptor, a galectin 9 receptor, a PD-1 receptor and combinations thereof. In certain embodiments, the presence or quantity of T-cell markers is assayed by detecting or quantitating in the T-cells nucleic acids encoding one or more T-cell markers. In particular embodiments, the nucleic acids are mRNA.

In particular embodiments, the present disclosure relates to a method of determining the presence or absence of memory for an epitope in T-cells of a subject comprising the steps of (a) exposing (i) an amount of T-cells from the subject, and (ii) an equal amount of control T-cells to the epitope in the presence of an epitope binding agent for a period of time to allow cell proliferation in the presence of a fluorescent cell-staining dye; (b) incubating (i) the cells of step(a)(i), and (ii) the cells of step (a)(ii) in the presence of the compound for a period of time to allow cell proliferation; (c) detecting by flow cytometry (i) an amount of an amount of T-cells of step (b)(i) having a low fluorescent cell-staining dye signal; and (ii) an amount of T-cells of step (b)(ii) having a low fluorescent cell-staining dye signal; (d) detecting or measuring (i) an amount of T-cells of step (c)(i) having a low fluorescent cell-staining dye signal (S₁); and (ii) an amount of T-cells of step (c)(ii) having a low fluorescent cell-staining dye signal (S₀) wherein an S₁/S₀>1 or an S₀/S₁≤1 identifies memory for the epitope in the T-cells from the subject, and wherein an S₁/S₀≤1 or an S₀/S₁≥1 identifies the absence of memory for the epitope in the T-cells from the subject. In various embodiments, the control T-cells are naïve T-cells. In certain embodiments, the control T-cells are from an individual that has cell memory for the epitope. In other embodiments, the control T-cells are trained in vitro to have cell memory for the epitope.

In particular embodiments, the present disclosure relates to a method of determining the presence or absence of memory for an epitope in T-cells of a subject comprising the steps of (a) exposing (i) an amount of T-cells from the subject, and (ii) an equal amount of control T-cells to the epitope in the presence of an epitope binding agent; (b) incubating (i) the cells of step (a)(i), and (ii) the cells of step (a)(ii) for a period of time sufficient to allow cell activation; (c) fixating and permeabilizing (i) the cells of step (b)(i), and (ii) the cells of step (b)(ii); (d) contacting (i) the cells of step (c)(i) and (ii) the cells of step (c)(ii) with a nucleic acid probe that is complementary to a nucleic acid coding for a T-cell marker; (e) detecting by flow cytometry (i) an amount of signal generated by the probe hybridized to the T-cells of step (d)(i); and (ii) an amount of signal generated by the probe hybridized to the T-cells of step (d)(ii); (f) detecting or measuring by flow cytometry (i) the amount of signal (S₁) generated by the T-cells of step (e)(i), and (ii) the amount of signal (S₀) generated by the T-cells of step (e)(ii), wherein an S₁/S₀>1 or an S₀/S₁≤1 identifies memory for the epitope in the T-cells from the subject, and wherein an S₁/S₀<1 or an S₀/S₁≥1 identifies the absence of memory for the epitope in the T-cells from the subject.

In certain embodiments, the present disclosure relates to a method of determining the presence or absence of memory for an epitope in T-cells of a subject the steps of (a) contacting one or more T-cells with a labeled compound comprising the epitope in the presence of an epitope binding agent; and (b) detecting or measuring by flow cytometry an amount of the labeled compound of step (a) bound to the T-cells (S₁), wherein the presence or quantity of the labeled compound of step (a) bound to the T-cells is indicative of T-cell memory for the epitope. In particular embodiments, the method further comprises after step (b), the steps of (c) contacting one or more control T-cells with the labeled compound comprising the epitope of step (a); and (d) detecting or measuring by flow cytometry an amount of the labeled compound of step (c) bound to the control T-cells (S₀), wherein an S₁/S₀>1 or an S₀/S₁≤1 identifies memory for the epitope in the T-cells from the subject, and wherein an S₁/S₀≤1 or an S₀/S₁≥1 identifies the absence of memory for the epitope in the T-cells from the subject.

### 6.8. Methods for Prognosticating, Diagnosing, Monitoring an Immune-Mediated Disorder, and Monitoring Efficacy of Therapy

In certain aspects, the present disclosure is directed to methods of prognosticating and/or diagnosing an immune-mediated disorder in a subject. In other aspects, the present disclosure is directed to monitoring the efficacy of therapy in a subject suffering from an immune-mediated disorder.

Accordingly, in some embodiments, the present disclosure relates to a method of predicting or diagnosing an immune-mediated disorder in a subject comprising (a) fixating and permeabilizing (i) responder T-cells from the subject and responder T-cells from a healthy individual, and (ii) regulatory T-cells from the subject and regulatory T-cells from a healthy individual; (b) contacting (i) the responder T-cells from step (a)(i) with a nucleic acid probe that is complementary to a nucleic acid sequence present in one or more responder T-cells and that is associated with an inflammatory response under conditions in which the nucleic acid probe hybridizes to the nucleic acid sequence in the responder T-cells, and (ii) the regulatory T-cells from step (a)(ii) with a nucleic acid probe that is complementary to a nucleic acid sequence that is present in one or more regulatory T-cells and that is associated with an anti-inflammatory response under conditions in which the nucleic acid probe hybridizes to the nucleic acid sequence in the responder T-cells; (c) detecting by flow cytometry an amount of signal from the responder T-cells of the healthy individual (RespH); (ii) an amount of signal from the responder T-cells of the subject (RespP); (iii) an amount of signal from the regulatory T-cells of the healthy individual (RegH); and (iv) an amount of signal from the regulatory T-cells of the subject (RegP); and (d) detecting or measuring (i) the amount of signal generated in step (e)(i) (RespH); (ii) the amount of signal generated in step (e)(ii) (RespP); (iii) the amount of signal generated in step (e)(iii) (RegH); and (iv) the amount of signal generated in step (e)(iv) (RegP), wherein a comparison of RespH and RespP, of Reg H and Reg P or of both RespH and RespP and RegH and RegP that indicates a deviation of the subject's response from that of the healthy individual is indicative of an immune-mediated disorder in the subject.

Accordingly, in certain embodiments, the nucleic acid sequence associated with an inflammatory response is part of a gene that codes for an inflammatory cytokine, such as INFγ, TNFα, IL-1, IL-6, IL-12, IL-17, IL-18 and combinations thereof or is part of a gene that codes for a receptor for a pro-inflammatory cytokine, such as an INFγ receptor, a TNFα receptor, an IL-1 receptor, an IL-6 receptor, an IL-12 receptor, an IL-17 receptor, such as IL-17RC, an IL-18 receptor, or combinations thereof. In various embodiments, the nucleic acid sequence associated with an anti-inflammatory response is part of a gene that codes for an anti-inflammatory cytokine, such as IL-4, IL-10, IL-11, IL-13, TGFβ and combinations thereof, or is part of a gene that codes for a receptor for an anti-inflammatory cytokine such as an IL-4 receptor, an IL-10 receptor, an IL-11 receptor, an IL-13 receptor, a TGFβ receptor and combinations thereof. Thus, in certain embodiments, the nucleic acid probe hybridizes to mRNA that encodes, e.g., a pro-inflammatory cytokine or a receptor for a pro-inflammatory cytokine, or an anti-inflammatory cytokine or a receptor for an anti-inflammatory cytokine. In other embodiments, the nucleic acid probe hybridizes to a non-coding RNA, such as miRNA that functions in RNA silencing and post-transcriptional regulation of gene expression. In various embodiments, the methods described herein employ probes to more than one target.

In particular embodiments a RespP>RespH, a RegP<RegH or both a RespP>RespH and a RegP<RegH is indicative of the presence of an immune-mediated disorder in the subject. In other embodiments, a RespP>RespH, a RegP<RegH or both a RespP>RespH and a RegP<RegH is indicative of future onset of an immune-mediated disorder in the subject.

In various embodiments, the present disclosure relates to a method of monitoring an immune-mediated disorder in a subject comprising (a) fixating and permeabilizing a first responder T-cell from the subject, (b) contacting the first responder T-cell from step (a) with a nucleic acid probe that is complementary to a nucleic acid sequence present in one or more responder T-cells and that is associated with an inflammatory response under conditions in which the nucleic acid probe hybridizes to the nucleic acid sequence in the responder T-cell, (c) measuring an amount of signal generated by the probe hybridized to the nucleic acid of interest in the first cell sample using flow cytometry(RespP₁); (d) fixating and permeabilizing a second responder T-cell from the subject; (e) contacting the second responder T-cell from step (d) with a nucleic acid probe that is complementary to a nucleic acid sequence present in one or more responder T-cells and that is associated with an inflammatory response under conditions in which the nucleic acid probe hybridizes to the nucleic acid sequence in the responder T-cell, (e) measuring an amount of signal generated by the probe hybridized to the nucleic acid of interest in the second cell sample using flow cytometry(RespP₂), and (f) comparing the amount of signal measured in step (c) (RespP₁) with the amount of signal measured in step (e) (RespP₂), wherein the level of the target nucleic acid measured in step (c) compared to the level of the target nucleic acid measured in step (e) is correlated with the presence or severity of the immune-mediated disorder.

In particular embodiments, the method further comprises a step of contacting the fixated and permeabilized cells in steps (b) and (e) with a control nucleic acid probe under conditions in which the probe hybridizes to a complementary nucleic acid sequence in the cell. In some embodiments, a RespP₂>RespP₁ is indicative of progression of the immune-mediated disorder. In other embodiments, a RespP₂<RespP₁ is indicative of a lack of progression of the immune-mediated disorder. In certain embodiments, the second cell sample is collected at least about 1 week, at least about 2 weeks, at least about 3 weeks, at least about 1 month, at least about 6 months, or at least about 1 year or more after collection of the first cell sample.

In other embodiments, the present disclosure relates to a method of monitoring an immune-mediated disorder in a subject comprising (a) fixating and permeabilizing a first regulatory T-cell from the subject, (b) contacting the first regulatory T-cell from step (a) with a nucleic acid probe that is complementary to a nucleic acid sequence present in one or more regulatory T-cells and that is associated with an anti-inflammatory response under conditions in which the nucleic acid probe hybridizes to the nucleic acid sequence in the regulatory T-cell, (c) measuring an amount of signal generated by the probe hybridized to the nucleic acid of interest in the first cell sample using flow cytometry(RegP₁); (d) fixating and permeabilizing a second regulatory T-cell from the subject; (e) contacting the second regulatory T-cell from step (d) with a nucleic acid probe that is complementary to a nucleic acid sequence present in one or more regulatory T-cells and that is associated with an anti-inflammatory response under conditions in which the nucleic acid probe hybridizes to the nucleic acid sequence in the regulatory T-cell, (e) measuring an amount of signal generated by the probe hybridized to the nucleic acid of interest in the second cell sample using flow cytometry (RegP₂), and (f) comparing the amount of signal measured in step (c) (RegP₁) with the amount of signal measured in step (e) (RegP₂), wherein the level of the target nucleic acid measured in step (c) (RegP₁) compared to the level of the target nucleic acid measured in step (e) (RegP₂) is correlated with the presence or severity of the immune-mediated disorder.

In particular embodiments, the method further comprises a step of contacting the fixated and permeabilized cells in steps (b) and (e) with a control nucleic acid probe under conditions in which the probe hybridizes to a complementary nucleic acid sequence in the cell. In some embodiments, a RegP₁>RegP₂ is indicative of progression of the immune-mediated disorder. In other embodiments, a RegP₁≤RegP₂ is indicative of a lack of progression of the immune-mediated disorder. In yet other embodiments, the second cell sample is collected at least about 1 week, at least about 2 weeks, at least about 3 weeks, at least about 1 month, at least about 6 months, or at least about 1 year or more after collection of the first cell sample.

In still other embodiments, the disclosure relates to methods of monitoring an immune-mediated disorder in a subject comprising the steps of (a) fixating and permeabilizing (i) a first regulatory T-cell from the subject and (ii) a first responsive T-cell from the subject, (b) contacting (i) the first regulatory T-cell from step (a)(i) with a nucleic acid probe that is complementary to a nucleic acid sequence present in one or more regulatory T-cells and that is associated with an anti-inflammatory response under conditions in which the nucleic acid probe hybridizes to the nucleic acid sequence in the regulatory T-cell, and (ii) the first responder T-cell from step (a)(ii) with a nucleic acid probe that is complementary to a nucleic acid sequence present in one or more responder T-cells and that is associated with a pro-inflammatory response under conditions in which the nucleic acid probe hybridizes to the nucleic acid sequence in the responder T-cell, (c) measuring (i) an amount of signal generated by the probe hybridized to the nucleic acid of interest in the first regulatory cell sample of step (a)(i) using flow cytometry(RegP₁), and (ii) an amount of signal generated by the probe hybridized to the nucleic acid of interest in the first responder cell sample of step (a)(ii) using flow cytometry (RespP₁); (d) fixating and permeabilizing (i) a second regulatory T-cell from the subject and (ii) a second responder T-cell from the subject; (e) contacting (i) the second regulatory T-cell from step (d)(i) with a nucleic acid probe that is complementary to a nucleic acid sequence present in one or more regulatory T-cells and that is associated with an anti-inflammatory response under conditions in which the nucleic acid probe hybridizes to the nucleic acid sequence in the regulatory T-cell, and (ii) the second responder T-cell from step(d)(ii) with a nucleic acid probe that is complementary to a nucleic acid sequence present in one or more responder T-cells and that is associated with a pro-inflammatory response under conditions in which the nucleic acid probe hybridizes to the nucleic acid sequence in the responder T-cell; (e) measuring (i) an amount of signal generated by the probe hybridized to the nucleic acid of interest in the second regulatory T-cell using flow cytometry (RegP₂), and (ii) an amount of signal generated by the probe hybridized to the nucleic acid of interest in the in the second responder T-cell using flow cytometry (RespP₂), and (f) comparing the amount of signal measured in step (c)(i) (RegP₁) with the amount of signal measured in step (e)(i) (RegP₂), and the amount of signal measured in step (c)(ii) (RespP₁), and the amount of signal measured in step (e)(ii) (RespP₂), wherein the level of the target nucleic acid measured in step (c)(i) (RegP₁) compared to the level of the target nucleic acid measured in step (e)(i) (RegP₂), the level of the target nucleic acid measured in step (c)(ii) (RespP₁) compared to the level of the target nucleic acid measured in step (e)(ii) (RespP₂), or the level both the target nucleic acid measured in step (c)(i) (RegP₁) compared to the level of the target nucleic acid measured in step (e)(i) (RegP₂) and the level of the target nucleic acid measured in step (c)(ii) (RespP₁) compared to the level of the target nucleic acid measured in step (e)(ii) (RespP₂) is correlated with the presence or severity of the immune-mediated disorder.

In other embodiments, the present disclosure relates to a method of determining the efficacy of treatment in a patient suffering from an immune-mediated disorder comprising the steps of: (a) fixating and permeabilizing a first regulatory T-cell sample from the subject before treatment; (b) contacting the first regulatory T-cell sample from step (a) with a nucleic acid probe that is complementary to a nucleic acid sequence present in one or more regulatory T-cells and that is associated with an anti-inflammatory response under conditions in which the nucleic acid probe hybridizes to the nucleic acid sequence in the regulatory T-cell; (c) measuring an amount of signal generated by the probe hybridized to the nucleic acid in the first regulatory T-cell sample using flow cytometry (RegP₁); (d) fixating and permeabilizing a second regulatory T-cell sample from the subject after treatment; (e) contacting the second cell sample with the nucleic acid probe of step (b); (f) measuring an amount of signal generated by the probe hybridized to the nucleic acid of interest in the second regulatory T-cell sample using flow cytometry (RegP₂); and (g) comparing the amount of signal measured in step (c) with the amount of signal measured in step (f), wherein the level of the target nucleic acid measured in step (c) compared to the level of the target nucleic acid measured in step (f) is correlated with the presence or severity of the disease. In certain embodiments a RegP₂≥RegP₁ is indicative of the efficacy of treatment of the immune-mediated disorder.

In other embodiments, the present disclosure relates to a method of determining the efficacy of treatment in a patient suffering from an immune-mediated disorder comprising the steps of: (a) fixating and permeabilizing a first responder T-cell sample from the subject before treatment; (b) contacting the first responder T-cell sample from step (a) with a nucleic acid probe that is complementary to a nucleic acid sequence present in one or more responder T-cells and that is associated with a pro-inflammatory response under conditions in which the nucleic acid probe hybridizes to the nucleic acid sequence in the responder T-cell; (c) measuring an amount of signal generated by the probe hybridized to the nucleic acid in the first responder T-cell sample using flow cytometry (RespP₁); (d) fixating and permeabilizing a second responder T-cell sample from the subject after treatment; (e) contacting the second cell sample with the nucleic acid probe of step (b); (f) measuring an amount of signal generated by the probe hybridized to the nucleic acid of interest in the second responder T-cell sample using flow cytometry (RespP₂); and (g) comparing the amount of signal measured in step (c) with the amount of signal measured in step (f), wherein the level of the target nucleic acid measured in step (c) compared to the level of the target nucleic acid measured in step (f) is correlated with the presence or severity of the disease. In certain embodiments a RespP₁≥RespP₂ is indicative of the efficacy of treatment of the immune-mediated disorder.

In other embodiments, the present disclosure relates to determining the efficacy of treatment in a patient suffering from an immune-mediated disorder comprising the steps of: (a) fixating and permeabilizing (i) a first responder T-cell sample from the subject and (ii) a first regulatory T-cell sample before treatment; (b) contacting (i) the first responder T-cell sample from step (a)(i) with a nucleic acid probe that is complementary to a nucleic acid sequence present in one or more responder T-cells and that is associated with a pro-inflammatory response under conditions in which the nucleic acid probe hybridizes to the nucleic acid sequence in the responder T-cell, and (ii) the first regulatory T-cell sample from step (a)(ii) with a nucleic acid probe that is complementary to a nucleic acid sequence present in one or more regulatory T-cells and that is associated with an anti-inflammatory response under conditions in which the nucleic acid probe hybridizes to the nucleic acid sequence in the responder T-cell; (c) measuring (i) an amount of signal generated by the probe hybridized to the nucleic acid in the first responder T-cell sample using flow cytometry (RespP₁), and (ii) an amount of signal generated by the probe hybridized to the nucleic acid in the first regulatory T-cell sample using flow cytometry; (d) fixating and permeabilizing (i) a second responder T-cell sample from the subject after treatment, and (ii) a second regulatory T-cell sample from the subject after treatment; (e) contacting (i) the second responder T-cell sample with the nucleic acid probe of step (a)(i), and (ii) the second regulator T-cell sample with the nucleic acid probe of step (a)(ii); (f) measuring (i) an amount of signal generated by the probe hybridized to the nucleic acid of interest in the second responder T-cell sample using flow cytometry (RespP₂); and (ii) an amount of signal generated by the probe hybridized to the nucleic acid of interest in the second regulatory T-cell sample using flow cytometry (RegP₂); and (g) comparing the amount of signal measured in step (c)(i) with the amount of signal measured in step (f)(i) and the amount of signal measured in step (c)(ii) with the amount of signal measured in step (f)(ii), wherein the level of the target nucleic acid measured in step (c)(i) (RespP₁) compared to the level of the target nucleic acid measured in step (f)(i) (RespP₂) and the level of target nucleic acid measured in step (c)(ii) (RegP₁) compared to the level of target nucleic acid measured in step (f)(ii) (RegP₂) are correlated with the presence or severity of the disease. In certain embodiments a RespP₂<RespP₁, a RegP₂≥RegP₁, or a RespP₂<RespP₁, and a RegP₂≥RegP₁ is indicative of the efficacy of treatment of the immune-mediated disorder.

In certain embodiments, the cell samples of (c)(ii) and (e)(ii) are collected at least about 1 day, at least about 1 week, at least about 2 weeks, at least about 3 weeks, at least about 1 month, at least about 6 months, or at least about 1 year or more after collection of the cell samples of (c)(i) and (e)(i), respectively.

Other methods of prognosticating, diagnosing, and/or monitoring the efficacy of therapy in a subject suffering from an immune-related disorder, and in particular, an autoimmune disease, can be found in U.S. patent application Ser. No. 13/871,730 filed Apr. 26, 2013 and PCT application no. PCT/US14/35549 filed Apr. 25, 2014, the contents of all of which are incorporated by reference herein in their entirety.

### 6.9. Immune Profiling and Therapy for Immune-Mediated Disorders

In various embodiments, the present disclosure relates to methods of profiling the immune state of a subject suffering from an immune-mediated disorder using flow cytometry. In particular embodiments, the present disclosure relates to methods of profiling a subject suffering from a disease such as cancer or an infection. In some embodiments, the present disclosure relates to profiling a subject who has a family history of immune-mediated disorders or cancer. In various embodiments, the subject is asymptomatic. In particular embodiments, the immune profile of the subject is compared to the immune profile of a healthy individual, i.e., an individual who is not suffering from an immune-mediated disorder. In other embodiments, the immune profile of the subject is compared to the immune profile of an individual who does not have active disease, of an individual in remission, of an individual in partial remission or of an individual in which the disease is has been eradicated.

In various embodiments, an immune profile includes one or more of (i) associating immune cell phenotype (e.g., CD4+ cells depleted of CD25+ cells, CD25+ cells, Foxp3+ cells or particular subsets thereof) with cell responses to an epitope that induces immune tolerance or immune responsiveness, and/or with cell responses to disease (e.g., when challenged with antigens, or infected or diseased cells); (ii) assaying the presence or absence of immune cell memory to specific antigens or epitopes; (iii) detecting and/or quantifying pro-inflammatory and/or anti-inflammatory cytokine production in specific cells and/or correlating the cytokine production with immune cell phenotypes; (iv) assaying the number of responder T-cells and/or regulatory T-cells and/or the state of the cells (e.g., active, inactive, proliferating, anergic, and the like); (v) determining the presence or absence on specific immune cells of a receptor for a particular epitope; and (vi) correlating the state of a subject's immune cells (e.g., active/inactive) with the state of the disease or disorder (e.g., dormant/active) in the subject. In various embodiments, methods for providing an immune profile of a subject are set forth in Section 5.8, above.

In certain embodiments, immune profiling is used to identify a specific disorder in a subject. In particular embodiments, the disorder is an immune-mediated disorder. In other embodiments, the disorder is an infection, such as a viral infection, or a disease such as cancer. In various embodiments, a disorder is identified before symptoms appear. Accordingly, in certain embodiments, immune profiling is used to predict a disorder in the subject. In other embodiments, immune profiling is used to identify specific types of cells that are activated or inactivated in the subject. In various embodiments, immune profiling is used to determine the strength of immune responses or of tolerance induction in a subject, for example, as compared to the strength of immune responses or tolerance induction in a healthy individual. In particular embodiments, immune profiling is used to identify a compound comprising an epitope that induces immune tolerance in a subject suffering from an immune-mediated disorder. In other embodiments, immune profiling is used to identify a compound comprising an epitope that induces immune responsiveness in a subject suffering from a disease, such as cancer, or a viral infection. In other particular embodiments, immune profiling is used to optimize the efficacy of a lead compound comprising an epitope that induces immune tolerance in the subject suffering from an immune-mediated disorder, or to optimize the efficacy of a lead compound comprising an epitope that induces immune responsiveness in a subject suffering from a disease or infection. In various embodiments, immune profiling is used to optimize the efficacy of tumor-specific antigens or disorder-related antigens (such as S-antigen). In yet other embodiments, immune profiling is used to identify a therapy that is efficacious in a particular subject, to optimize a therapy for a particular subject or a specific disorder, and/or to monitor the effects of therapy in the subject.

In certain embodiments the present disclosure is directed to methods of treating a subject suffering from an immune-mediated disorder. In some embodiments, the immune-mediated disorder arises from an inappropriate immune response of a subject's body to self-antigens or foreign antigens, e.g., autoimmune disorders. In other embodiments, the immune-mediated disorder arises from a reduced or absent immune response to foreign antigens, e.g., infectious agents, and cancer (immunodeficiency disorders). In particular embodiments, a subject is treated by administering one or more compounds comprising an epitope that induces immune tolerance or immune responsiveness as described herein. When administered to a subject, a compound identified by the methods described herein can be administered as a component of a composition that comprises a pharmaceutically acceptable carrier or excipient. Compositions comprising the compound can be administered by injection or absorption through mucocutaneous linings (e.g., oral, rectal, and intestinal mucosa, etc.). Administration can be systemic or local. Methods of administration include, but are not limited to, intravenous, intramuscular, oral, sublingual, intravaginal, rectal, by inhalation and parenterally. Descriptions of various components that can be included in the compositions comprising a compound described herein can be found in U.S. patent application Ser. No. 13/871,730, filed Apr. 26, 2013, the contents of which are incorporated by reference herein in their entirety.

Specific examples of pharmaceutically acceptable carriers and excipients that can be used to formulate various dosage forms are described in the Handbook of Pharmaceutical Excipients, American Pharmaceutical Association (1986). In various embodiments, a composition of the invention is prepared by a method comprising admixing a compound or a pharmaceutically acceptable derivative thereof with a pharmaceutically acceptable carrier or excipient. Admixing can be accomplished using methods known for admixing compounds, e.g., a peptide, and a pharmaceutically acceptable carrier or excipient. In one embodiment, the compound is present in the composition in an effective amount.

In particular embodiments in which the compound comprises an epitope that induces immune tolerance, the compound is administered with an enhancer. As used herein, an “enhancer” is any compound or mixture of compounds that potentiates the immune suppressive response of T-reg cells. In some embodiments, the enhancer is high molecular weight hyaluronic acid. As used herein, the term “high molecular weight hyaluronic acid” refers to hyaluronic acid having a molecular weight of at least about 1×10⁶ Da, such as of at least about 2×10⁶ Da, at least about 3×10⁶ Da, at least about 4×10⁶ Da, or more. See e.g., Bollyky et al. (2007) J. Immunol. 179:744-747. Other enhancers include, but are not limited to, anti-inflammatory cytokines such as IL-2, TGF-β, IL-4, IL-10, IL-11, IL-13, IL-35, LIF, CTLA-4, CD39, galectin 1, galectin 3, galectin 9, PD-L1, all-trans retinoic acid, cyclosporine A, rapamycin, FK506, anti-CD3, anti-CD28, vitamin D3, dexamethasone, IL-10, idolamine-2,3-dioxygenase, FTY720, a sphingosine kinase 1 inhibitor, cholera toxin B subunit, ovalbumin, Flt2L, sirolimus and anti-thymocyte globulin, CTLA-4/Ig, and mixtures thereof. See, e.g., Viney et al. (1998) J. Immunol. 160(12):5815-25; Horwitz et al. (2004) Seminars in Immunol. 16:135-143; Daniel et al. (2007) J. Immunol. 178(2): 458-68; Weiner et al. (2011) Immunol Rev. 241(1):241-259; Ma et al. (2011) Int. Immunopharmacol. 11(5):618-29; Adriouch et al. (2011) Front. Microbiol. 2:199; Dons et al. (2012) Human Immunol. 73:328-334. In certain embodiments, the enhancer is a sphingosine kinase 1 inhibitor as disclosed in U.S. Pat. No. 8,872,888.

In other embodiments in which the compound comprises an epitope that induces immune responsiveness in a subject, the compound is administered with an adjuvant that boosts an immune response by, e.g., extending the presence of antigen in the blood, helping absorb the antigen presenting cells antigen, activating macrophages and lymphocytes, and supporting the production of cytokines. In various embodiments, the adjuvant is selected from alum, aluminum hydroxide, aluminum phosphate, calcium phosphate hydroxide, paraffin oil, squalene, thimerosal, detergents, pro-inflammatory cytokines such as IFNγ, TNFα, I, IL-6, IL-12, IL-17, IL-18, IL-15, IL-8, IL-21, IL-25, Freund's complete adjuvant, Freund's incomplete adjuvant, and products of infectious agents, e.g., killed bacteria or viruses. In further embodiments, the adjuvant is an inhibitor of anti-inflammatory cytokines, including but not limited to inhibitors of IL-2, TGF-β, IL-4, IL-10, IL-11, IL-13, IL-35, LIF, CTLA-4, CD39, galectin 1, galectin 3, galectin 0, PD-L1 or a receptor of any of the foregoing cytokines.

The amount of compound that is effective for the treatment or prevention of an immune-mediated disorder or a disease such as cancer or a viral infection can be determined by standard clinical techniques. In addition, in vitro and/or in vivo assays can optionally be employed to help identify optimal dosage ranges. The precise dose to be employed will also depend on, e.g., the route of administration and the seriousness of the condition, and can be decided according to the judgment of a practitioner and/or each subject's circumstances. In other examples thereof, variations will necessarily occur depending upon the weight and physical condition (e.g., hepatic and renal function) of the subject being treated, the affliction to be treated, the severity of the symptoms, the frequency of the dosage interval, the presence of any deleterious side-effects, and the particular compound utilized, and the number of administered compounds, among other things.

Administration can be as a single dose or as a divided dose. In one embodiment, an effective dosage is administered once per month until the condition is abated. In another embodiment, the effective dosage is administered once per week, or twice per week or three times per week until the condition is abated. In another embodiment, an effective dosage amount is administered about every 24 hours until the condition is abated. In another embodiment, an effective dosage amount is administered about every 12 hours until the condition is abated. In another embodiment, an effective dosage amount is administered about every 8 hours until the condition is abated. In another embodiment, an effective dosage amount is administered about every 6 hours until the condition is abated. In another embodiment, an effective dosage amount is administered about every 4 hours until the condition is abated. The effective dosage amounts described herein refer to total amounts administered; that is, if more than one compound is administered, the effective dosage amounts correspond to the total amount administered.

In various embodiments, the compound can be administered together with a second therapeutically active agent. In some embodiments, the additional agent is a dietary supplement such as a vitamin, a mineral, or an ω-3 fatty acid. In other embodiments in which the compound comprises an epitope that induces immune tolerance in the subject, the second therapeutically active agent is an anti-inflammatory agent, e.g., a corticosteroid. In further embodiments, the second therapeutically active agent is a synthetic drug, such as a non-steroidal anti-inflammatory drug (NSAID). In various embodiments in which the compound comprises an epitope that induces immune responsiveness in the subject, the second therapeutically active agent is a pro-inflammatory agent, e.g., lipopolysaccharide.

In one embodiment, a compound is administered concurrently with a second therapeutic agent as a single composition comprising an effective amount of the compound and an effective amount of the second therapeutic agent. Alternatively, a composition comprising an effective amount of a compound and a second composition comprising an effective amount of the second therapeutic agent are concurrently administered. In another embodiment, an effective amount of a compound is administered prior or subsequent to administration of an effective amount of the second therapeutic agent. In this embodiment, the compound is administered while the second therapeutic agent exerts its therapeutic effect, or the second therapeutic agent is administered while the compound exerts its therapeutic effect for treating or preventing a condition.

An effective amount of the second therapeutic agent will be known to the art depending on the agent. However, it is well within the skilled artisan's purview to determine the second therapeutic agent's optimal effective-amount range. In some embodiments of the invention, where a second therapeutic agent is administered to a subject for treatment of a condition, the minimal effective amount of the compound will be less than its minimal effective amount would be where the second therapeutic agent is not administered. In this embodiment, the compound and the second therapeutic agent can act synergistically to treat or prevent a condition.

In various embodiments, the present disclosure relates to methods of identifying an effective therapy for a subject suffering from an immune-mediated disorder or from a disease or infection. Accordingly, the present disclosure relates to a method of identifying an effective therapy in a subject suffering from an immune-mediated disorder, a disease or an infection comprising the steps of. (a) obtaining (i) a first cell sample from the subject taken before treatment, and (ii) a second cell sample from the subject taken after treatment; (b) fixating and permeabilizing (i) the cells of step (a)(i) and (ii) the cells of step (a)(ii); (c) contacting (i) the cells of step (b)(i), and (ii) the cells of step (b)(ii) with a nucleic acid probe that is complementary to a nucleic acid of interest; and a control nucleic acid probe, wherein the nucleic acid probes hybridize to complementary nucleic acid sequences in the cells; (d) detecting using flow cytometry (i) an amount of signal generated by the probe of step (c)(i); and (ii) an amount of signal generated by probe of step (c)(ii); (e) detecting or measuring by flow cytometry (i) an amount of signal generated in step (d)(i) (Sig₀), and (ii) an amount of signal generated in step (d)(ii) (Sig₁), wherein a Sig₁>Sig₀ identifies a therapy that is effective in the subject. In certain embodiments in which the subject is suffering from a disease or infection, the nucleic acid probe is complementary to a nucleic acid encoding a pro-inflammatory cytokine or a receptor for a pro-inflammatory cytokine. In other embodiments in which the subject is suffering from an immune-mediated disorder, the nucleic acid probe is complementary to a nucleic acid encoding an anti-inflammatory cytokine or a receptor for an anti-inflammatory cytokine.

In yet other embodiments, the present disclosure relates to a method of identifying an effective therapy in a subject suffering from an immune-mediated disorder, a disease or an infection comprising the steps of. (a) exposing (i) a first cell sample from the subject taken before treatment, and (ii) a second cell sample from the subject taken after treatment to a fluorescent cell-staining dye; (b) incubating (i) the cells of step (a)(i), and (ii) the cells of step (a)(ii) for a period of time to allow cell proliferation; (c) detecting or measuring by flow cytometry (i) an amount of cells of step (b)(i) having a low fluorescent cell-staining dye signal (Sig₀); and (ii) an amount of cells of step (b)(ii) having a low fluorescent cell-staining dye signal (Sig₁), wherein a Sig₁<Sig₀ identifies a therapy that is effective in the subject.

In various embodiments, the present disclosure is directed to methods of treating a subject suffering from an immune-mediated disorder or a disease or infection by administering T-reg cells that have been trained in the presence of a compound that comprises an epitope that induces immune tolerance in the T-reg cells or by administering T-resp cells that have been trained in the presence of a compound that comprises an epitope that induces immune responsiveness in the T-resp cells. In particular embodiments, the T-reg cells and/or the T-resp cells are from the subject. In other embodiments, the T-reg cells and/or the T-resp cells are from a healthy individual, from a subject that does not have active disease or from a subject in which the disease has been eradicated.

In particular embodiments, T-reg cells are trained in vitro in the presence of a compound comprising an epitope that induces immune tolerance. In other particular embodiments, T-resp cells are trained in vitro in the presence of a compound comprising an epitope that induces immune responsiveness. In other embodiments, T-reg cells are trained in vivo in the presence of a compound comprising an epitope that induces immune tolerance. In various embodiments, T-resp cells are trained in vivo in the presence of a compound comprising an epitope that induces immune responsiveness. In other embodiments, T-reg cells are trained in vivo in the presence of a compound comprising an epitope that induces immune tolerance. In these embodiments, the subject or a healthy individual is dosed with the compound comprising an epitope for a period of time to train the cells. In certain embodiments, T-cells trained either in vitro or in vivo are identified by the presence or quantity of a receptor for the epitope on the cell surface. In other embodiments, trained T-cells are identified by an increase in cytokine production. In additional embodiments, trained T-cells are identified by (i) the presence and/or quantity of a receptor for the epitope on the cell surface, and (ii) the presence of increased cytokine production. In particular embodiments, trained regulatory T-cells are identified by an increase in anti-inflammatory cytokine production. In other particular embodiments, trained responder T-cells are identified by an increase in pro-inflammatory cytokine production. In various embodiments, the presence and/or quantity of a receptor on the T-cell surface and/or detection and quantification of cytokine production can be measured using flow cytometry.

In certain embodiments, the present disclosure relates to a method of treating an immune-mediated disorder, a disease or infection in a subject comprising isolating regulatory T-cells or responder T-cells from the subject or from a healthy individual, training in vitro the regulatory T-cells or the responder T-cells in the presence of a compound comprising an epitope that induces immune tolerance in the regulatory T-cells or immune responsiveness in the responder T-cells, and then introducing the trained T-cells into the subject. In certain embodiments, responder T-cells or regulatory T-cells can be administered together with the compound comprising and epitope.

In various embodiments, the regulatory T-cells can be administered with an enhancer. In other embodiments, the responder T-cells can be administered with an adjuvant. In certain embodiments, the T-cells are expanded before being administered. In other embodiments, the T-cells are not expanded before being administered. In certain embodiments, T-cells are infused at a dose of at least about 0.1×10⁵/kg body weight, such as a dose of at least about 5×105 cells/kg body weight, at least about 10×10⁵ cells/kg, at least about 20×10⁵ cells/kg body weight, at least about 30×10⁵ cells/kg body weight, at least about 40×10⁵ cells/kg body weight, at least about 50×10⁵ cells/kg body weight, at least about 60×10⁵ cells/kg body weight, at least about 70×10⁵ cells/kg body weight, at least about 80×105 cells/kg body weight, at least about 90×105 cells/kg body weight, at least about 10×10⁶ cells/kg body weight, at least about 15×10⁶ cells/kg body weight, or at least about 20×10⁶ cells/kg body weight or more. T-reg cells are typically administered by injection or intravenous infusion. For infusion, T-cells are administered in a sterile, isotonic solution, for example, normal saline (e.g., 0.9% NaCl) and 5% human albumin or lactated Ringer's solution. Various aspects of T-cell therapies also can be found in U.S. patent application Ser. No. 13/871,730 filed Apr. 26, 2013, the contents of which are incorporated by reference herein in their entirety.

In particular embodiments, therapies contemplated herein include genetically modifying T-cells in order to, e.g., impart new antigen specificity, new antigen recognition and the like to the T-cells. In various embodiments, the T-cells are from the subject. In other embodiments, the T-cells are from a healthy individual. In particular embodiments, antigens on diseased cells, e.g., cancer cells, virally infected cells, are identified and receptors for one or more of the disease-associated or disorder associated antigens are cloned into the T-cells in vitro, which can then be grown, expanded and reintroduced into the subject in order to target and lyse the aberrant or infected cells. In yet other embodiments, responder T-cells or regulatory T-cells can be genetically modified to confer new or enhanced antigen specificity or to produce markers, e.g., CD3-zeta/CD137 that increase anti-tumor activity of the T-cells.

### 6.1. Identification and Correction of Ambiguity in Flow Cytometry Results

Methods and compositions are also provided that can reduce or eliminate false signals when homogeneous probe systems such as molecular beacons are used to detect and/or quantify cellular components in the interior of cells. It has been known that even though molecular beacons are only supposed to generate signals after hybridizing with complementary nucleic acids, they can generate signals by non-specific binding to cellular membranes and proteins in addition to nucleic acids that may be only partially complementary to the beacon sequences. The latter problem has been especially apparent in flow cytometry detection of nucleic acids in living cells where the lack of fixation leaves proteins and cellular matrices relatively intact. Non-specific binding of molecular beacons can result in different background levels from sample to sample, leading to large differences in data collected from sample to sample that interferes with calculations of the average target signals. As such, a simple subtraction of a single “background” value is not always sufficient to estimate the “true signal” value compared to the total signal detected.

Accordingly, since diagnostic flow cytometry methods should produce quality results that are reliable and measurable, it is essential to reduce any ambiguity that arises when using molecular beacons in an environment prone to generating false signals by minimizing signals from non-target molecules while retaining the ability to detect specific signal from the target of interest. Generation of non-specific signal from non-target molecules can either result in false negatives by losing the specific signal in the noise of the system, or by generating a spurious signal, a false positive result can be created, in either case leading to an inconclusive or erroneous diagnosis. In addition, when there is higher background in healthy cells, the difference between signals from healthy cells and high grade lesion cells is diminished possibly obscuring differential results that are diagnostic for the disease condition. To achieve medically relevant measurements using homogeneous probe systems inside of cells, the non-specific interactions of cell components and probes should be reduced as much as possible so that the signals and results are consistent across every patient sample.

Reduction of background for in situ detection of nucleic acids on slides is accomplished with the washing steps that are intrinsic to such systems. Such washing steps can involve stringency wash conditions to eliminate binding of probes with partial homology, but in addition, reagents such as salts and detergents are used to reduce sequence independent binding between probes and various constituents of the cell. Thus, for the mixed phase hybridizations that are part of traditional in situ hybridization assays, multiple washing steps with a variety of additives have been used as a common mode for maintaining specificity of signal generation. On the other hand, in systems that have used probes to detect nucleic acids in living cells, accessibility to the interior of target cells is a transient feature where cellular membranes reseal soon after introduction of the molecular probes. Thus, after introduction of the probes into the cells, there is no accessibility for any further manipulations of the ongoing hybridization and subsequent signal detection. As such, in the absence of being able to use wash steps, these systems have employed homogeneous probe systems that can evade the inability of removing unhybridized probes and signal generation is intended to take place strictly on the basis of binding of the probes with complementary sequences in targets. However, although stringency of hybridization can be carried out by means of appropriate selection of probe sequences, means for attenuating sequence-independent probe/matrix interactions are not readily approachable. Furthermore, another consequences of the vitality of the cells being probed is that the lack of a fixation step for in vivo studies allows continuous activity of nucleases such as RNases that may destroy targets and DNAses that may open up Molecular Beacon homogeneous probes where the first activity may allow true signals to be lost and the latter allowing false signals to be generated. Consequently, since washing reagents for decreasing intracellular interactions and/or reducing nuclease action have been impractical for living cells, most efforts to mitigate such problems have been to alter the constituents of the nucleic acids themselves by using various nucleotide analogues and modifications. This may have some effect on reduction of vulnerability to nuclease action but there has not been as much success at interactions of the probe with the cellular environment. In the present invention, the use of fixed cells has allowed inactivation of most nuclease activity and we now disclose the application of various reagents that can significantly reduce interactions between homogeneous signaling probes and cellular constituents when carrying out analysis by flow cytometry of fixed cells.

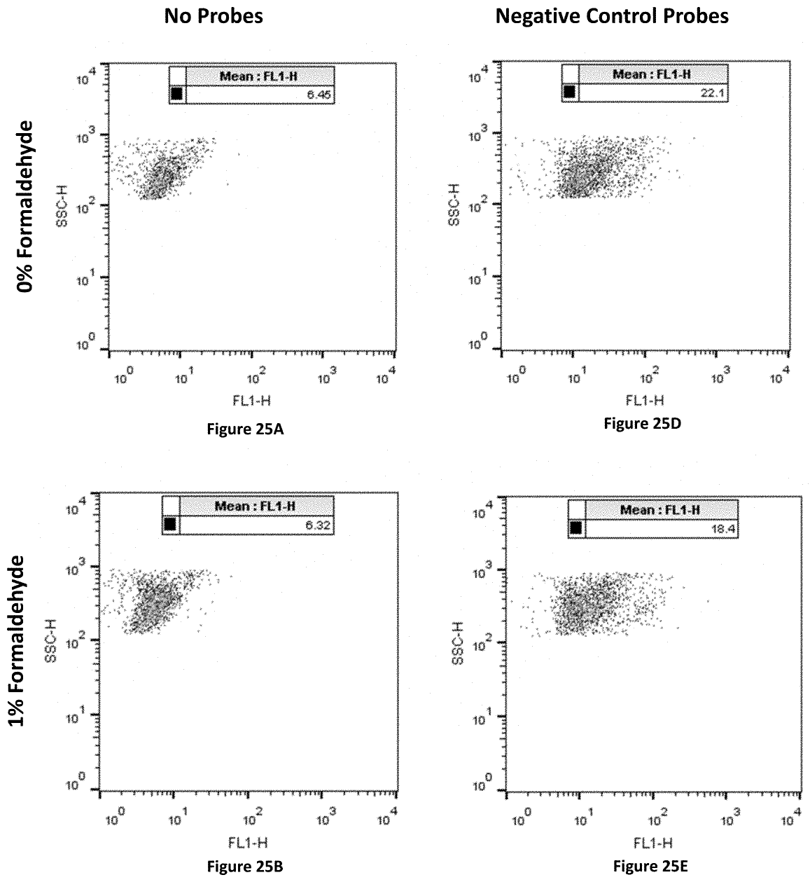

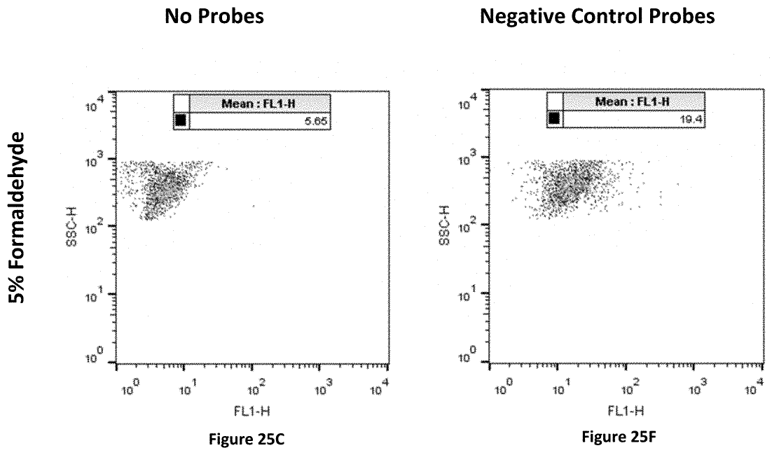

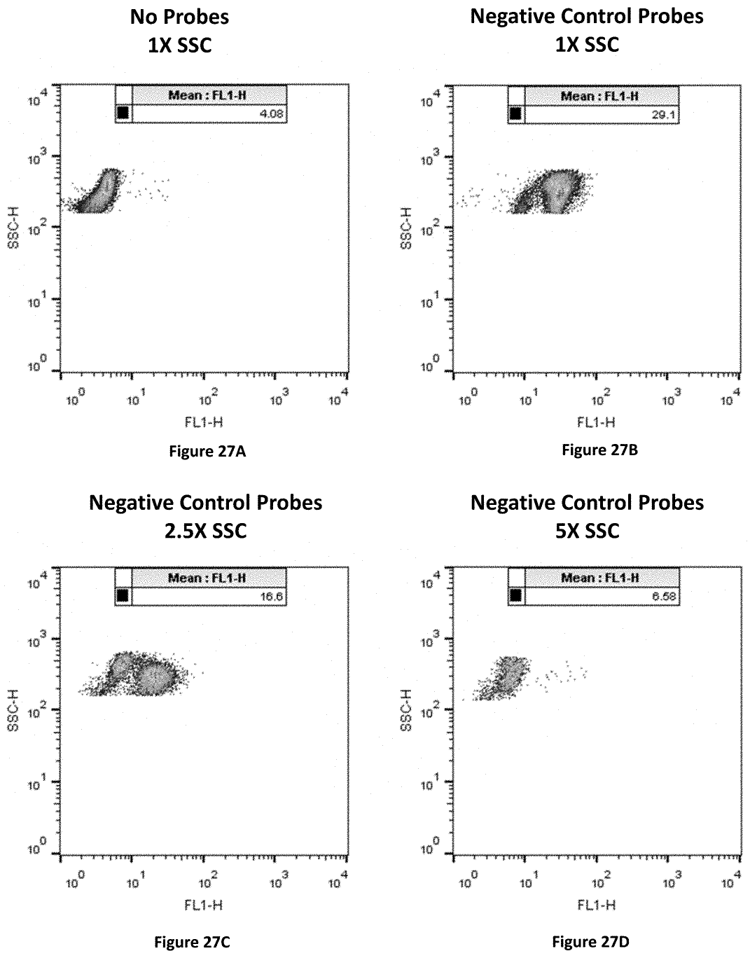

In order to lessen and/or eliminate the observed ambiguity, the present invention discloses that even in the absence of washing steps, the presence of various reagents during hybridization of homogeneous probes can reduce or eliminate sequence-independent signal generation. It is a discovery of the present invention, that although these reagents may have previously been used as part of a washing program, when using homogenous probes systems that do not require washing step, these reagents may be successfully employed during the hybridization process rather than the traditional post-hybridization usage. By separately analyzing “no probe” samples and “negative control probe” treatments, background derived from autofluorescence can be separated from background derived from interactions of labeled probes with the cellular environment and the influence of various reagents/treatments on each of these processes can be individually assessed. For instance, as seen in Example 28 and FIG. 25, alterations in formaldehyde concentrations has only minor effects upon background in the absence of any labeled probes (autofluorescence) but a stronger effect can be seen when using formaldehyde with the “negative control probe” which lacks homology with any RNA targets present in the cells. In this particular instance, it can be seen that the probe derived background is stronger in the HSIL patient as compared to cells from a healthy subject, but with treatment with either 1% or 5% formaldehyde, the normal cells exhibit more than 50% reduction in readings from non-target signal generation. In this particular instance, the formaldehyde is not being used during hybridization for stringency purposes but rather as a treatment of cells prior to the introduction and hybridization with homogenous probes and reduces the ability of these cells to bind nucleic acid probes non-specifically. Probe derived background can also be reduced during hybridization by raising the salt concentration where background that is seen at 1×SSC/2% Triton with the “Negative Control Probe” is increasingly eliminated as the salt concentration is raised (see Example 29, FIG. 27). In this particular example, at the highest level of salt tested (5×SSC) the probe derived non-specific signal is only slightly higher than the autofluorescence seen with the “no probe” control.

One of the explanation that has frequently been given for the background seen in live cells has been interactions of the Molecular Beacon probes with proteins that are resident in the cells. The potential for this effect can be seen in a description of an assay (Li et al., 2000 Angrew Chem Int Ed 39; 1049-1052) where Molecular Beacons have been used to detect and quantify Single-Stranded Binding (SSB) protein where binding between a Molecular Beacon and the SSB protein leads to signal generation presumably by opening up the stem. Since there are numerous DNA binding proteins in both fixated and unfixated cells, this is a possible explanation for some probe derived sequence independent signal generation. Indeed, it's not just with regard to proteins whose specific function is in binding to nucleic acids, observations have been made that even metabolic enzymes such as lactic dehydrogenase can interact with Molecular Beacons to generate signals (Fang et al., 2000 Anal Chem 72; 3280-3285) thus increasing the amount of potential partners in a cell. Consequently, it is a consideration of the present patent that reagents that provide disruption of protein-nucleic acid interactions can reduce background signal generation derived from homogeneous probes binding to cellular constituents.

Since nucleic acids represent a class of polyanions due to the presence of multiple phosphates, competitive inhibition can potentially be provided by polyanions other than nucleic acids that can reduce or eliminate signals caused by nucleic acid probes binding to cellular proteins or matrices. As such, examples of polyanions that could be used for this purpose can comprise negatively charged proteins, polysaccharides, glycosaminoglycans, proteoglycans and other molecules. A description of various polyanions that may be of interest are described in La Toya et al., “Polyanions and the Proteome” 2004 Molecular & Cellular Proteomics 3; 746-769; Luscher Mattli 2000 “Polyanions—a lost chance in the fight against HIV and other viral diseases?” Antiviral Chemistry & Chemotherapy 11; 249-259, both of which are incorporated by reference. Examples of negatively charged polypeptides can include but not be limited to actin, tubulin, casein, polyaspartic acid and human serum albumin modified by addition of either succinic acid or aconitic acid. Examples of polyanionic polysaccharides can include but not be limited to heparin, heparan sulfate, heparanoids, dermatan sulfate, dextran sulfate, chondroitin sulfates, carrageenan, pentosan polyphosphate, calcium spirulan and sulfated fucans. In addition, organic molecules that may find use can include but not be limited to large organic polyanions, such as polyethylene sulfonate, polycarboxylate pyran copolymer, divinylether maleic anhydride copolymer, poly(styrenesulfonate) and smaller organic polyanions such as suramin, Aurintricarboxylic acid (ATA), phytic acid and sucrose octasulfate.

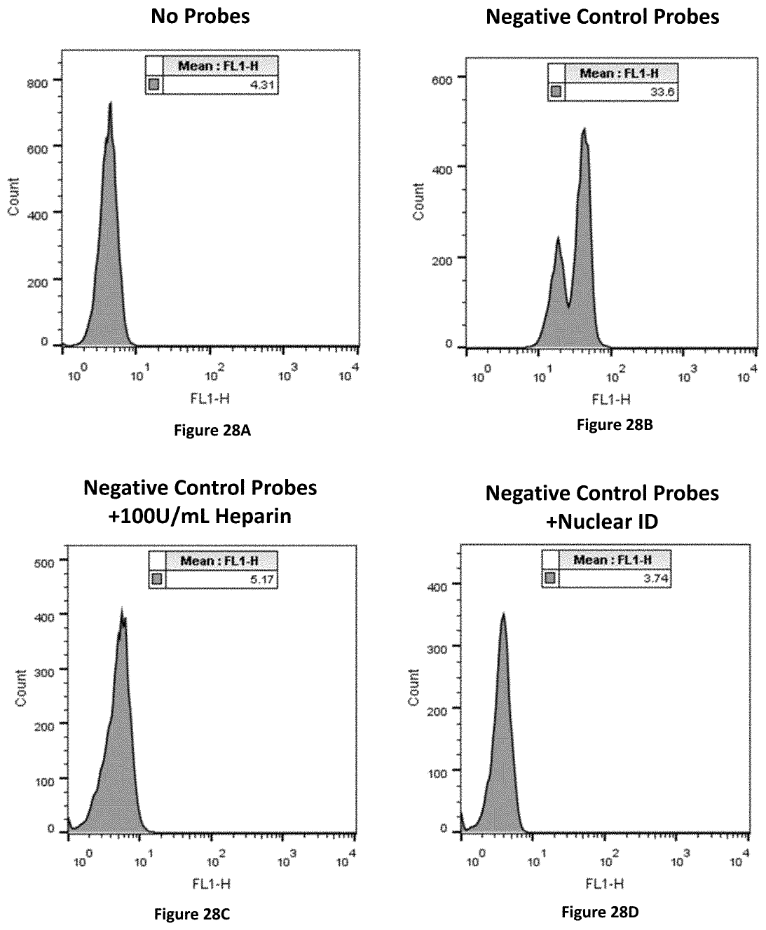

In Examples 29 and 30 two different polyanions were used with flow cytometry and homogeneous probes. In Example 29, the polyanion being used was heparin. It can be seen in FIG. 28 that the presence of 100 U/ml of heparin reduced the target independent signal generated by the “Negative Control Probe” Molecular Beacons to almost the same as the no-probe control.

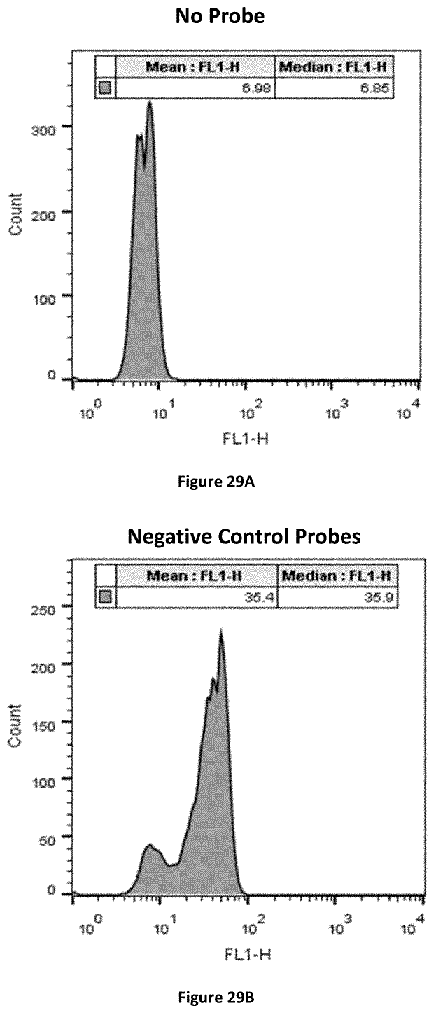

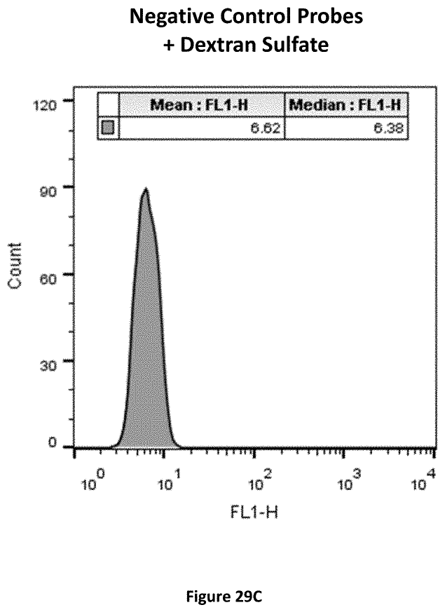

In Example 30 another polysaccharide polyanion, dextran sulfate, was used in conjunction with a Molecular Beacon specific for E6/E7. Dextran sulfate is commonly used as an exclusion agent in hybridization processes to create an environment where kinetics are sped up due to increased effective concentrations of probes and targets. In Example 30, it can be seen that the data in FIG. 29 demonstrates the existence of another effect of dextran sulfate, where it can be observed that the addition of this polyanionic reagent brought the background levels with the “Negative Control Probe” down enough to match the “No Probe” control.

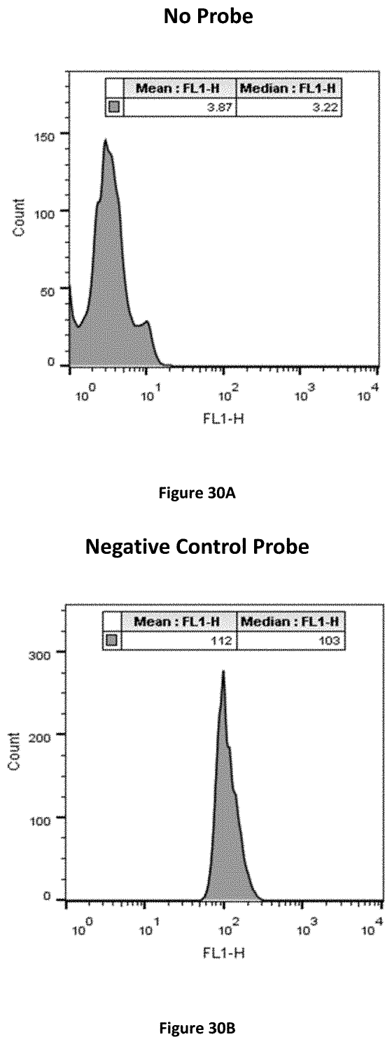

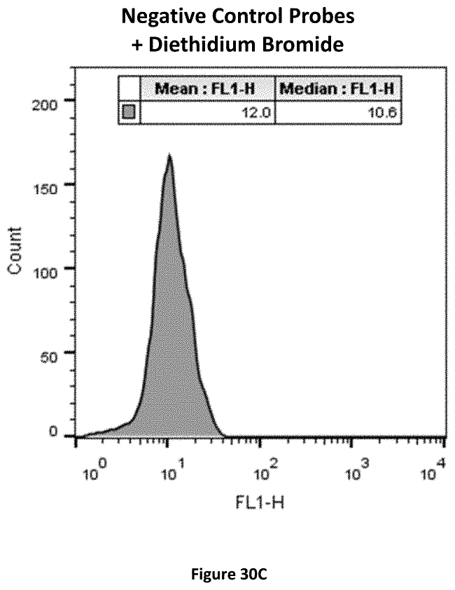

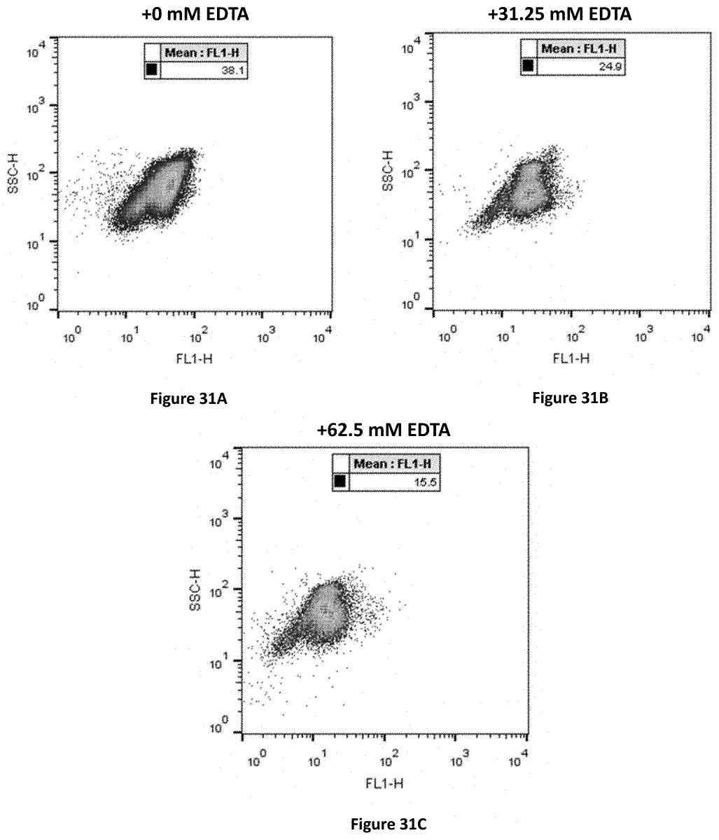

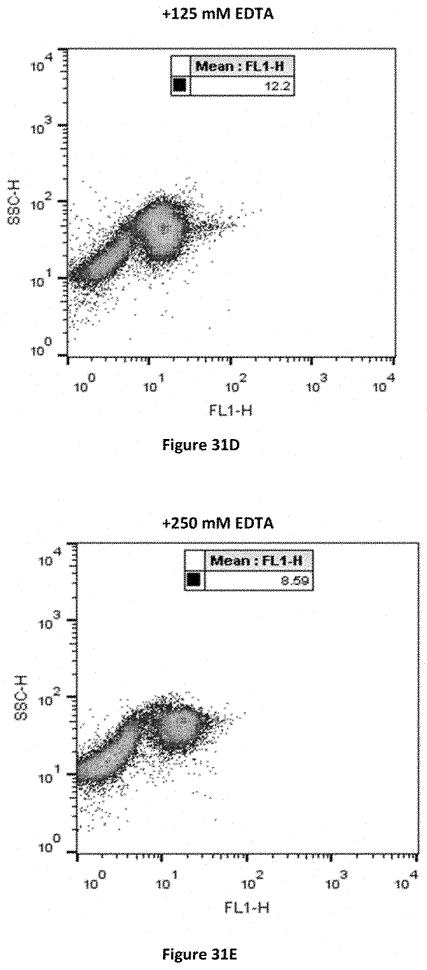

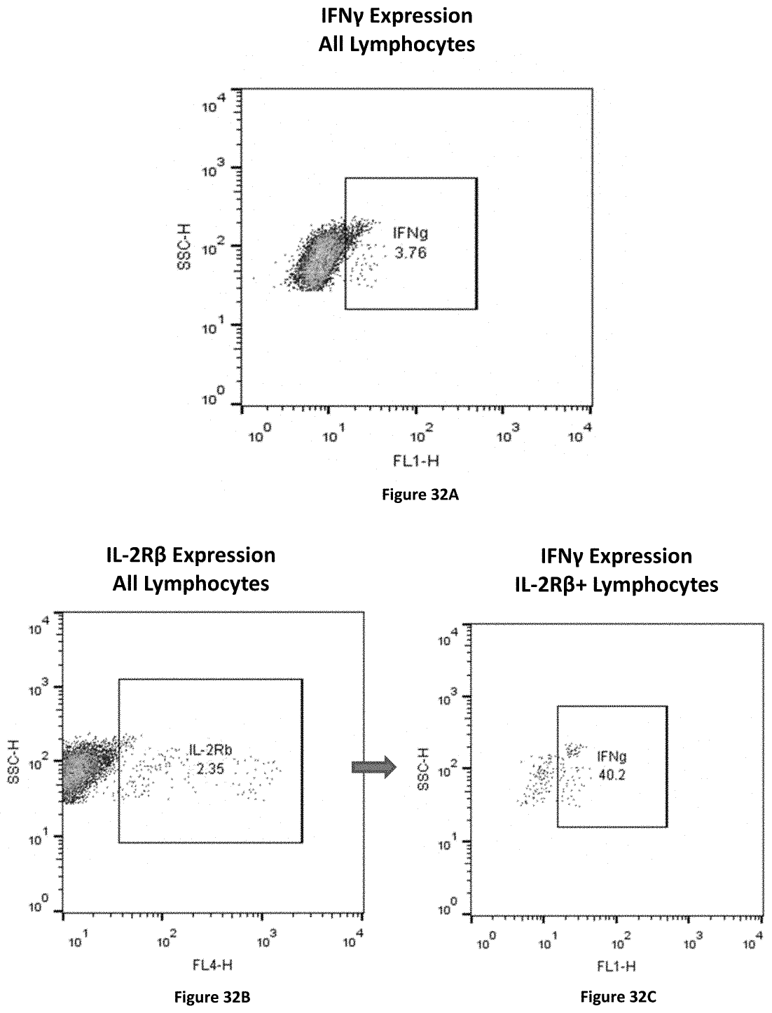

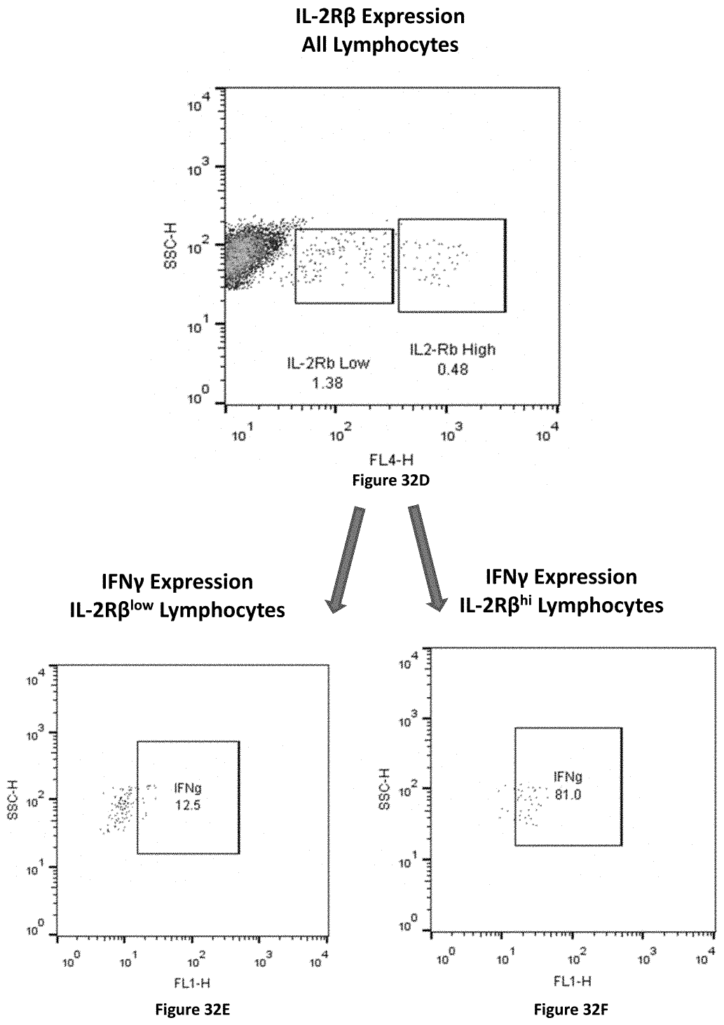

Also, in Example 29, as part of the same experiment with heparin, an intercalator was used in conjunction with the homogenous probes and detected by flow cytometry. In this particular Example, (FIG. 28) hybridization and detection carried out in the presence of NuclearID resulted in a twofold effect. First off, there was a major reduction of the false signal generated by the “Negative Control Probe”. In the second place, the resultant signal with the probe in the presence of Nuclear ID was even lower than the “no probe” control, a possible indication of damping down autofluorescence in the cells. A different intercalator was also tested for its effects upon noise generation from a Molecular Beacon and it can be seen in FIG. 30 that in the presence of 2 □M Ethidium homodimer, noise generation was brought down significantly. Although the level of signal remaining was still higher than background levels seen with the no probe control, only a single value was tried and it is possible that the use of other concentrations could bring the signal down to match the “no probe control” or even possibly attain even lower signals as seen previously with Nuclear ID.

With a reduction in the ambiguity of flow cytometry results derived from intra-cellular probing, the methods described herein allow for, but are not limited to, (a) profiling of patient immune systems, (b) determining whether infection is viral versus bacterial, (c) determining whether an individual has been vaccinated for is infected, (d) determining whether an individual has an infection or an autoimmune disease, (e) examining changes in immune response both to outside factors such as pathogens and allergens, and to internal factors such as auto-antigens and tumor cells, (f) identifying the factors that lead to bacterial resistance, (g) profiling gene expression, (h) monitoring expression of tumor biomarkers, (i) quantifying expression of non-coding RNAs, (j) identifying the toxicity factors in bacteria and determining bacterial pathogenicity, and (k) identifying the different signatures of viruses or bacteria in a cell.

### 7. EXAMPLES

This section describes various different working examples that will be used to highlight the features of the invention(s).

### 7.1. Example 1: Molecular Beacon Probe Designs

For the molecular beacon sequences described herein, stem sequences are shown in small letters and loop sequences are in capital letters. Molecular beacons specific for HPV16 and HPV 18 E6/E7 mRNA were labeled with 6-FAM at the 5′ end and a BHQ1a-Q quencher at the 3′ end. HPV16 sequences were designed using GenBank Accession no. K02718.1 (SEQ ID NO:63; Human papillomavirus type 16) and HPV18 sequences were designed using GenBank Accession no. X05015.1 (SEQ ID NO:64; Human papillomavirus type 18 E6, E7, E1, E2, E4, E5, L1 & L2 genes). Molecular beacons specific for housekeeping genes β-actin, GAPDH and 28S RNA were labeled with Quasar670 at the 5′ end and a BHQ3 quencher at the 3′ end. β-actin sequences were taken from NCBI Reference Sequence NM_001101.3 (SEQ ID NO:65). Molecular beacons specific for cancer marker p16 mRNA were labeled with Cy3 at the 5′ end and a BHQ2a-Q quencher at the 3′ end. P16 sequences were taken from NCBI Reference Sequence NM_000077 (SEQ ID NO:66). Molecular beacons for E6/E7 and P16 were ordered from MWG/Eurofins Genomics, Ebersburg, Germany and molecular beacons for β-actin were ordered from Biosearch Technologies, Petaluma, CA. The target-specific molecular beacon probes used in the experiments disclosed in the following Examples are set forth in SEQ ID NOs: 1-23, below:

HPV 16 specific probes
HPV 16-5
(SEQ ID NO: 1)
(6-FAM)-ccgaccAGTACTGTTGCTTGCAGTACACACAggtcgg-(Q)
HPV 16-7
(SEQ ID NO: 2)
(6-FAM)-cctggCTAAACATTTATCACATACAGCATGccagg-(Q)
HPV 16-8
(SEQ ID NO: 3)
(6-FAM)-ccacgaTTACAGCTGGGTTTCTCTACGtcgtgg-(Q)
HPV 18 specific probes
H18-671B
(SEQ ID NO: 4)
(6-FAM)-caccacgTCCTCTGAGTCGCTTAATTGCTcgtggtg-(Q)
H18-749B
(SEQ ID NO: 5)
(6-FAM)-ccagccATTGTGTGACGTTGTGGTTCggctgg-(Q)
H18-870B
(SEQ ID NO: 6)
(6-FAM)-cctcccacACCACGGACACACAAGGACAgtgggagg-(Q)
β-actin specific probes
Actin-1
(SEQ ID NO: 7)
(Q670)-cctcccacTTGCACATGCCGGAGCCGTTgtgggagg-(Q)
Actin-2
(SEQ ID NO: 8)
(Q670)-cctcccacTGACCCATGCCCACCATCACgtgggagg-(Q)
Actin-3
(SEQ ID NO: 9)
(Q670)-ccaccgAAGGTAGTTTCGTGGATGCCgtgggagg-(Q)
Actin-4
(SEQ ID NO: 10)
(Q670)-ccaccgTCTTGATCTTCATTGTGCTGcggtgg-(Q)
GAPDH specific probes
GAPDH1
(SEQ ID NO: 11)
(Q670)-ccaggagATTTTGGAGGGATCTCGctcctgg-(Q)
GAPDH2
(SEQ ID NO: 12)
(Q670)-ccgtggGACTGTGGTCATGAGTCCTTccacgg-(Q)
28S specific probes
28S-1
(SEQ ID NO: 13)
(Q670)-cgctccCTCTTAAAATCCCGCGGACggagcg-(Q)
28S-2
(SEQ ID NO: 14)
(Q670)-cgacgcTGTAGGAGAGGGAGTTCCgcgtcg-(Q)
28S-2S
(SEQ ID NO: 15)
(Q670)-ccacgcGGAACTCCCTCTCCTACAgcgtgg-(Q)
p16 specific probes
P16-674
(SEQ ID NO: 16)
(Cy3)-cccagaGGTACCGTGCGACATCGCGAtctggg-(Q)
P16-711
(SEQ ID NO: 17)
(Cy3)-ccgccATGGTTACTGCCTCTGGTGCCCggcgg-(Q)
P16-1113
(SEQ ID NO: 18)
(Cy3)-ccgccTGTCATGAAGTCGACAGCTTCCGGggcgg-(Q)
P16-1173
(SEQ ID NO: 19)
(Cy3)-ccgccAGCAGTGTGACTCAAGAGAAGCCAggcgg-(Q)
P16-1
(SEQ ID NO: 20)
(Cy3)-cctccgTGTCCAGGAAGCCCTCCCggagg-(Q)
P16-2
(SEQ ID NO: 21)
(Cy3)-ccgtgTCCTCAGCCAGGTCcacgg-(Q)
P16-1S
(SEQ ID NO: 22)
(Cy3)-ccgggAGGGCTTCGTGGACAcccgg-(Q)
P16-2S
(SEQ ID NO: 23)
(Cy3)-ccgacCCGTGGACGTGGCTGAGGAgtcgg-(Q)

### 7.2. Example 2: Preparation of Control Cells

The following cell lines were used for preparation of control cells: C33a (ATCC HTB-31), an HPV negative cervical carcinoma cell line, and Ect1/E6/E7 (ATCC CRL-2614), a cell line established from normal epithelial tissue with a retrovirus expressing the HPV16 E6/E7 genes. See Fichirova et al., 1997 Biol Reprod 57; 847-855. Cells were grown to 80-90% confluency, trypsinized, washed and resuspended in PBS. Cells were centrifuged at 750×g for 6 minutes, resuspended in 10 ml of 1% formaldehyde/PBS and incubated at 4° C. for 1 hour. Cells were again centrifuged down at 750 ×g, washed with PBS and resuspended in 10 ml of ThinPrep. Cells were left at 4° C. overnight and then transferred for storage at −20° C. Cells were fixated in the last step of this protocol in order to stabilize the cellular contents, such as E6/E7 mRNAs, for analysis when used later in conjunction with clinical specimens suspended in ThinPrep solution.

### 7.3. Example 3: Detection of HPV E6/E7 and β-Actin mRNA in Cell Lines

Fixation/permeabilization procedure. A 500 μl sample of either HPV-positive Ect/E6/E7 cells or HPV-negative C33A cells in ThinPrep prepared as described in Example 2 were added to 1 ml of 1.5% formaldehyde in PBS. After vortexing to mix the contents, the solution was incubated at 22° C. (room temperature) for 1 hour. Cells were then centrifuged at 1000×g for 6 minutes and the supernatant was removed by aspiration. Cells were resuspended in 1 ml of 1% Triton X-100 in PBS, vortexed and incubated at 22° C. (room temperature) for 30 minutes. Cells were centrifuged at 1000×g for 6 minutes, the supernatant was removed and the cells were resuspended in 1 ml of 0.05% Tween 20, 2 mM MgCl₂ in 1×SSC and incubated at 22° C. (room temperature) for 10 minutes.

Hybridization procedure. The cell preparation above was centrifuged at 1000×g for 6 minutes, the supernatant was removed and the cells resuspended in 300 μl Beacon Hybridization Mix: 4.17 nM each of HPV E6/E7 molecular beacons (HPV 16-5 (SEQ ID NO: 1), HPV 16-7 (SEQ ID NO: 2), HPV 16-8 (SEQ ID NO: 3), H18-671B (SEQ ID NO: 4), H18-749B (SEQ ID NO: 5), H18-870B (SEQ ID NO: 6)) and 3-actin molecular beacons (Actin-1 (SEQ ID NO: 7), Actin-2 (SEQ ID NO: 8), Actin-3 (SEQ ID NO: 9) and Actin-4 (SEQ ID NO: 10)) in 0.05% Tween 20, 2 mM MgCl₂ in 1×SSC. Hybridization was carried out at 65° C. for 30 minutes and then left at 22° C. (room temperature) for 1 hour.

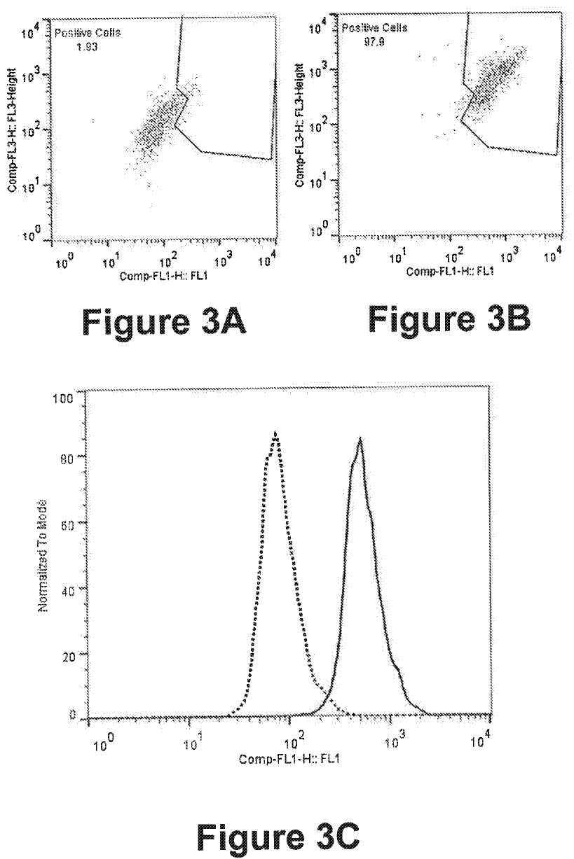

Flow Cytometry. As shown in FIG. 3A and FIG. 3B, samples were analyzed for signals from the HPV (FL-1 channel) and β-actin (FL-3 channel) beacons using a FACS Calibur flow cytometer (BD Biosciences, San Jose Calif.). When gating was applied (heavy lines in FIGS. 3A and 3B), 97.9% of the HPV Ect E6/E7 cells gave a positive signal while only 1.9% of the C33A cells gave a positive signal, showing the ability of the present invention to separately identify HPV-positive and HPV-negative cells. The results of this experiment were also analyzed using the Mean Fluorescent Intensity (MFI) program of the flow cytometer. These results are set forth in FIG. 3C, and show a clear separation of signals from HPV-positive and HPV-negative cell lines, as peaks from these cell lines are widely separated with little overlap.

Conclusion. The use of molecular beacons for 3-actin as internal controls in this experiment allowed gating out of cells with low β-actin signals. These cells, if not excluded, could generate false negative signals due to destruction of internal mRNA by nuclease degradation or by loss of targets due to a loss of integrity of cellular membranes. It also allows elimination of false negatives due to incomplete permeabilization where probes are unable to gain access to cells. As such, only cells that have appropriately high levels of β-actin signals are considered when calculating the number of HPV positive cells in an assay.

### 7.4. Example 4: Flow Cytometry Results with a Clinical Specimen Using β-Actin for Gating

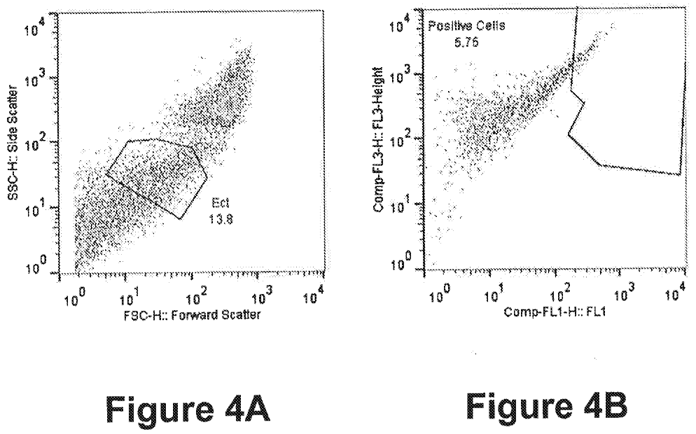

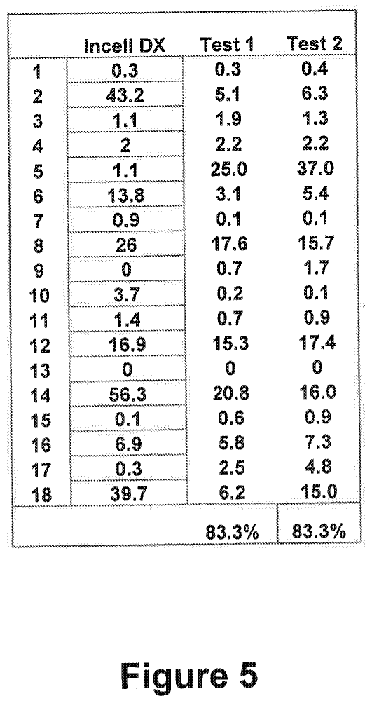

A clinical specimen previously known to be HPV-positive was processed as described above. Since the biological sample contained a mixed population of various cell types from a biological sample, gating was first used for side scatter and forward scatter. Previous experiments had shown the appropriate parameters using anti-cytokeratin and anti-CD45 to exclude ectocervical cells as described in Grundhoefer and Patterson (2001) Cytometry 46:340-344. The heavy lines in FIG. 4A represent the ectocervical cells delineated by this method, which represent 13.8% of the entire cell population. This population was then evaluated for signals from the HPV E6/E7 (FL1) and the β-actin (FL3) molecular beacon probes as described in Example 3. These results are shown in FIG. 4B and 5.75% of the cells were positive for HPV E6/E7, well above the 2% cutoff used to define positivity.

### 7.5. Example 5: Flow Cytometry Results with Molecular Beacons for Clinical Specimens Using β-Actin for Gating and Comparison with incellDx HPV OncoTect™ E6, E7 mRNA Kit

Cells from clinical cervical cell specimens and control cells from Example 2 in ThinPrep solution were processed as described in sections A) and B) of Example 3. The hybridization reaction solution contained a mixture of the six E6/E7 molecular beacons (SEQ ID NOs: 1-6) and the β-actin molecular beacons (SEQ ID NOs: 7-10) described in Example 1. Detection was carried out in duplicate on separate days. (Test 1 and Test 2 of FIG. 5) For comparison, cells from the same samples were processed and tested according to the method described by IncellDX. See Grundhoefer and Patterson (2001) Cytometry 46:340-344. Detection of signals from the molecular beacons by flow cytometry was carried out as described in Example 3 and 3-actin was used for gating to normalize the results. The percentages set forth in FIG. 5 reflect the portion of the assayed cell populations that exhibited a signal above threshold. As described for the incellDX protocol and as seen in Example 3 above, the cutoff for positivity was more than 2% of the cells being above the threshold. Although the percentage of cells inside the positive gate for each duplicated measurement showed some variation, the samples were consistently either positive or negative in each test. In addition, the results of assays performed according to the protocol described in this Example showed a 83.3% correlation with the incellDX assignations, sample 10 tested positive in the incellDX assay but was negative when assayed according to the protocol described herein, and samples 5 and 17 were negative in the incellDX assay, but tested positive when assayed according to the protocol described herein. The detected variations between the incellDX and beacon assays could reflect differential cross-reactivity of probes with HPV strains that are not HPV16 or HPV18, but that are phylogenetically related.

### 7.6. Example 6: Analysis of Ability of Molecular Beacon Probes to Detect Housekeeping Gene mRNA Targets in Cells by Flow Cytometry

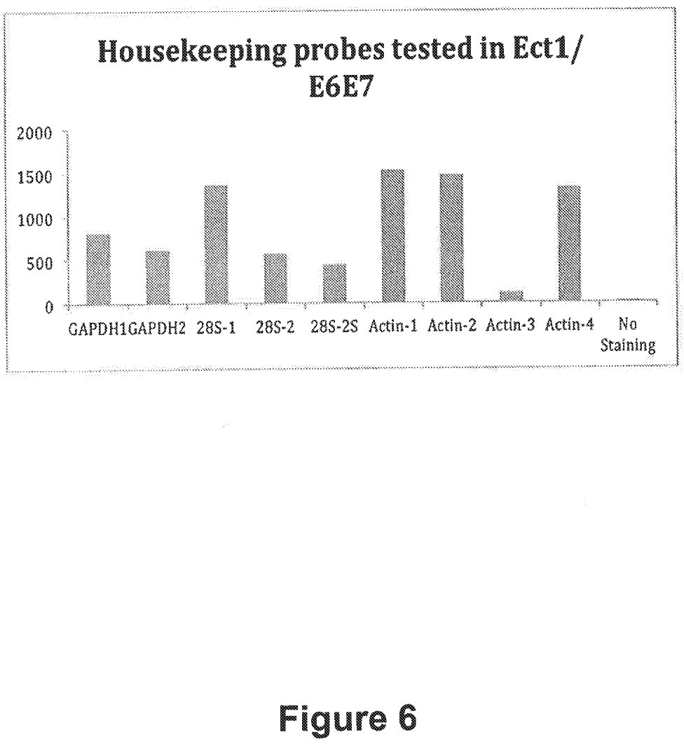

Although used as a pool of probes in previous Examples, molecular beacons for 3-actin as well as beacons for GAPDH and 28S were tested individually in Ect1/E6E7 cells by flow cytometry. The sequences of the probes are shown in Example 1 and cell preparation and hybridizations were carried out as described in Example 3, except that the final concentration of each probe was 8 nM. The MFI for each of the probes is presented in FIG. 6, which shows that there are varying levels of performance for each of the probes. Since there are different levels of each target in a cell, comparisons between probes for different targets do not convey much information about the efficiencies of probe design. However, when comparing different probes for the same target, such as GAPDH1 (SEQ ID NO: 11) and GAPDH2 (SEQ ID NO: 12), the measured levels of the target were about the same. On the other hand, 28S-1 (SEQ ID NO: 13) performed better than 28S-2 (SEQ ID NO: 14), but 28S-2 itself did not produce signal that was much more than background, as evidenced by the similar levels of signals seen for 28S-2 and 28S-2S (SEQ ID NO: 15) (28S-2S is the sense sequence of 28S rRNA and therefore should not hybridize to the rRNA). More striking are the results using the actin probes where when used as a pool, Actin-1 (SEQ ID NO: 7), Actin-2 (SEQ ID NO: 8) and Actin-4 (SEQ ID NO: 10) were effective, and Actin-3 (SEQ ID NO: 9) contributed little if any signal from hybridization to β-actin mRNA in cells comprising Ect1/E6E7.

### 7.7. Example 7: Analysis of Ability of Molecular Beacon Probes to Detect p16 mRNA Targets in Cells by Flow Cytometry

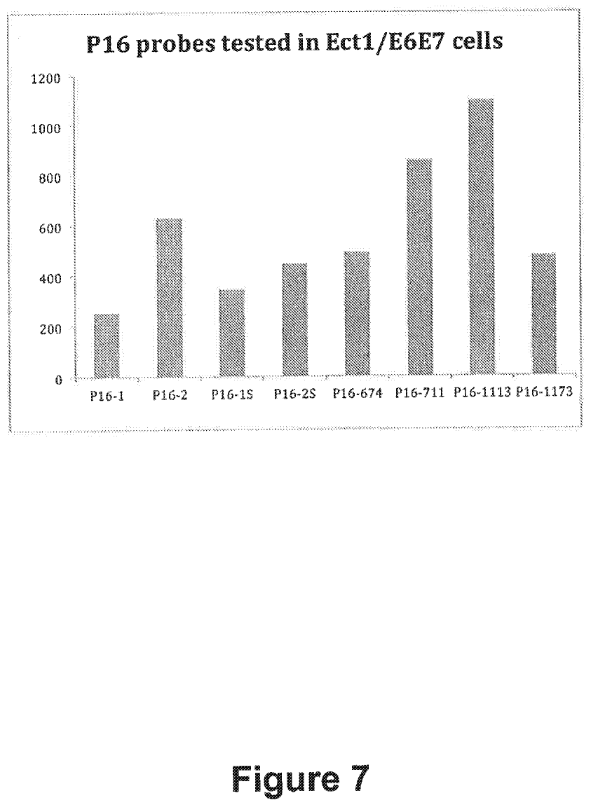

In the same way that individual molecular beacon probes for detection of housekeeping genes were tested in Example 6, individual molecular beacons for p16 mRNA (SEQ ID NOs: 16-23) were tested in Ect1/E6E7 cells by flow cytometry. Cell preparation as well as hybridizations were carried out as described in Example 3 except the concentration of each probe was 8 nM and the MFI was measured. As shown in FIG. 7, varying levels of signal were detected for the various probes, and probes P16-2 (SEQ ID NO: 21), P16-711 (SEQ ID NO: 17) and P16-1113 (SEQ ID NO: 18) beacons gave the best MFI.

### 7.8. Example 8: Detection of HPV E6/E7 and P16 mRNA in Clinical Samples and Comparison with Pathology

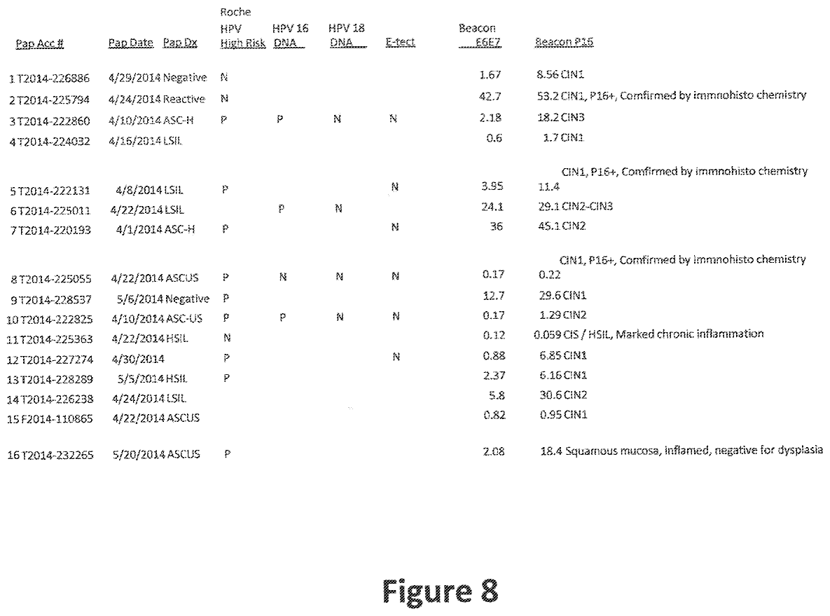

A number of clinical specimens were evaluated by pathology and tested by flow cytometry analysis of specimens with molecular beacon probes for HPV E6/E7 (SEQ ID NOs: 1-6), β-actin (SEQ ID NOs: 7-10) and p16 carried out as described in Example 5 except that the number of P16 probes were reduced to p16-674 (SEQ ID NO: 16), p16-711 (SEQ ID NO: 17), p16-1113 (SEQ ID NO: 18) and P16-1173 (SEQ ID NO: 19). In addition, commercially available assays the Roche HIPV High Risk assay, Roche HPV 16 Assay, Roche HPV18 Assay, and the E-tect (incellDX HPV OncoTect™ E6, E7 mRNA) assay as validated and performed in Enzo Clinical Labs, Farmingdale, N.Y.—were also used with some of the samples. The Cobas® HPV Test is able to collectively identify the presence of high-risk HPV and also to identify HPV16 and HPV18 individually using real-time PCR. Aliquots from samples were processed and assayed according to the manufacturers' instructions. The results of these tests are tabulated in FIG. 8, where for those samples that were evaluated as CIN 1, 4 were positive and 5 were negative and for those samples evaluated as CIN 2 or CIN 3, 4 were positive and 1 was negative using the molecular beacon methods described herein. If specimens collected from the subjects at a later time were available, the methods described herein could be used to assay such specimens using the E6/E7 and p16 molecular beacons in order to follow the progression/regression of the disease.

### 7.9. Example 9: Creation of Plasmids Coding for HPV E6/E7 Transcripts from a Variety of Different HPV Types

Sequences for the E6/E7 region of a number of different HPV types were used to make a series of plasmids that could be used to make in vitro transcripts of the E6/E7 region. Transcription cassettes were designed with the following structures:

5′-ATGTAATACGACTCACTATAGGGCGAATTGGGTACC-E6/E7-3′

where the underlined nucleotides represent a T7 promoter and “−E6/E7” represents the coding sequence of the E6/E7 genes of a variety of different HPV types, including HPV6, HPV11, HPV16, HPV18, HPV26, HPV31, HPV33, HPV35, HPV39, HPV42, HPV43, HPV44, HPV45, HPV51, HPV52, HPV53, HPV56, HPV58, HPV59, HPV66, HPV67, HPV68, HPV70, HPV73 and HPV82. In addition, either a Hind III (AAGCTT) or a Pst I (CTGCAG) restriction site was added to the 5′ end and an EcoR1 restriction site (GAATTC) was added to the 3′ end of the cassette so that the cassette could be excised out of a plasmid prior to carrying out a transcription reaction. See FIG. 20, SEQ ID NOs. 34-58. The Pst 1 sequence was used for HPV26, HPV43, HPV51 and HPV67 at the 5′ end while all other HPV types used the Hind III sequence at the 5′ end. Sizes of the complete cassette ranged from 735 nucleotides (HPV42) to 865 nucleotides (HPV68). Synthesis of the cassettes and insertion into pUC57 plasmid was carried out by Genscript USA, Inc., Piscataway N.J.

### 7.10. Example 10: Preparation of Artificial E6/E7 Transcripts

2 μg of the plasmid for each HPV type described in Example 9 were digested with either EcoR1/Hind III or EcoR1/Pst 1 to remove the transcription cassette from the vector, and were purified using the QIAquick Purification Kit (Qiagen), and recovery was measured with the NanoDrop spectrophotometer (Thermo Scientific). 0.5 μg of the DNA from each digested plasmid was used in a 20 μl reaction mix with the MEGAscript T7 transcription kit (Life Technologies) with 0.2 μl of 10× biotin-labeled ribonucleotides (from ENZO BioArray HighYield RNA Transcript Labeling kit). The reaction proceeded overnight at 37° C. and the RNeasy Mini Kit (Qiagen) was used to purify the biotin labeled transcript products. Quantification was carried out using the NanoDrop spectrophotometer (Thermo Scientific).

### 7.11. Example 11: Cross-Reactivity of E6/E7 Molecular Beacons from HPV16 and HPV18 with Various HPV Types

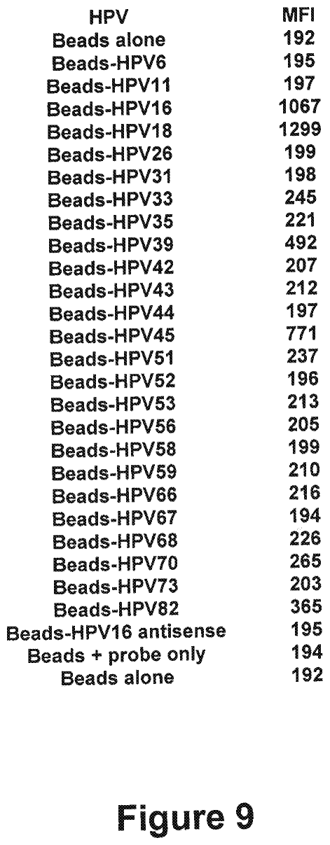

For each HPV type, 250 ng of biotinylated transcript were mixed with the HPV16 and HPV18 E6/E7 molecular beacons from Example 1. The final concentration of probes was 0.25 QM for each probe in a volume of 40 □l of Etech 2.0 Buffer C. Samples were mixed well and incubated at 65° C. for 30 minutes. After cooling the mixture down to room temperature, 0.5 □l of M-280 Dynabeads from the Dynabeads Streptavidin Trial Kit (Life Technologies) was added, mixed well and shaken for 3 hours at room temperature to allow binding of biotinylated transcript/molecular beacon complexes to the beads. One ml of Buffer C was added to the mixture and then placed on a magnetic stand to collect the magnetic beads. The supernatant was removed and the beads were resuspended in 300 □l of Buffer C. Samples were run on the flow cytometer and MFI was measured for the beads in each sample preparation. The results of this protocol are shown in FIG. 9. While showing high signal strength with molecular beacons derived from HPV 16 and 18 as expected, there was also significant signal generation with molecular beacons derived from HPV39 and HPV45, which are known to be closely related to HPV18 and are considered to be high risk HPV types.

### 7.12. Example 12: Expansion of E6/E7 Detection Probes

The results of Example 11 showed that the E6/E7 molecular beacons derived from HPV16 and HPV 18 showed minimal reactivity with HPV31 and HPV33. However, it may be advantageous to have molecular beacon probes that can detect more targets than displayed by the probes used in Example 11, where essentially only HPV16, HPV18, HPV39 and HPV45 gave appreciable levels of signal. Accordingly, molecular beacons were designed based on the sequences of HPV31 and HPV33 and had the following sequences:

HPV-31
HPV31-1
(SEQ ID NO: 24)
(6-FAM)-ccgacgtTTCTAATGTTGCTCCATACACacgtcgg-
(BHQ1a-Q)
HPV31-2
(SEQ ID NO: 25)
(6-FAM)-cctggcaACGTCCTGTCCACCTTCCACCTAtgccagg-
(BHQ1a-Q)
HPV-33
HPV33-1
(SEQ ID NO: 26)
(6-FAM)-ccaactAATTGCTCATAGCAGTATAGGTCagttgg-
(BHQ1a-Q)
HPV33-2
(SEQ ID NO: 27)
(6-FAM)-cctgcacAGCGCCCTGTCCAACGACCCGAAATAgtgcagg-
(BHQ1a-Q)

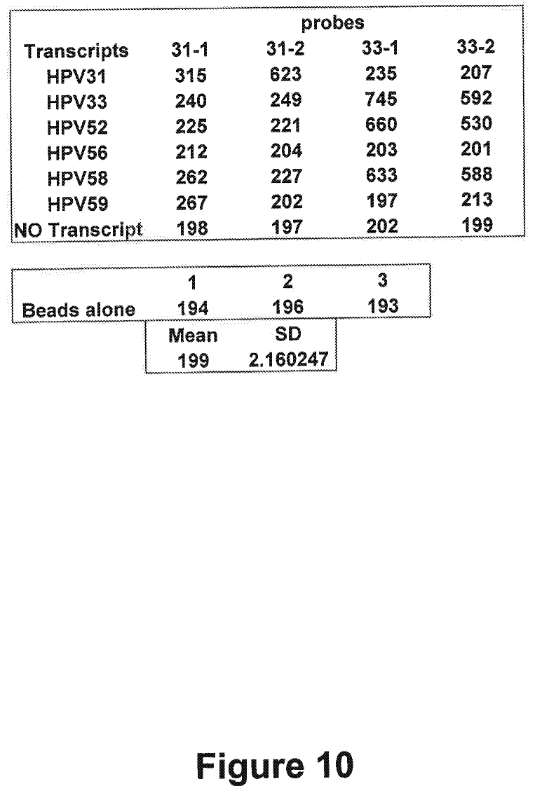

In these probes, the stem sequences are shown in small letters and the loop sequences are in capital letters. These molecular beacons were tested with transcripts from a variety of different HPV types in the same manner as described in Example 11 and the results are shown in FIG. 10. It can be seen here that the 31-2 probe (SEQ ID NO: 25) was effective at generating signals from the HPV31 transcripts while showing minimal signal from the other HPV types tested. In contrast, the molecular beacons derived from HPV33 (SEQ ID NOs: 26, 27) showed a wider spectrum of efficient detection that included HPV33, HV52 and HPV58. Consequently, at least two different probe cocktails can be used for E6/E7 evaluation using flow cytometry: (i) a mixture of the HPV16 E6/E7 molecular beacon probes (SEQ ID NOs: 1-3) and HPV18 E6/E7 molecular beacon probes (SEQ ID NOs: 4-6) for detecting the presence of HPV16, HPV18, HPV39 and HP45, or (ii) a mixture of probes that include HPV16 and HPV 18 E6/E7 probes, HPV31-1E6/E7 probe (SEQ ID NO: 24), and HPV33-1 and HPV 33-2 E6/E7 probes (SEQ ID NOs: 26, 27) for efficient detection of one or more HPV16, HPV18, HPV31, HP33, HPV39, HPV45, HIPV52 and HPV58 E6/E7 transcripts in clinical samples.

### 7.13. Example 13: Comparison of Protocols for Permeabilizing and Staining Fixated Cells

Fixation of positive and negative control cells. Caski cells (an adherent HPV+ cell line) were used as positive control cells. The positive control cells were fixated in ThinPrep solution with 1% formaldehyde for 1 hour, and were then washed and stored in ThinPrep solution alone at −20° C. H9 cells (a T-cell line negative for HPV) were used as negative control cells. The negative control cells were fixated for 2 days in ThinPrep solution with 6% formaldehyde. After fixation, the cells were washed and stored at −20° C. in ThinPrep alone. Patient samples were provided either in ThinPrep (methanol) or SurePath (ethanol) solutions.

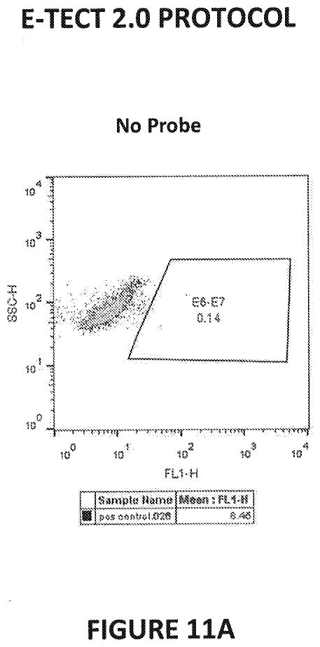

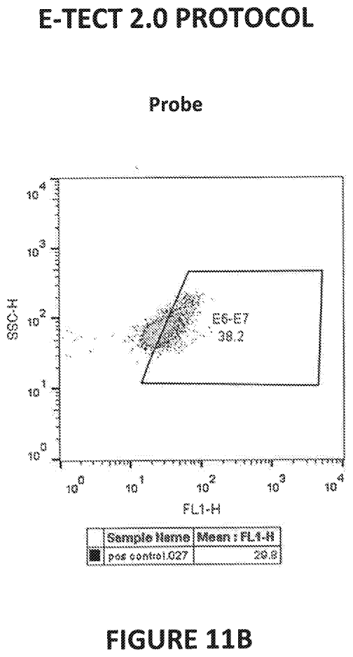

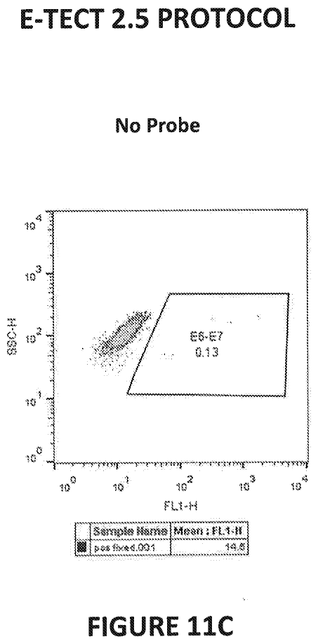

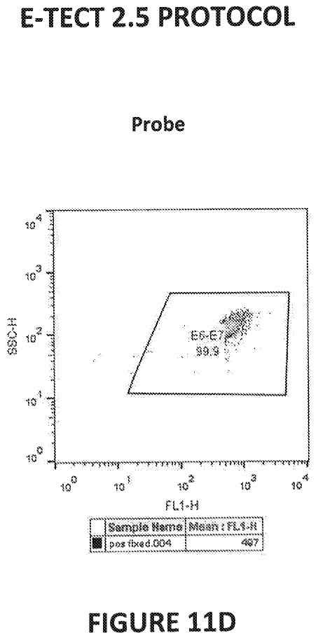

Protocol E-Tect 2.0. Positive control cells, negative control cells and sample cells were treated as follows. 500 μL of fixated cells were suspended in 1 mL of 1.5% formaldehyde in DPBS (buffer A) and incubated for 1 hour at room temperature. The cells were washed and 1 mL of 1% Triton X-100 in DPBS (Buffer B), resuspended and incubated for 30 minutes at room temperature. The cells were then washed and 1 mL of 0.05% Tween and 2 mM MgCl₂ in 1×SSC (Buffer C) was added, after which the cells were mixed and incubated for 10 minutes at room temperature. After incubation, the cells were washed and 300 μL of a probe solution containing 1 mL of Buffer C with E6/E7 molecular beacon probes (1 μL probe/600 μL buffer) were added to each sample. The samples were resuspended and incubated for 30 minutes at 65° C. in a light-protected environment. After incubation, the cells were mixed and then incubated at room temperature for 1 hour in a light-protected environment, after which the cells were assayed using flow cytometry. The results are shown in FIGS. 11A-11B.

Protocol E-Tect 2.5. Positive control cells, negative control cells and sample cells were treated as follows. 500 μL of fixated cells were washed and resuspended in 1 mL of hybridization buffer (1% Triton X-100 in 1×SSC) and washed again. 150 μL of hybridization buffer with E6/E7 molecular beacon probes (1 μL probe/600 μL buffer) were added, and the cells were mixed and incubated for 1 hour at 65° C. in a light-protected environment. Samples were mixed and put on ice at 4° C. for 1 hour in a light-protected environment, after which the cells were assayed using flow cytometry. The results are shown in FIGS. 11C-11D.

Results. Protocol E-Tect 2.5 is shorter and produces stronger signal (20 times more signal) and has less chance of errors. The final step performed at 4° C. greatly reduced background staining due to unbound probes not closing properly.

### 7.14. Example 14: INF-γ Expression in Jurkat Cells

Wells in a 6-well plate were each coated with 1 mL of anti-CD3 coating buffer (0.1 g/mL in PBS) and incubated in a 37° C. incubator overnight. The next morning Jurkat cells were collected. 2×10⁶ cells were fixated immediately (the non-activated time point), and 2×10⁶ cells were plated in wells of the 6-well plate after removing the anti-CD3 coating buffer. At the 18 and 36 hour time points, cells were collected from individual wells and fixated. Cells were fixated by resuspending the cells in 1 mL of 5% formaldehyde in PBS and incubating at 4° C. for 30 minutes, after which time they were washed twice in PBS and resuspended in 1 mL PBS for storage at 4° C.

Cells were stained by resuspending in 2% Triton X-100 in 1×SSC containing 33 nM of the IFN-γ probe cocktail and a 1:1000 dilution of NuclearID. Cells were incubated for 1 hour in the dark at 65° C., then cooled for 30 min at 4° C. Samples were run on a FACSCalibur flow cytometer using NuclearID to gate on healthy cells, and IFN-γ mRNA using the INF-γ specific probe cocktail containing the molecular beacon probes having the following sequences and labels:

(SEQ ID NO: 28)
6-FAM-ccagccAATGACCTGCATTAAAATATTTCTTAAGGTTTTCTggct
gg-BHQ2
(SEQ ID NO: 29)
6-FAM-ccagccATTATTTTTCTGTCACTCTCCTCTTTCCAATTCggctg
g-BHQ2
(SEQ ID NO: 30)
6-FAM-ccagcACCTGCATTAAAATATTTCTTAAGGTTTTCTGGGgctgg-
BHQ2
(SEQ ID NO: 31)
6-FAM-ccagccACTCTCCTCTTTCCAATTCTTCAAAAGggctgg-BHQ2
(SEQ ID NO: 32)
6-FAM-ccagcGGACATTCAAGTCAGTTACCGAATAATGGgctgg-BHQ2

Probes were designed to bind human INF-γ mRNA across exon-exon junctions to insure specificity to mRNA only.

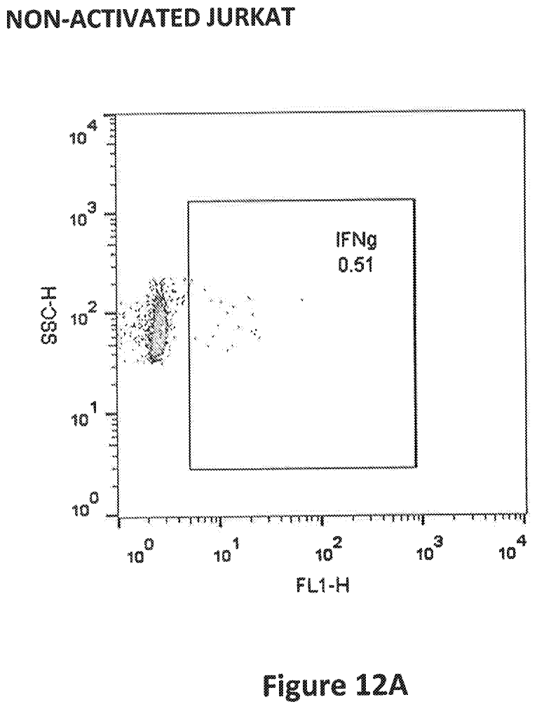

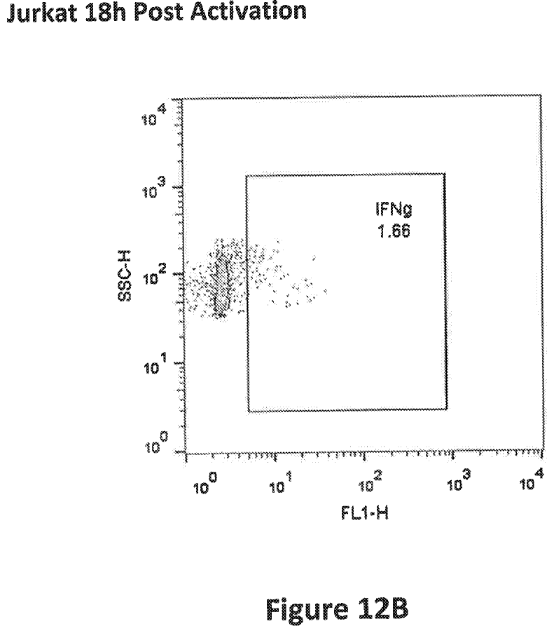

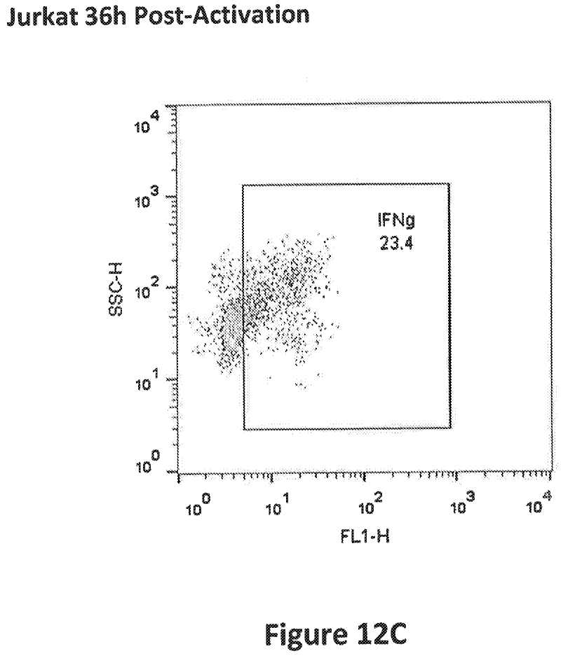

Each population was analyzed for INF-γ mRNA separately. Flow cytometry results show that INF-γ mRNA is increasing over time after activation of Jurkat cells with anti-CD3 as shown in FIGS. 12A-12C, demonstrating that TCR activation induces INF-γ expression.

### 7.15. Example 15: Analysis of E6/E7 HPV mRNA Content in Cervical Cells in Different Phases of the Cell Cycle

Cell cycle protocol. Samples were pap smears provided fixed in ThinPrep. Aliquots of each sample were taken and plated into a 96-well U-bottom plate for staining, spun down and supernatant was discarded. Cells were then fixed in 5% formaldehyde in PBS (150 μL/sample), and incubated at room temperature for 30 minutes. Cells were washed twice with 300 μL of hybridization buffer (2% Triton X-100 in 1×SSC) and resuspended in 150 μL/sample of hybridization buffer containing 10 nM E6/E7 probe cocktail and a 1:1000 dilution of NuclearID. Cells were incubated for 1 hour at 65° C. in the dark, then cooled for 30 minutes at 4° C. in the dark. Cells were then run on a FACSCalibur flow cytometer, using NuclearID to gate on the G1, S, and G2 phase populations of cells.

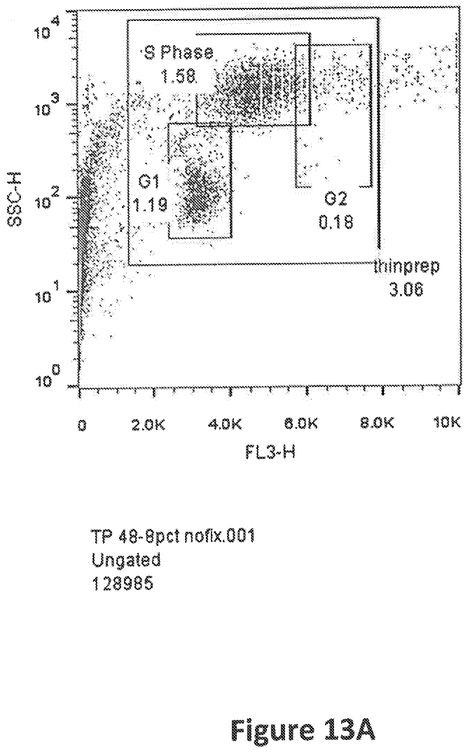

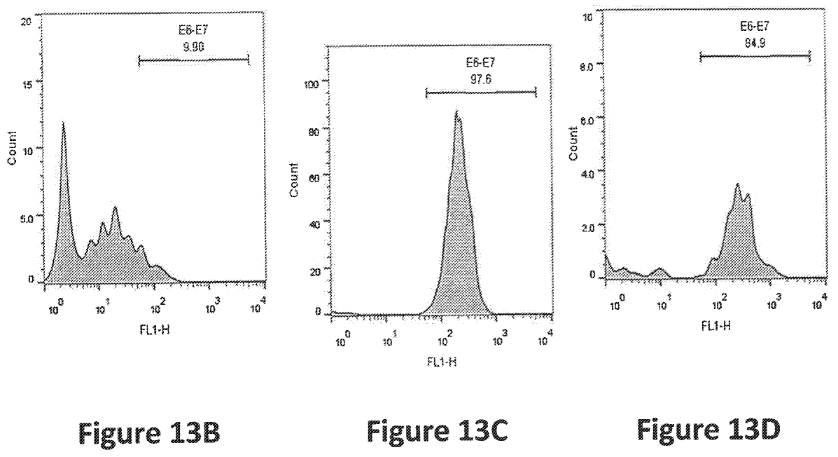

Flow cytometry results indicated that resting G1 cells had little to no viral mRNA (FIGS. 13A and 13D), while almost all of proliferating S phase and G phase cells (respectively FIG. 13B and FIG. 13C, and FIG. 13D) had high levels of viral mRNA.

### 7.16. Example 16: bDNA for the Detection of Biotin Labeled Antibodies Using Flow Cytometry

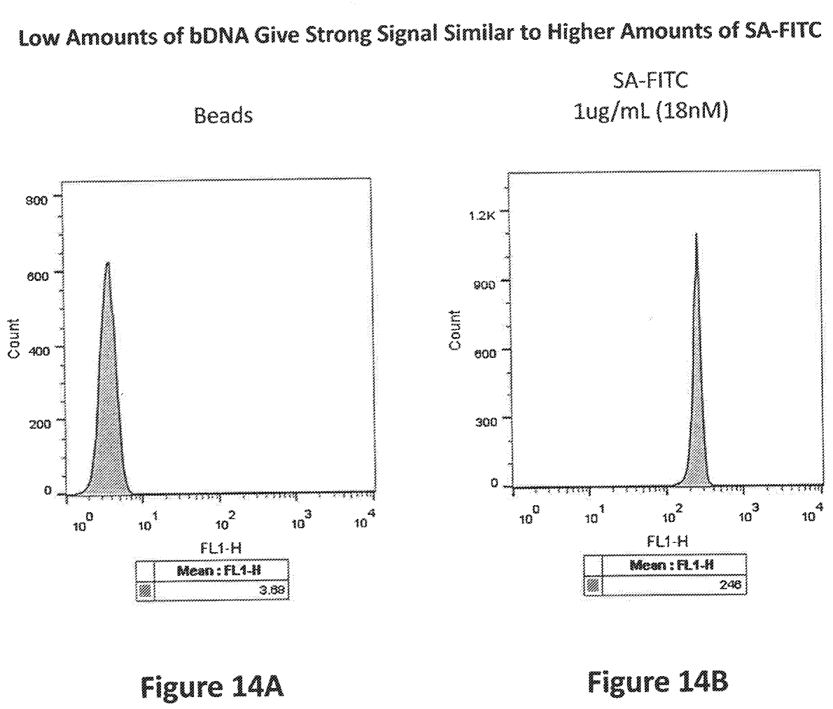

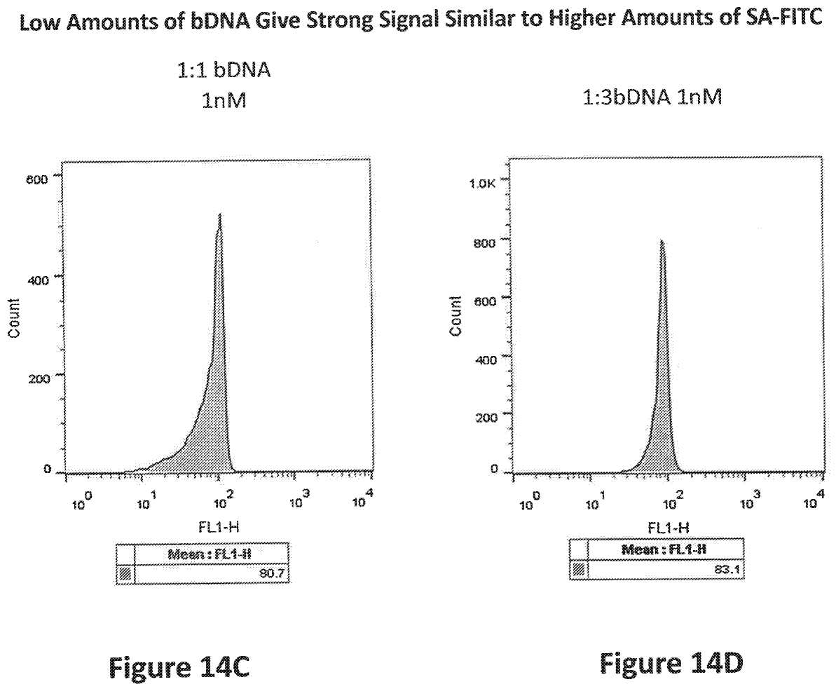

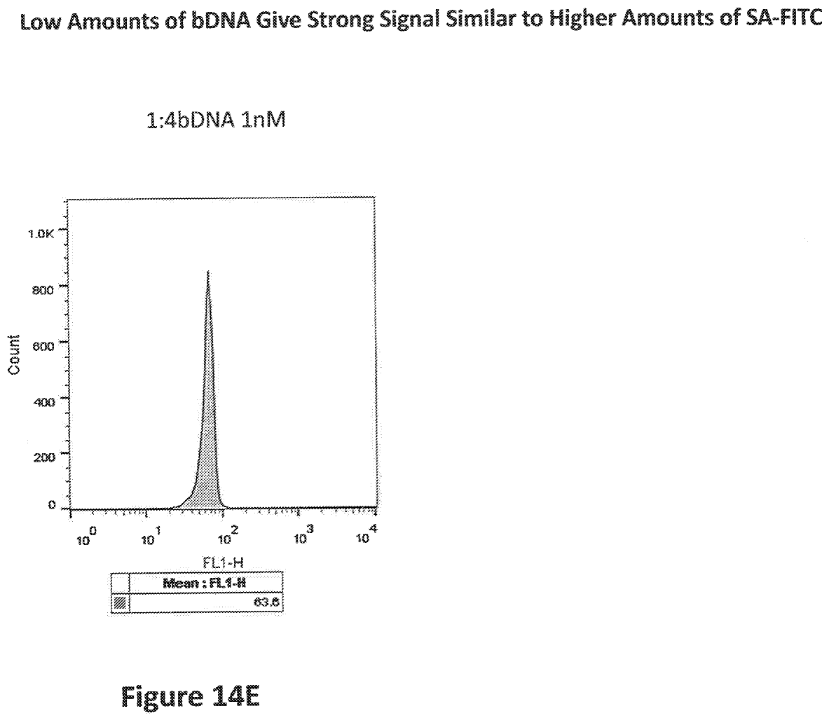

bDNA labeled antibodies. Brightness of bDNA-streptavidin (bDNA-SA) constructs made in different ratios of DNA to fluorophore (1:1, 1:3 and 1:4) at 1 nM were compared to SA-FITC at 18 nM as the positive control. Biotin-labeled beads were used as a negative control to compare brightness. As shown in FIG. 14, bDNA constructs at 1 nM (FIGS. 14C-14E, respectively 1:1 bDNA, 1:3 bDNA and 1:4 bDNA) provided brightness similar to SA-FITC (FIG. 14B) that was used in much higher concentrations (18 nM).

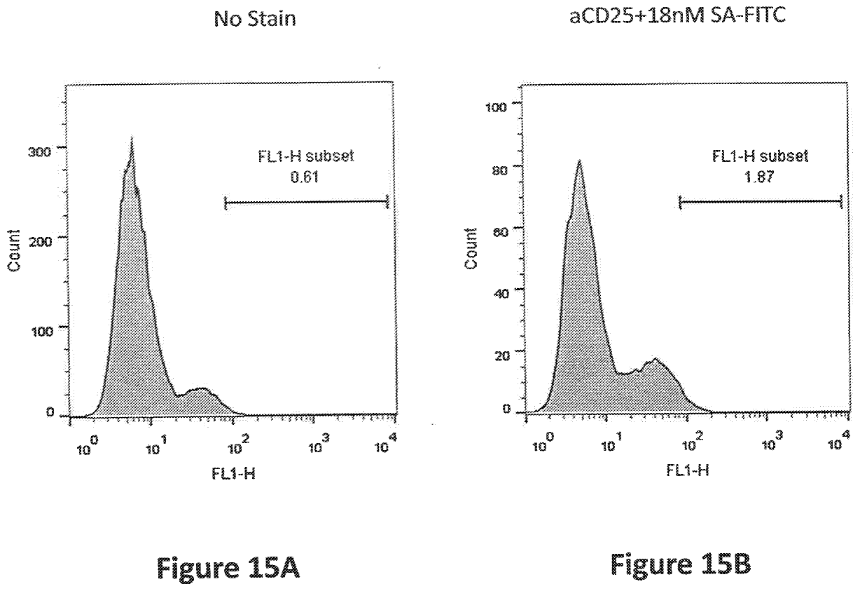

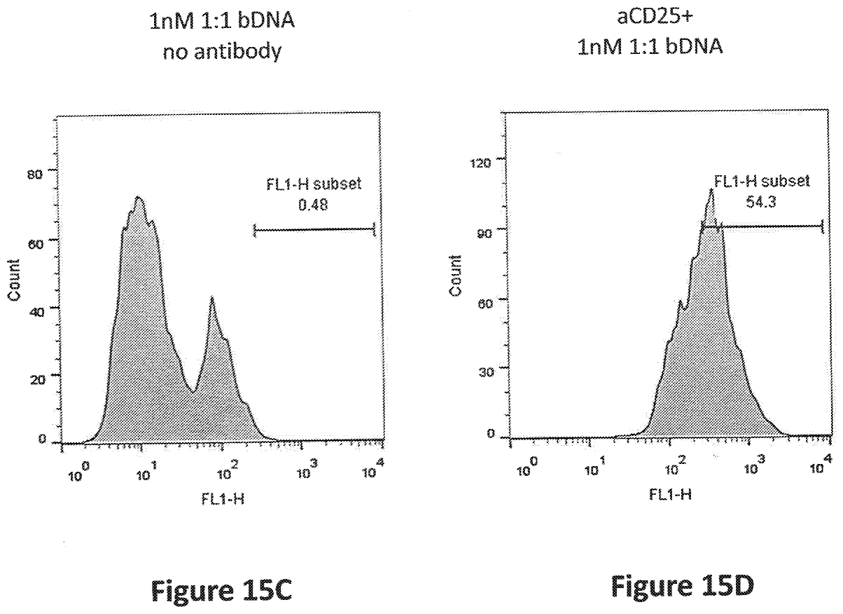

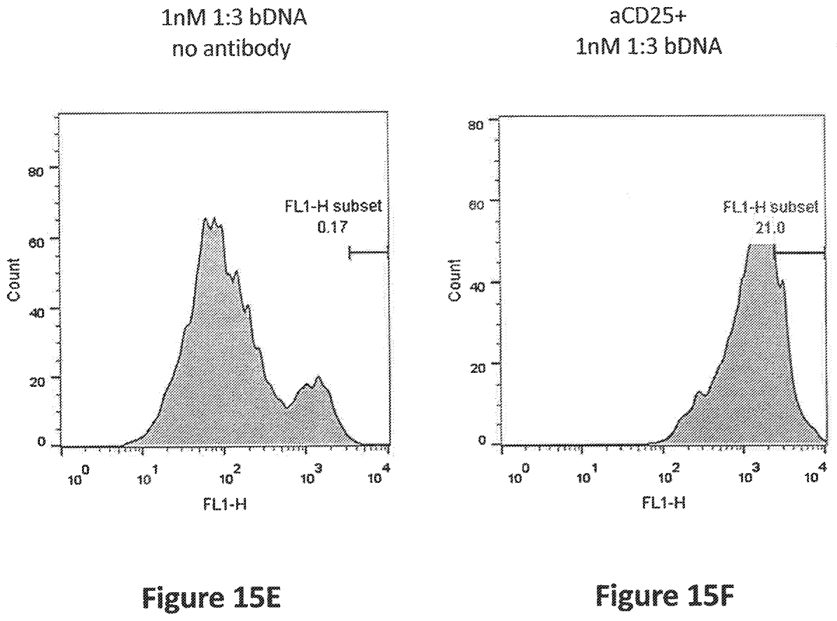

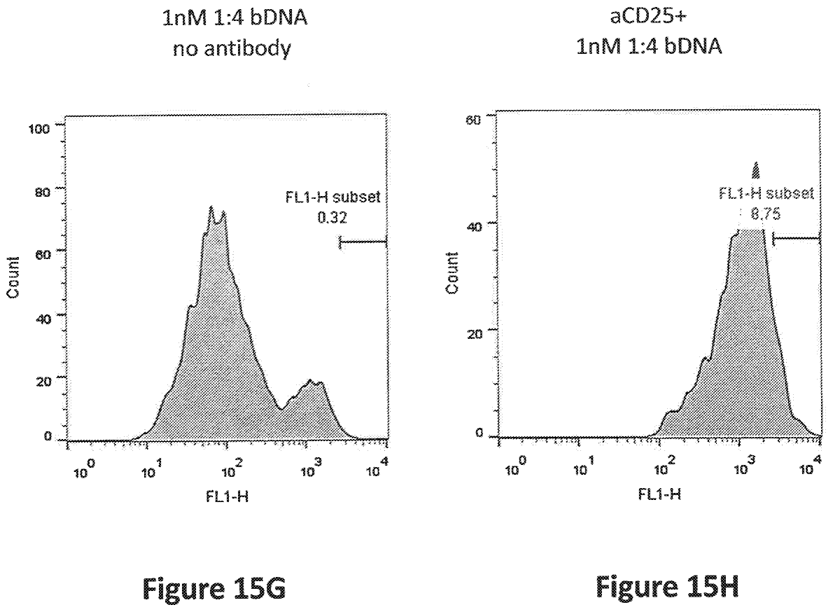

bDNA labeled cells. bDNA-SA and SA-FITC were compared using a CD25+ T-cell line labeled with biotin labeled anti-CD25. As shown in FIGS. 15D, 15F, and 15H, stepwise addition of antibody followed by fluorophore, bDNA gave much higher signal at 1 nM than SA-FITC at 18 nM (FIG. 15B). As shown in FIGS. 15C, 15E and 15G, even though antibody was not added to certain samples, bDNA was found to have background fluorescence when staining cells.

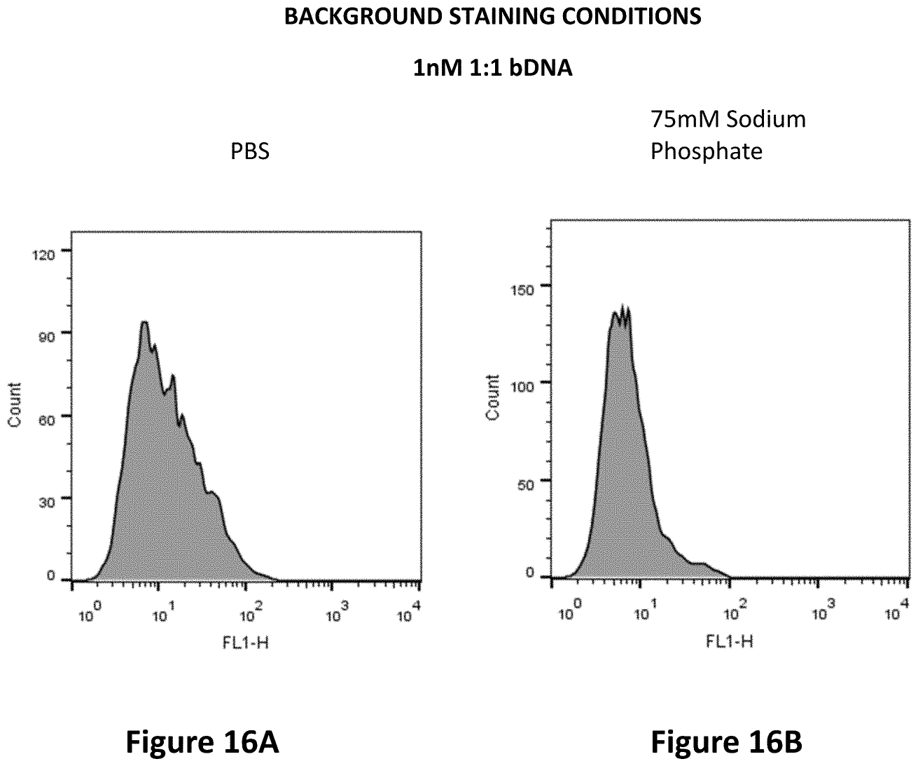

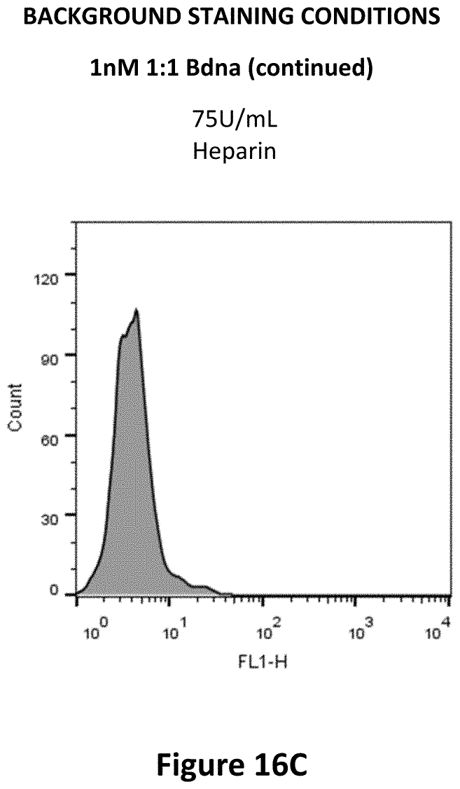

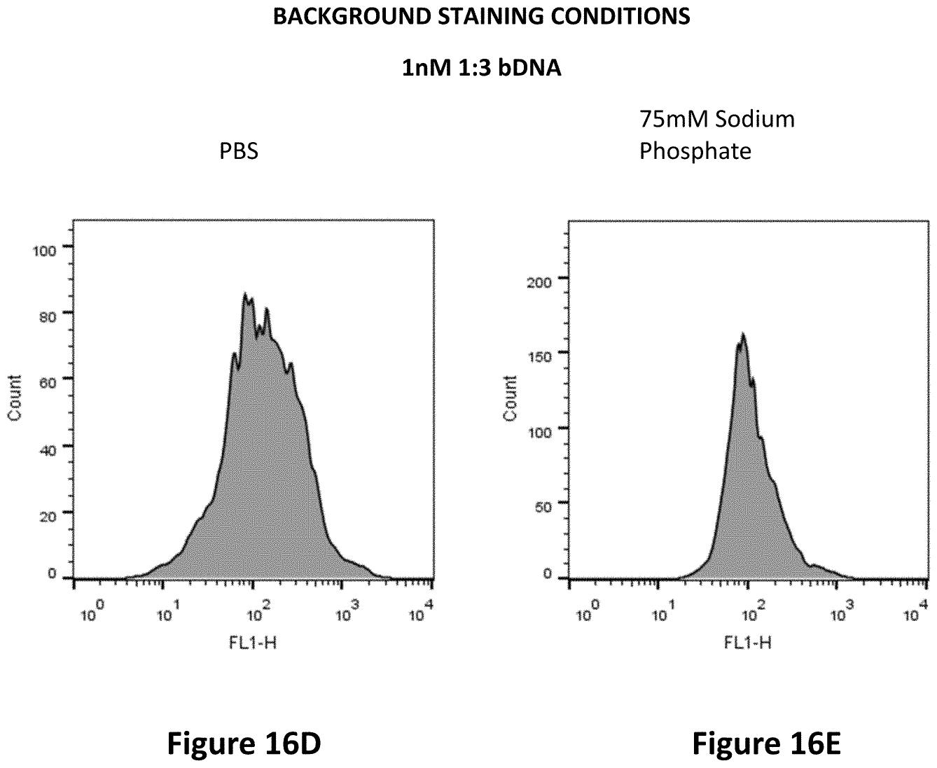

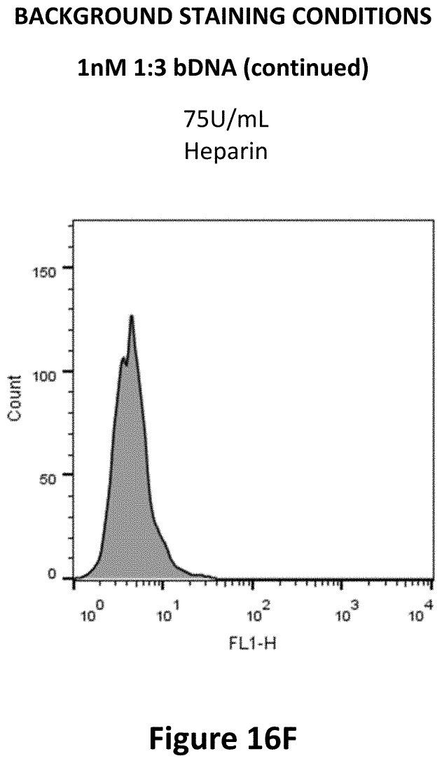

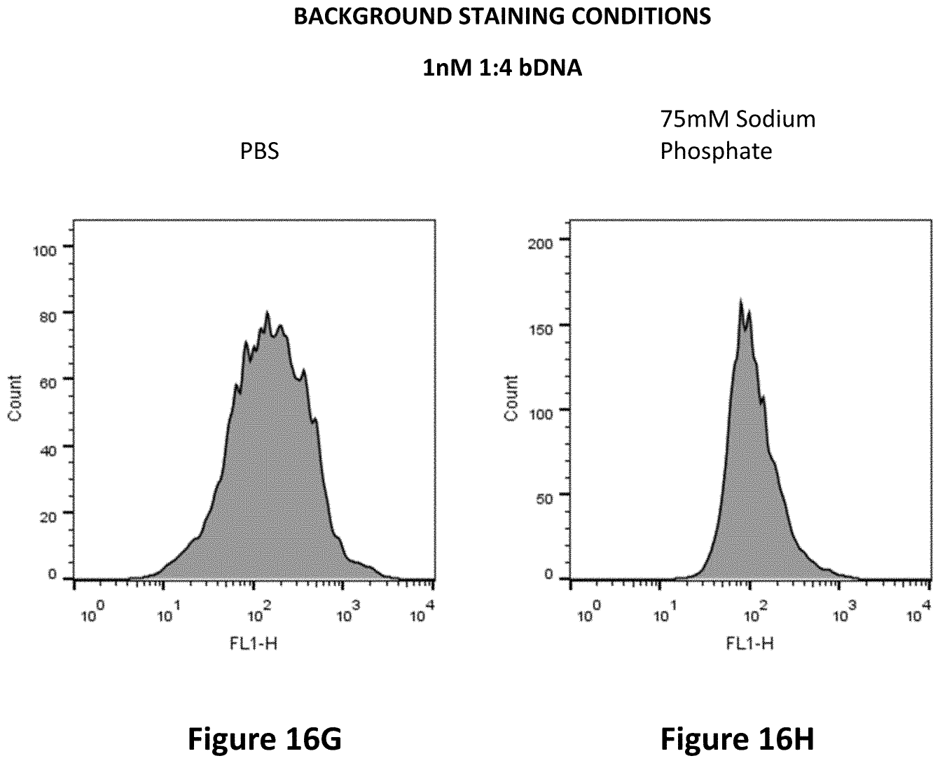

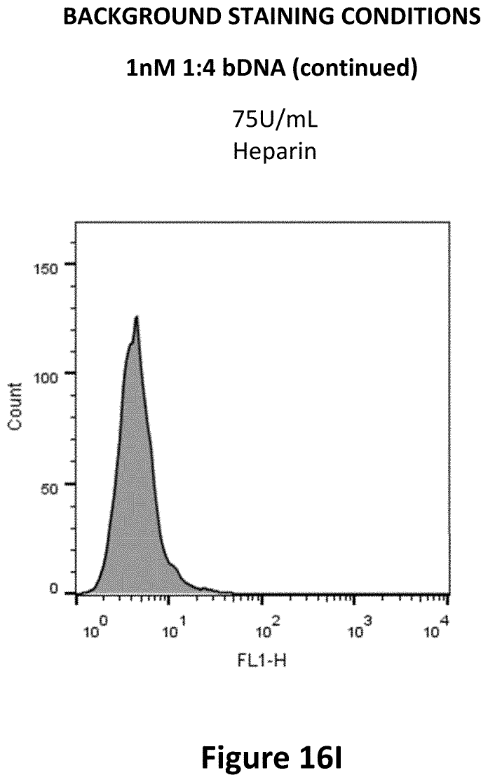

Removal of background signal. Different conditions were tested for the ability to remove background staining of 1:1 bDNA, 1:3 bDNA and 1:4 bDNA. Cells were assayed in PBS alone (FIGS. 16A, 16D, 16G) (control), in PBS with 75 mM sodium phosphate (FIGS. 16B, 16E, 16H) or in PBS with 75 U/mL of heparin (FIGS. 16C, 16F, 16I). As shown in FIGS. 16B, 16E and 16H, increased phosphate concentration resulted in moderate reduction in background as compared to PBS alone, while the addition of heparin completely removed background (FIGS. 16C, 16F, 16I). Accordingly, background fluorescence from bDNA staining of cells is easily removed.

### 7.17. Example 17: Flow Cytometry Detection of GAPDH Housekeeping Genes Using Molecular Beacons

GAPDH is a gene expressed in almost all cells of the body and is commonly used as a housekeeping gene in numerous assays. Molecular beacon probes that were a mixture of GAPDH 1 (SEQ ID NO: 11) and GAPDH 2 (SEQ ID NO: 12) probes as set forth in Example 1 (10 μM stock solution of both probes) were used to detect GAPDH in the H9 human T-cell line and in the CaSki human ectocervical cell line. Cells from both cell lines were fixated in Thinprep, washed, fixated in 5% formaldehyde in PBS for 30 minutes and washed again. The cells were then resuspended in 2% Triton X-100 in 1×SS containing 17 nM of the GAPDH specific probes (0.5 μL of probe stock solution added per 300 μL buffer). Cells were incubated for 1 hour at 65° C., then for 30 minutes at 4° C., and were analyzed using flow cytometry.

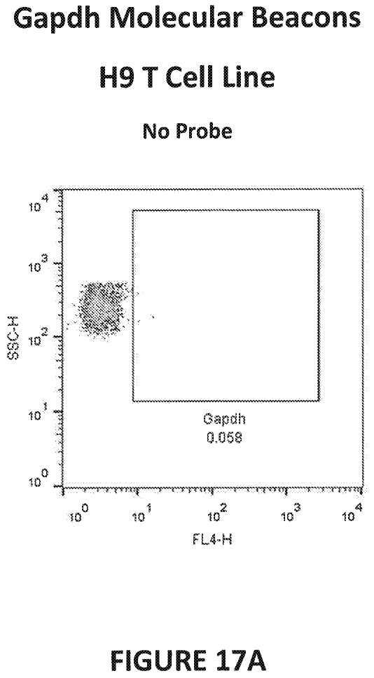

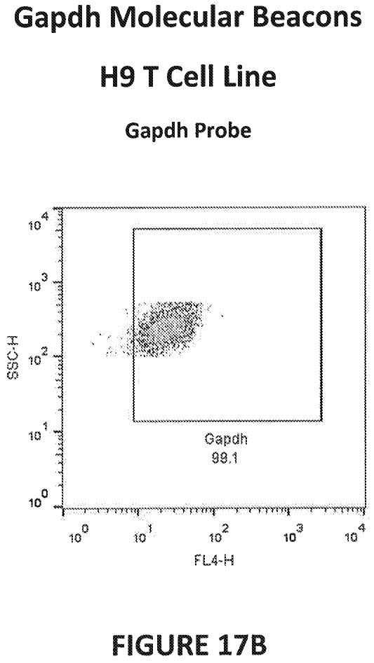

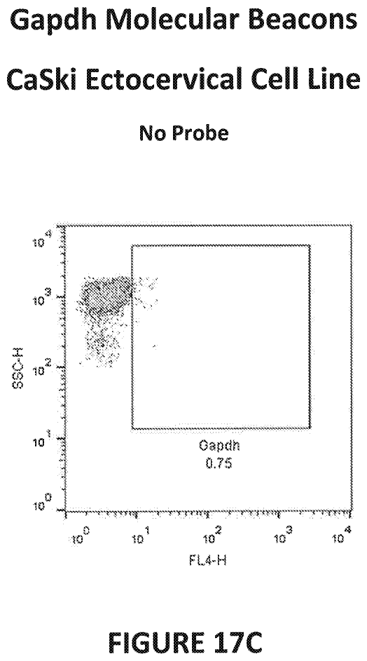

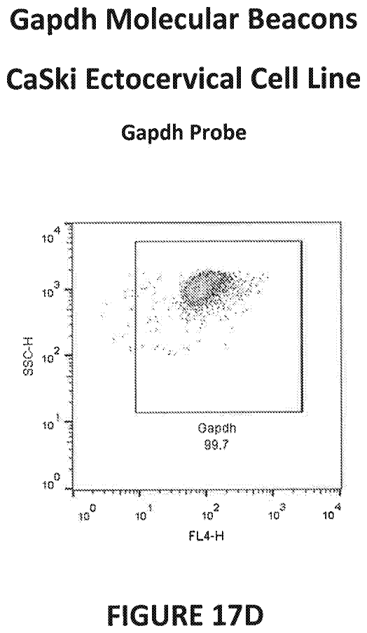

As shown in FIG. 17, the GAPDH molecular beacon probes successfully labeled GAPDH mRNA in both H9 (FIG. 17B) and CaSki (FIG. 17D) human cells. As shown in FIG. 17B, H9 cells express lesser amounts of GAPDH than CaSki cells. The results of this experiment are in keeping with previous observations that expression of housekeeping genes can vary between tissue types, between different individuals or between different cell lines, which often accrue mutations and abnormal karyotypes that affect gene expression. However, expression within one tissue type and from one subject is expected to show minimal variation.

### 7.18. Example 18: Staining of Neuronal Cell Line with Molecular Beacon Probes

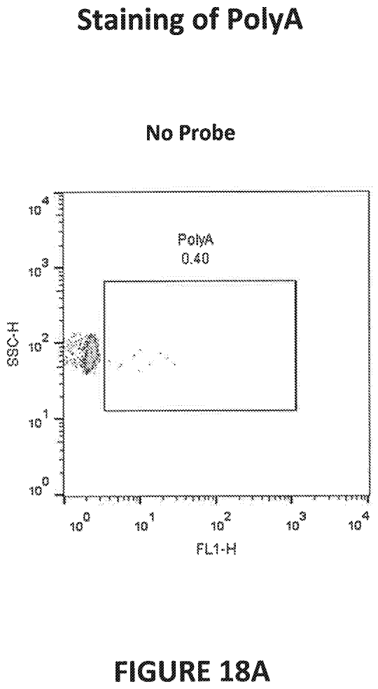

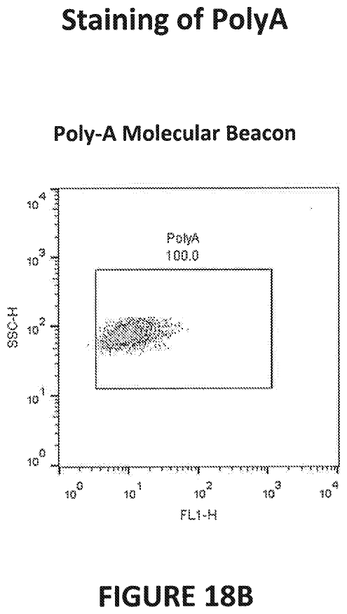

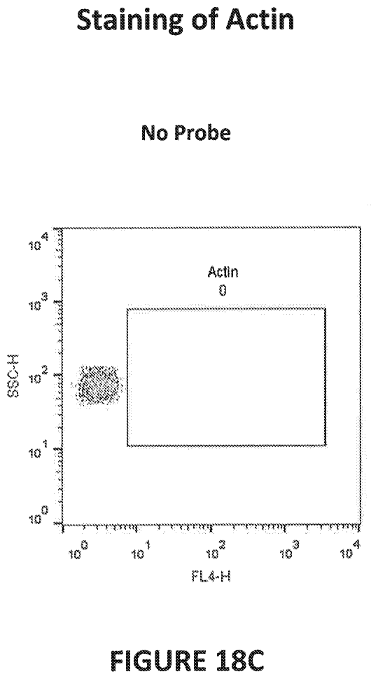

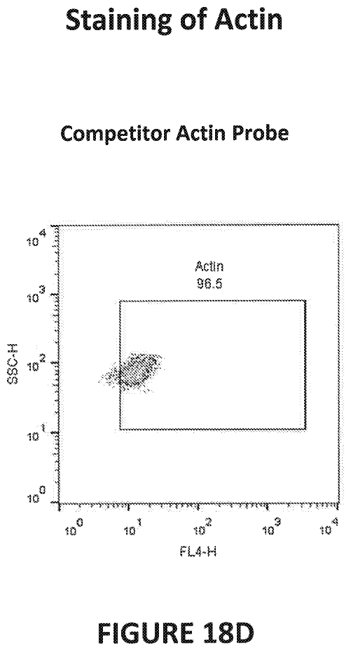

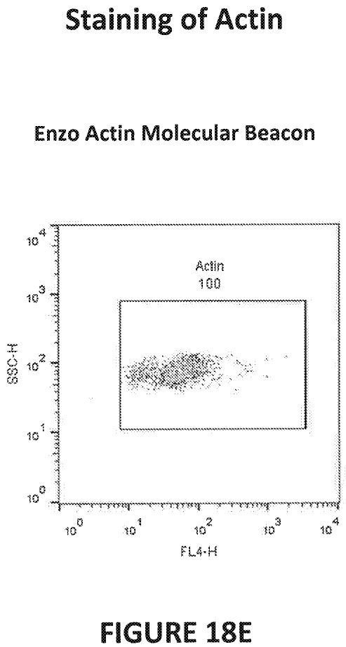

Staining of poly-A. SH-SY5Y cells, a human neuroblastoma cell line, were fixated in 5% formaldehyde in PBS for 30 minutes, and then washed. Cells were resuspended in 150 μL of 2% Triton X-100 in 1×SSC containing 17 nM (0.5 μL of probe stock solution added per 300 μL buffer) of a poly-A probe having the sequence: 6-FAM-ccagccTTTTTTTTTTTTTTTTTTTTTTTTTTTTTTTTTTTTTTTTTTTTTTTTTTggctgg-BHQ1 (SEQ ID NO: 33), which includes 50 T nucleotides flanked by labeled probe sequences, and was designed to hybridize to poly-A regions of diverse mRNAs present in the cells. Cells were then incubated in the dark at 65° C. for one hour, then at 4° C. for 30 minutes. Cells were analyzed by flow cytometry to assess the capability of molecular beacon probes to label housekeeping genes in neuronal cells. FIG. 18B shows that the poly-A probe (SEQ ID NO: 33) successfully labeled SH-SY5Y neuroblastoma cells and is a good candidate for a housekeeping gene control probe.

Staining of actin. SH-SY5Y cells were fixated and washed as set forth in the previous paragraph. Cells were resuspended in 150 μL of 2% Triton X-100 in 1×SSC containing 17 nM of a mixture of β-actin molecular beacon probes having the sequences set forth in SEQ ID NOs: 7, 8, 9 and 10 as shown in Example 1(0.5 μL of probe stock solution added per 300 μL buffer), which mixture labels multiple locations on actin mRNA transcripts, or 17 nM of a 3-actin specific SmartFlare™ probe (EMD Millipore), which labels a single part of the actin mRNA. Cells were analyzed by flow cytometry for actin housekeeping genes to assess the capability of molecular beacons to label neuronal cells. As shown in FIG. 18E, the molecular beacon cocktail provided a brighter signal than the SmartFlare™ probe (FIG. 18D). The brighter signal from the molecular beacon cocktail indicates that different cells in the population express different amounts of actin mRNA, while the SmartFlare™ lacks sensitivity to show such results. Accordingly, molecular beacons specific for actin provide excellent housekeeping control probes for labeling mRNA for flow cytometry detection.

### 7.19. Example 19: P16 Molecular Beacons as Cancer Markers

P16 is commonly overexpressed in cervical cancer, and therefore, is often used as a marker for this cancer. The H9 human T-cell line and CaSki human ectocervical cell line were used to test P16 molecular beacon probes having the sequences set forth in SEQ ID NOs: 16, 17, 18, 19, 20, 21, 22 and 23 of Example 1. H9, which is not of cervical cancer origin, was used as a negative control, while CaSki cells, a cervical cancer cell line with HPV integration, were used as a positive control. Cells were fixated in Thinprep, after which they were washed and fixated in 5% formaldehyde in PBS for 30 minutes, and were washed again. Cells were then resuspended in 2% Triton X-100 in 1×SSC containing 17 nM P16 probes (probe stock solution was an 8 μM mixture of all 7 P16 probes; 0.5 μL of stock solution were added per 300 μL of buffer). Cells were incubated for 1 hour at 65° C., then for 30 minutes at 4° C., after which they were analyzed by flow cytometry.

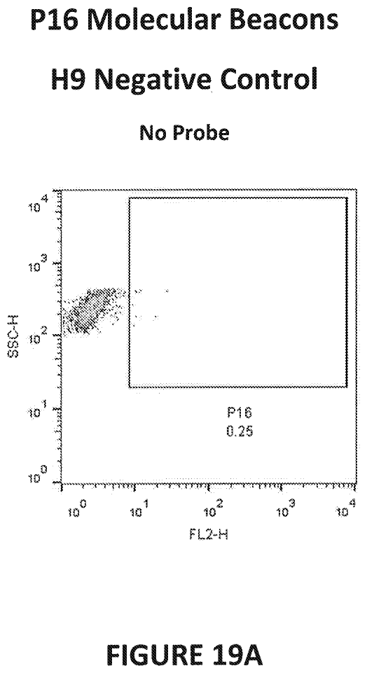

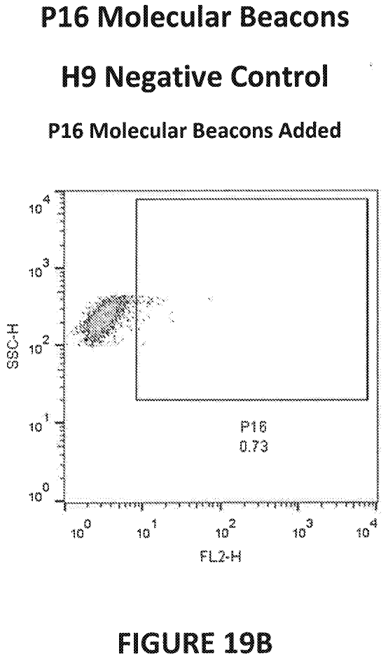

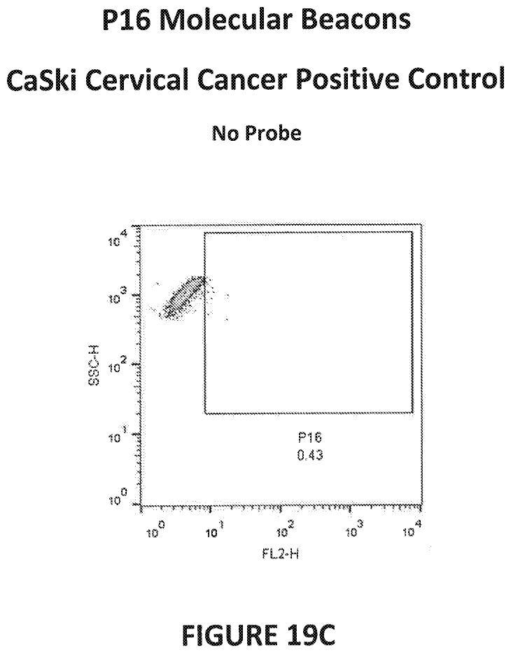

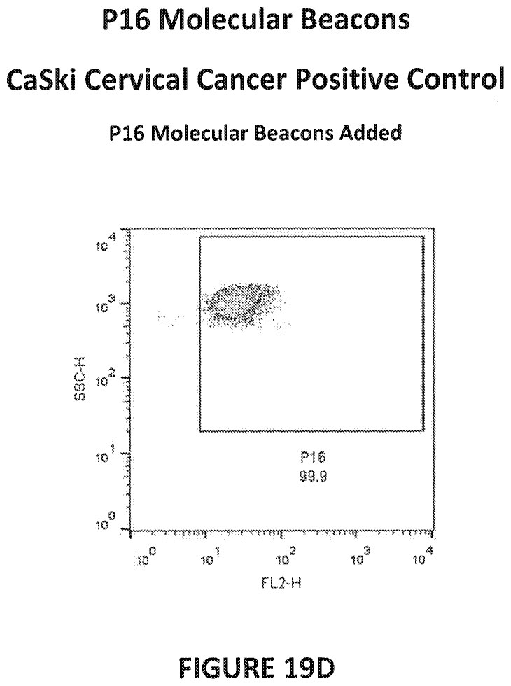

H9 cells showed minimal P16 expression (FIG. 19B), while CaSki cells showed significant levels of P16 (FIG. 19D). These results support the use of P16 as a marker for cervical cancer, and show that use of the P16-specific molecular beacon probes having the sequences set forth in Example 1 in the flow cytometry methods described herein allows for efficient detection of P16 overexpression in cancer cells.

### 7.20. Example 20: Use of Bead-Bound Probes for Detection of HPV

Preparation of polystyrene beads. A 1:200 dilution of 1% w/v streptavidin coated polystyrene beads was made with DPBS to a volume of 200 μL. 1 μL of a 1 μM solution of either a collection of HPV E6/E7 sequences (HPV Positive Control) or a set of non-target controls (Negative Control) were added to the solution of beads and mixed well by vortexing. The bead-target mixtures were incubated at room temperature for 1 hour to allow for attachment of targets to the beads. Bead mixtures were spun for 5 minutes at 2000 g to pellet the beads. Supernatant was removed to remove unbound target sequences, and beads coated with the target sequences were resuspended to a volume of 100 μL.

Sequences for the HPV Positive Control were:

(SEQ ID NO: 59)
Bio-TTTTGTGTGTACTGCAAGCAACAGTTACT
(SEQ ID NO: 60)
Bio-TTTATGCTGTATGTGTGATAAATGTTTA
(SEQ ID NO: 61)
Bio-TTTACGTAGAGAAACCCAGCTGTAAT
(SEQ ID NO: 62)
Bio-TTTGAGCAATTAAGCGACTCAGAGGAC
(SEQ ID NO: 63)
Bio-TTTGAACCACAACGTCACACAAT
(SEQ ID NO: 64)
Bio-TTTCACTGTCCTTTGTGTGTCCGTGGTGTG

Sequences for the Negative Control were:

(SEQ ID NO: 65)
Bio-TTTCACAACGGCTCCGGCATGTGCAAGTG
(SEQ ID NO: 66)
Bio-CACGTGATGGTGGGCATGGGTCAGTG
(SEQ ID NO: 67)
Bio-TTTGCAGCACAATGAAGATCAAGAC

Streptavidin coated polystyrene beads without attached target sequences, polystyrene beads attached to the Negative Control sequences and polystyrene beads attached to HIPV E6E7 target sequences were mixed with HPV E6/E7 molecular beacons (SEQ ID NOs: 1-6) and then assayed by flow cytometry.

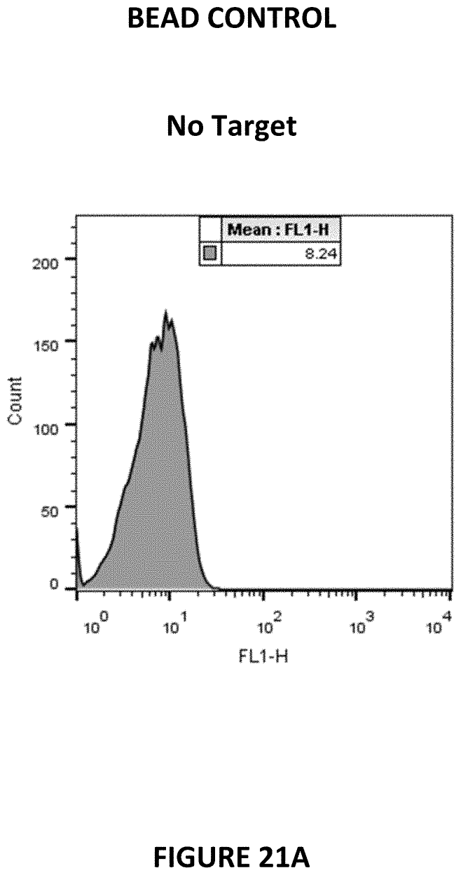

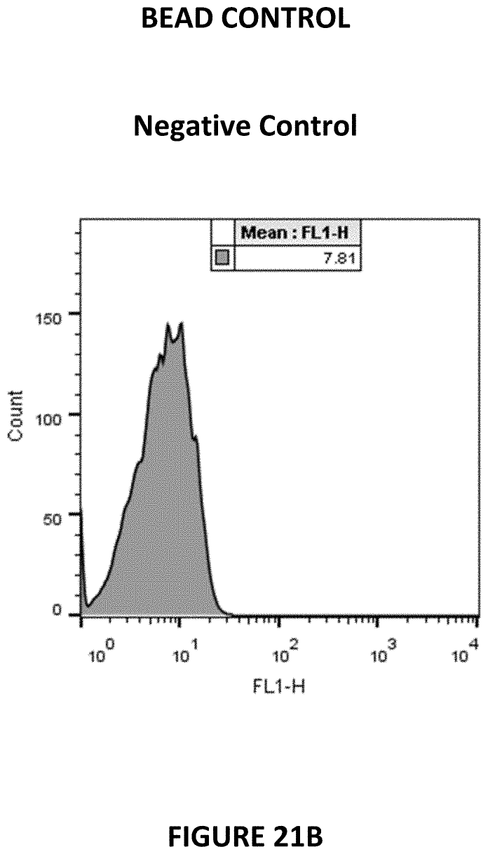

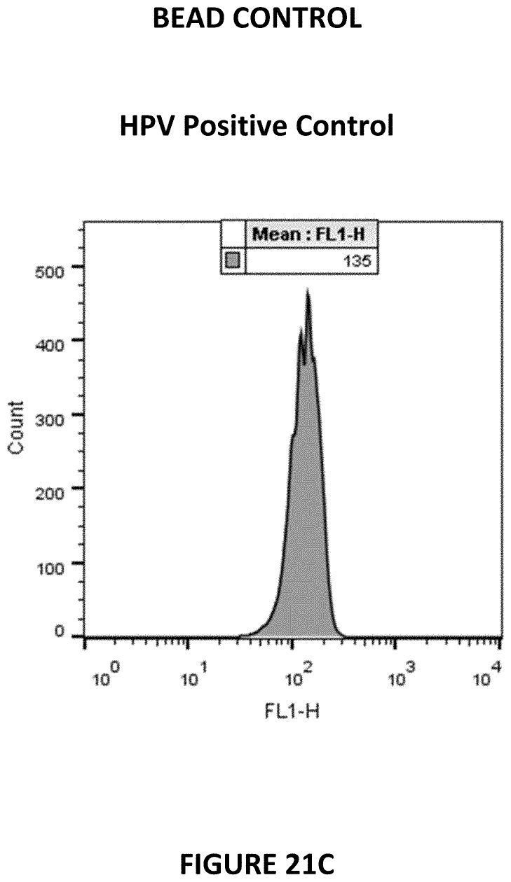

FIG. 21A shows a histogram of streptavidin coated polystyrene beads without target to show baseline fluorescence. In FIG. 21B the Negative Control sequences lacking complementarity to the HPV target sequences shows fluorescence levels that are similar to the beads without any target shown in FIG. 21A. FIG. 21C shows a greater than 17-fold increase in fluorescence over the streptavidin coated beads (FIG. 21A) and the Negative Control coated beads (FIG. 21). This experiment verifies that hybridization of molecular beacons specific for E6/E7 bound target sequences took place and that the beacons were specific for their targets, i.e. signal generation only took place through complementary binding between beacons and target sequences.

### 7.21. Example 21: Use of 18S Probes as Housekeeping Genes

In previous experiments, actin mRNA expression showed negative in E6/E7 negative ectocervical cells. See Example 3, above. Accordingly, E6/E7 patient samples were tested to identify housekeeping genes that were more actively expressed in ectocervical cell populations (low SSC population) and endocervical/PBMC cells (high SSC population). Cells were assayed for expression of 28S, GAPDH, polyA and 18S housekeeping genes.

Cervical cell samples in ThinPrep were stained for housekeeping gene mRNA. 1×10⁵ fixed cells per sample were stained for the selected mRNAs using the FlowScript protocol as follows. Cells were spun down and resuspended in 1% formaldehyde in DPBS and incubated for 30 minutes. Cells were washed in hybridization buffer (2% Triton X-100 in 1×SSC) twice, followed by addition of molecular beacons for housekeeping genes actin (SEQ ID NOs: 7-10), GAPDH (SEQ ID NOs: 11-12), 28S (SEQ ID NOs: 13-15) and 18S (SEQ ID NOs: 59-62), and PolyA probe (SEQ ID NO: 33) using 0.5 μL of 5 μM stock probe concentration added per 300 L hybridization buffer in the staining protocol. As a negative control, some samples were incubated and analyzed without probe addition.

Molecular beacons specific for 18S are shown below:

18S-1
(SEQ ID NO: 68)
(Cy3)-ccagccCCAAGATCCAACTACGAGCTTggctgg-(EQ1)
18S-2
(SEQ ID NO: 69)
(Cy3)-ccagccCCATCCAATCGGTAGTAGCGggctgg-(EQ1)
18S-3
(SEQ ID NO: 70)
(Cy3)-ccagccCGGCTGCTGGCACCAGACTTgcggct-(EQ1)
18S-4
(SEQ ID NO: 71)
(Cy3)-ccagccCCCGTGTTGAGTCAAATTAAGCggctgg-(EQ1)

in which EQ1 is a quencher with the formula (E)-2,5-dioxopyrrolidin-1-yl 4-(7-methoxy-2,2,4-trimethyl-6-((4-nitrophenyl)diazenyl)quinolin-1(2H)-yl)butanoate. See U.S. application Ser. No. 14/672,944 filed Mar. 30, 2015.

Cells were hybridized at 65° C. for 1 hour, then at 4° C. for 30 minutes while protected from light. Cells were then analyzed for mRNA expression by flow cytometry.

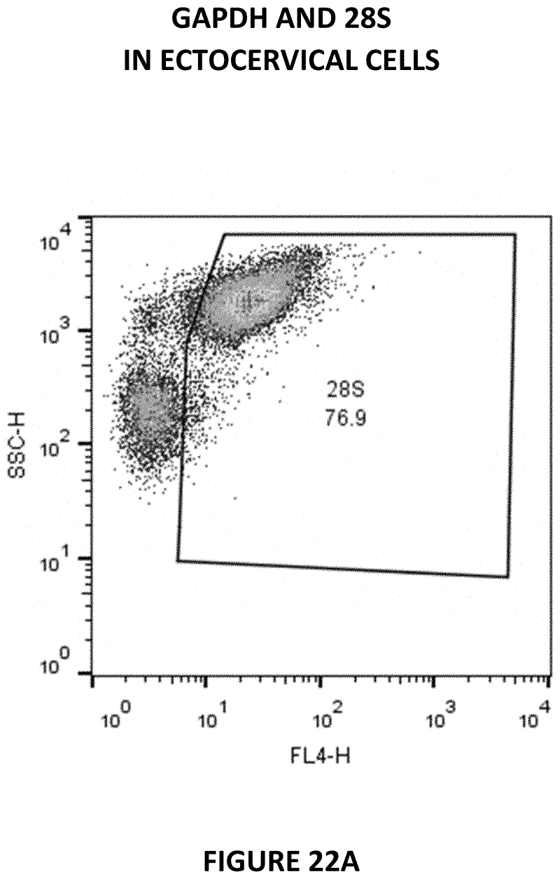

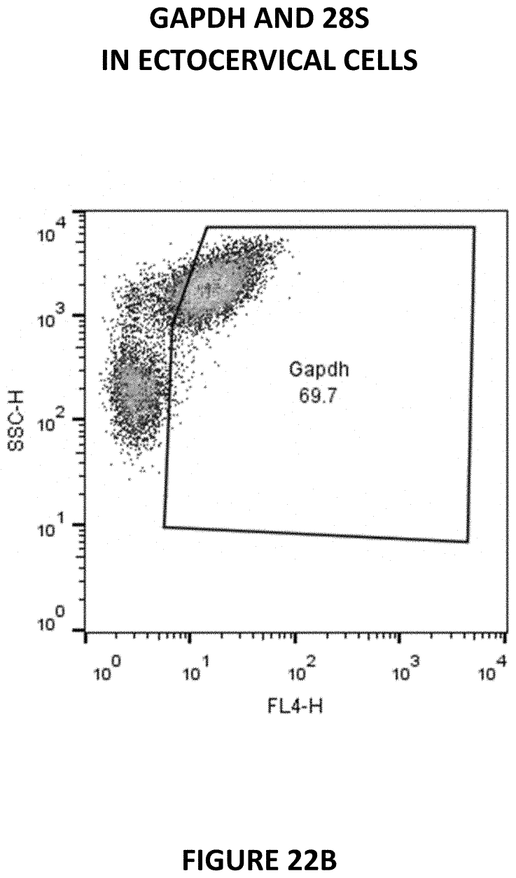

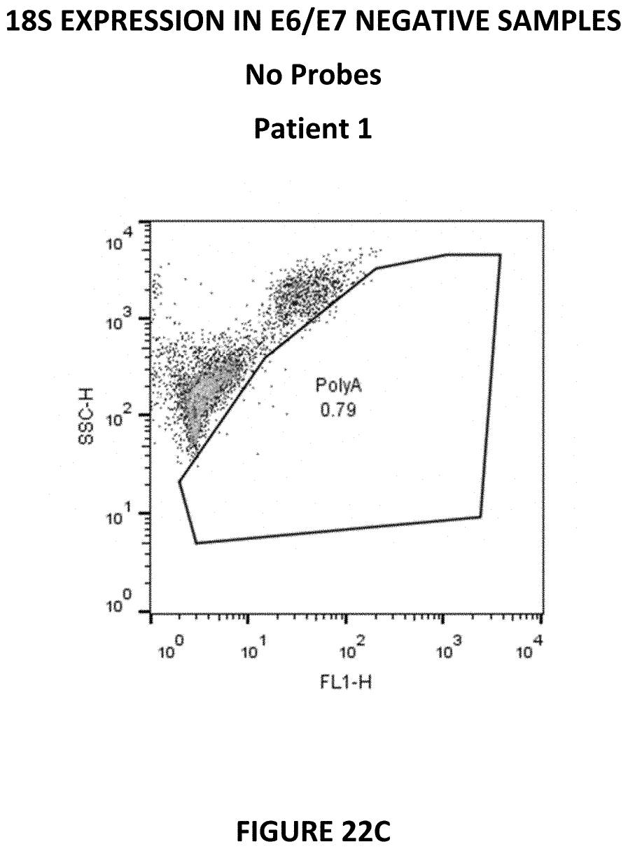

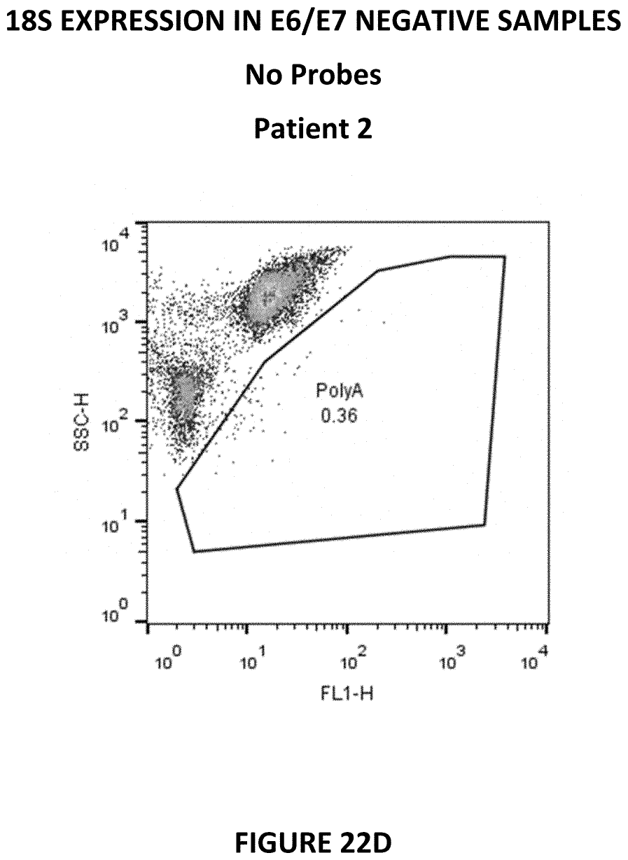

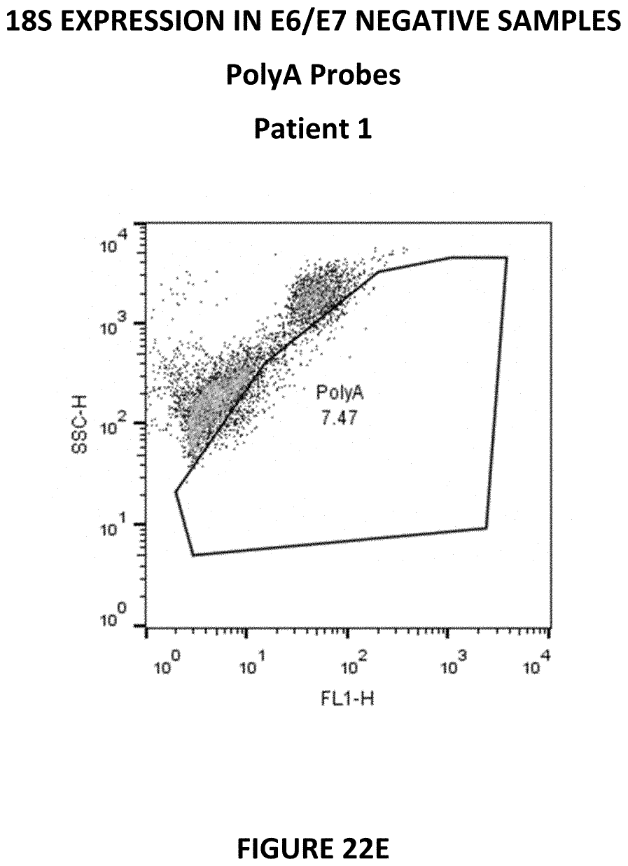

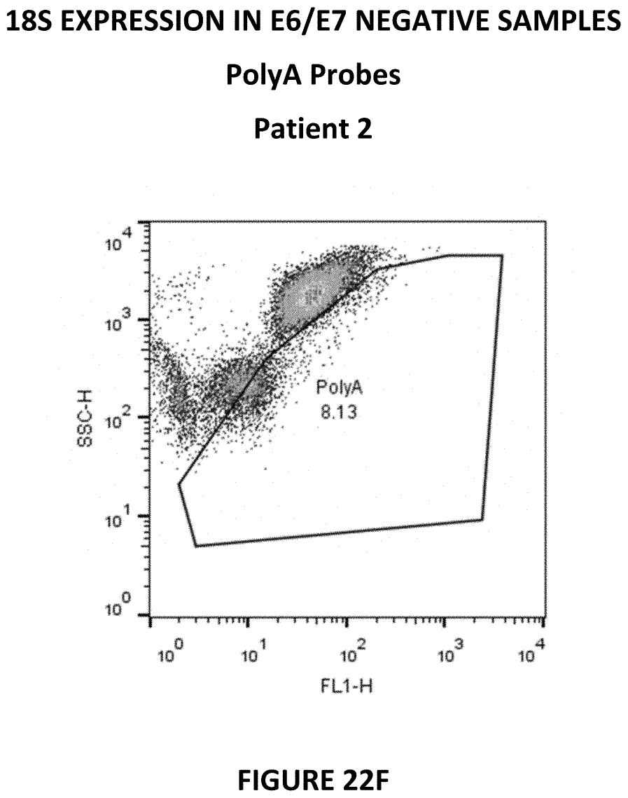

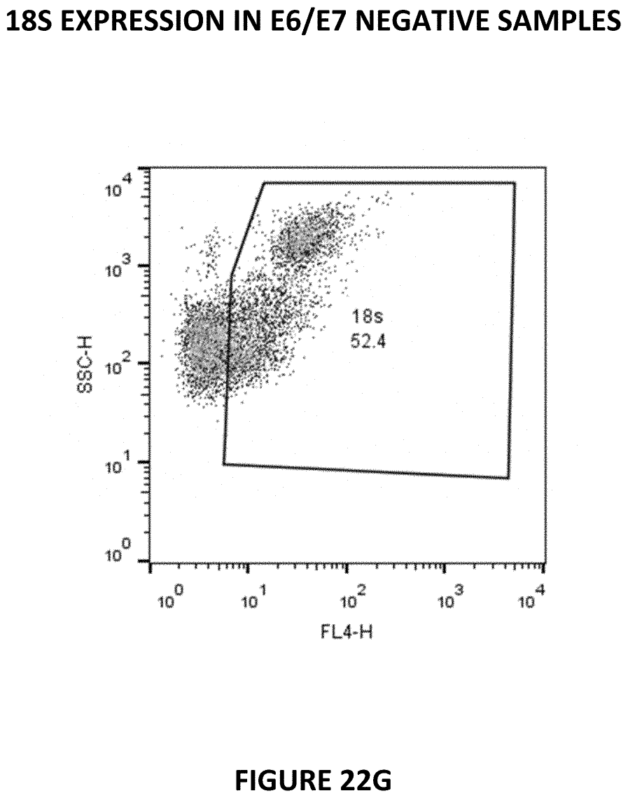

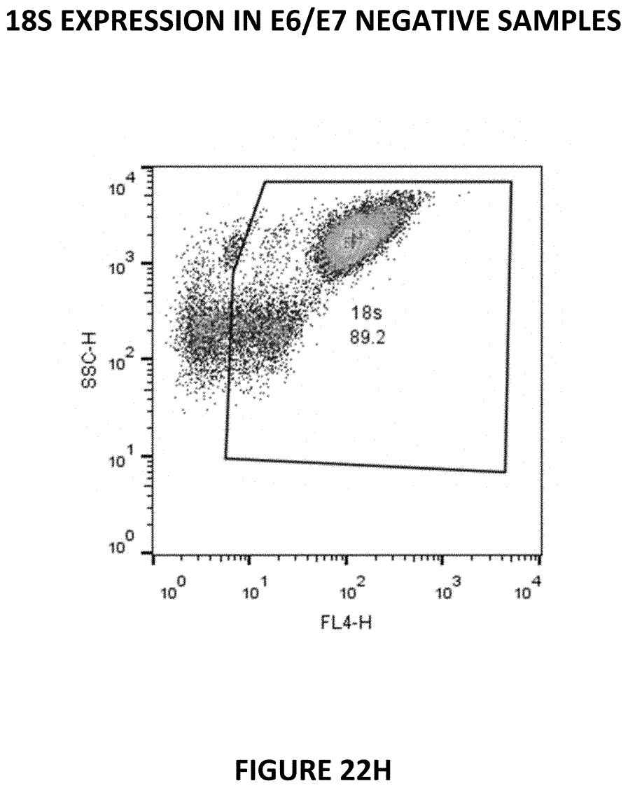

Of the housekeeping genes assayed in the E6/E7 negative patient sample, expression of 18S and GAPDH genes were comparable to actin expression, i.e., as shown in FIG. 22A and FIG. 22B they were mostly negative in the low SSC population (ectocervical cells) and well expressed in the high SSC population (endocervical cells). With regard to polyA expression, there was high levels of signal seen in the two patient samples with the no probe control (FIGS. 22C and 22D), while addition of the PolyA probes to the same patient samples gave detectable but low signal for both the low SSC populations and high SSC populations (FIGS. 22E and 22F).

In contrast, as set forth in FIGS. 22G-22I, in three different patient samples, 18S was strongly expressed in both the ectocervical (low SSC population) as well as the endocervical cells (high SSC population) demonstrating that these probes can be successfully used with both populations. Accordingly, we have identified the 18S housekeeping gene as a robust hybridization control for flow cytometry detection of RNA that can be used to select or reject cells for analysis of markers of interest.

### 7.22. Example 22: Design and Testing of Cancer Marker Probes

Molecular beacon probes specific for cancer markers KI67 and Top2A were designed for use alone, in combination with HPV E6/E7 probes, or in combination with other cancer markers such as P16 previously used in Example 7. As discussed previously, P16 is a marker associated with HPV-induced cancer. KI67 and Top2A are related to cell cycle progression and are common markers for many different cancers. Probes were tested on patient pap smear cervical samples.

Molecular beacons specific for KI67 are shown below:

KI67-1
(SEQ ID NO: 72)
(FAM)-ccagccGCACCACTTCTTCTTTTGGAACATAggctgg-(BHQ1)
KI67-2
(SEQ ID NO: 73)
(FAM)-ccagccCAAAGGTATTCCCTCACTCTCATCAggctgg-(BHQ1)
KI67-3
(SEQ ID NO: 74)
(FAM)-ccagccCAAAGGTATTCCCTCACTCTCATCAggctgg-(BHQ1)
KI67-4
(SEQ ID NO: 75)
(FAM)-ccagccACTCTTTCTCCTCCTCTCTTAGGAAggctgg-(BHQ1)
KI67-5
(SEQ ID NO: 76)
(FAM)-ccagccGACTCTTGTTTTCCTGATGGTTGAGggctgg-(BHQ1)

Molecular beacons specific for Top2A are shown below:

Top2A-1
(SEQ ID NO: 77)
(FAM)-ccagccTCCGCAGCATTAACTAGAATCTCATggctgg-(BHQ1)
Top2A-2
(SEQ ID NO: 78)
(FAM)-ccagccTTGTTTTCCGGATCAATTGTGACTCggctgg-(BHQ1)
Top2A-3
(SEQ ID NO: 79)
(FAM)-ccagccATTTTCATTTACAGGCTGCAATGGTggctgg-(BHQ1)
Top2A-4
(SEQ ID NO: 80)
(FAM)-ccagccTCCATCCATGTCTGTTTGAACATTTggctgg-(BHQ1)
Top2A-5
(SEQ ID NO: 81)
(FAM)-ccagccTACGAAATCCTTTTACTGGCAGTTTggctgg-(BHQ1)

ThinPrep cervical clinical samples were stained for cervical cancer marker mRNA in addition to E6/E7 mRNA using the FlowScript protocol below. 1×10⁵ fixed cells per sample were then stained for the indicated mRNAs using the FlowScript protocol. Specifically, cells were spun down and resuspended in 1% formaldehyde in DPBS and incubated for 30 minutes. Cells were washed in hybridization buffer (2% Triton X-100 in 1×SSC) twice, after which molecular beacons for the indicated genes were added. HPV and P16 specific probes used in this Example were described previously in Example 1 (SEQ ID NOs: 1-3 and SEQ ID NOs: 16-23 respectively). Cells were hybridized at 65° C. for 1 hour, then at 4° C. for 30 minutes, protected from light. Cells were then analyzed for mRNA expression by flow cytometry.

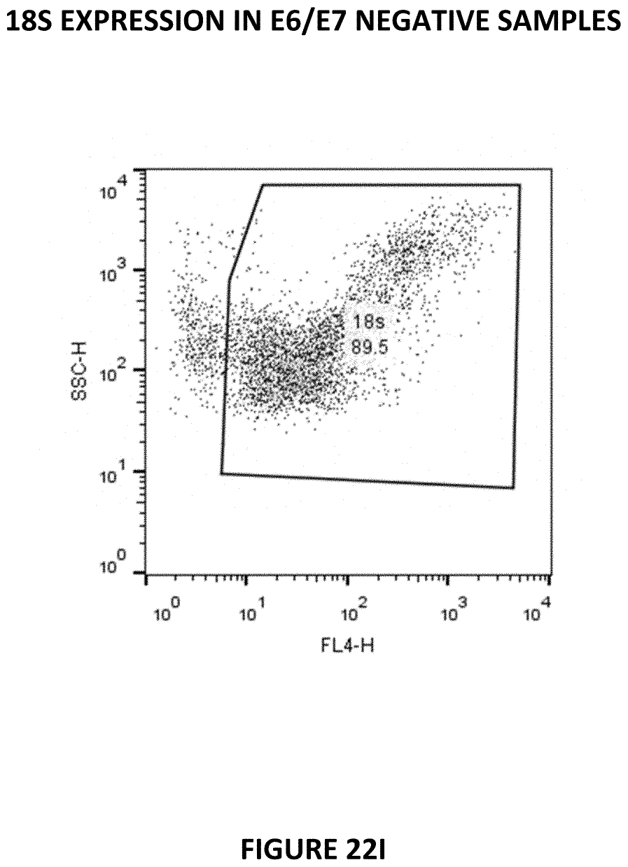

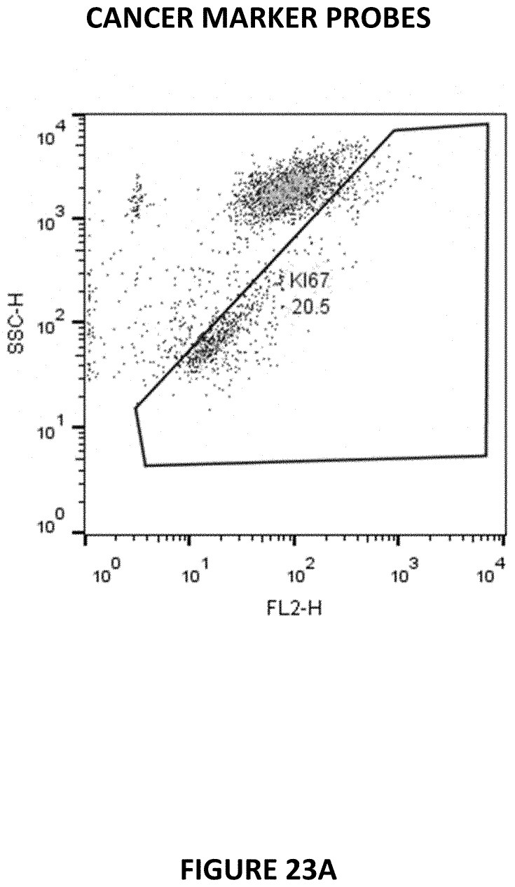

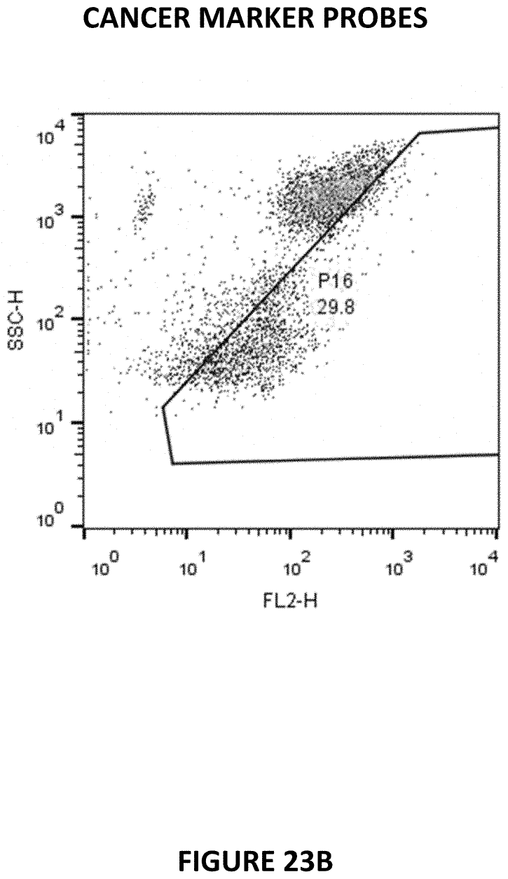

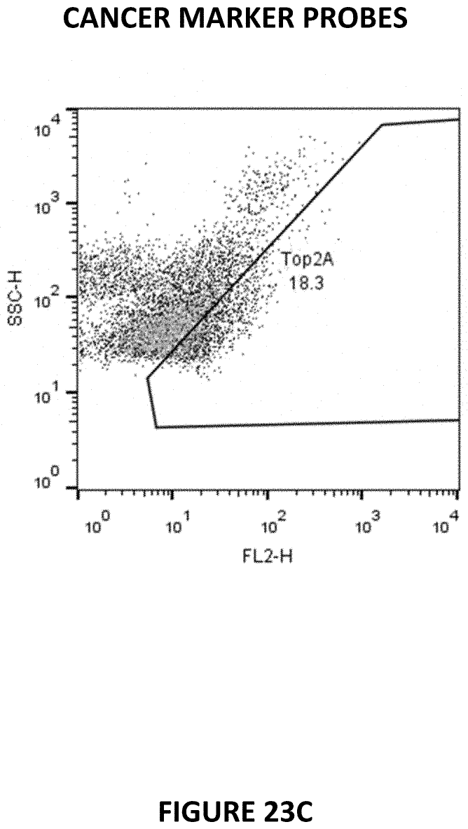

As shown in FIGS. 23A-23C, P16 positive cells (FIG. 23B) also tested positive for cancer markers KI67 (FIG. 23A) and Top2A (FIG. 23C). KI67, Top2A and P16 mRNA were verified as expressed in cervical samples of high risk HPV infected patients, supporting their use as cervical cancer markers.

### 7.23. Example 23: Design and Testing of Probes for Genes Related to Inflammation

Molecular beacon probes specific for genes involved in inflammatory processes were tested on patient samples in order to assay expression of inflammatory markers in immune cells, such as PBMCs. In this Example, naïve PBMCs from healthy individuals and PBMCs from patients with various immune-mediated disorders were assayed using the following molecular beacons:

Molecular beacons specific for INFγ were as described in Example 14

Molecular beacons specific for IL-10 are shown below:

I10-75
(SEQ ID NO: 82)
(Cy3)-ccgtgtcAAGTGGGTGCAGCTGTTCTCAgacacgg-(BHQ2a)
I10-104
(SEQ ID NO: 83)
(Cy3)-cgcaacTCTCGAAGCATGTTAGGCAGgttgcg-(BHQ2a)
I10-132
(SEQ ID NO: 84)
(Cy3)-cccgagTTCACTCTGCTGAAGGCATctcggg-(BHQ2a)
I10-212
(SEQ ID NO: 85)
(Cy3)-ctggagACCCAGGTAACCCTTAAAGTCctccag-(BHQ2a)
I10-239
(SEQ ID NO: 86)
(Cy3)-ccgccTGGATCATCTCAGACAAGGCTTggcgg-(BHQ2a)
I10-287
(SEQ ID NO: 87)
(Cy3)-ccccaaCTGGGTCTTGGTTCTCAGCttgggg-(BHQ2a)
I10-341
(SEQ ID NO: 88)
(Cy3)-ccaagaTCAGCCTGAGGGTCTTCAGGTtcttgg-(BHQ2a)
I10-371
(SEQ ID NO: 89)
(Cy3)-cggcAGGGAAGAAATCGATGACAGCgccg-(BHQ2a)
I10-414
(SEQ ID NO: 90)
(Cy3)-cgtggaAGGCATTCTTCACCTGCtccacg-(BHQ2)
I10-445
(SEQ ID NO: 91)
(Cy3)-ccctccaaTGGCTTTGTAGATGCCTTTCTCttggaggg-
(BHQ2a)

Molecular beacons specific for Foxp3 are shown below:

(SEQ ID NO: 92)
(FAM)-ccagccGTTGAGAGCTGGTGCATGAAATGggctgg-(BHQ1)
(SEQ ID NO: 93)
(FAM)-ccagccATGTTGTGGAGGAACTCTGGGAATGggctgg-(BHQ1)
(SEQ ID NO: 94)
(FAM)-ccagccCATGTTGTGGAGGAACTCTGGGAATggctgg-(BHQ1)
(SEQ ID NO: 95)
(FAM)-ccagccAGGAACTCTGGGAATGTGCTGTTTggctgg-(BHQ1)
(SEQ ID NO: 96)
(FAM)-ccagccATGTTGTGGAGGAACTCTGGGAATgggctgg-(BHQ1)

Molecular beacons specific for IL-17 are shown below:

I17-199
(SEQ ID NO: 97)
(FAM)-ccttccAAGGTGAGGTGGATCGGTTGTAggaagg-(BHQ2a)
I17-259
(SEQ ID NO: 98)
(FAM)-ccggcACTTTGCCTCCCAGATCACAGAgccgg-(BHQ2a)
I17-51
(SEQ ID NO: 99)
(FAM)-ccggagATTCCTGCCTTCACTATGGCctccgg-(BHQ2a)
I17-126
(SEQ ID NO: 100)
(FAM)-ccggaTTCAGGTTGACCATCACAGtccgg-(BHQ2a)
I17-91
(SEQ ID NO: 101)
(FAM)-ccaggTTGTCCTCAGAATTTGGGCATcctgg-(BHQ2a)
I17-341
(SEQ ID NO: 102)
(FAM)-cgccatcAGGACCAGGATCTCTTGCTGgatggcg-(BHQ2a)
I17-300
(SEQ ID NO: 103)
(FAM)-cagtccaCGTTCCCATCAGCGTtggactg-(BHQ2a)
I17-407
(SEQ ID NO: 104)
(FAM)-cctagccCACGGACACCAGTATCTTCTggctagg-(BHQ2a)
I17-230
(SEQ ID NO: 105)
(FAM)-caccgTATCTCTCAGGGTCCTCATTGcggtg-(BHQ2a)

Naive peripheral blood mononuclear cells (PBMCs) of healthy subjects and subjects suffering from an immune-mediated disorder were isolated from whole blood by density gradient separation, washed, and fixed in ThinPrep solution. 1×10⁵ fixed cells per sample were then stained for the indicated mRNAs using the FlowScript protocol. Cells were spun down and resuspended in 1% formaldehyde in DPBS and incubated for 30 minutes. Cells were washed in hybridization buffer (2% Triton X-100 in 1×SSC) twice, after which molecular beacons for the indicated genes were added. Cells were hybridized at 65° C. for 1 hour, then at 4° C. for 30 minutes while protected from light. Cells were then analyzed for mRNA expression by flow cytometry.

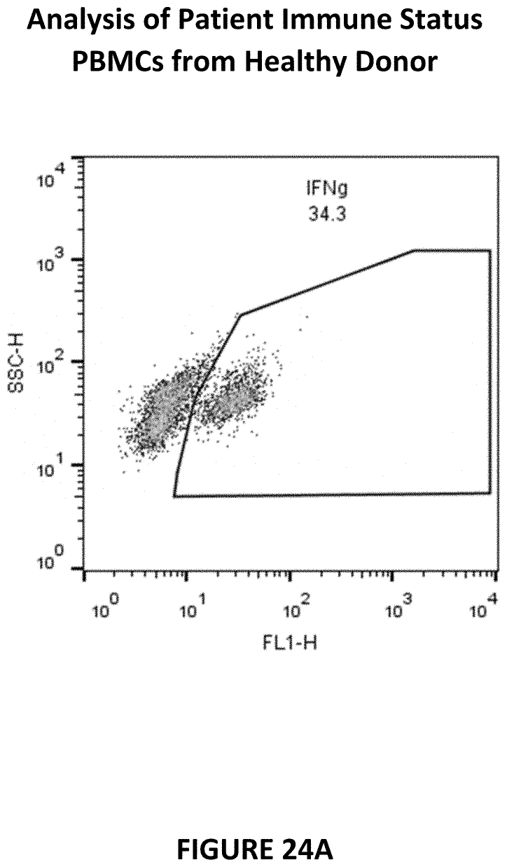

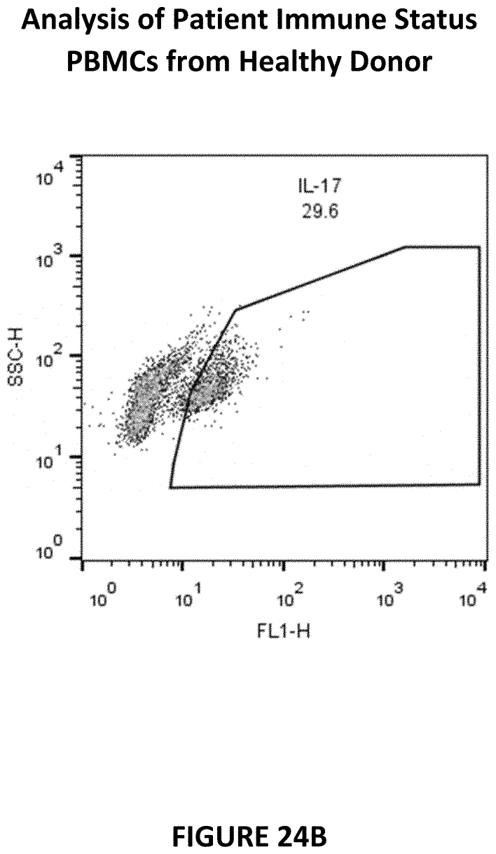

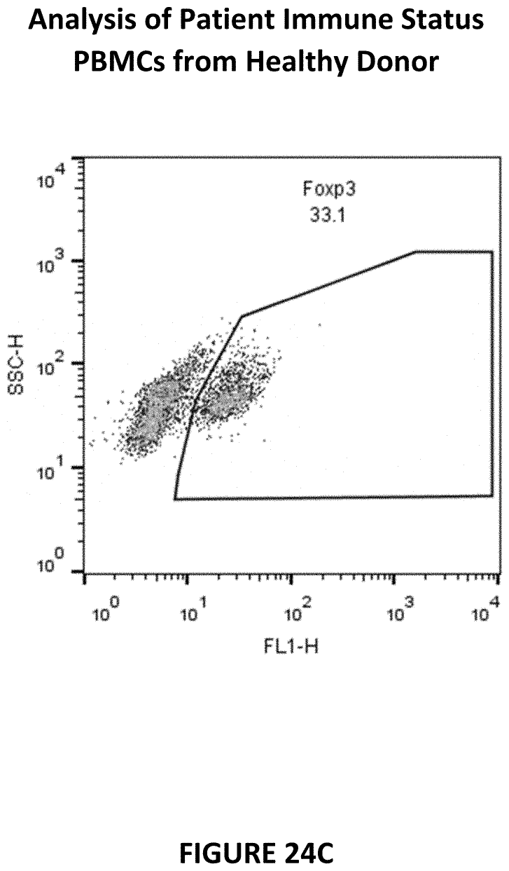

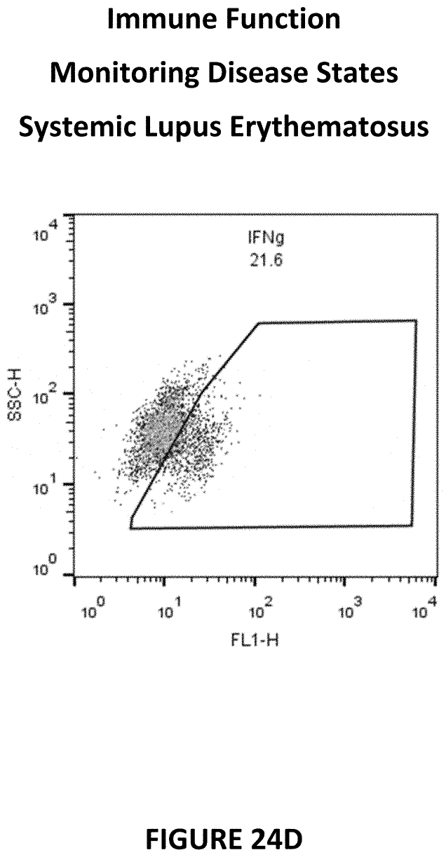

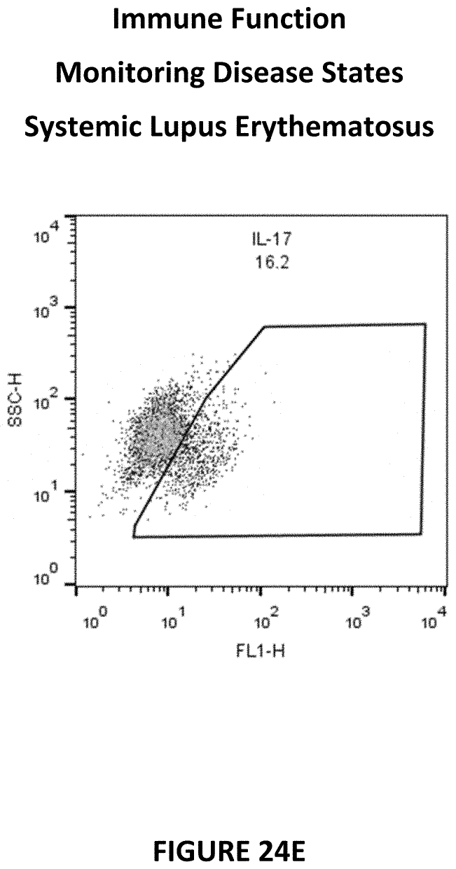

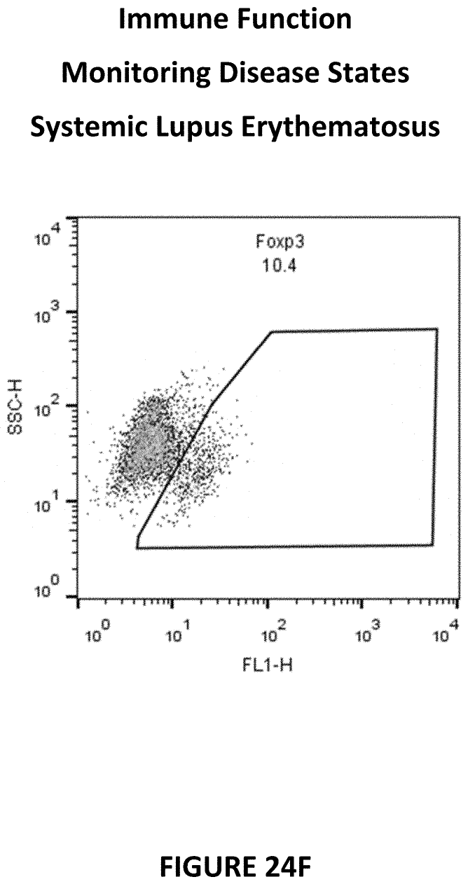

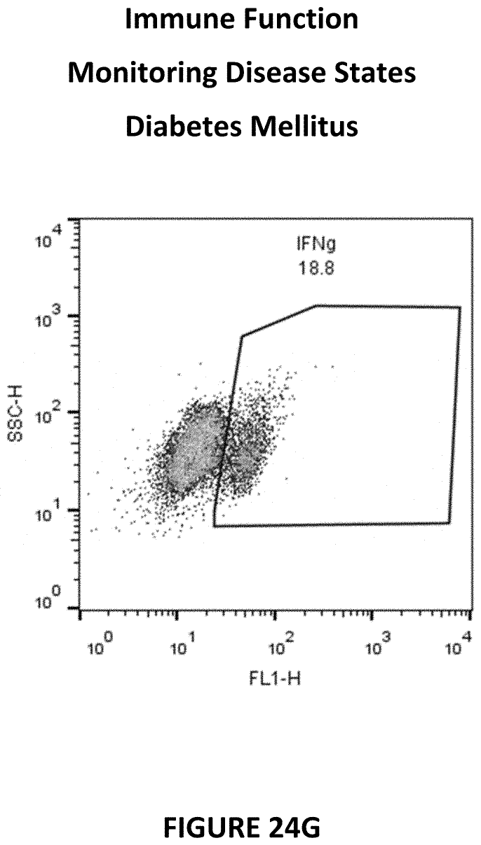

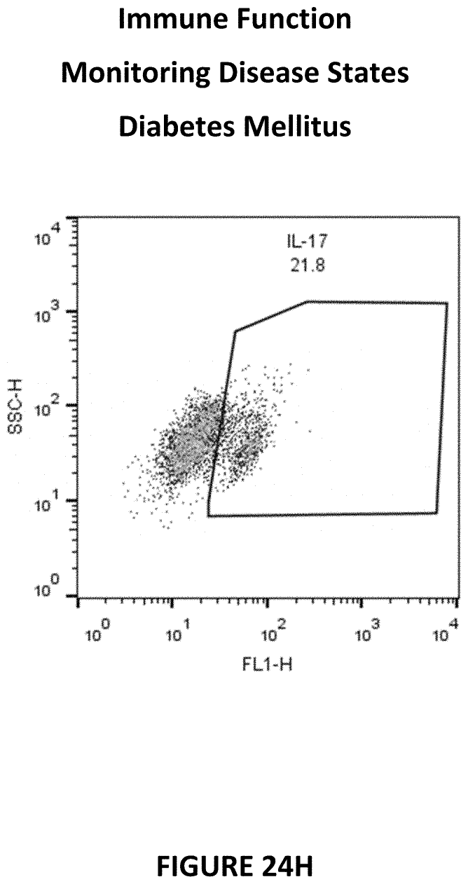

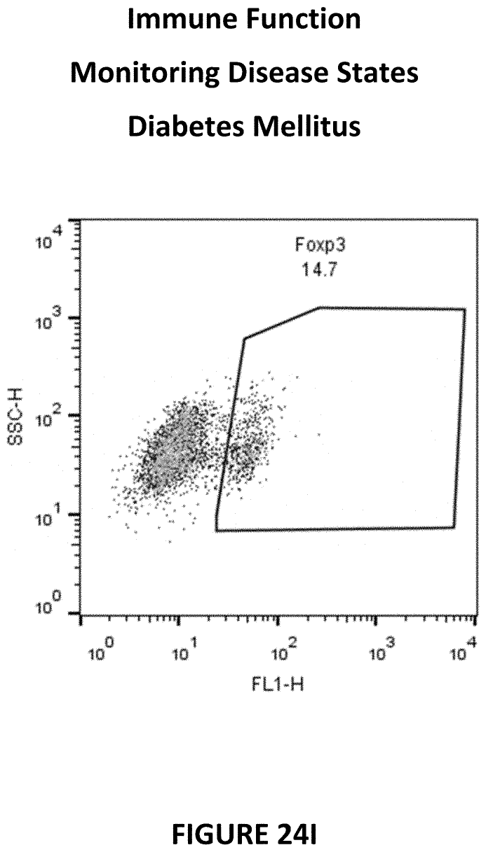

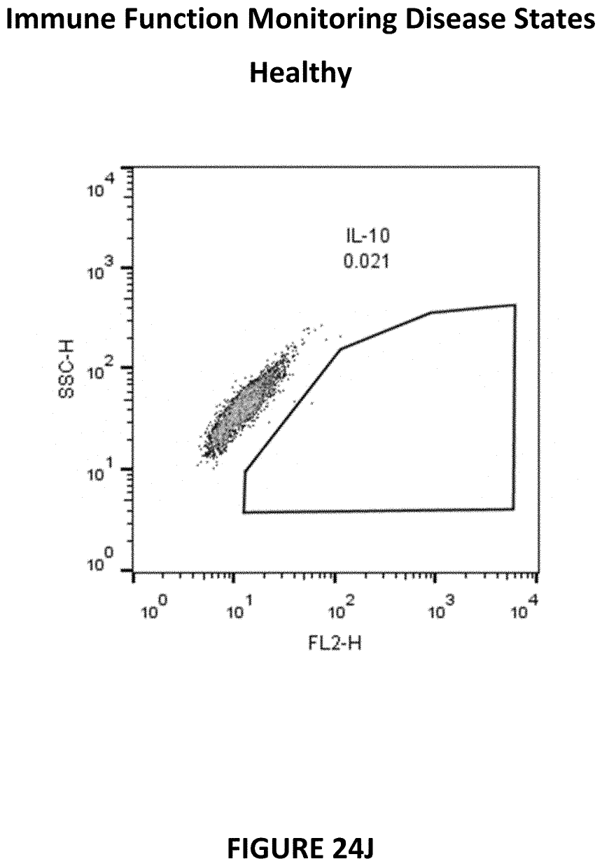

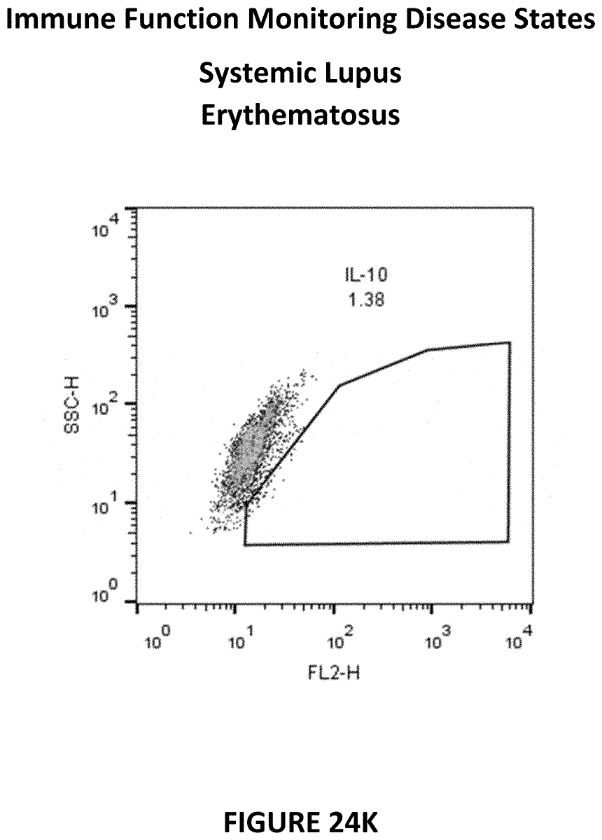

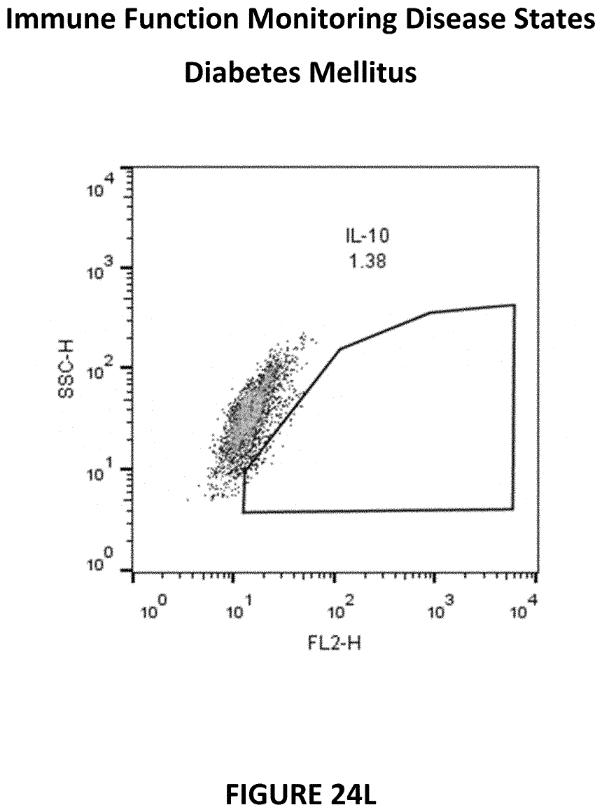

As shown in FIGS. 24A-24C, PBMCs from a healthy donor express greater amounts of pro-inflammatory cytokines IFNγ and IL-17 and greater amounts of Foxp3 transcription factor compared to PBMCs from a subject suffering from systemic lupus erythematosus (FIGS. 24D-24F) or diabetes mellitus (FIGS. 24G-24I). In addition, PBMCs from a healthy donor express lower amounts of anti-inflammatory cytokine IL-10 (FIG. 24J) in comparison to PBMCs from a subject suffering from systemic lupus erythematousus (FIG. 24K) or diabetes mellitus (FIG. 24L). These results show that flow cytometry can identify the immune status of a patient which could be useful in diagnosing and/or monitoring a patient's condition and/or response to treatments. Furthermore, the present method offers more accurate assessments since it evaluates transcription levels instead of the previous methods that measured protein levels. Since cytokines are designed to be secreted from cells, flow cytometric measurements of cytokine levels by antibody staining of target proteins can only take place by measurements of cytokines that may have bound to cellular surfaces or after treatment with a drug that prevents secretion; either protein measurement method would be prone to artefacts and/or misestimations of target levels.

### 7.24. Example 24: Activation of Unprimed T-Cells

Preparation of responder T-cell suspensions from mouse spleen, thymus and lymph node. Freshly removed organs are placed in 6-×15-mm petri dishes (one for each organ to be removed) containing 3 mL complete RMPI-5 or DMEM-5. Organs are cut in several places with autoclaved scissors. Pieces are pressed against the bottom of the petri dish with the autoclaved plunger of a 6-mL syringe using a circular motion until mostly fibrous tissue remains. Clumps in the suspension are dispersed by drawing up and expelling the suspension several times through a 6-mL syringe equipped with a 19-G needle. Suspension is expelled into a centrifuge tube through a 200-am mesh nylon screen. Petri dish is washed with 4 mL complete medium containing 5% FCS. Wash is repeated if necessary and is added to the tube. Suspension is centrifuged 10 minutes in Sorvall H-1000B rotor at 1000 rpm (200 ×g) and supernatant is discarded. Pellet is resuspended in 20 mL complete medium, centrifuged again, and resuspended in a volume suitable for counting.

If the cell suspension is from spleen, red blood cells (RBC) may be removed as follows. The pellet of spleen cells is resuspended in about 3 mL AKC lysing buffer and is incubated for 5 minutes at room temperature with occasional shaking. Wash medium is used to fill the tube to 50 mL, which is then spun for 10 minutes at 200 ×g in a low-speed centrifuge. Supernatant is discarded, and pellet is washed again and resuspended in appropriate medium and volume for the next procedure. AKC lysing buffer is mad as follows: combine 8.29 g NH₄Cl (0.15M), 1 g KHCO₃ (10.0 mM), 37.2 mg Na₂EDTA (0.1 mM), add 800 ml H₂O and adjust pH to 7.2-7.4 with 1 N HCl, add H₂O to 1 liter, filter sterilize through a 0.2-μm filter and store at room temperature. Next procedures can include dead cell removal, cell counting or fractionation, or proceed directly to activation.

Dead cell removal. Cells are resuspended in complete RPMI-5 and distributed into centrifuge tubes at 0.5-1×10⁸ cells/3 mL (12-mL tube) or 1-5×10⁸ cells/5 mL (50-ml tube). 3 mL (for 12-mL tubes) or 5 mL (for 50-mL tubes) are layered on high density solution (Ficoll-Paque (Ficoll and sodium diatrizoate; Pharmacia LKB) or Lympholyte-M, Cedarlane) under the cell suspension by drawing the high-density solution into a pipette, placing the tip of the pipette at the bottom of the tube, and slowly letting the solution flow under the cell suspension. Mixture is centrifuged 15 minutes in Sorvall H-1000B rotor at 2000 rpm (800 ×g) at room temperature. Live cells floating on top of the high-density solution are isolated by slowly moving the tip of the pipette over the surface of the high-density layer and drawing cells up in a 5 mL pipette and are transferred to another tube. RPMI-5 (10 mL for small volume, 40 mL for larger volume) are added to the live cells, the mixture is centrifuged 10 minutes at 200 ×g at room temperature, and the washing procedure is repeated to yield single responder T-cell suspensions.

Activation of unprimed responder T-cells with antibodies. Single-cell suspensions are centrifuged in 15 mL conical tubes for 10 minutes in Sorvall H-1000B rotor at about 1000 rpm (200 ×g) at room temperature and supernatant is discarded. Cell pellets are resuspended in 37° C. PBS containing 0.5% FBS containing a fluorescent cell dye such as CFSE in a concentration of 1×10⁷ cells/mL. Cells are incubated in 50 mL conical tubes protected from light at 37° C. for 10 minutes, then quenched by the addition of 5× total staining volume of ice cold complete RPMI-10. Cells are centrifuged in a Sorvall H-1000B rotor at about 1000 rpm (200 ×g) for 10 minutes at 4° C. Supernatant is discarded and resuspended in 50 mL ice cold complete RPMI-10, and once again centrifuged in a Sorvall H-1000B rotor at about 1000 rpm (200 ×g) for 10 minutes at 4° C. Supernatant is discarded and cell pellets are resuspended in complete RPMI-10, and responder T-cells are counted and adjusted to about 106 cells/mL with complete RPMI-10. Working solutions of activating agents in 4 mL conical tubes at room temperature are prepared. For a monoclonal antibody, toxin or lectin, a series of four dilutions of 1 mg/mL stock solution with PBS is made. For a pharmacological agent such as PMA, single dilutions of 100 ng/mL solution of PMA and 1 μg/mL A23187 (or 4 μg/mL ionomycin) in PBS are made. 20 μL of each dilution of activating reagent (MAb, enterotoxin or lectin) are added to each of three wells of a 96-well flat- or round-bottom microtiter plate. Control wells with 20 μL of PBS only are included. 20 μL of PMA or calcium ionophore at the single concentration are added, since dose-response curves for these agents are extremely narrow. 2×10⁵ cells in 0.2 mL are added to the wells of the 96-well plate containing the activating agent. Microtiter plates are placed in a humidified 37° C., 5% CO₂ incubator for 2-4 days. Degree of activation is measured by flow cytometry, with a low fluorescence indicating greater proliferation.

Activation of unprimed responder T-cells with plate-bound antibodies. A series of four dilutions of either 1 mg/mL purified anti-CD3 or 1 mg/mL anti-TCR monoclonal antibodies are made in 4 mL conical polystyrene tubes using PBS at room temperature to yield, for example, antibody concentrations of 100, 10, 1 and 0.1 μg/mL. 50 μL of each concentration of antibody solution is added to each of three wells of a 96-well round-bottom microtiter plate, and control wells with 30 μL PBS are included. Plates are covered and gently tapped on its side to insure complete coverage of the bottom of the wells and incubated for 120 minutes at 37° C. Responder T-cell suspensions are prepared as set forth in the previous paragraph. Antibody coating buffer is removed from each well of the 96-well round-bottom microtiter plate by aspiration. About 2×10⁵ cells in 0.2 mL are added to wells of the plates. Microtiter plates are placed in a humidified 37° C., 5% CO₂ incubator for 2-3 days. Degree of activation is measured by flow cytometry, with a low fluorescence indicating greater proliferation.

Activation of unprimed responder T-cells in mixed lymphocyte cultures. Responder T-cells in a mixed population of cells are prepared as set forth above in this Example, or responder T-cells can be purified T-cells or T-cell subsets. Responder T-cells in an amount of 5×10⁴ to 5×10⁵ in 0.1 mL are added to each well of a 96-well plate such that each experimental group has three replicate wells. Single cell suspension of (i) irradiated stimulator cells, (ii) mitomycin C-treated stimulator cells, or (iii) T-cell depleted stimulator cells is prepared. 0.1 mL of stimulator cells is prepared, and 0.1 mL of stimulator cells is added to each well of the plates containing responder T-cells. Microtiter plates are placed in a humidified 37° C., 5% CO₂ incubator for 3-6 days. Degree of activation is measured by flow cytometry, with a low fluorescence indicating greater proliferation. Methods for depleting T-cells from antigen-presenting/stimulatory cell suspensions are set forth in Kruisbeek, A. M., Shevach, E. and Thornton, A. M. 2004. Proliferative Assays for T Cell Function. Current Protocols in Immunology. 60:III:3.12:3.12.1-3.12.20, at 60:III:3:12.7-3.12.8. Methods for blocking cellular division of accessory/stimulator cells can be found in Kruisbeek, A. M., Shevach, E. and Thornton, A. M. 2004. Proliferative Assays for T Cell Function. Current Protocols in Immunology. 60:III:3.12:3.12.1-3.12.20, at 60:III:3.12.8-3.12.9.

### 7.25. Example 25: Activation of Primed T-Cells

As used herein, primed T-cells are those that recognize certain epitopes because they have already encountered the epitopes in vivo, for example, when the subject is immunized with the epitope, or in vitro, for example, in a coculture with antigen presenting cells that have been loaded with an epitope. Accordingly, primed T-cells have cell memory which can be assayed in vitro. Responder T-cells are prepared as set forth in Example 24. Four-fold dilution series of the antigens in 4-mL conical tubes are made using complete RPMI-10. Antigens are added to 96-well flat-bottom microtiter plates at 50 μL/well for protein antigens or 100 μL/well for cellular antigens. Three replicate wells are set up for each experimental group, and control wells with medium only (no antigen) are included. Responder T-cells in 0.1 mL are added to each well. If purified lymph node T-cells specific for protein antigens are used, 0.1 mL of accessory spleen cells syngeneic to the donor of the responder T-cells are added at 5×10⁵ cells per well. Microtiter plates are placed in a humidified 37° C., 5% CO₂ incubator for 2-4 days. Degree of activation is measured by flow cytometry, with a low fluorescence indicating greater proliferation.

### 7.26. Example 26: Activation of CD4+CD25+ T-Cells and Analysis of their Suppressive Function

Preparation of cells and solutions. CD4+CD25- and CD4+CD25+ T-cell suspensions in RPMI-10 are prepared as described in unit 3.5A in Thornton, A. M. 2003. Fractionation of T and B Cells Using Magnetic Beads. Current Protocols in Immunology. 55:I:3.5A:3.5A.1-3.5A.11. Cells are counted and CD4+CD25- and CD4+CD25+ cells or a portion thereof are adjusted to 1×10⁶ cells/mL with RPMI-10 medium. CD4+CD25− cells only are then labeled with a fluorescent dye as described in Example 24. Accessory cells are prepared in RPMI-10 medium as described in Support Protocol 1 in unit 3.5A in Thornton, A. M. 2003. Fractionation of T and B Cells Using Magnetic Beads. Current Protocols in Immunology. 55:I:3.5A:3.5A.1-3.5A.11. Accessory cells are counted and accessory cells (or a portion thereof) are adjusted to 1×10⁶ cells/mL with RPMI-10. The following solutions are prepared: 1 μg/mL anti-CD3 in RPMI-10, 200 U/mL IL-2 in RPMI-10, and 2 μg/mL anti-CD28 in RPMI-10.

Preparation to measure non-responsive state of CD4+CD25+ T-cells. 50 μL of CD4+CD25− cells are added to each of nine wells of a 96-well flat-bottom microtiter plate and 50 μL of CD4+CD25+ cells are added to each of nine wells of a 96-well flat-bottom microtiter plate. 50 μL of accessory cells and 50 μL of 1 μg/mL anti-CD3 are added to each of the wells. 50 μL of 200 U/mL IL-2 are added to three wells of the CD25− cells and three wells of the CD25+ cells. 50 μL of 2 μg/mL anti-CD28 are added to three wells of the CD25− cells and three wells of the CD25+ cells. 50 μL of RPMI-10 are added to the remaining three wells of each group.

Assay for suppressive function of CD4+CD25+ cells. 50 μL of CD4+CD25+ cells are added to three wells of a 96-well microtiter plate. A series of three to four dilutions of CD4+CD25+ cells are made, and control wells with 50 μL of RPMI-10 medium only are made. Into each well 50 μL of CD4+CD25-, 50 μL of accessory cells, and 50 μL of 1 μg/mL anti-CD3 are added. Microtiter plates are placed in a 37° C., 5%-7% CO₂ humidified incubator for 3 days (about 66 hours). Degree of activation is measured by flow cytometry, with a low fluorescence indicating greater proliferation. See Thornton and Shevach (1998) J. Experimental Medicine 188(2):287-296.

### 7.27. Example 27: Short-Term Activation and Expansion of CD4+CD25+ T-Cells and Analysis of their Suppressive Function

Preparation of cells. CD4+CD25+ T-cells in complete RPMI-10 medium supplemented with 100 U/mL IL-2 are purified as set forth in Unit 3.5A in Thornton, A. M. 2003. Fractionation of T and B Cells Using Magnetic Beads. Current Protocols in Immunology. Cells are counted and CD4+CD25+ cells are adjusted to 1×10⁶ cells/mL with RPMI-10/IL-2. A working solution of 5 g/mL anti-CD3 in PBS is prepared. 300 μL of antibody solution is added to each well of a 24-well plate. The number of wells to be coated is based on the anticipated yield of CD4+CD25+ cells. The plate is incubated for 120 minutes in a 37° C., 5% to 7% CO₂ humidified incubator. Antibody is removed from the plates and wells are washed two times with PBS to remove excess antibody. 1 mL (1×10⁶) of CD4+CD25+ cells are added to the wells of the washed plate. Plates are placed in a 37° C., 5% to 7% CO₂ humidified incubator for 3 days to yield activated regulatory T-cells that have acquired suppressive function, but that are not greatly expanded. After the 3 days, cells are split 1:3 or 1:4 in RPMI-10 medium supplemented with 100 U/mL IL-2, and returned to the 37° C., 5% to 7% CO₂ humidified incubator.

Assay of suppressive function of CD4+CD25+ cells. Activated CD4+CD25+ cells are harvested by pipetting up and down rigorously. Cells are centrifuged 10 minutes at 200 ×g (Sorvall H-1000B rotor at about 1000 rpm) at 4° C. Cells are washed two times to completely remove any remaining IL-2 and are resuspended in RPMI-10. Cells are adjusted to 1×10⁶ cells/mL with RPMI-10. Suppressor function of CD4+CD25+ cells is measured as set forth in the last paragraph of Example 26. A CD4+ T-cell suspension in RPMI-10 from transgenic mice is prepared as set forth in Unit 3.5A in Thornton, A. M. 2003. Fractionation of T and B Cells Using Magnetic Beads. Current Protocols in Immunology. Cells are counted and CD4+ cells, or a portion thereof, are adjusted to 1×10⁶ cells/mL with RPMI-10. Accessory cells in RPMI-10 are prepared according to Support Protocol 1, Kruisbeek, A. M., Shevach, E. and Thornton, A. M. 2004. Proliferative Assays for T Cell Function. Current Protocols in Immunology. 60:III:3.12:3.12.1-3.12.20, at 3:12.7-3.12.8, or Thornton, A. M. 2003. Fractionation of T and B Cells Using Magnetic Beads. Current Protocols in Immunology. 55:I:3.5A:3.5A.1-3.5A.11. Cells are counted and accessory cells (or a portion thereof) are adjusted to 1×10⁶ cells/mL with RPMI-10. A solution of antigen at 4× in RPMI-10 is prepared. 50 μL of CD4+CD25+ cells are added to three wells of a 96-well microtiter plate. A series of three to four two-fold dilutions of CD4+CD25+ cells is made. Control wells with 50 L of RPMI-10 only are included. To each well add 50 μL of TCR Tg CD4+ cells, 50 μL of accessory cells, and 50 μL of antigen to each of the wells. Plates are placed in a 37° C., 5% to 7% CO₂ humidified incubator for 3 days to yield activated regulatory T-cells that have acquired suppressive function, but that are not greatly expanded. After the 3 days, cells are split 1:3 or 1:4 in RPMI-10 medium supplemented with 100 U/mL IL-2, and returned to the 37° C., 5% to 7% CO₂ humidified incubator. See Thornton and Shevach (1998) J. Experimental Medicine 188(2):287-296.

### 7.28. Example 28: Reduction of False Signals from Nucleotide Probes by Formaldehyde Treatment

Nucleotide probes such as RNA and DNA probes are highly charged molecules. The phosphate backbone of these probes provide a strong negative charge which can lead to binding to positively-charged molecules in cells. In addition, there may be physical trapping of probes by cells after fixation steps. In this particular Example we describe a method of decreasing background by the use of formaldehyde during preparation of cells prior to hybridization with homogeneous probes and detection by flow cytometry. Subsequent examples of background reduction reagents are simple additions to the hybridization buffer which do not require additional washing steps.

Test protocol. Human cervical cell samples that had been fixed in ThinPrep® solution were resuspended in 150 μL of 0%, 1% or 5% formaldehyde in PBS for 30 minutes, then diluted to 300 μL with PBS, and then spun down. Cervical cells were then washed with 300 μL PBS a second time. Each group of cervical cells was divided into two. One sample from each group was resuspended in hybridization buffer (2% Triton-X100 in 1×SSC) without probes, and the other sample was resuspended in hybridization buffer with 0.5 μL negative control probe per 300 μL buffer (stock probe concentration was 5 μM). The Negative Control probe used in this Example (and the following background reduction Examples) consists of a set of three Molecular Beacons lacking homology with mRNA in the test cells. The Negative Control probes have the following sequences:

NP1
(SEQ ID NO: 106)
(FAM)-cctcccacTTGCACATGCCGGAGCCGTTgtgggagg-(BHQ2a)
NP2
(SEQ ID NO: 107)
(FAM)-cctcccacTGACCCATGCCCACCATCACgtgggagg-(BHQ2a)
NP3
(SEQ ID NO: 108)
(FAM)-ccaccgTCTTGATCTTCATTGTGCTGcggtgg-(BHQ2a)

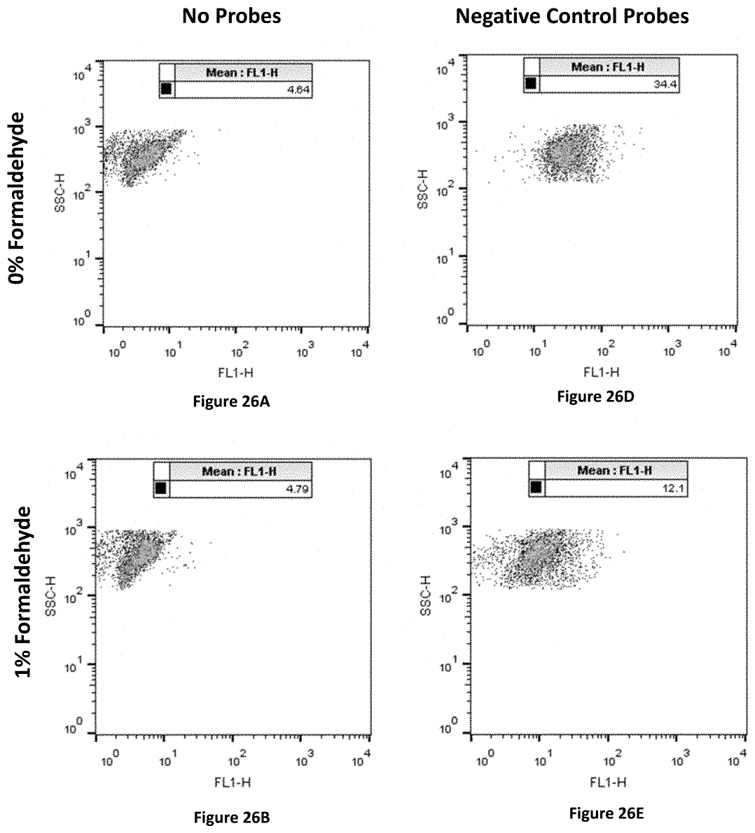

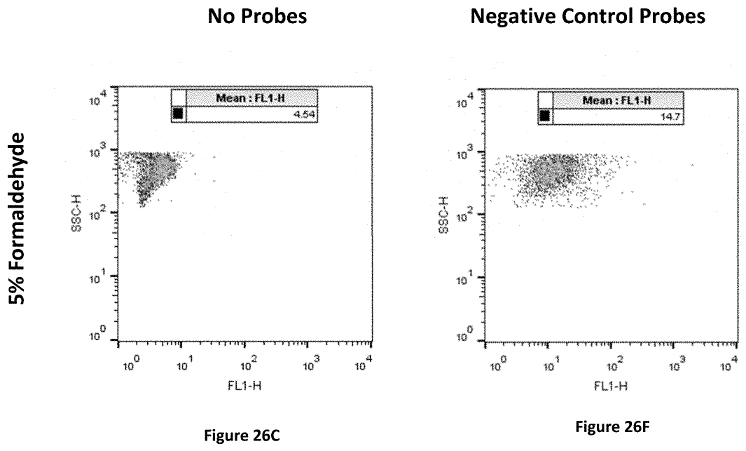

Samples were incubated at room temperature for 1 hour and analyzed by flow cytometry with gating on ectocervical cells. All cervical cell samples, regardless of formaldehyde treatment, looked similar without addition of probes. See FIGS. 25A-25C (high grade squamous intraepithelial lesion (HISL) patients) and FIGS. 26A-26C (double negative for high risk HPV infection and negative for cytology (N/N) patients). With probes added, high false signal was generated from non-specific binding by the Negative Control probe (25D and 26D), which was reduced by formaldehyde treatment. See FIGS. 25E, 25F 26E and 26F. The trend that was noted was that N/N samples exhibited higher false signal than HSIL samples, which is an example showing that this false signal seen with DNA probes can provide misleading information and skew results to reduce clinical correlation of results. Without being bound by any particular theory, formaldehyde crosslinks several types of molecules, including proteins, which potentially decrease the charge on those molecules in the cells which could be involved in the subsequent reduction of non-specific interactions with the molecular beacon probe.

### 7.29. Reduction of False Signals from Nucleotide Probes by Alterations of SSC Concentrations

Human PBMCs fixed in 5% formaldehyde were resuspended in hybridization buffers that contained 2% Triton-X 100, but with varying concentrations of SSC. Two 1×SSC samples were prepared, one without probes to show baseline fluorescence (FIG. 27A), and one with 0.5 μL of a Negative Control probe per 300 μL buffer (stock probe concentration of 5 μM) (FIG. 27B). The Negative Control probes was the same set of Molecular Beacons used in Example 28. Samples with higher SSC concentrations were prepared with the same amount of negative probes (FIGS. 27C and 27D). Samples were incubated at room temperature for 1 hour and evaluated as usual by flow cytometry.

As seen in FIG. 27B a large amount of false signal was seen in the 1×SSC buffer with the negative control probes (FIG. 27B), the false signal decreases when SSC concentration is increased to 2.5× (FIG. 27C) and is reduced further to baseline fluorescence with the 5×SSC concentration (FIG. 27D). Accordingly, this experiment demonstrates that in the presence of 5×SSC, false signal has been almost completely eliminated. SSC exhibits significant interactions with phosphate groups on nucleotides which could block non-specific interactions of nucleotide probes with positively charged molecules in the cells. Other salts that interact with phosphate groups on nucleotides may also show similar reductions in false signal. In addition to its ionic properties, SSC is also a chelator, which may also be a factor in its ability to reduce false signal generation.

### 7.30. Reducing False Signals from Nucleotide Probes by Addition of a Polyanionic Compound and Nuclear Binding Dye

Human cervical cells fixed in 5% formaldehyde were resuspended in hybridization buffers consisting of 2% Triton-X 100 in 1×SSC. Samples had either (A) no probe, (B) Negative Control probe alone, (C) Negative Control probe+10 U/ml unfractionated heparin, a polyanionic glycosaminoglycan polymer (Sigma-Aldrich Cat. No. H3393) or (D) Negative Control probe with X/ml of Nuclear ID™ (Enzo LifeSciences, Farmingdale, N.Y.). Samples with Negative Control probe had 0.5 μL of probe per 300 μL buffer (stock probe concentration was 5 μM). The Negative Control probe was the same set of Molecular Beacons used in Example 28 (NP1, NP2 and NP3). Samples were incubated at room temperature for 1 hour and evaluated as usual by flow cytometry

FIG. 28A show the background without probe added and FIG. 28B shows the amount of non-specific signal generated by addition of the Negative Control probe. It can be seen that either the addition of heparin (FIG. 28C) or Nuclear ID (FIG. 28D) returned the signal level to that seen without any probe. Accordingly, both a polyanionic compound (heparin) and a DNA binding dye (nuclear ID) are useful for elimination or reduction of non-specific signal generation.

### 7.31. Background Reduction by Treatment with Dextran Sulfate

Human PBMCs were fixed in ThinPrep, washed with PBS and resuspended in hybridization buffers that contained 2% Triton-X 100 and 1×SSC. Samples had either (A) no probe, (B) Negative Control probe or (C) Negative Control probe with 5% (w/v) Dextran sulfate, a polyanionic polymer of sulfated glucose (Sigma-Aldrich Cat. No. D8906, average Mw >500,000). Samples with Negative Control probe had 0.5 μL of a per 300 μL buffer (stock probe concentration was 5 μM). The negative probe control was the same set of Molecular Beacons used in Example 28. Samples were incubated at room temperature for 1 hour and analyzed as described previously, gating on the lymphocyte population. As seen in FIG. 29B there is very high background contributed by the presence of the Negative Control probe (FIG. 29A). However, the presence of the Dextran Sulfate reduced the signal from the Negative Control probe to the same level seen without the probe, demonstrating an effect similar the heparin, the polyanionic compound tested in Example 30.

### 7.32. Background Reduction by Treatment with Ethidium Homodimer

Caski cells fixed in ThinPrep were resuspended in hybridization buffers that contained 2% Triton-X 100 and 1×SSC. Samples had either (A) no probe, (B) Negative Control probe or (C) Negative Control probe with 2 μM Ethidium homodimer, an intercalator with a very high affinity for DNA. Samples with Negative Control probe had 0.5 μL of probe per 300 μL buffer (stock probe concentration was 5 μM). The negative probe control was the same set of Molecular Beacons used in Example 28. Samples were incubated at room temperature for 1 hour and analyzed as described previously. In this Example, high backgrounds were seen for the Negative Control Probe sample (FIG. 30B) compared to the no probe sample (FIG. 30A) with the MFI being 30× higher for the Negative Control Probe. However, the presence of the Ethidium homodimer allowed significant reduction in the background signal. In this Example, only a single concentration of Ethidium homodimer was used and varying the amount could increase the effect further.

### 7.33. Treatment with EDTA for False Signal Reduction

Human PBMCs fixed in 5% formaldehyde were resuspended in hybridization buffers that contained 2% Triton-X 100 and 1×SSC, and various concentrations of EDTA. Samples were prepared with 0.5 μL of the Negative Control probe (described in Example 28) per 300 μL of buffer (stock probe concentration was 5 μM) in each of the indicated buffers. Samples were incubated at room temperature for 1 hour and analyzed as described previously

Results. While a large amount of false signal was seen in the absence of EDTA (FIG. 29A), the target independent signal decreases as the EDTA concentration is increased. See FIG. 29B (EDTA concentration at 31.25 mM), FIG. 29C (EDTA concentration at 62.5 mM), FIG. 29D (EDTA concentration at 123 mM) and FIG. 29E (EDTA concentration at 250 mM). EDTA is a chelator, which may remove metals that affect non-specific binding of probes. Deprotonated EDTA has a highly negative charge that may interact with molecules which otherwise may interact with DNA or RNA probes.

### 7.34. Example 32: Memory T-Cell Analysis Protocol

T-cells can be divided into two categories based on their exposure to their cognate antigen after leaving the thymus. T-cells that have never encountered their antigen are called naïve. After recognition of antigen, T-cells are activated, and they proliferate, secrete cytokines, and may exhibit cytotoxicity, depending on their type. At the end of this process of activation, some T-cells die, while others become memory T-cells. Memory T-cells are long-lived lymphocytes that, when they recognize their antigen in the future, can be activated more quickly and strongly than naïve cells. These memory T-cells are an important part of the adaptive immune system that allows us to become less sick, or even avoid becoming sick, when exposed to a pathogen we have already experienced, and are highly important for survival. The presence of these cells is also an indicator of what antigens to which a person has been exposed and has reacted to in the past. Accordingly, memory T-cells can be used to identify if a person has been exposed to a virus or other pathogen, if the person has been vaccinated against an antigen, or if a person has an autoimmune condition toward a self-antigen.

Memory T-cell analysis protocol. Human PBMCs fixed in 5% formaldehyde were resuspended in hybridization buffer consisting of 2% Triton-X 100 in 1×SSC, with 50 U/mL Reagent-X to remove false signal. The buffer contained 0.5 μL per 300 μL buffer of probes for IFNγ and IL-2Rβ. Cells were then incubated protected from light at 65° C. for one hour, then at 4° C. for 30 minutes before analysis by flow cytometry.

Probes for IFNγ were as described previously in Example 14; sequences for IL-2Rβ probes are:

IL2Rb1
SEQ ID NO: 109
(Cy5)-ccagccAGATTTCGTTGTGGGTTTCCACCATggctgg-(EQ2)
IL2Rb2:
(SEQ ID NO: 110)
(Cy5)-ccagccTGCTGTATTTCTGGTACAGCTCCACggctgg-(EQ2)
IL2Rb3:
(SEQ ID NO: 111)
(Cy5)-ccagccGTAGTGAACCCGTTGATGTCCACTTggctgg-(EQ2)
IL2Rb4:
(SEQ ID NO: 112)
(Cy5)-ccagccGTGGAGCTGAAGCAATAGTTGGTGTggctgg-(EQ2)
IL2Rb5:
(SEQ ID NO: 113)
(Cy5)-ccagccCTACAGTAGTGTTCCCCACTGGTCCggctgg-(EQ2)
IL2Rb6:
(SEQ ID NO: 114)
(Cy5)-ccagccCAATGACACAGAGATCCGCAGTCCggctgg-(EQ2)

Results. IL-2Rβ was not expressed on naïve T-cells, and is up-regulated upon antigen exposure. Memory T-cells can retain IL-2Rβ expression for months or longer, making them ideal markers for T-cell memory in quiescent T-cells. A small percentage of the total lymphocyte population was seen to express IL-2Rβ mRNA. While less than 4% of the lymphocyte population was positive for IFNγ mRNA (FIG. 30A), IL-2Rβ mRNA positive cells (FIG. 30B) were over 40% positive for IFNγ mRNA (FIG. 30C), showing that these memory cells are primed for IFNγ production. Further division of this group (FIG. 30D) into IL-2Rβ low (FIG. 30E) and IL-2Rβ high expressing cells (FIG. 30F) showed that the IL-2Rβ low population had a more modest amount of cells (12.5%) that were positive for IFNγ RNA (FIG. 30E), while the IL-2Rβ high population of cells was over 80% positive for IFNγ mRNA (FIG. 30F), suggesting that the IL-2Rβ high population is the more strongly primed set of memory cells.

### 7.35. Example 33: Interfering and Non-Interfering Intercalators

Streptavidin coated beads were coated with target transcripts for E6/E7 or 18s. E6/E7 probes are labeled with FAM, and these were hybridized to their targets on E6/E7 beads. 18s probes are labeled with Cy3 and were hybridized to their targets on 18s beads.

Both were done according to the standard FlowScript protocol of 65° C. heating for 1 hour and 4° C. for 30 minutes in a buffer of 2% Triton X-100 in 1×SSC with 50 U/mL Heparin, with 0.5 uL/probe cocktail added per 300 uL buffer.

Beads were run through the protocol with the following conditions: without probes or intercalators, with probes alone, and with probes plus each type of intercalator.

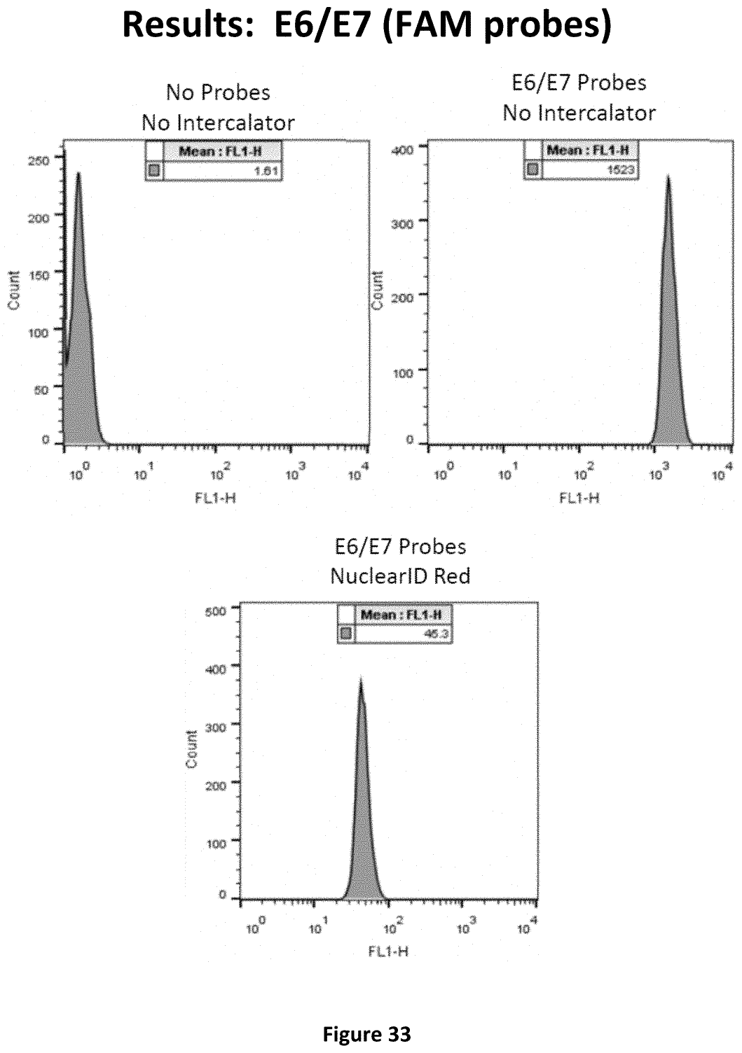

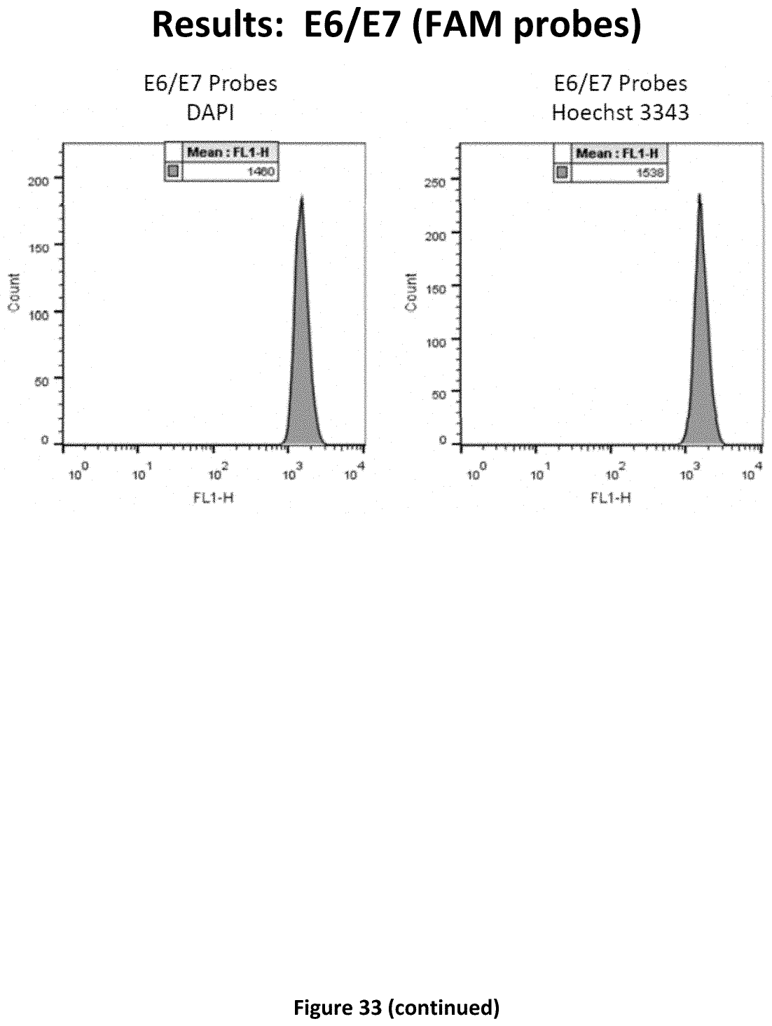

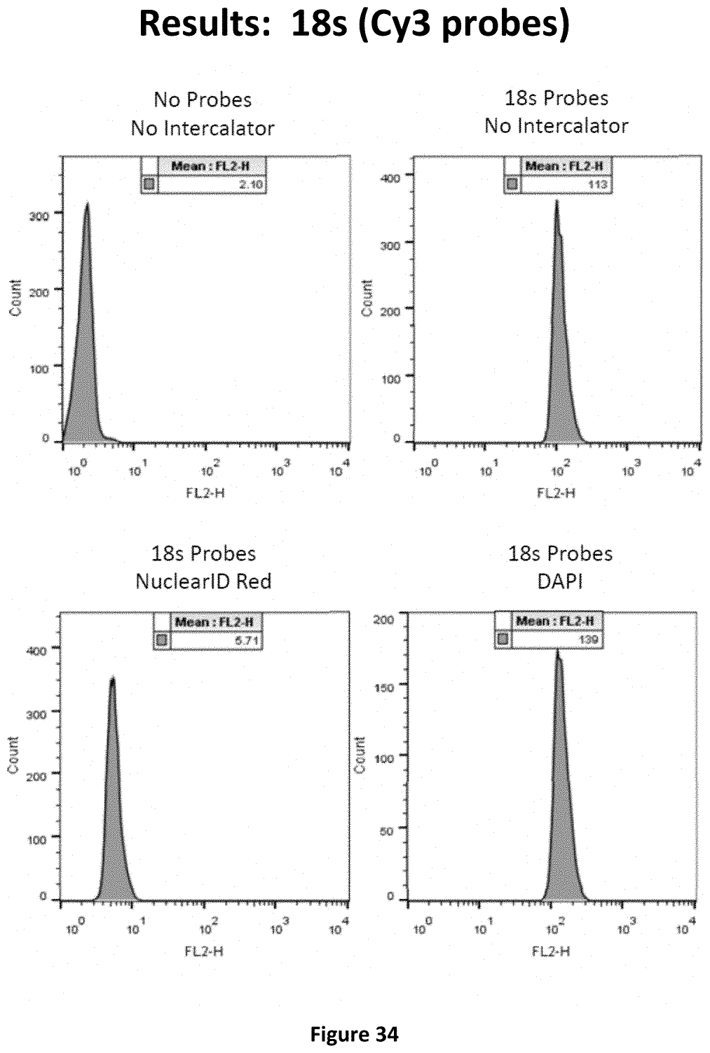

FIG. 33 shows the results for different intercalators with the E6/E7 FAM probes. FIG. 34 shows the results for different intercalators with 18s Cy3 probes.

It was discovered that intercalating dyes can affect probe signal. The close proximity of the intercalating dyes which can bind between the probes and their target sequence to the fluorophores attached to the probes allows the intercalating dyes to quench the signal from the probes if the intercalator absorbs light in the wavelength which the fluorophore emits. Because of this, the use of intercalating dyes which emit in the red wavelengths are a poor choice for use with certain probes for detection of nucleic acids by flow cytometry, because these intercalating dyes tend to have absorbances in the range which typically used fluorophores on probes will emit. NuclearID Red was found to quench signal from FAM and Cy3 probes, while DAPI and Hoechst 3343, which absorb light in the UV and violet spectrums, did not.

### 8. ADDITIONAL EMBODIMENTS

This section includes additional embodiments.

1. A method of detecting or quantifying an amount of a target RNA in a eukaryotic cell comprising the steps of (a) fixating and permeabilizing a eukaryotic cell; (b) contacting the cell from step (a) with (i) a nucleic acid probe that is complementary to a nucleic acid of interest, and (ii) a natural or artificially introduced control nucleic acid probe, wherein the nucleic acid probes hybridize to complementary nucleic acid sequences in the eukaryotic cells; (c) detecting using flow cytometry (i) an amount of signal generated by the probe hybridized to a complementary target nucleic acid sequence in the eukaryotic cell, and (ii) an amount of signal generated by the control probe hybridized to a complementary nucleic acid sequence in the eukaryotic cell; and (d) detecting or measuring the nucleic acid of interest in the eukaryotic cell by detecting or measuring the amount of signal generated from the probes of step (b), wherein the probes of step (b) are part of a homogeneous probe system that generates a signal when bound to a complementary sequence in the target nucleic acid, and wherein the presence or quantity of the target RNA is associated with the presence of or susceptibility to a disease.

2. A method of detecting or quantifying an amount of a target RNA in a eukaryotic cell comprising the steps of (a) fixating and permeabilizing a eukaryotic cell; (b) fixating and permeabilizing a eukaryotic control cell; (c) contacting (i) the cell from step (a) with a nucleic acid probe that is complementary to a nucleic acid of interest; and (ii) the cell of step (b) with a nucleic acid probe, wherein the nucleic acid probe hybridizes to complementary nucleic acid sequences in the eukaryotic control cell; (d) detecting using flow cytometry (i) an amount of signal generated by the probe hybridized to a complementary target nucleic acid sequence in the eukaryotic cell of step (c)(i), and (ii) an amount of signal generated by the probe hybridized to a complementary target nucleic acid sequence in the eukaryotic control cell of step (c)(ii); and (e) detecting or measuring the nucleic acid of interest in the eukaryotic cell by detecting or measuring the amount of signal generated from the probe of step (c)(i), (f) detecting or measuring the nucleic acid in the eukaryotic control cell by detecting or measuring the amount of signal generated from the probe of step (c)(ii); and (g) comparing the results of step (e) and step (f), wherein the probes of steps (c)(i) and (c)(ii) are part of a homogeneous probe system that generates a signal when bound to a complementary sequence in the target nucleic acid, and wherein the presence or quantity of the target RNA is associated with the presence of or susceptibility to a disease.

3. The method of embodiment 1 or embodiment 2, wherein the nucleic acid is mRNA.

4. The method of embodiment 1 or embodiment 2, further comprising adding a nucleic acid probe to the mixture of step (b), step (c) or both step (b) and step (c), wherein the nucleic acid probe comprises at least one probe that is part of a homogeneous probe system that generates a signal when bound to a complementary sequence in a control nucleic acid.

5. The method of embodiment 1 wherein the control nucleic acid comprises a sequence from a housekeeping gene.

6. The method of embodiment 2 wherein the control cell is selected from a cell from a healthy individual when the cell of step (a) is a diseased cell, a cell with inactive disease when the cell of step (a) is a cell with active disease, a cell that does not respond to an antigen when the cell of step (a) is a cell that responds to the antigen, a cell that has not been subjected to treatment when the cell of step (a) has been treated, or combinations thereof.

7. The method of embodiment 3 wherein the target nucleic acid is an mRNA that is expressed from a virus in the eukaryotic cell.

8. The method of embodiment 7 wherein the virus is selected from HIV, HBV, HCV, HPV and a Herpesvirus.

9. The method of embodiment 8, wherein the virus is HPV and the target nucleic acid is an mRNA comprising a sequence from the HPV E6 gene, the HPV E7 gene, the HPV E2 gene or any combination thereof.

10. The method of embodiment 5 wherein the control nucleic acid is a non-viral mRNA expressed by the eukaryotic cell.

11. The method of embodiment 1 wherein at least one probe provided in step (b)(i) is complementary to a viral mRNA, wherein at least one probe of step (b)(i) is complementary to a sequence in a eukaryotic mRNA from a gene that changes expression during viral infection, and wherein the probes are part of a homogeneous probe system that generates a signal when bound to a complementary sequence in the eukaryotic mRNA.

12. The method of embodiment 11, wherein the gene that changes expression during viral infection is selected from p53. Rb, P16^{INK4a},Ki67, TOP2a, MCM2, CK13, CK14, MCM5, CDC6, survivin, CEA, p63, pRb, p21WAF1, MYC cellular oncogene, CDK4, cyclin A, Cyclin B, cyclin D, cyclin E, telomerase, minichromosome maintenance protein 2, minichromosome maintenance protein 4, minichromosome maintenance protein 5), heat shock protein 40, heat shock protein 60, heat shock protein 70, CA9/MN protein, and a combination thereof.

13. The method of embodiment 1, further comprising in step (b)(i) adding at least one antibody.

14. The method of embodiment 13, wherein the antibody recognizes a surface antigen of the eukaryotic cell.

15. The method of embodiment 13, wherein at least one probe provided in step (b)(i) is complementary to a viral mRNA in the eukaryotic cell and is part of a homogeneous probe system that generates a signal when bound to a complementary sequence in the viral mRNA, and wherein the antibody recognizes an antigen from a protein expressed from a eukaryotic gene that changes expression during infection by the virus.

16. The method of embodiment 15 wherein the gene that changes expression during infection by the virus is selected from p53. Rb, P16^{INK4a}, Ki67, TOP2a, MCM2, CK13, CK14, MCM5, CDC6, surviving, CEA, p63, pRb, p21WAF1, MYC cellular oncogene, CDK4, cyclin A, Cyclin B, cyclin D, cyclin E, telomerase, minichromosome maintenance protein 2, minichromosome maintenance protein 4, minichromosome maintenance protein 5), heat shock protein 40, heat shock protein 60, heat shock protein 70, CA9/MN protein, and a combination thereof.

17. The method of embodiment 13, wherein the antibody is unlabeled.

18. The method of embodiment 13, wherein the antibody is labeled.

19. The method of embodiment 18, wherein the antibody is labeled with a ligand, a fluorescent compound, a quantum dot, an electron dense component, a magnetic component, a hormone component, a chelating group, a chelated compound, an antigen or a combination thereof.

20. The method of embodiment 1, embodiment 2 or embodiment 3, wherein a change in the target nucleic acid level is associated with a cancerous state.

21. The method of embodiment 1, embodiment 2 or embodiment 3, wherein the homogeneous probe system is selected from a Binary Probe system, a Yin Yang Probe System, a Binary Yin Yang Probe System, a Lightup Probe System, a Molecular Beacon System, A Stemless Beacon System, a Binary Molecular Beacon System and an Extended Molecular Probe System.

22. A method of determining the effectiveness of a medical treatment in a subject suffering from a disease comprising the steps of (a) obtaining a first cell sample from the subject, wherein the first cell sample is taken before the medical treatment; (b) fixating and permeabilizing the cell of step (a); (c) contacting the cell of step (b) with (i) a nucleic acid probe that is complementary to a nucleic acid of interest; and (ii) a natural or artificially introduced control nucleic acid probe wherein the nucleic acid probe hybridizes to complementary nucleic acid sequences in the eukaryotic cells in the first cell sample; (d) detecting using flow cytometry (i) an amount of signal generated by the probe hybridized to the complementary nucleic acid in the first cell sample, and (ii) an amount of signal generated by a control probe hybridized to a complementary nucleic acid sequence in the eukaryotic cell; (e) detecting or measuring the nucleic acid of interest in the eukaryotic cell by detecting or measuring the amount of signal generated from the probes of step (c); (f) obtaining a second cell sample from the subject after treatment; (g) carrying out steps (b)-(e) with the second cell sample; and (h) comparing the level of signal from the first cell sample measured in step (d) and the second cell sample measured in step (g), wherein the nucleic acid probes of step (c) comprise at least one probe that is part of a homogeneous probe system that generates a signal when bound to a complementary sequence in the target nucleic acid, and wherein the level of the target nucleic acid is correlated with the presence or severity of the disease, and wherein a change in gene expression indicates the effectiveness of treatment.

23. The method of embodiment 22, further comprising after step (g) a step of obtaining one or more additional cell samples from the subject at selected intervals during or after treatment.

24. The method of embodiment 22, wherein the disease is an infection selected from a parasitical infection or a viral infection.

25. The method of embodiment 22, wherein said disease is cancer.

26. The method of embodiment 22, wherein said disease is an immune mediated disorder.

27. A method of determining malignant transformation of eukaryotic cells capable of being infected with human papilloma virus comprising the steps of (a) contacting a specimen of fixated and permeabilized eukaryotic cells with (i) an HPV E6/E7 mRNA-specific molecular beacon, and (ii) a eukaryotic cell mRNA-specific control probe molecular beacon; and (b) measuring using flow cytometry (i) an amount of signal generated by the molecular beacon of step (a)(i), and (ii) an amount of signal generated by the control molecular beacon of step (a)(ii); and (c) identifying a number of cells in the specimen having E6/E7 mRNA copies above a positive determined cutoff point, wherein a cutoff point of at least 100 copies of HPV E6/E7 mRNA per cell is indicative of malignant transformation of said eukaryotic cells.

28. A method of determining malignant transformation of eukaryotic cells capable of being infected with human papilloma virus comprising the steps of (a) contacting (i) fixated and permeabilized eukaryotic cells from a specimen, and (ii) fixated and permeabilized eukaryotic control cells with an HPV E6/E7 mRNA-specific molecular beacon, (b) measuring using flow cytometry (i) an amount of signal generated by the molecular beacon of step (a)(i), and (ii) an amount of signal generated by the molecular beacon of step (a)(ii); and (c) identifying (i) a number of cells in the specimen having E6/E7 mRNA copies above a positive determined cutoff point, and (ii) a number of control cells having E6/E7 mRNA copies above a positive determined cutoff point; and (d) comparing the amount of cells identified in step (c)(i) with the amount of cells identified in step (c)(ii).

29. The method of embodiment 27 or embodiment 28, wherein a cutoff point of 200 copies of HPV E6/E7 mRNA per cell is indicative of a viral infection in the eukaryotic cell.

30. The method of embodiment 27 or embodiment 28, wherein the HPV specific mRNA molecular beacon is capable of binding to HPV 16, HPV 18 or both HPV 16 and HPV 18 mRNA to generate a signal.

31. The method of embodiment 27 or embodiment 28, wherein the eukaryotic cells are cervical cells, anal cells or head and neck cells.

32. The method of embodiment 28, wherein the control cells are from a cell line containing an integrated copy of the HPV genome.

33. The method of embodiment 28, wherein the control cells are from a cell line that is not infected with HPV.

34. The method of embodiment 27, further comprising determining the percentage of cervical cells in the specimen having E6, E7 mRNA copies above the positive predetermined cutoff point, wherein the percentage is an indication of the presence of malignant transformation.

35. The method of embodiment 27, wherein the eukaryotic cell mRNA comprises a sequence of a housekeeping gene.

36. The method of embodiment 27, wherein the eukaryotic cell mRNA comprises a sequence of a gene associated with the development of cancer.

37. The method of embodiment 36, wherein the gene is selected from p53, Rb, P16^{INK4a}, Ki67, TOP2a, MCM2, CK13, CK14, MCM5, CDC6, survivin, CEA, p63, pRb, p21WAF1, MYC cellular oncogene, CDK4, cyclin A, Cyclin B, cyclin D, cyclin E, telomerase, minichromosome maintenance protein 2, minichromosome maintenance protein 4, minichromosome maintenance protein 5), heat shock protein 40, heat shock protein 60, heat shock protein 70 or CA9/MN protein, and a combination thereof.

38. A method of diagnosing a disease in a subject comprising the steps of (a) fixating and permeabilizing (i) eukaryotic cells from the subject suspected of having a disease; and (ii) eukaryotic cells from a healthy individual; (b) contacting the cells from step (a) with a nucleic acid probe that is complementary to a nucleic acid sequence present in one or more cells under conditions in which the nucleic acid probe hybridizes to a complementary nucleic acid sequence in the eukaryotic cell; (c) detecting using flow cytometry (i) an amount of signal generated by the probe that is hybridized to a complementary target nucleic acid in the eukaryotic cell from the subject, and (ii) an amount of signal generated by the probe that is hybridized to a complementary target nucleic acid sequence in the eukaryotic cell from a healthy individual; and (c) detecting or measuring the nucleic acid in the eukaryotic cell from the subject and in the eukaryotic cell from the healthy individual, wherein a deviation in the level of the nucleic acid of interest detected in step (c)(i) from the level of the nucleic acid of interest detected in step (c)(ii) indicates the presence of a disease in the subject, and wherein the nucleic acid probe of step (b) comprises at least one probe that is part of a homogeneous probe system that generates a signal when bound to a complementary sequence in the target nucleic acid.

39. The method of embodiment 38, wherein the target nucleic acid is an mRNA from a parasite or a virus capable of infecting the subject.

40. The method of embodiment 38, wherein the subject is a human being.

41. The method of embodiment 38, wherein the nucleic acid probe is a panel of probes, wherein a specific pattern of expression levels is indicative of a particular disease condition.

42. The method of embodiment 41, wherein the disease is cancer.

43. The method of embodiment 41, wherein the disease is an immune mediated disorder.

44. The method of embodiment 38, wherein the cells are from blood, lymphatic fluid, spinal fluid, urine or tissues.

45. The method of embodiment 44 wherein the tissue cells are from vaginal tissue, anal tissue, nose tissue, ear tissue, or throat tissue.

46. A method of diagnosing an immune-mediated disorder in a subject, comprising the steps of (a) fixating and permeabilizing (i) responder T-cells from the subject and responder T-cells from a healthy individual, and (ii) regulatory T-cells from the subject and regulatory T-cells from a healthy individual; (b) contacting (i) the responder T-cells from step (a)(i) with a nucleic acid probe that is complementary to a nucleic acid sequence present in one or more responder T-cells and that is associated with a pro-inflammatory response under conditions in which the nucleic acid probe hybridizes to the nucleic acid sequence in the responder T-cells; (ii) the regulatory T-cells from step (a)(ii) with a nucleic acid probe that is complementary to a nucleic acid sequence present in one or more regulatory T-cells and that is associated with an anti-inflammatory response under conditions in which the nucleic acid probe hybridizes to the nucleic acid sequence in the regulatory T-cells; (c) detecting by flow cytometry (i) an amount of signal from the responder T-cells of the healthy individual (RespH); (ii) an amount of signal from the responder T-cells of the subject (RespP); (iii) an amount of signal from the regulatory T-cells of the healthy individual (RegH); and (iv) an amount of signal from the regulatory T-cells of the subject (RegP); and (c) detecting or measuring (i) the amount of signal generated in step (e)(i) (RespH) and the amount of signal generated in step (e)(ii) (RespP); (ii) the amount of signal generated in step (e)(iii) (RegH) and the amount of signal generated in step (e)(iv) (RegP); or (iii) the amount of signal generated in step (d)(i) and (d)(ii), wherein a comparison of RespH and RespP, of RegH and RegP or of both RespH and RespP and RegH and RegP that indicates a deviation of the subject's response from that of a healthy individual is indicative of an immune-mediated disorder in the subject.

47. The method of embodiment 46, wherein the nucleic acid sequence associated with an anti-inflammatory response is part of a gene coding for an anti-inflammatory cytokine or a receptor for an anti-inflammatory cytokine present in one or more regulatory T-cells.

48. The method of embodiment 46, wherein the nucleic acid sequence associated with a pro-inflammatory response is part of a gene coding for a pro-inflammatory cytokine or a receptor for a pro-inflammatory cytokine present in one or more responder T-cells.

49. A composition comprising a fixated immune cell comprising two or more labeled probes that bind to (i) a target; and (ii) a control element in the immune cell, wherein the probes are part of a homogeneous probe system that generates a signal when bound to a target, and wherein the target is associated with an immune-related function.

50. The composition of embodiment 49 wherein the fixated immune cell is from a subject suffering from an immune-mediated disorder.

51. The composition of embodiment 49 wherein the fixated immune cell is from a healthy individual.

52. A method of identifying a compound comprising an epitope from a library or collection of epitopes that induces immune tolerance in a subject suffering from an immune mediated disorder comprising the steps of (a) exposing live T-reg cells from the subject to the compound in the presence of an epitope binding agent; (b) incubating (i) the T-reg cells of step (a), and (ii) control T-reg cells that have not been exposed to the compound for a period of time to allow cell activation; (c) permeabilizing and fixating (i) the T-reg cells of step (b)(i) and (ii) the control T-reg cells of step (b)(ii); (d) contacting (i) the T-reg cells of step (c)(i) and (ii) the control T-reg cells of step (c)(ii) with a nucleic acid probe that is complementary to a nucleic acid sequence associated with an anti-inflammatory response under conditions in which the nucleic acid probe hybridizes to a complementary nucleic acid sequence in the T-cells; (e) detecting using flow cytometry (i) an amount of signal (S₁) generated by the probe that is hybridized to the complementary target nucleic acid sequence in the T-reg cells of step (d)(i), and (ii) an amount of signal (S₀) generated by the probe that is hybridized to the control T-reg cells of step (d)(ii); and (f) detecting or measuring (i) the amount of signal (S₁) generated in step (e)(i), and (ii) the amount of signal (S₀) generated in step (e)(ii), wherein the compound that induces a S₁/S₀≥1 or a S₀/S₁<1 is identified as a candidate compound for inducing immune tolerance in the subject.

53. The method of embodiment 52, further comprising before step (c) a step of washing the cells of step (b) to remove unbound compound.

54. The method of embodiment 52, wherein the nucleic acid sequence associated with an anti-inflammatory response is part of a gene coding for an anti-inflammatory cytokine or a receptor for an anti-inflammatory cytokine present in one or more regulatory T-cells.

55. The method of embodiment 52, which is performed in the presence of IL-2.

56. The method of embodiment 52, wherein the probes are part of a homogeneous probe system that generates a signal when bound to a complementary sequence in the T-cell mRNA.

57. A method of identifying a compound comprising an epitope from a library or collection of epitopes that induces immune tolerance in a subject suffering from an immune-mediated disorder comprising the steps of (a) exposing (i) live T-resp cells from the subject, and (ii) live T-resp cells from a healthy individual to the compound in the presence of an epitope binding agent; (b) incubating the cells for a period of time to allow cell activation; (c) permeabilizing and fixating the cells of step (b); (d) contacting the cells of step (c) with a nucleic acid probe that is complementary to a nucleic acid sequence that is part of a gene associated with a pro-inflammatory response under conditions in which the nucleic acid probe hybridizes to a complementary nucleic acid sequence in the T-cells; (e) detecting using flow cytometry (i) an amount of signal generated by the probe that is hybridized to the complementary target nucleic acid sequence associated with a pro-inflammatory response under conditions in which the nucleic acid probe hybridizes to a complementary nucleic acid sequence in the T-resp cells of the subject (RespP), and (ii) an amount of signal generated by the probe that is hybridized to the complementary target nucleic acid sequence associated with a pro-inflammatory response under conditions in which the nucleic acid probe hybridizes to a complementary nucleic acid sequence in the T-resp cells of the healthy individual (RespH); and (f) detecting or measuring (i) the amount of signal (RespP) measured in step (e)(i), and (ii) the amount of signal (RespH) measured in step (e)(ii), wherein the compound that induces a RespH/RespP≥1 or a RespP≤RespH is identified as a candidate compound for inducing immune tolerance in the subject.

58. The method of embodiment 57, further comprising after step (b) a step of washing the cells of step (b) to remove unbound compound.

59. The method of embodiment 57, further comprising the steps of (g) determining the total number of T-cells (P1) from the healthy individual that provides an amount of regulatory T-cells that induces 50% suppression of responder T-cell activity in the presence of the compound; and (h) determining the total number of T-cells (P2) from the subject that provides an amount of regulatory T-cells that induces 50% suppression of responder T-cell activity in the presence of the compound; wherein the compound that induces a RespH/RespP≥1, a P1/P2>1 or RespH/RespP≥1 and a P1/P2>1 is identified as a candidate compound for inducing an immune tolerance.

60. The method of embodiment 57, wherein the nucleic acid sequence associated with a pro-inflammatory response is part of a gene coding for a pro-inflammatory cytokine or a receptor for a pro-inflammatory cytokine present in one or more responder T-cells.

61. The method of embodiment 57, wherein the probes are part of a homogeneous probe system that generates a signal when bound to a complementary sequence in the T-cell mRNA.

62. A method of identifying a compound comprising an epitope from a library or collection of epitopes that induces immune tolerance in a subject suffering from an immune mediated disorder comprising the step of (a) exposing (i) live T-resp cells from the subject and live T-resp cells from a healthy individual; and (ii) live T-reg cells from the subject and live T-reg cells from a healthy individual; to the compound in the presence of an epitope binding agent; (b) incubating the cells for a period of time to allow cell activation; (c) fixating and permeabilizing the cells of step (b); (d) contacting (i) the T-resp cells of step (c) with a nucleic acid probe that is complementary to a nucleic acid that is part of a gene associated with a pro-inflammatory response under conditions in which the nucleic acid probe hybridizes to a complementary nucleic acid sequence in the responder T-resp cells; and (ii) the T-reg cells of step (c) with a nucleic acid probe that is complementary to a nucleic acid that is part of a gene associated with an anti-inflammatory response under conditions in which the nucleic acid probe hybridizes to a complementary nucleic acid sequence in the T-reg cells; (e) detecting using flow cytometry (i) an amount of signal generated by the probe of step (d)(i) hybridized to the T-resp cells of the healthy individual (RespH), (ii) an amount of signal generated by the probe of step (d)(i) hybridized to the T-resp cells of the subject (RespP), (iii) an amount of signal generated by the probe of step (d)(ii) hybridized to the T-reg cells of the healthy individual (RegH), and (iv) an amount of signal generated by the probe of step (d)(ii) hybridized to the T-reg cells of the subject (RegP), and (vi) detecting or measuring (i) the amount of signal generated in step (e)(i) (RespH), (ii) the amount of signal generated in step (e)(ii) (RespP), (iii) the amount of signal generated in step (e)(iii) (RegH), and (iv) the amount of signal generated in step (e)(iv) (RegP), wherein the compound that induces a RespH/RespP<l, a RegH/RegP≥1 or a RespH/RespP<l and a RegH/RegP≥1 is identified as a candidate compound for inducing immune tolerance.

63. The method of embodiment 62, further comprising before step (b) a step of washing the cells of step (a) to remove unbound compound.

64. The method of embodiment 62, further comprising after step (b) a step of washing the cells of step (b) to remove unbound compound.

65. The method of embodiment 62, wherein the nucleic acid sequence associated with a pro-inflammatory response is part of a gene coding for a pro-inflammatory cytokine or a receptor for a pro-inflammatory cytokine present in one or more responder T-cells.

66. The method of embodiment 62, wherein the nucleic acid sequence associated with an anti-inflammatory response is part of a gene coding for an anti-inflammatory cytokine or a receptor for an anti-inflammatory cytokine present in one or more regulatory T-cells.

67. The method of embodiment 62, wherein the probes are part of a homogeneous probe system that generates a signal when bound to a complementary sequence in the T-cell mRNA.

68. A method of identifying a compound from a library or collection of compounds that induces immune tolerance in a human subject suffering from age-related macular degeneration or uveitis comprising the steps of (a) exposing (i) live responder T-cells from the subject, and (ii) live responder T-cells from a healthy individual the T-cells of step (a) to the compound in the presence of an epitope binding agent; (b) incubating the cells for a period of time to allow cell activation; (c) fixating and permeabilizing the T-cells of step (b); (d) contacting (i) the responder T-cells of the subject with a nucleic acid probe that is complementary to a nucleic acid sequence associated with a pro-inflammatory response present in one or more responder T-cells from the subject under conditions in which the nucleic acid probe hybridizes to a complementary nucleic acid sequence in the responder T-cells, and (ii) the responder T-cells of the healthy individual with a nucleic acid probe that is complementary to a nucleic acid sequence associated with a pro-inflammatory response present in one or more responder T-cells from the healthy individual under conditions in which the nucleic acid probe hybridizes to a complementary nucleic acid sequence in the responder T-cells; (e) detecting by flow cytometry (i) i. an amount of signal generated by the probe of step (d)(i) hybridized to the responder T-cells of the subject (RespP), and (ii) ii. an amount of signal generated by the probe of step (d)(ii) hybridized to the responder T-cells of the healthy individual (RespH); (f) detecting or measuring (i) the amount of signal generated in step (e)(i) (RespP), and (ii) the amount of signal generated in step (e)(ii) (RespH); wherein the compound that induces a RespH<RespP or RespP≥RespH is identified as a candidate compound for inducing immune tolerance in the subject.

69. The method of embodiment 68, further comprising after step (b) a step of washing the cells of step (b) to remove unbound compound.

70. The method of embodiment 68, wherein the nucleic acid sequence associated with a pro-inflammatory response is part of a gene coding for a pro-inflammatory cytokine or a receptor for a pro-inflammatory cytokine present in one or more responder T-cells.

71. The method of embodiment 68, wherein the probes are part of a homogeneous probe system that generates a signal when bound to a complementary sequence in the T-cell mRNA.

72. A method of identifying a compound that induces immune tolerance in a human subject suffering from age-related macular degeneration or uveitis from a library or collection of compounds comprising the steps of (a) exposing (i) live regulatory T-cells from the subject, and (ii) live regulatory T-cells from a healthy individual; to the compound in the presence of an epitope binding agent; (b) incubating the cells of step (a) for a period of time to allow cell activation; (c) fixating and permeabilizing the cells of step (b); (d) contacting (i) the regulatory T-cells of the subject with a nucleic acid probe that is complementary to a nucleic acid sequence associated with an anti-inflammatory response that is present in one or more regulatory T-cells from the subject under conditions in which the nucleic acid probe hybridizes to a complementary nucleic acid sequence in the regulatory T-cells, and (ii) the regulatory T-cells of the healthy individual with a nucleic acid probe that is complementary to a nucleic acid sequence associated with an anti-inflammatory response that is present in one or more regulatory T-cells from the healthy individual under conditions in which the nucleic acid probe hybridizes to a complementary nucleic acid sequence in the regulatory T-cells; (e) detecting by flow cytometry (i) an amount of signal generated by the probe of step (d)(i), and (ii) an amount of signal generated by the probe of step (d)(ii); (f) detecting or measuring (i) the amount of signal generated in step (e)(i) (RegS); and (ii) the amount of signal generated in step (e)(ii) (RegH), wherein the compound that induces a RegH>RegS is identified as a candidate compound for inducing immune tolerance in the subject.

73. The method of embodiment 72, further comprising after step (b) a step of washing the cells of step (b) to remove unbound compound.

74. The method of embodiment 72, wherein the nucleic acid sequence associated with an anti-inflammatory response is part of a gene coding for an anti-inflammatory cytokine or a receptor for an anti-inflammatory cytokine present in one or more regulatory T-cells.

75. The method of embodiment 72, wherein the probes are part of a homogeneous probe system that generates a signal when bound to a complementary sequence in the T-cell mRNA.

76. The method of embodiment 72 wherein the regulatory T-cells are CD4+CD25+ T-cells.

77. An in vitro method of identifying from a library or collection of compounds a compound comprising an epitope that induces immune tolerance in a human subject suffering from an immune-mediated disorder comprising the steps of (a) contacting one or more live immune cells of the subject with (i) a labeled compound comprising the epitope in the presence of an epitope binding agent under conditions in which the compound binds to a T-cell receptor on a regulatory T-cell; and (ii) a labeled probe specific for a regulatory T-cell-specific marker; (b) identifying or isolating regulatory T-cells bound to the probe of step (a)(ii) by flow cytometry; and (c) detecting or measuring an amount of the labeled compound of step (a)(i) bound to the T-cell receptors of the regulatory T-cells of step (b) by flow cytometry, wherein the presence or quantity of the labeled compound of step (a)(i) bound to the subject's regulatory T-cells identifies an epitope that elicits immune tolerance in the subject.

78. The method of embodiment 77, wherein the library is a library of biological epitopes.

79. The method of embodiment 77, wherein the library is a library of HLA epitopes.

80. The method of embodiment 77, wherein the library is a library of HLA variant epitopes.

81. The method of embodiment 80, wherein the library is a library of HLA-B27 epitopes.

82. The method of embodiment 78, wherein the library is a library of S-antigen epitopes.

83. The method of embodiment 78, wherein the library is a library of self biological epitopes.

84. The method of embodiment 78, wherein the library is a library of non-self biological epitopes.

85. The method of embodiment 78, wherein the library is a library of self and non-self biological epitopes.

86. The method of embodiment 78, wherein the collection includes all permutations of epitope pentamers.

87. The method of embodiment 78, wherein the collection includes all permutations of epitope tetramers.

88. The method of embodiment 77, wherein the probe of step (a)(ii) is an antibody.

89. The method of embodiment 88, wherein the antibody binds to a marker selected from CD25, CD39, Foxp3, CTLA-4, HLA-DR, CD45RA, CD73, GITR, TGFβ, GARP and LAP, or a combination thereof.

90. An in vitro method of identifying from a library or collection of compounds a compound comprising an epitope that induces immune tolerance in a human subject suffering from an immune-mediated disorder comprising the steps of (a) contacting one or more live immune cells of the subject with (i) a labeled compound comprising the epitope in the presence of an epitope binding agent under conditions in which the compound binds to a T-cell receptor on a regulatory T-cell; and (ii) a labeled probe specific for a regulatory T-cell-specific surface marker; (b) identifying or isolating regulatory T-cells bound to the probe of step (a)(ii) by flow cytometry; (c) detecting or measuring an amount of the labeled compound of step (a)(i) bound to the T-cell receptors of the regulatory T-cells of step (b) by flow cytometry; (d) fixating and permeabilizing the cells of step (c); (e) contacting the regulatory T-cells of step (d) with (i) a labeled nucleic acid probe that is complementary to a target nucleic acid that is part of a gene that codes for an anti-inflammatory cytokine or for a receptor for an anti-inflammatory cytokine; and (ii) an antibody to an intracellular target; (f) detecting or measuring an amount of signal generated by the probe of step (e)(i) and the antibody of step (e)(ii) that is hybridized to the target nucleic acid in the cell using flow cytometry, wherein the presence or quantity of the target nucleic acid, the intracellular target or both the target nucleic acid and the intracellular target in the cell identifies an epitope that induces immune tolerance in the subject.

91. The method of embodiment 90, wherein the nucleic acid of step (e)(i) is mRNA.

92. The method of embodiment 90, wherein the target nucleic acid encodes an anti-inflammatory cytokine selected from IL-4, IL-10, IL-11, IL-13, TGFβ, IL-35, LIF, CTLA-4, CD39, galectin 1, galectin 3, galectin 9, PD-L1 and combinations thereof.

93. The method of embodiment 90, wherein the target nucleic acid encodes a receptor for an anti-inflammatory cytokine selected from an IL-4 receptor, an IL-10 receptor, an IL-11 receptor, an IL-13 receptor, a TGFβ receptor, an IL-35 receptor, a LIF receptor, a CTLA-4 receptor, a CD39 receptor, a galectin 1 receptor, a galectin 3 receptor, a galectin 9 receptor, a PD-1 receptor and combinations thereof.

94. An in vitro method of identifying a compound from a library or collection of compounds comprising an epitope that induces immune tolerance in a human subject suffering from an immune-mediated disorder comprising the steps of (a) exposing (i) an amount of CD4+CD25+ T-cells from a healthy individual; and (ii) an equal amount of CD4+CD25+ T-cells from the subject to the compound; (b) fixating and permeabilizing the cells of step (a); (c) contacting the cells from step (a) with a nucleic acid probe that is complementary to a nucleic acid coding for a CD4+CD25+ T-cell marker; (d) detecting using flow cytometry (i) an amount of signal generated by the probe of step (c) hybridized to CD4+CD25+ T-cells from the healthy individual (RegH); and (ii) an amount of signal generated by the probe of step (c) hybridized to CD4+CD25+ T-cells from the subject (RegS); (e) detecting or measuring (i) the amount of signal generated in step (d)(i) (RegH); and (ii) the amount of signal generated in step (d)(ii)(RegS); wherein the compound that induces a RegH/RegS≤1 or a RegS/RegH>1 is identified as a candidate compound for inducing immune tolerance in the subject.

95. The method of embodiment 94, wherein the CD4+CD25+ T-cell marker is a cytokine selected from IL-4, IL-10, IL-11, IL-13, TGFβ, IL-35, LIF, CTLA-4, CD39, galectin 1, galectin 3, galectin 9, PD-L1 and combinations thereof.

96. The method of embodiment 94, wherein the CD4+CD25+ T-cell marker is selected from an IL-4 receptor, an IL-10 receptor, an IL-11 receptor, an IL-13 receptor, a TGFβ receptor, and IL-35 receptor, a LIF receptor, a CTLA-4 receptor, a CD39 receptor, a galectin 1 receptor, a galectin 3 receptor, a galectin 9 receptor, a PD-1 receptor, and combinations thereof.

97. The method of embodiment 94, wherein the CD4+CD25+ T-cell marker is selected from CD25, CD39, Foxp3, CTLA-4, HLA-DR, CD45RA, CD73, GITR, GARP and LAP and combinations thereof.

98. An in vitro method of identifying from a library or collection of compounds a compound comprising an epitope that induces immune tolerance in a subject suffering from an immune-mediated disorder comprising the steps of (a) exposing (i) an amount of T-cells from the subject, and (ii) an equal amount of T-cells with CD25+ cells depleted from the subject to a fluorescent cell-staining dye; (b) exposing (i) the T-cells of step (a)(i) and (ii) the T-cells of step (a)(ii) to the compound in the presence of an epitope binding agent; (c) incubating the T-cells of (i) step (b)(i) and (ii) step (b)(ii) for a period of time to allow cell proliferation; (d) fixating and permeabilizing (i) the cells of step (c)(i) and (ii) the cells of step (c)(ii); (e) contacting (i) the T-cells from the subject of step (d)(i) with a CD4+ cell-surface probe and a labeled nucleic acid probe that is complementary to a target nucleic acid that is part of a gene that codes for a pro-inflammatory cytokine or for a receptor of a pro-inflammatory cytokine under conditions in which the nucleic acid probe hybridizes to a complementary nucleic acid sequence in the cells; and (ii) the T-cells with CD25+ cells depleted from the subject of step (d)(ii) with a CD4 cell-surface probe and a labeled nucleic acid probe that is complementary to a target nucleic acid that is part of a gene that codes for a pro-inflammatory cytokine or for a receptor of a pro-inflammatory cytokine under conditions in which the nucleic acid probe hybridizes to a complementary nucleic acid sequence in the cells; (f) detecting by flow cytometry (i) a CD4+ signal, a fluorescent cell-staining dye signal, and a signal from the labeled nucleic acid probe in the cells of step (e)(i); and (ii) a CD4+ signal, a fluorescent cell-staining dye signal, and a signal from the labeled nucleic acid probe in the cells of step (e)(ii); (g) detecting or measuring by flow cytometry (i) an amount of CD4+ T-cells in step (f)(i) having a low fluorescent cell-staining dye signal (D₁) and a signal from the labeled nucleic acid probe (T-Resp₁); and (ii) an amount of CD4+ T-cells in step (f)(ii) having a low fluorescent cell-staining dye signal (D₂) and a signal from the labeled probe (T-Resp₂), wherein the compound that induces a T-Resp₁≤T-Resp₂ or a T-Resp₂>T-Resp₁ or the compound induces a D₁≥D₂ or a D₂<D₁ or both a T-Resp₁≤T-Resp₂ or a T-Resp₂>T-Resp₁ and a D₁≥D₂ or a D₂<D₁ is identified as a candidate compound for inducing immune tolerance in the subject.

99. The method of embodiment 98, wherein the T-cells of step (a)(ii) are depleted using anti-CD25 beads.

100. The method of embodiment 98, further comprising before step (c) a step of washing the T-cells of step (b) to remove unbound compound.

101. An in vitro method of identifying from a library or collection of compounds a compound comprising an epitope that induces immune tolerance in a human subject suffering from an immune-mediated disorder comprising the steps of (a) exposing (i) an amount of CD4+CD25+ T-cells (T-reg) from the subject, and (ii) an equal amount of CD4+CD25− T-cells (T-resp) from the subject to a fluorescent cell-staining dye; (b) exposing (i) the cells of step (a)(i) and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of T-reg cells of step (c)(i) having a low fluorescent cell-staining dye signal (RegS); and (ii) an amount of T-resp cells in step (c)(ii) having a low fluorescent cell-staining dye signal (RespS), wherein the compound that induces a RegS≥RespS or a RespS<RegS is identified as a candidate compound for inducing immune tolerance in the subject.

102. The method of embodiment 101, further comprising before step (c) a step of washing the cells of step (b) to remove unbound compound.

103. The method of embodiment 101, further comprising after step (c) a step of fixating and permeabilizing the cells of step (c), and a step of contacting the fixated T-reg cells and the fixated T-resp cells with a probe that is specific for Foxp3.

104. The method of embodiment 101, wherein step (a) is performed in the presence of IL-2.

105. An in vitro method of identifying from a library or collection of compounds a compound comprising an epitope that induces immune tolerance in a human subject suffering from an immune-mediated disorder comprising the steps of (a) exposing (i) an amount of CD4+CD25+Foxp3+ T-cells from the subject (RegS) and an equal amount of CD4+CD25+Foxp3+ T-cells from a healthy individual (RegH), and (ii) an amount of CD4+CD25−Foxp3− T-cells from the subject (RespS) and an equal amount of CD4+CD25−Foxp3− T-cells from a healthy individual (RespH) to a fluorescent cell-staining dye; (b) exposing (i) the cells of step (a)(i) and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of RegS cells of step (c)(i) having a low fluorescent cell-staining dye signal (RegS); (ii) an amount of RegH cells of step (c)(i) having a low fluorescent cell-staining dye signal (RegH); (iii) an amount of RespS cells of step (c)(ii) having a low fluorescent cell-staining dye signal (RespS); and (iv) an amount of RespH cells of step (c)(ii) having a low fluorescent cell-staining dye signal (RespH); wherein the compound that induces a RespH/RespS≥1, a RegH/RegS≤1 or a RespH/RespS≥1 and a RegH/RegS≤1 is identified as a candidate compound for inducing immune tolerance in the subject.

106. The method of embodiment 105, further comprising before step (c) a step of washing the cells of step (b) to remove unbound compound.

107. The method of embodiment 105, further comprising after step (c) a step of fixating and permeabilizing the cells of step (c), and a step of contacting the fixated T-reg cells and the fixated T-resp cells with a probe that is specific for Foxp3.

108. The method of embodiment 105, wherein step (a)(i) is performed in the presence of IL-2.

109. An in vitro method of identifying from a library or collection of compounds a compound comprising an epitope that induces immune tolerance in a human subject suffering from an immune-mediated disorder comprising the steps of (a) exposing (i) an amount of CD4+CD25−Foxp3− T-cells from the subject in the presence of CD4+CD25+Foxp3+ T-cells from the subject, and (ii) an equal amount of CD4+CD25−Foxp3− T-cells from the subject in the absence of CD4+CD25+Foxp3+ T-cells to a fluorescent cell-staining dye; (b) exposing (i) the cells of step (a)(i), and (ii) the cells of step (a)(ii) to the compound in the presence of an epitope binding agent; (c) incubating (i) the cells of step (b)(i) and (ii) the cells of step (b)(ii) for a period of time to allow cell proliferation; (d) detecting or measuring by flow cytometry (i) an amount of cells of step (c)(i) having a low fluorescent cell-staining dye signal (RespS₁); and (ii) an amount of cells of step (c)(ii) having a low fluorescent cell-staining dye signal (RespS₀); wherein the compound that induces a RespS₁≤RespS₀ is identified as a candidate compound for inducing immune tolerance in the subject.

110. The method of embodiment 109, further comprising before step (c) a step of washing the cells of step (b) to remove unbound compound.

111. The method of embodiment 109, further comprising before step (a) the steps of isolating using flow cytometry or magnetic beads CD4+CD25−Foxp3− T-cells from peripheral blood mononuclear cells of the subject.

112. An in vitro method of identifying a compound from a library or collection of compounds comprising an epitope that induces immune tolerance in a human subject suffering from a disease (a) exposing (i) an amount of CD4+CD25−Foxp3− T-cells from the subject, and (ii) an equal amount of CD4+CD25-Foxp3− T-cells from the subject to a fluorescent cell staining dye; (b) exposing the cells of step (a)(i) to the compound in the presence of an epitope binding agent; (c) incubating (i) the cells of step (a)(ii) and (ii) the cells of step (b) for a period of time to allow cell proliferation; (d) detecting or measuring by flow cytometry (i) an amount of cells of step (c)(ii) having a low fluorescent cell-staining dye signal (RespS); and (ii) an amount of cells of step (c)(i) having a low fluorescent cell-staining dye signal (RespS₀); wherein the compound that induces a RespS/RespS₀≤1 is identified as a candidate compound for inducing immune tolerance.

113. The method of embodiment 112, further comprising before step (c) a step of washing the cells to remove unbound compound.

114. The method of embodiment 112, further comprising before step (a) a step of isolating using flow cytometry CD4+CD25−Foxp3− T-cells or magnetic beads from peripheral blood mononuclear cells of the subject.

115. An in vitro method of identifying a compound from a library or collection of compounds comprising an epitope that induces immune tolerance in a human subject suffering from an immune-mediated disorder comprising the steps of (a) exposing (i) an amount of CD4+CD25+ T-cells from a healthy individual to the compound in the presence of CD4+CD25− T-cells from the healthy individual, and (ii) an equal amount of CD4+CD25+ T-cells from the subject to the compound in the presence of an equal amount of CD4+CD25− T-cells from the subject to a fluorescent cell-staining dye; (b) exposing (i) the cells of step (a)(i); and (ii) the cells of step (a)(ii) to the compound in the presence of an epitope binding agent; (c) incubating (i) the cells of step (b)(i), and (ii) the cells of step (b)(ii) for a period of time to allow cell proliferation; (d) detecting or measuring by flow cytometry (i) an amount of CD4+CD25+ cells of step (c)(i) having a low fluorescent cell-staining dye signal (RegH); and (ii) an amount of CD4+CD25+ cells of step (c)(ii) having a low fluorescent cell-staining dye signal (RegS), wherein the compound that induces a RegH/RegS≤1 or a RegS/RegH>1 is identified as a candidate compound for inducing immune tolerance in the subject.

116. The method of embodiment 115, further comprising before step (c) a step of washing the cells of step (b) to remove unbound compound.

117. An in vitro method of identifying a compound comprising an epitope from a library or collection of compounds that elicits immune responsiveness in a human subject suffering from a disease comprising (a) contacting one or more immune cells of the subject with (i) a labeled compound comprising the epitope in the presence of an epitope binding agent under conditions in which the compound binds to a T-cell receptor on a responder T-cell; and (ii) a labeled probe specific for a responder T-cell-specific surface marker; (b) identifying or isolating responder T-cells bound to the labeled probe of step (a)(ii) by flow cytometry; and (c) detecting or measuring an amount of the labeled compound of step (a)(i) bound to the T-cell receptors on the responder T-cells of step (b) by flow cytometry, wherein the presence or quantity of the labeled compound of step (a)(i) bound to the subject's responder T-cells identifies an epitope that elicits an immune response in the subject.

118. The method of embodiment 117, wherein the library is a library of biological epitopes.

119. The method of embodiment 117, wherein the library is a library of HLA epitopes.

120. The method of embodiment 117, wherein the library is a library of HLA variant epitopes.

121. The method of embodiment 120, wherein the library is a library of HLA-B27 epitopes.

122. The method of embodiment 118, wherein the library is a library of S-antigen epitopes.

123. The method of embodiment 118, wherein the library is a library of self biological epitopes.

124. The method of embodiment 118, wherein the library is a library of non-self biological epitopes.

125. The method of embodiment 118, wherein the library is a library of self and non-self biological epitopes.

126. The method of embodiment 118, wherein the collection includes all permutations of epitope pentamers.

127. The method of embodiment 118, wherein the collection includes all permutations of epitope tetramers.

128. The method of embodiment 117, wherein the probe of step (a)(ii) is an antibody.

129. The method of embodiment 128, wherein the antibody binds to a marker selected from CD8, CD16, CD56, CD4, CD3, CD69, CD45RO, Tbet, perforin, Granzyme B, Nk1.1, NKG2D and combinations thereof.

130. An in vitro method of identifying from a library or collection of compounds a compound comprising an epitope that induces immune tolerance in a human subject suffering from a disease comprising the steps of (a) contacting one or more live immune cells of the subject with (i) a labeled compound comprising an epitope in the presence of an epitope binding agent under conditions in which the compound binds to a T-cell receptor on a responder T-cell; and (ii) a labeled probe specific for a responder T-cell-specific surface marker; (b) identifying or isolating responder T-cells bound to the probe of step (a)(ii) by flow cytometry; (c) detecting or measuring an amount of the labeled compound of step (a)(i) bound to the T-cell receptors of the responder T-cells of step (b) by flow cytometry; (d) fixating and permeabilizing the cells of step (c); (e) contacting the responder T-cells of step (d) with (i) a labeled nucleic acid probe that is complementary to a target nucleic acid that is part of a gene that codes for a pro-inflammatory cytokine or for a receptor of a pro-inflammatory cytokine; and (ii) an antibody to an intracellular target; and (f) detecting or measuring an amount of signal generated by the probe of step (e)(i) and the antibody of step (e)(ii) that is hybridized to the target nucleic acid in the cell using flow cytometry, wherein the presence or quantity of the target nucleic acid, the intracellular target or both the target nucleic acid and the intracellular target in the cell identifies an epitope that induces immune tolerance in the subject.

131. The method of embodiment 130, wherein the nucleic acid of step (e)(i) is mRNA.

132. The method of embodiment 130, wherein the target nucleic acid of step (e)(i) encodes a pro-inflammatory cytokine selected from IFNγ, TNFα, I, IL-6, IL-12, IL-17, IL-18, IL-15, IL-8, IL-21, IL-25 and combinations thereof.

133. The method of embodiment 130, wherein the target nucleic acid of step (e)(i) encodes a receptor for a pro-inflammatory cytokine selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof.

134. The method of embodiment 130, wherein the probe of step (e)(i) is part of a homogeneous probe system that generates a signal when bound to a complementary sequence in the target nucleic acid.

135. The method of embodiment 130, wherein the antibody of step (e)(ii) binds to a marker selected from CD8, CD16, CD16, CD56, CD4, CD3, CD69, CD45RO, Tbet, perforin, Granzyme B, Nk1.1, NKG2D and combinations thereof.

136. An in vitro method of identifying a compound from a library or collection of compounds comprising an epitope that induces immune responsiveness in a human subject suffering from a disease comprising the steps of (a) exposing (i) an amount of CD4+CD25− T-cells from a healthy individual, and (ii) an amount of CD4+CD25− T-cells from the subject; to the compound in the presence of an epitope binding agent; (b) incubating the cells of step (a) for a period of time sufficient to allow cell activation; (c) fixating and permeabilizing the cells of step (b); (d) contacting the cells of step (c) with a nucleic acid probe that is complementary to a nucleic acid coding for a CD4+CD25− T-cell marker; (e) detecting using flow cytometry (i) an amount of signal generated by the probe of step (d) hybridized to CD4+CD25− T-cells from the healthy individual (RespH); and (ii) an amount of signal generated by the probe of step (d) hybridized to CD4+CD25− T-cells from the subject (RespS); (f) detecting or measuring (i) the amount of signal generated in step (e)(i) (RespH); and (ii) the amount of signal generated in step (e)(ii)(RespS); wherein the compound that induces a RespS/RespH≤1 is identified as a candidate compound for inducing immune tolerance in the subject.

137. The method of embodiment 136, further comprising before step (b) a step of washing the cells of step (a) to remove unbound compound.

138. The method of embodiment 136, wherein the CD4+CD25− T-cell marker is selected from IFNγ, TNFα, IL1, IL-6, IL-12, IL-17, IL-18, IL-15, IL-8, IL-21, IL-25 and combinations thereof.

139. The method of embodiment 136, wherein the CD4+CD25− T-cell marker is a receptor for a pro-inflammatory cytokine selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof.

140. A method of identifying a compound comprising an epitope from a library or collection of epitopes that induces immune responsiveness in a subject suffering from a disease comprising the steps of (a) exposing responder T-cells from the subject to the compound in the presence of an epitope binding agent; (b) incubating the cells of step (a) for a period of time sufficient to allow cell activation; (c) fixating and permeabilizing (i) the responder T-cells of step (b), and (ii) an equal amount of control responder T-cells that have not been exposed to the compound; (d) contacting (i) the responder T-cells of step (c)(i) and (ii) the control responder T-cells of step (c)(ii) with a nucleic acid probe that is complementary to a nucleic acid sequence associated with a pro-inflammatory response under conditions in which the nucleic acid probe hybridizes to a complementary nucleic acid sequence in the T-cells; (e) detecting using flow cytometry (i) an amount of signal (S₁) generated by the probe that is hybridized to the complementary target nucleic acid sequence in the responder T-cells of step (d)(i), and (ii) an amount of signal (S₀) generated by the probe that is hybridized to the control responder T-cells of step (d)(ii); (f) detecting or measuring (i) the amount of signal (S₁) generated in step (e)(i), and (ii) the amount of signal (S₀) generated in step (e)(ii), wherein the compound that induces a S₁/S₀>1 or a S₀/S₁≤1 is identified as a candidate compound for inducing immune responsiveness in the subject.

141. The method of embodiment 140, further comprising before step (b) a step of washing the cells of step (a) to remove unbound compound.

142. A method of identifying a compound comprising an epitope from a library or collection of epitopes that induces immune responsiveness in a subject suffering from a disease comprising the steps of (a) exposing (i) responder T-cells from the subject, and (ii) responder T-cells from a healthy individual to the compound in the presence of an epitope binding agent; (b) incubating the cells of step (a) for a period of time sufficient to allow cell activation; (c) fixating and permeabilizing the cells of step (b); (d) contacting the cells of step (c) with a nucleic acid probe that is complementary to a nucleic acid sequence that is part of a gene associated with a pro-inflammatory response under conditions in which the nucleic acid probe hybridizes to a complementary nucleic acid sequence in the T-cell; (e) detecting using flow cytometry (i) an amount of signal generated by the probe that is hybridized to the complementary target nucleic acid sequence associated with a pro-inflammatory response in the responder T-cells of the subject (RespP), and (ii) an amount of signal generated by the probe that is hybridized to the complementary target nucleic acid sequence associated with a pro-inflammatory response in the responder T-cells of the healthy individual (RespH); and (f) detecting or measuring (i) the amount of signal (RespP) measured in step (e)(i), and (ii) the amount of signal (RespH) measured in step (e)(ii), wherein the compound that induces a RespH/RespP<1 or a RespP≥RespH is identified as a candidate compound for inducing immune responsiveness.

143. The method of embodiment 142, further comprising before step (b) a step of washing the cells of step (a) to remove unbound compound.

144. The method of embodiment 142, wherein the responder T-cells of step (a)(i) are in the presence of regulatory T-cells from the subject, and wherein the responder T-cells of step (a)(ii) are in the presence of an equal amount of regulatory T-cells from the healthy individual.

145. A method of identifying from a library or collection of compounds a compound comprising an epitope that induces immune responsiveness in a human subject suffering from a disease comprising the steps of (a) exposing a portion of CD4+CD25− cells to the compound in the presence of an epitope binding agent; (b) incubating the cells of step (a) for a period of time sufficient to allow cell activation; (c) fixating and permeabilizing the cells of step (b) and an equal amount of CD4+CD25− cells that were not exposed to the compound; (d) contacting the cells of step (c) with a nucleic acid probe that is complementary to a nucleic acid sequence present in one or more CD4⁺CD25⁻ cells that is associated with a pro-inflammatory response in the cells under conditions in which the nucleic acid probe hybridizes to a complementary nucleic acid sequence in the CD4⁺CD25⁻ cells; (e) detecting by flow cytometry (i) an amount of signal generated by the probe that is hybridized to the complementary target nucleic acid sequence associated with a pro-inflammatory response in the responder T-cells that were exposed to the compound, and (ii) an amount of signal generated by the probe that is hybridized to the complementary target nucleic acid sequence associated with a pro-inflammatory response in the equal amount of responder T-cells that were not exposed to the compound; and (f) detecting or measuring (i) the amount of signal generated in step (e)(i) (S₁), and (ii) the amount of signal generated in step (e)(ii) (S₀), wherein the compound that induces S₁/S₀>1 is identified as a candidate compound for inducing immune responsiveness in the subject.

146. The method of embodiment 145, further comprising before step (b) a step of washing the cells to remove unbound compound.

147. An in vitro method of identifying from a library or collection of compounds a compound comprising an epitope that induces immune responsiveness in a subject suffering from a disease comprising the steps of (a) exposing (i) an amount of CD4+ T-cells from the subject to the compound, and (ii) an equal amount of T-cells with CD25+ cells depleted from the subject to the compound in the presence of an epitope binding agent and a fluorescent cell-staining dye; (b) incubating (i) the cells of step (a)(i), and (ii) the cells of step (a)(ii) for a period of time sufficient to allow cell proliferation; (c) fixating and permeabilizing (i) the cells of step (b)(i); and (ii) the cells of step (b)(ii); (d) contacting (i) the cells of step (c)(i), and (ii) the cells of step (c)(ii); with a cell surface marker for CD4, and a nucleic acid probe that is complementary to a target nucleic acid sequence that is part of a gene associated with a pro-inflammatory response present in one or more cells; (e) detecting by flow cytometry (i) a CD4 signal, and a fluorescent cell-staining dye signal in the cells of step (d)(i); and (ii) a CD4 signal, and a fluorescent cell-staining dye signal in the cells of step (d)(ii); and (f) detecting or measuring by flow cytometry (i) an amount of CD4+ T-cells in step (e)(i) having a low fluorescent cell-staining dye signal (T-Resp1); and (ii) an amount of CD4+ T-cells in step (e)(ii) having a low fluorescent cell-staining dye signal (T-Resp2), wherein the compound that induces a T-Resp1≤T-Resp2 is identified as a candidate compound for inducing immune responsiveness in the subject.

148. The method of embodiment 147, further comprising before step (b) a step of washing the cells of step (a) to remove unbound compound.

149. An in vitro method of identifying from a library or collection of compounds a compound comprising an epitope that induces immune responsiveness in a human subject suffering from a disease comprising the steps of (a) exposing (i) an amount of CD4+CD25+Foxp3+ T-cells (T-reg) from the subject, and (ii) an equal amount of CD4+CD25−Foxp3− T-cells (T-resp) from the subject to the compound in the presence of an epitope binding agent and a fluorescent cell-staining dye; (b) incubating (i) the cells of step (a)(i), and (ii) the cells of step (a)(ii) for a period of time sufficient to allow cell proliferation; (c) detecting by flow cytometry (i) a CD4 signal, and a fluorescent cell-staining dye signal in the cells of step (b)(i); and (ii) a CD4 signal, and a fluorescent cell-staining dye signal in the cells of step (b)(ii); and (d) detecting or measuring by flow cytometry (i) an amount of T-reg cells in step (c)(i) having a low fluorescent cell-staining dye signal (RegS); and (ii) an amount of T-resp cells in step (c)(ii) having a low fluorescent cell-staining dye signal (RespS), wherein the compound that induces an RegS≤RespS or an RespS>RegS is identified as a candidate compound for inducing immune responsiveness in the subject.

150. The method of embodiment 149, further comprising before step (b) a step of washing the cells to remove unbound compound.

151. An in vitro method of identifying from a library or collection of compounds a compound comprising an epitope that induces immune responsiveness in a human subject suffering from a disease (a) exposing (i) an amount of CD4+CD25+Foxp3+ T-cells from the subject (RegS), (ii) an equal amount of CD4+CD25+Foxp3+ T-cells in step (a)(i) from a healthy individual (RegH), (iii) an amount of CD4+CD25−Foxp3− T-cells from the subject (RespS), and (iv) an equal amount of CD4+CD25−Foxp3− T-cells in step (a)(iii) from a healthy individual (RespH) to the compound in the presence of an epitope binding agent and a fluorescent cell-staining dye; (b) separately incubating the cells of steps (a)(i), (a)(ii), (a)(iii) and (a)(iv) for a period of time sufficient to allow cell proliferation; (c) detecting by flow cytometry (i) a CD4 signal, and a fluorescent cell-staining dye signal in the cells of step (a)(i); (ii) a CD4 signal, and a fluorescent cell-staining dye signal in the cells of step (a)(ii); (iii) a CD4 signal, and a fluorescent cell-staining dye signal in the cells of step (a)(iii); and (iv) a CD4 signal, and a fluorescent cell-staining dye signal in the cells of step (a)(iv); and (d) detecting or measuring by flow cytometry (i) an amount of T-reg cells from the subject having a low fluorescent cell-staining dye signal (RegS); (ii) an amount of T-reg cells from the healthy individual having a low fluorescent cell-staining dye signal (RegH); (iii) an amount of RespS cells having a low fluorescent cell-staining dye signal (RespS); and (iv) an amount of RespH cells having a low fluorescent cell-staining dye signal (RespH); wherein the compound that induces a RespH/RespS<1, a RegH/RegS≥1 or a RespH/RespS≤1 and a RegH/RegS≥1 is identified as a candidate compound for inducing immune responsiveness.

152. The method of embodiment 151, further comprising before step (b) a step of washing the cells to remove unbound compound.

153. An in vitro method of identifying from a library or collection of compounds a compound comprising an epitope that induces immune responsiveness in a human subject suffering from an immune-related disorder comprising the steps of (a) exposing (i) an amount of CD4+CD25−Foxp3− T-cells from the subject in the presence of CD4+CD25+Foxp3+ T-cells from the subject; and (ii) an equal amount of CD4+CD25−Foxp3− T-cells from the subject in the absence of CD4+CD25+Foxp3+ T-cells to the compound in the presence of an epitope binding agent and a fluorescent cell-staining dye; (b) incubating (i) the CD4+CD25−Foxp3− T-cells in the presence of CD4+CD25+Foxp3+ T-cells of step (a)(i), and (ii) the CD4+CD25−Foxp3− T-cells of step (a)(ii) in the presence of the compound for a time sufficient to allow cell proliferation; (c) detecting by flow cytometry (i) an amount of CD4+CD25−Foxp3− T-cells of step (b)(i) having a low fluorescent cell-staining dye signal; and (ii) an amount of CD4+CD25−Foxp3− T-cells of step (b)(ii) having a low fluorescent cell-staining dye signal; (d) detecting or measuring by flow cytometry (i) an amount of CD4+CD25-Foxp3− T-cells of step (c)(i) having a low fluorescent cell-staining dye (RespS₁), and (ii) an amount of CD4+CD25-Foxp3− T-cells of step (c)(ii) having a low fluorescent cell-staining dye signal (RespS₀); wherein the compound that induces a RespS₀≥RespS₁ is identified as a candidate compound for inducing immune responsiveness in the subject.

154. The method of embodiment 153, further comprising before step (b) a step of washing the cells to remove unbound compound.

155. An in vitro method of identifying a compound from a library or collection of compounds comprising an epitope that induces immune responsiveness in a subject suffering from an immune-mediated disorder comprising the steps of (a) exposing (i) an amount of CD4+CD25− T-cells from a healthy individual to the compound in the presence of CD4+CD25+ T-cells from the healthy individual for a period of time to allow cell proliferation, and (ii) an equal amount of CD4+CD25− T-cells from the subject to the compound in the presence of an equal amount of CD4+CD25+ T-cells from the subject for a period of time to allow cell proliferation in the presence of a fluorescent cell-staining dye; (b) incubating (i) the CD4+CD25− T-cells of step (a)(i), and (ii) the CD4+CD25− T-cells of step (a)(ii) in the presence of the compound for a time sufficient to allow cell proliferation; (c) detecting by flow cytometry (i) an amount of CD4+CD25− T-cells of step (b)(i) having a low fluorescent cell-staining dye signal; and (ii) an amount of CD4+CD25− T-cells of step (b)(ii) having a low fluorescent cell-staining dye signal; (d) detecting or measuring by flow cytometry (i) an amount of CD4+CD25− cells of step (c)(i) having a low fluorescent cell-staining dye signal (RespH); and (ii) an amount of CD4+CD25− cells of step (c)(ii) having a low fluorescent cell-staining dye signal (RespS); wherein the compound that induces a RespH/RespS≤1 or a RespS/RespH>1 is identified as a candidate compound for inducing immune responsiveness in the subject.

156. The method of embodiment 155, further comprising before step (c) a step of washing the cells to remove unbound compound.

157. A method of determining the presence or absence of memory for an epitope in T-cells of a subject comprising the steps of (a) exposing (i) an amount of T-cells from the subject, and (ii) an equal amount of control T-cells to the epitope in the presence of an epitope binding agent for a period of time to allow cell proliferation in the presence of a fluorescent cell-staining dye; (b) incubating (i) the cells of step (a)(i), and (ii) the cells of step (a)(ii) in the presence of the compound for a period of time to allow cell proliferation; (c) detecting by flow cytometry (i) an amount of T-cells of step (b)(i) having a low fluorescent cell-staining dye signal; and (ii) an amount of T-cells of step (b)(ii) having a low fluorescent cell-staining dye signal; (d) detecting or measuring by flow cytometry (i) an amount of T-cells of step (c)(i) having a low fluorescent cell-staining dye signal (S₁); and (ii) an amount of T-cells of step (c)(ii) having a low fluorescent cell-staining dye signal (S₀) wherein an S₁/S₀>1 or an S₀/S₁≤1 identifies memory for the epitope in the T-cells from the subject, and wherein an S₁/S₀<1 or an S₀/S₁≥1 identifies the absence of memory for the epitope in the T-cells from the subject.

158. The method of embodiment 157, wherein the control T-cells of step (a)(ii) are naïve T-cells with respect to the epitope.

159. The method of embodiment 157, wherein the control T-cells of step (a)(ii) are T-cells from an individual that has cell memory for the epitope or are T-cells that were trained in vitro to have cell memory for the epitope.

160. The method of embodiment 157, further comprising before step (b) a step of washing the cells of step (a) to remove unbound compound.

161. A method of determining the presence or absence of memory for an epitope in T-cells of a subject comprising the steps of (a) exposing (i) an amount of T-cells from the subject, and (ii) an equal amount of control T-cells to the epitope in the presence of an epitope binding agent; (b) incubating (i) the cells of step (a)(i), and (ii) the cells of step (a)(ii) for a period of time sufficient to allow cell activation; (c) fixating and permeabilizing (i) the cells of step (b)(i), and (ii) the cells of step (b)(ii); (d) contacting (i) the cells of step (c)(i) and (ii) the cells of step (c)(ii) with a nucleic acid probe that is complementary to a nucleic acid coding for a T-cell marker; (e) detecting by flow cytometry (i) an amount of signal generated by the probe hybridized to the T-cells of step (d)(i); and (ii) an amount of signal generated by the probe hybridized to the T-cells of step (d)(ii); (f) detecting or measuring by flow cytometry (i) the amount of signal (S₁) generated by the T-cells of step (e)(i), and (ii) the amount of signal (S₀) generated by the T-cells of step (e)(ii), wherein an S₁/S₀>¹ or an S₀/S₁<1 identifies memory for the epitope in the T-cells from the subject, and wherein an S₁/S₀≤1 or an S₀/S₁>1 identifies the absence of memory for the epitope in the T-cells from the subject.

162. The method of embodiment 161 wherein the T-cells are T-resp cells and the T-cell marker is a pro-inflammatory cytokine selected from INFγ, TNFα, IL-1, IL-6, IL-12, IL-17, IL-18, IL-15, IL-8, IL-21, IL-25 and combinations thereof.

163. The method of embodiment 161, wherein the T-cells are T-resp cells and the T-cell marker is a receptor for a pro-inflammatory cytokine selected from an IFNγ receptor, a TNFα receptor, an IL1 receptor, an IL-6 receptor, an IL-12 receptor, an IL-17 receptor, an IL-18 receptor, an IL-15 receptor, an IL-8 receptor, an IL-21 receptor, an IL-25 receptor and combinations thereof.

164. The method of embodiment 161, wherein the T-cells are T-reg cells and the T-cell marker is an anti-inflammatory cytokine selected from IL-4, IL-10, IL-11, IL-13, TGFβ, IL-35, LIF, CTLA-4, CD39, galectin 1, galectin 3, galectin 9, PD-L1 and combinations thereof.

165. The method of embodiment 161, wherein the target nucleic acid encodes a receptor for an anti-inflammatory cytokine selected from an IL-4 receptor, an IL-10 receptor, an IL-I1 receptor, an IL-13 receptor, a TGFβ receptor, an IL-35 receptor, a LIF receptor, a CTLA-4 receptor, a CD39 receptor, a galectin 1 receptor, a galectin 3 receptor, a galectin 9 receptor, a PD-1 receptor and combinations thereof.

166. A method of determining memory for an epitope in T-cells comprising the steps of (a) contacting one or more T-cells with a labeled compound comprising the epitope in the presence of an epitope binding agent; and (b) detecting or measuring by flow cytometry an amount of the labeled compound of step (a) bound to the T-cells (S₁); wherein the presence or quantity of the labeled compound of step (a) bound to the T-cells is indicative of T-cell memory for the epitope.

167. The method of embodiment 166, further comprising (c) contacting one or more control T-cells with the labeled compound comprising the epitope of step (a); and (d) detecting or measuring by flow cytometry an amount of the labeled compound of step (c) bound to the control T-cells (S₀); wherein an S₁/S₀>¹ or an S₀/S₁≤1 identifies memory for the epitope in the T-cells from the subject, and wherein an S₁/S₀<1 or an S₀/S₁≥1 identifies the absence of memory for the epitope in the T-cells from the subject.

168. The method of embodiment 167, wherein the control T-cells of step (c) are naïve T-cells with respect to the epitope.

169. The method of embodiment 167, wherein the control T-cells of step (c) are T-cells from an individual that has cell memory for the epitope or are T-cells that were trained in vitro to have cell memory for the epitope.

170. A method of identifying an effective therapy in a subject suffering from an immune-mediated disorder, a disease or an infection comprising the steps of (a) obtaining (i) a first cell sample from the subject taken before treatment, and (ii) a second cell sample from the subject taken after treatment; (b) fixating and permeabilizing (i) the cells of step (a)(i) and (ii) the cells of step (a)(ii); (c) contacting (i) the cells of step (b)(i), and (ii) the cells of step (b)(ii) with a nucleic acid probe that is complementary to a nucleic acid of interest; and a control nucleic acid probe, wherein the nucleic acid probes hybridize to complementary nucleic acid sequences in the cells; (e) detecting using flow cytometry (i) an amount of signal generated by the probe of step (c)(i); and (ii) an amount of signal generated by probe of step (c)(ii); (f) detecting or measuring by flow cytometry (i) an amount of signal generated in step (d)(i) (Sig₀), and (ii) an amount of signal generated in step (d)(ii) (Sig₁), wherein a Sig₁>Sig₀ identifies a therapy that is effective in the subject.

171. The method of embodiment 170, wherein the subject is suffering from a disease or infection and the nucleic acid probe is complementary to a nucleic acid encoding a pro-inflammatory cytokine or a receptor for a pro-inflammatory cytokine.

172. The method of embodiment 170, wherein the subject is suffering from an immune-mediated disorder and the nucleic acid probe is complementary to a nucleic acid encoding an anti-inflammatory cytokine or a receptor for an anti-inflammatory cytokine.

173. The method of embodiment 170, wherein the cells in the first cell sample and the second cell sample are selected from hematopoietic cells, blood cells, epithelial cells, fibroblast cells, liver cells, lymph node cells or a combination thereof.

174. A method of identifying an effective therapy in a subject suffering from an immune-mediated disorder, a disease or an infection comprising the steps of (a) exposing (i) a first cell sample from the subject taken before treatment, and (ii) a second cell sample from the subject taken after treatment to a fluorescent cell-staining dye; (b) incubating (i) the cells of step (a)(i), and (ii) the cells of step (a)(ii) for a period of time to allow cell proliferation; (c) detecting or measuring by flow cytometry (i) an amount of cells of step (b)(i) having a low fluorescent cell-staining dye signal (Sig₀); and (ii) an amount of cells of step (b)(ii) having a low fluorescent cell-staining dye signal (Sig₁), wherein a Sig₁<Sig₀ identifies a therapy that is effective in the subject.

175. The method of embodiment 174, wherein the cells in the first cell sample and the second cell sample are selected from hematopoietic cells, blood cells, epithelial cells, fibroblast cells, liver cells, lymph node cells or a combination thereof.

176. A method of identifying an effective therapy in a subject suffering from an immune-mediated disorder comprising the steps of (a) obtaining (i) a first T-reg cell sample from the subject taken before treatment, and (ii) a second T-reg cell sample from the subject taken after treatment; (b) exposing T-resp cells from a healthy individual to a fluorescent cell-staining dye in the presence of a compound for a period of time to induce immune responsiveness; (c) incubating the cells of step (b) for a period of time to allow cell proliferation; (d) exposing (i) an amount of T-resp cells of step (c) to an amount of T-reg cells of step (a)(i); and (ii) an equal amount of T-resp cells of step (c) to an amount of T-reg cells of step (a)(ii), for a period of time to allow proliferation of T-cells, and wherein the amount of T-reg cells in step (c)(i) is the same as the amount of T-reg cells in step (c)(ii); (e) detecting using flow cytometry (i) an amount of signal generated by the T-resp cells of step (d)(i) (Sig₀); and (ii) an amount of signal generated by the T-resp cells of step (d)(ii)(Sig₁); (f) detecting or measuring by flow cytometry (i) an amount of signal generated in step (e)(i) (Sig₀), and (ii) an amount of signal generated in step (e)(ii) (Sig₁), wherein a Sig₁<Sig₀ identifies a therapy that is effective in the subject.

In any of the embodiments of the invention and variations thereof disclosed herein, background may be reduced and signal:background ratio increased by one or more of the following, in any and all combinations of inclusion and exclusion:

1. Where the initial fixation or preservation of a cell sample occurs in a solution without a cross-linking agent or in an alcohol-based solution with or without a cross-linking agent (such as an aldehyde), for example, a solution of methanol (30-60%) and water (40-70%), switching the cells to an aqueous treatment solution including a cross-linking agent, such as an aldehyde, such as formaldehyde or glutaraldehyde (for example, 1-5% of the aldehyde in a buffered saline solution) for, for example, at least several minutes before the fixed cells are hybridized with probe and subjected to flow cytometry. Following treatment with the aqueous treatment solution that includes a cross-linking agent, the solution that the cells are suspended in may be changed to one without a cross-linking agent prior to the hybridization with probe and flow cytometry.
2. Including in the probe hybridization buffer and/or flow cytometry buffer one or more DNA intercalators such as but not limited to ethidium homodimer, Nuclear ID™ (Enzo Biochem), a blue intercalator, Hoechst (blue intercalator) and DAPI (blue intercalator). Intercalators and fluorophore-labeled probes may be mutually selected such that the intercalators used do not absorb at or around the wavelengths at which the probe fluorophore(s) emit. For example, with the use of probes having a 6-carboxyfluorescein (FAM) donor fluorophore, blue intercalators such as Hoechst and DAPI are non-interfering.
3. Including in the probe hybridization buffer and/or flow cytometry buffer a chelating agent such as EDTA or EGTA.
4. Increasing the salt concentration and, in general, the ionic strength of the hybridization buffer and/or the flow cytometry buffer. For example, instead of 1×SSC, at least 2×SSC, at least 2.5×, at least SSC, at least 3.0×SSC, at least 3.5×SSC, at least 4.0×SSC, at least 4.5×SSC, at least 5.0×SSC, 2×-6×SSC, 2×-5×SSC or 2.5×-5×SSC hybridization buffer and/or flow cytometry buffer may be used. Similarly, instead of using a 1×DPBS hybridization buffer, the same aforementioned increased minimum concentrations and concentrations but for DPBS may be used. Irrespective of the particular salt buffer used for hybridization, the buffer may have an ionic strength that is the same as, that is about, or that is at least the ionic strength of 2.0×SSC, 2.5×SSC, 3.0×SSC, 3.5×SSC, 4.0×SSC, 4.5×SSC, or 5.0×SSC or within the range of ionic strength of 2×-6×SSC such as 2×-5×SSC or 2.5×-5×SSC.
5. Including in the probe hybridization buffer and/or flow cytometry buffer, one or more polyanionic polymers such as heparin, for example, unfractionated heparin, dextran sulfate and/or any of the other polyanionic polymers or compounds described herein. In one variation, the polyanionic polymer is not a nucleic acid polymer.
In addition to providing methods that incorporate any one or combination of the aforementioned improvements, the invention also provides the isolated buffers, compositions of matter including (or consisting essentially of, or consisting of) the nucleic acid probe(s) and/or the eukaryotic cells in the buffers, and flow cytometry apparatuses with or without cell sorting capability that are loaded with said compositions of matter or loaded with eukaryotic cells prepared and/or hybridized with probe in any of the aforementioned manners and under any of the aforementioned conditions.

In order to obtain the most accurate snap-shot of a biological system at a time point, the cells to be examined according to the methods of the invention should be fixed as quickly as possible following the time point. The manner of collecting and isolating a sample of a particular class or type of cells may also impact the quality of information that can be obtained regarding the state of the cells of interest at the time of collection, for example, due to prolonged contact with other cell types during the isolation process. PBMCs such as T-cells are often isolated from whole blood by Ficoll gradient centrifugation in a collection vial in a manner that colocalizes platelets in the resulting PBMC layer. For conventional laboratory purposes, the colocalization of PBMCs and platelets may be prolonged. However, platelet factors can impact the state of T-cells. Accordingly, exposure of PBMCs to platelets as well as exposure of PBMCs to cell lysis products should be minimized in order to maximize the quality of the state information obtained from the PBMCs according to the invention. In isolation methods that colocalize PBMCs and platelets, a process of further isolation of the PBMCs from the platelets may, for example, begin at least substantially immediately upon completion of the initial isolation process resulting in the colocalization or, for example, within 10 minutes, within 15 minutes, within 30 minutes, within 1 hour, or within 2 hours of said completion. Alternatively, or in addition, exposure of PBMCs to platelets may be minimized by isolating the PBMC and platelet-containing layer and diluting it immediately after, or as soon as possible after, the colocalization.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s). Moreover, features and limitations described in connection with one embodiment of the invention may be used in conjunction with other embodiments, even if not explicitly exemplified in combination within.

What is claimed is:

1. A method of detecting or quantifying an amount of a target RNA in a eukaryotic cell comprising the steps of:
a. fixating and permeabilizing a eukaryotic cell;
b. contacting the cell from step (a) with
i. a nucleic acid probe that is complementary to a nucleic acid sequence of the target RNA, wherein the nucleic acid probe specifically hybridizes to the target RNA if present, and
ii. a control nucleic acid probe that is complementary to a nucleic acid sequence of a control nucleic acid that is naturally or non-naturally occurring in the eukaryotic cell, wherein the control nucleic acid probe specifically hybridizes to the control nucleic acid in the eukaryotic cell;
c. detecting using flow cytometry′
i. an amount of signal generated by the probe hybridized to the complementary nucleic acid sequence of the target RNA in the eukaryotic cell, and
ii. an amount of signal generated by the control probe hybridized to the complementary nucleic acid sequence of the control nucleic acid in the eukaryotic cell; and
d. detecting or measuring the target RNA in the eukaryotic cell by detecting or measuring the amount of signal generated from the probes of step (b),
wherein the probes of step (b) are part of a hybrid molecular probes (HEMfPs) that generate a signal when bound to a complementary sequence in the target nucleic acid, and
wherein the presence or quantity of the target RNA is associated with the presence of or susceptibility to a disease.

2. A method of detecting or quantifying an amount of a target RNA in a eukaryotic cell comprising the steps of:
a. fixating and permeabilizing a eukaryotic test cell;
b. fixating and permeabilizing a eukaryotic control cell;
c. contacting
i. the test cell from step (a) with a nucleic acid probe that is complementary to a nucleic acid of interest; and
ii. the control cell from step (b) with a nucleic acid probe, wherein the nucleic acid probe hybridizes to complementary nucleic acid sequences in the eukaryotic control cell;
d. detecting using flow cytometry
i. an amount of signal generated by the probe hybridized to a complementary target nucleic acid sequence in the eukaryotic cell of step (c)(i), and
ii. an amount of signal generated by the probe hybridized to a complementary target nucleic acid sequence in the eukaryotic control cell of step (c)(ii); and
e. detecting or measuring the nucleic acid of interest in the eukaryotic cell by detecting or measuring the amount of signal generated from the probe of step (c)(i),
f. detecting or measuring the nucleic acid in the eukaryotic control cell by detecting or measuring the amount of signal generated from the probe of step (c)(ii); and
g. comparing the results of step (e) and step (f),
wherein the probes of steps (c)(i) and (c)(ii) are part of a hybrid molecular probes (HMPs) that generates a signal when bound to a complementary sequence in the target nucleic acid, and
wherein the presence or quantity of the target RNA is associated with the presence of or susceptibility to a disease.

3. The method of claim 2, wherein the target RNA is an mRNA.

4. The method of claim 2, further comprising adding a nucleic acid probe to the mixture of step (b), step (c) or both step (b) and step (c), wherein the nucleic acid probe comprises at least one probe that is part of a homogeneous probe system that generates a signal when bound to a complementary sequence in a control nucleic acid.

5. The method of claim 1 wherein the control nucleic acid comprises a sequence from a housekeeping gene.

6. The method of claim 2 wherein the control cell is selected from a cell from a healthy individual when the cell of step (a) is a diseased cell, a cell with inactive disease when the cell of step (a) is a cell with active disease, a cell that does not respond to an antigen when the cell of step (a) is a cell that responds to the antigen, a cell that has not been subjected to treatment when the cell of step (a) has been treated, or combinations thereof.

7. The method of claim 3 wherein the target nucleic acid is an mRNA that is expressed from a virus in the eukaryotic cell.

8. The method of claim 7 wherein the virus is selected from HIV, HBV, HCV, HPV and a Herpesvirus.

9. The method of claim 8, wherein the virus is HPV and the target nucleic acid is an mRNA comprising a sequence from the HPV E6 gene, the HPV E7 gene, the HPV E2 gene or any combination thereof.

10. The method of claim 5 wherein the control nucleic acid is a non-viral mRNA expressed by the eukaryotic cell.

11. The method of claim 1 wherein at least one probe provided in step (b)(i) is complementary to a viral mRNA, wherein at least one probe of step (b)(i) is complementary to a sequence in a eukaryotic mRNA from a gene that changes expression during viral infection, and wherein the probes are part of a homogeneous probe system that generates a signal when bound to a complementary sequence in the eukaryotic mRNA.

12. The method of claim 11, wherein the gene that changes expression during viral infection is selected from p53. Rb, P16^{INK4a}, Ki67, TOP2a, MCM2, CK13, CK14, MCM5, CDC6, survivin, CEA, p63, pRb, p21WAF1, MYC cellular oncogene, CDK4, cyclin A, Cyclin B, cyclin D, cyclin E, telomerase, minichromosome maintenance protein 2, minichromosome maintenance protein 4, minichromosome maintenance protein 5), heat shock protein 40, heat shock protein 60, heat shock protein 70, CA9/MN protein, and a combination thereof.

13. The method of claim 1, further comprising in step (b)(i) adding at least one antibody.

14. The method of claim 13, wherein the antibody recognizes a surface antigen of the eukaryotic cell.

15. The method of claim 13, wherein at least one probe provided in step (b)(i) is complementary to a viral mRNA in the eukaryotic cell and is part of a homogeneous probe system that generates a signal when bound to a complementary sequence in the viral mRNA, and wherein the antibody recognizes an antigen from a protein expressed from a eukaryotic gene that changes expression during infection by the virus.

16. The method of claim 15 wherein the gene that changes expression during infection by the virus is selected from p53. Rb, P16^{INK4}a, Ki67, TOP2a, MCM2, CK13, CK14, MCM5, CDC6, surviving, CEA, p63, pRb, p21WAF1, MYC cellular oncogene, CDK4, cyclin A, Cyclin B, cyclin D, cyclin E, telomerase, minichromosome maintenance protein 2, minichromosome maintenance protein 4, minichromosome maintenance protein 5), heat shock protein 40, heat shock protein 60, heat shock protein 70, CA9/MN protein, and a combination thereof.

17. The method of claim 13, wherein the antibody is unlabeled.

18. The method of claim 13, wherein the antibody is labeled.

19. The method of claim 18, wherein the antibody is labeled with a ligand, a fluorescent compound, a quantum dot, an electron dense component, a magnetic component, a hormone component, a chelating group, a chelated compound, an antigen or a combination thereof.

20. The method of claim 2, wherein a change in the target nucleic acid level is associated with a cancerous state.


SEQUENCE LISTING (69 sequences)

SEQ ID NO: 1 - DNA, 37 bp, Artificial Sequence
  Description of Artificial Sequence Synthetic probe
  5′ 6-FAM
  3′ BHQ1a-Q
ccgaccagtactgttgcttgcagtacacacaggtcgg

SEQ ID NO: 2 - DNA, 35 bp, Artificial Sequence
  Description of Artificial Sequence Synthetic probe
  5′ 6-FAM
  3′ BHQ1a-Q
cctggctaaacatttatcacatacagcatgccagg

SEQ ID NO: 3 - DNA, 33 bp, Artificial Sequence
  Description of Artificial Sequence Synthetic probe
  5′ 6-FAM
  3′ BHQ1a-Q
ccacgattacagctgggtttctctacgtcgtgg

SEQ ID NO: 4 - DNA, 36 bp, Artificial Sequence
  Description of Artificial Sequence Synthetic probe
  5′ 6-FAM
  3′ BHQ1a-Q
caccacgtcctctgagtcgcttaattgctcgtggtg

SEQ ID NO: 5 - DNA, 32 bp, Artificial Sequence
  Description of Artificial Sequence Synthetic probe
  5′ 6-FAM
  3′ BHQ1a-Q
ccagccattgtgtgacgttgtggttcggctgg

SEQ ID NO: 6 - DNA, 36 bp, Artificial Sequence
  Description of Artificial Sequence Synthetic probe
  5′ 6-FAM
  3′ BHQ1a-Q
cctcccacaccacggacacacaaggacagtgggagg

SEQ ID NO: 7 - DNA, 36 bp, Artificial Sequence
  Description of Artificial Sequence Synthetic probe
  5′ Quasar670
  3′ BHQ3
cctcccacttgcacatgccggagccgttgtgggagg

SEQ ID NO: 8 - DNA, 36 bp, Artificial Sequence
  Description of Artificial Sequence Synthetic probe
  5′ Quasar670
  3′ BHQ3
cctcccactgacccatgcccaccatcacgtgggagg

SEQ ID NO: 9 - DNA, 34 bp, Artificial Sequence
  Description of Artificial Sequence Synthetic probe
  5′ Quasar670
  3′ BHQ3
ccaccgaaggtagtttcgtggatgccgtgggagg

SEQ ID NO: 10 - DNA, 32 bp, Artificial Sequence
  Description of Artificial Sequence Synthetic probe
  5′ Quasar670
  3′ BHQ3
ccaccgtcttgatcttcattgtgctgcggtgg

SEQ ID NO: 11 - DNA, 31 bp, Artificial Sequence
  Description of Artificial Sequence Synthetic probe
  5′ Quasar670
  3′ BHQ3
ccaggagattttggagggatctcgctcctgg

SEQ ID NO: 12 - DNA, 32 bp, Artificial Sequence
  Description of Artificial Sequence Synthetic probe
  5′ Quasar670
  3′ BHQ3
ccgtgggactgtggtcatgagtccttccacgg

SEQ ID NO: 13 - DNA, 31 bp, Artificial Sequence
  Description of Artificial Sequence Synthetic probe
  5′ Quasar670
  3′ BHQ3
cgctccctcttaaaatcccgcggacggagcg

SEQ ID NO: 14 - DNA, 30 bp, Artificial Sequence
  Description of Artificial Sequence Synthetic probe
  5′ Quasar670
  3′ BHQ3
cgacgctgtaggagagggagttccgcgtcg

SEQ ID NO: 15 - DNA, 30 bp, Artificial Sequence
  Description of Artificial Sequence Synthetic probe
  5′ Quasar670
  3′ BHQ3
ccacgcggaactccctctcctacagcgtgg

SEQ ID NO: 16 - DNA, 32 bp, Artificial Sequence
  Description of Artificial Sequence Synthetic probe
  5′ Cy3
  3′ BHQ2a-Q
cccagaggtaccgtgcgacatcgcgatctggg

SEQ ID NO: 17 - DNA, 32 bp, Artificial Sequence
  Description of Artificial Sequence Synthetic probe
  5′ Cy3
  3′ BHQ2a-Q
ccgccatggttactgcctctggtgcccggcgg

SEQ ID NO: 18 - DNA, 34 bp, Artificial Sequence
  Description of Artificial Sequence Synthetic probe
  5′ Cy3
  3′ BHQ2a-Q
ccgcctgtcatgaagtcgacagcttccggggcgg

SEQ ID NO: 19 - DNA, 34 bp, Artificial Sequence
  Description of Artificial Sequence Synthetic probe
  5′ Cy3
  3′ BHQ2a-Q
ccgccagcagtgtgactcaagagaagccaggcgg

SEQ ID NO: 20 - DNA, 29 bp, Artificial Sequence
  Description of Artificial Sequence Synthetic probe
  5′ Cy3
  3′ BHQ2a-Q
cctccgtgtccaggaagccctcccggagg

SEQ ID NO: 21 - DNA, 24 bp, Artificial Sequence
  Description of Artificial Sequence Synthetic probe
  5′ Cy3
  3′ BHQ2a-Q
ccgtgtcctcagccaggtccacgg

SEQ ID NO: 22 - DNA, 25 bp, Artificial Sequence
  Description of Artificial Sequence Synthetic probe
  5′ Cy3
  3′ BHQ2a-Q
ccgggagggcttcgtggacacccgg

SEQ ID NO: 23 - DNA, 29 bp, Artificial Sequence
  Description of Artificial Sequence Synthetic probe
  5′ Cy3
  3′ BHQ2a-Q
ccgacccgtggacgtggctgaggagtcgg

SEQ ID NO: 24 - DNA, 35 bp, Artificial Sequence
  Description of Artificial Sequence Synthetic probe
  5′ 6-FAM
  3′ BHQ1a-Q
ccgacgtttctaatgttgctccatacacacgtcgg

SEQ ID NO: 25 - DNA, 37 bp, Artificial Sequence
  Description of Artificial Sequence Synthetic probe
  5′ 6-FAM
  3′ BHQ1a-Q
cctggcaacgtcctgtccaccttccacctatgccagg

SEQ ID NO: 26 - DNA, 35 bp, Artificial Sequence
  Description of Artificial Sequence Synthetic probe
  5′ 6-FAM
  3′ BHQ1a-Q
ccaactaattgctcatagcagtataggtcagttgg

SEQ ID NO: 27 - DNA, 40 bp, Artificial Sequence
  Description of Artificial Sequence Synthetic probe
  5′ 6-FAM
  3′ BHQ1a-Q
cctgcacagcgccctgtccaacgacccgaaatagtgcagg

SEQ ID NO: 28 - DNA, 47 bp, Artificial Sequence
  Description of Artificial Sequence Synthetic probe
  5′ 6-FAM
  3′ BHQ2
ccagccaatgacctgcattaaaatatttcttaaggttttctggctgg

SEQ ID NO: 29 - DNA, 45 bp, Artificial Sequence
  Description of Artificial Sequence Synthetic probe
  5′ 6-FAM
  3′ BHQ2
ccagccattatttttctgtcactctcctctttccaattcggctgg

SEQ ID NO: 30 - DNA, 45 bp, Artificial Sequence
  Description of Artificial Sequence Synthetic probe
  5′ 6-FAM
  3′ BHQ2
ccagccctgcattaaaatatttcttaaggttttctgcttggctgg

SEQ ID NO: 31 - DNA, 39 bp, Artificial Sequence
  Description of Artificial Sequence Synthetic probe
  5′ 6-FAM
  3′ BHQ2
ccagccactctcctctttccaattcttcaaaagggctgg

SEQ ID NO: 32 - DNA, 39 bp, Artificial Sequence
  Description of Artificial Sequence Synthetic probe
  5′ 6-FAM
  3′ BHQ2
ccagcggacattcaagtcagttaccgaataatgggctgg

SEQ ID NO: 33 - DNA, 62 bp, Artificial Sequence
  Description of Artificial Sequence Synthetic probe
  5′ 6-FAM
  3′ BHQ1
ccagccttttttttttttttttttttttttttttttttttttttttttttttttttggctgg

SEQ ID NO: 34 - DNA, 773 bp, Human papillomavirus
aagcttatgtaatacgactcactatagggcgaattgggtaccatggaaagtgcaaatgcctccacgtctgcaacgaccatagaccagttgtgcaagacgtttaatctatctatgcatacgttgcaaattaattgtgtgttttgcaagaatgcactgaccactgcagagatttattcatatgcatataaacagctaaaggtcctgtttcgaggcggctatccatatgcagcctgcgcgtgctgcctagaatttcatggaaaaattaaccaatatagacactttgattatgctggatatgcaacaactgttgaagaagaaactaaacaagacattttagacgtgctaattcggtgctacctgtgtcacaaaccgctgtgtgaagtagaaaaggtaaaacatatactaaccaaggcacggttcataaagctaaattgtacgtggaagggtcgctgcctacactgctggacaacatgcatggaagacatgttaccctaaaggatattgtattagacctgcaacctccagaccctgtagggttacattgctatgagcaattagtagacagctcagaagatgaggtggacgaagtggacggacaagattcacaacctttaaaacaacattaccaaatagtgacctgttgctgtggatgtgacagcaacgttcgactggttgtgcagtgtacagaaacagacatcagagaagtgcaacagcttctgttgggaacactaaacatagtgtgtcccatctgcgcaccgaagacataagaattc

SEQ ID NO: 35 - DNA, 773 bp, Human papillomavirus
aagcttatgtaatacgactcactatagggcgaattgggtaccatggaaagtaaagatgcctccacgtctgcaacatccatagaccagttgtgcaagacgtttaatctttctttgcacactctgcaaattcagtgcgtgttttgcaggaatgcactgaccaccgcagagatatatgcatatgcctataagaacctaaaggttgtgtggcgagacaactttccctttgcagcgtgtgcctgttgcttagaactgcaagggaaaattaaccaatatagacactttaattatgctgcatatgcacctacagtagaagaagaaactaatgaagatattttaaaagtgttaattcgttgttacctgtgtcacaagccgttgtgtgaaatagaaaaactaaagcacatattgggaaaggcacgcttcataaaactaaataaccagtggaagggtcgttgcttacactgctggacaacatgcatggaagacttgttaccctaaaggatatagtactagacctgcagcctcctgaccctgtagggttacattgctatgagcaattagaagacagctcagaagatgaggtggacaaggtggacaaacaagactcacaacctttaacacaacattaccaaatactgacctgttgctgtggatgtgacagcaacgtccgactggttgtggagtgcacagacggagacatcagacaactacaagaccttttgctgggcacactaaatattgtgtgtcccatctgcgcaccaaaaccataagaattc

SEQ ID NO: 36 - DNA, 824 bp, Human papillomavirus
aagcttatgtaatacgactcactatagggcgaattgggtaccatgcaccaaaagagaactgcaatgtttcaggacccacaggagcgacccagaaagttaccacagttatgcacagagctgcaaacaactatacatgagataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttcccagaaaccataagaattc

SEQ ID NO: 37 - DNA, 851 bp, Human papillomavirus
aagcttatgtaatacgactcactatagggcgaattgggtaccatggcgcgctttgaggatccaacacggcgaccctacaagctacctgatctgtgcacggaactgaacacttcactgcaagacatagaaataacctgtgtatattgcaagacagtattggaacttacagaggtatttgaatttgcattcaaagatttatttgtagtgtatagagacagtataccgcatgctgcatgccataaatgtatagatttttattctagaattagagaattaagacattattcagactctgtgtatggagacacattagaaaaactaactaacactgggttatacaatttattaataaggtgcctgcggtgccagaaaccgttgaatccagcagaaaaacttagacaccttaatgaaaaacgacgattccacaaaatagctgggcactatagaggccagtgccattcgtgctgcaaccgagcacgacaggagagactccaacgacgcagagaaacacaagtataatattaagtatgtatggacctaaggcaacattgcaagacattgtattgcatttagagcctcaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaaaaccctgtcctttgtgtgtccgtggtgtgcatcccagcagtaagaattc

SEQ ID NO: 38 - DNA, 822 bp, Human papillomavirus
ctgcagatgtaatacgactcactatagggcgaattgggtaccatgttcgaggatcctagagaacgacccagaacgctacatgagctatgtgaaagcttgaatactactttgcaaaatttgcaggtacagtgtgtatattgcaaggaaaccttacaatgggctgatgtatataattttgcaatttgtgacctaagagtagtatatagagataggagtccgtatgctgcatgcaaaagatgtgtaatattttattcaaaaataacagagtatagacgctatacatgttctgtgtatggtgcaacattagaagccttaactaaaaaaagtttatgtaatttgttaataaggtgtcatagatgtcaaatgccattggggccagaagaaaaacaaagaattgtggatgaaaagcgacgatttcacgaaatagcagggcagtggaaagggttgtgtacaaattgttggagaccaaggcgccaaacagaaacacaagtgtaaagaacaatgcatggaaacataattaatattgaagatgtaatactagatctggtgccgcaacccgaaattgacctacgctgctacgaacaattggactatgaacaatttgacagctcagatgaggatgaaacagataatatgcgtgaccagcaggccagacaagctggacaagaagtgtgttacagaattgaagcacaatgttgtatgtgtaatagtatagtgcagctagctgtgcagagcagtcgacagaacgttcgagtgctggagcagatgttaatggaagacgtgtccttggtgtgccatcagtgtgctgcacagtaagaattc

SEQ ID NO: 39 - DNA, 797 bp, Human papillomavirus
aagcttatgtaatacgactcactatagggcgaattgggtaccatgttcaaaaatcctgcagaaagacctcggaaattgcatgaactaagctcggcattggaaataccctacgatgaactaagattgaattgtgtctactgcaaaggtcagttaacagaaacagaggtattagattttgcatttacagatttaacaatagtatatagggacgacacaccatacggagtgtgtacaaaatgtttaagattttattctaaagtaagtgaatttagatggtatagatatagtgtgtatggaacaacattagaaaaattgacaaacaaaggtatatgtgatttattaattaggtgtataacgtgtcagagaccgttgtgtccagaagaaaaacaaagacatttggataaaaagaaacgattccacaacataggaggaaggtggacaggacgttgcatagtatgttggagaaaacctcgtactgaaacccaagtgtaaacatgcgtggagaaacacctacattgcaagactatgtgttagatttgcaacctgaggcaactgacctccactgttatgagcaattacccgacagcttagatgaggaggatgttatagacagtccagctggacaagcaaaaccggacacatccaattacaatatcgttaccttttgttgtcagtgtgagtctacacttcgtttgtgtgtacagagcacacaagtagatattcgcatattgcaagagctgttaatgggctcatttggaatcgtgtgccccaactgttctactagactgtaagaattc

SEQ ID NO: 40 - DNA, 806 bp, Human papillomavirus
aagcttatgtaatacgactcactatagggcgaattgggtaccatgtttcaagacactgaggaaaaaccacgaacattgcatgatttgtgccaagcattggagacaactatacacaacattgaactacagtgcgtggaatgcaaaaaccctttgcaacgatctgaggtatatgattttgcatttgcagatttaacagttgtatatagagagggaaatccatttggaatatgtaaactgtgtttgcggttcttatctaaaattagtgaatatagacattataattattctgtatatggaaatacattagaacaaacagttaaaaaacctttaaatgaaatattaattaggtgtattatatgtcaaagacctttgtgtcctcaagaaaaaaaacgacatgtggatttaaacaaacgatttcataatatttcgggtcgttgggcagggcgctgtgcggcgtgttggaggtcccgacgtagagaaactgcactgtgacgtgtaaaaacgccatgagaggacacaagccaacgttaaaggaatatgttttagatttatatcctgaaccaactgacctatactgctatgagcaattaagtgacagctcagatgaggatgaaggcttggaccggccagatggacaagcacaaccagccacagctgattactacattgtgacctgttgtcacacttgtaacaccacagttcgtttatgtgtcaacagtacagcaagtgacctacgaaccatacagcaactacttatgggcacagtgaatattgtgtgccctacctgtgcacaactataagaattc

SEQ ID NO: 41 - DNA, 800 bp, Human papillomavirus
aagcttatgtaatacgactcactatagggcgaattgggtaccatgtttcaggacccagctgaaagaccttacaaactgcatgatttgtgcaacgaggtagaagaaagcatccatgaaatttgtttgaattgtgtatactgcaaacaagaattacagcggagtgaggtatatgactttgcatgctatgatttgtgtatagtatatagagaaggccagccatatggagtttgcatgaaatgtttaaaattttattcaaaaataagtgaatatagatggtatagatatagtgtgtatggagaaacgttagaaaaacaatgcaacaaacagttatgtcatttattaattaggtgtattacatgtcaaaaaccgctgtgtccagttgaaaagcaaagacatttagaagaaaaaaaacgattccataacatcggtggacggtggacaggtcggtgtatgtcctgttggaaaccaacacgtatagaaaccgaggtgtaatcatgcatggagaaataactacattgcaagactatgttttagatttggaacccgaggcaactgacctacactgttatgagcaattgtgtgacagctcagaggaggaggaagataccattgacggtccagctggacaagcaaaaccagacacctccaattataatattgtaacgtcctgttgtaaatgtgaggcgacactacgtctgtgtgtacagagcacacacattgacatacgtaaattggaagatttattaatgggcacatttggaatagtgtgccccggctgttcacagagagcataagaattc

SEQ ID NO: 42 - DNA, 863 bp, Human papillomavirus
aagcttatgtaatacgactcactatagggcgaattgggtaccatggcgcgatttcacaatcctgcagaacggccatacaaattgccagacctgtgcacaacgctggacaccaccttgcaggacattacaatagcctgtgtctattgcagacgaccactacagcaaaccgaggtatatgaatttgcatttagtgatttatatgtagtatatagggacggggaaccactagctgcatgccaatcatgtataaaattttatgctaaaatacgggagctacgatattactcggactcggtgtatgcaactacattagaaaatataactaatacaaagttatataatttattaataaggtgcatgtgttgtctgaaaccgctgtgtccagcagaaaaattaagacacctaaatagcaaacgaagatttcataaaatagcaggaagctatacaggacagtgtcgacggtgctggaccacaaaacgggaggaccgcagactaacacgaagagaaacccaagtataacatcagatatgcgtggaccaaagcccaccttgcaggaaattgtattagatttatgtccttacaatgaactacagccggttgaccttgtatgtcacgagcaattaggagagtcagaggatgaaatagatgaacccgaccatgcagttaatcaccaacatcaactactagccagacgggatgaaccacagcgtcacacaatacagtgttcgtgttgtaagtgtaacaacacactgcagctggtagtagaagcctcacgggatactctgcgacaactacagcagctgtttatggactcactaggatttgtgtgtccgtggtgtgcaactgcaaaccagtaagaattc

SEQ ID NO: 43 - DNA, 758 bp, Human papillomavirus
aagcttatgtaatacgactcactatagggcgaattgggtaccatgtcaggtacatctgcctcatcacagccacgcacattataccaattgtgtaaggaatttgggctgacattgcggaatttacagatttcctgcatttggtgcaaaaagcacttaacaggcgcagaggtgctcgcgtaccattttaaagatttggtagtggtgtggaggaaggactttccatatgctgcatgtgcattttgtttagaatttaattctaaaatttgtgcactgcgacactacgaaagatcagcattttggtatacagtggagaaagaaactggactacttttagaagaacaacaaattagatgtgccttgtgtcaaaagccgttatcacagagcgaaaaaaaccatcatattgatacaggtacaagatttcaatttatattgtgtcagtggacgggtcggtgtacgcattgcagaggacaatgcgtggagagacgcctaccctaaaggacattgttttgtttgacataccaacgtgtgagacacccattgacctgtattgctatgaacaattggacagctcagatgaagatgaccaagccaaacaggacatacagcgttacagaatactgtgtgtgtgtacacagtgttacaagtctgttaaactcgttgtgcagtgtacagaggcggacataagaaacctgcaacagatgcttttgggcacactggatattgtgtgtcctttgtgtgcccgcgtggagtaagaattc

SEQ ID NO: 44 - DNA, 776 bp, Human papillomavirus
ctgcagatgtaatacgactcactatagggcgaattgggtaccatgactgcacgtagctgctcccaaaacgcacggactatatttgagttgtgtgatgagtgtaacataactttgcctactctgcaaattgggtgcatattttgcaagaagtggttacttaccacggaagtattatcgtttgcatttagagatttaaaggttgtgtggcgcgacggatatccgtttgctgcatgcttggcctgtctacagtttcatggaaaaataagtcaatataggcactttgactacgcagcatatgcagatactgtagaagaagaaactaagcaaacagtgtttgatttgtgcattagatgctgtaagtgccacaagccattatcaccagtggaaaaagtacagcatattgtgcaaaaggcacaattctttaaaatacatagcgtgtggaaaggatactgcctacattgctggaaatcatgcatggaaaaaagccgacgatcagagactatgtgctaactatgcaaccagaacctagaagcttaacctgtaacgagcaattagacagctcagactcagaggatgagcgtgagcaacccacgcaacaggaccagcaagtgaatctacaagtttacagagttgtaacagagtgcacatcctgtctgtgtgtaattcgacttgttgtgcagtgctctgacagtgacatcaagaagctagaagacctgctgttgggcacattaaaaattgtgtgcccactgtgtaccaccacagcggtgtgagaattc

SEQ ID NO: 45 - DNA, 770 bp, Human papillomavirus
aagcttatgtaatacgactcactatagggcgaattgggtaccatggaaagtgcaaatgcctccacgtctgcacaaagtatagaccagttgtgcaaggagtgcaacattcctatgcacaatctgcaaattttatgcgtgttttgcagaaaaacgttaagtactgcagaggtttattcattcgcatataaacagttatatgtagtgtaccgaggaaactttccatttgcagcctgtgccatttgtttagaactacaaggtaaggtcaatcaatttaggcattttaactacgcgggatatgcagtaacagtggaagaagaaacaaataagtcaattctggacgtgctgatacgctgctatttgtgccacaaaccattgtgccacgtggaaaaggtgcgccacatattggacaaggcgcgattcattaaattacaagatacctggaagggtcgctgcttccattgttggacatcatgcatggaaactatactaccttaaaggaaattgttttacagctggaacctcctgaccctgtaggcctacattgcaatgagcaattagacagctcagaagatgaggtggatgaactagccacgcaagccacgcaagacgttacacagccttaccaaatagtaaccacctgtggtacatgtagtcggaaggttcggctggttgtgcagtgcacaggaacagacatccatcacctacatacgcttctgctgggttcactggatatattgtgtcctgtgtgtgcgcccaaaacctaagaattc

SEQ ID NO: 46 - DNA, 854 bp, Human papillomavirus
aagcttatgtaatacgactcactatagggcgaattgggtaccatggcgcgctttgacgatccaacgcaacgaccctacaagctaccagatctgtgcacagaattgaatacatcactacaagacgtatctattgcctgtgtatattgcaaagcaacattggaacgcacagaggtatatcaatttgcttttaaagatttatttatagtgtatagagactgtatagcatatgctgcatgccataaatgtatagacttttattccagaattagagaattaagatattattcaaactctgtatatggagagacactggaaaaaataactaatacagagttgtataatttgttaataaggtgcctgcggtgccagaaaccattgaacccagcagaaaaacgtagacaccttaaggacaaacgaagattccacagcatagctggacagtaccgagggcagtgtaatacatgttgtgaccaggcacggcaagaaagacttcgcagacgtagggaaacacaagtatagcaataagtatgcatggaccccgagcaacactgcaagaaattgtattgcatttggaacctcagaatgaattagatcctgttgacctgttgtgttacgagcaattaagcgagtcagaggaggaaaacgatgaagcagatggcgttagtcatgcacaactaccagcccgacgagccgaaccacagcgtcacaaaattttgtgtgtatgttgtaagtgtgacggcagaattgagcttacagtagagagctcggcagatgaccttagaacactacagcagctgtttttgagcaccttgtcctttgtgtgtccgtggtgtgcaactaaccaataagaattc

SEQ ID NO: 47 - DNA, 814 bp, Human papillomavirus
ctgcagatgtaatacgactcactatagggcgaattgggtaccatgttcgaagacaagagggaaagaccacgaacgctgcatgaattatgtgaagctttgaacgtttctatgcacaatatacaggtagtgtgtgtgtattgtaaaaaggaattatgtagagcagatgtatataatgtagcatttactgaaattaagattgtatatagggataataatccatatgcagtatgcaaacaatgtttactgttttattcaaaaattagagagtatagacgttatagcaggtctgtgtatggtactacattagaggcaattactaaaaaaagcttatatgatttatcgataaggtgtcatagatgtcaaagaccacttgggcctgaagaaaagcaaaaattggtggacgaaaaaaaaaggttccatgaaatagcgggacgttggacggggcaatgcgctaattgctggcaacgtacacgacaacgtaacgaaacccaagtgtaataaagccatgcgtggtaatgtaccacaattaaaagatgtagtattgcatttaacaccacagactgaaattgacttgcaatgctacgagcaatttgacagctcagaggaggaggatgaagtagataatatgcgtgaccagctaccagaaagacgggctggacaggctacgtgttacagaattgaagctccgtgttgcaggtgttcaagtgtagtacaactggcagtggaaagcagtggagacacccttcgcgttgtacagcagatgttaatgggcgaactaagcctggtttgcccgtgttgtgcgaacaacgaattc

SEQ ID NO: 48 - DNA, 799 bp, Human papillomavirus
aagcttatgtaatacgactcactatagggcgaattgggtaccatgtttgaggatccagcaacacgaccccggaccctgcacgaattgtgtgaggtgctggaagaatcggtgcatgaaataaggctgcagtgtgtccagtgcaaaaaagagctacaacgaagagaggtatacaagtttctatttacagatttacgaatagtatatagagacaataatccatatggcgtgtgtattatgtgcctacgctttttatctaagataagtgaatataggcattatcaatattcactttatgggaaaacattagaagagagggtacgaaaaccattaagtgaaataactattagatgtataatttgtcaaacgccattatgtcctgaagaaaaagaaagacatgttaatgcaaaaaagcgatttcataatattatgggtcgttggacagggcgctgttcagagtgttggagaccccgacctgtgacccaagtgtaacgtcatgcgtggagacaaagcaactataaaagattatatattagatctgcaacctgaaacaactgacctacactgctatgagcaattaggtgacagctcagatgaggaggatacagatggtgtggaccggccagatggacaagcagaacaagccacaagcaattactacattgtgacatattgtcacagttgtgatagcacattacggctatgcattcatagcactgcgacggaccttcgtactctacagcagatgctgttgggcacattacaagttgtgtgccccggctgtgcacggctataagaattc

SEQ ID NO: 49 - DNA, 833 bp, Human papillomavirus
aagcttatgtaatacgactcactatagggcgaattgggtaccatggatcgtcggttatttgaaaatacacaggagcgaccacgtacattgcatcagctctgtgaagttgtgaatcaaccattgctggagctgcaacttggctgtgtgttctgcaagaaggcattgacagcttcagaggtatataattttgcatataaagatttaagagtagtgtatagagacgggtatccgtatggagtgtgcaaattctgtttgctattttatagtaaggtccgaaaattaagacattacaattgttcagtgtacggggctagcctggaagcactaactaaaaaaaagttatctgatttattaataaggtgctacagatgtcaacatccgttgacaccagaggaaaaacagttacactgtgactataagaaacggtttcacaaaatttcacatatgtggaccgggtcgtgcctgacatgctggagacacacaacagcaactgaatcagcagtataatcatgcacggtaacgtaccaacacttccacaatatattatagaacttataccacaaactgagattgacctgcaatgccatgagcaattgaacagctcagaggatgaggatgaggatgaagtagaccatttgcaggagcagccacagcaagctagacgggacgaacaacatccttgttacctaattgaaacacagtgttgtaggtgtgagtcgttggtgcagttggctgttcagagtccaacaaaagagctgcgtatgttacaacaaatgcttatgggcacagtggagcttgtgtgccccctctgcgcaacaaggcgataagaattc

SEQ ID NO: 50 - DNA, 836 bp, Human papillomavirus
aagcttatgtaatacgactcactatagggcgaattgggtaccatggagccacaattcaacaatccacaggaacgtccacgaagcctgcaccacttgagtgaggtattagaaatacctttaattgatcttagattatcatgtgtatattgcaaaaaagaactaacacgtgctgaggtatataattttgcatgcactgaattaaaattagtgtatagggatgattttccttatgcagtgtgcagagtatgtttattgttttatagtaaagttagaaaatataggtattatgactattcagtgtatggagctacactagaaagtataactaaaaaacagttatgtgatttattaataaggtgctacagatgtcaaagtccgttaactccggaggaaaagcaattgcattgtgacagaaaaagacgatttcatctaatagcacatggttggaccgggtcatgtttggggtgctggagacaaacatctagagaacctagagaatctacagtataatcatgcatggtaaagtgccaacgctgcaagacgttgtattagaactaacacctcaaacagaaattgacctacagtgcaatgagcaattggacagctcagaggatgaggatgaggatgaagtagaccatttgcaggagcggccacagcaagctagacaagctaaacaacatacgtgttacctaatacacgtaccttgttgtgagtgtaagtttgtggtgcagttggacattcagagtaccaaagaggacctgcgtgttgtacaacagctgcttatgggtgcgttaacagtaacgtgcccactctgcgcatcaagtaactaagaattc

SEQ ID NO: 51 - DNA, 809 bp, Human papillomavirus
aagcttatgtaatacgactcactatagggcgaattgggtaccatgttccaggacgcagaggagaaaccacggacattgcatgatttgtgtcaggcgttggagacatctgtgcatgaaattgaattgaaatgcgttgaatgcaaaaagactttgcagcgatctgaggtatatgactttctatttgcagatttaagaatagtgtatagagatggaaatccatttgcagtatgtaaagtgtgtttacgattgctatctaaaataagtgagtatagacattataattattcgctatatggagaaacattagaacaaacactaaaaaagtgtttagatgaaatattaattagatgtattatttgtcaaagaccattgtgtccacaagaaaaaaaaaggcatgtggatttaaacaaaaggtttcataatatttcgggtcgttggacagggcgctgtgcagtgtgttggagaccccgacgtagacaaacacaagtgtaacctgtaacaacgccatgagaggaaacaacccaacgctaagagaatatattttagatttacatcctgaaccaactgacctattctgctatgagcaattatgtgacagctcagacgaggatgaaataggcttggacgggccagatggacaagcacaaccggccacagctaattactacattgtaacgtgttgttacacttgtggcaccacggttcgtttgtgtatcaacagtacagcaactgaagtacgaaccctacagcagctgcttatgggcacatgtactattgtgtgccccagctgtgcacagcaataagaattc

SEQ ID NO: 52 - DNA, 859 bp, Human papillomavirus
aagcttatgtaatacgactcactatagggcgaattgggtaccatggcacgctttgaggatcctacacaacgaccatacaaactgcctgacttgagcacaacattgaatattcctctgcatgatattcgcatcaattgtgtgttttgcaaaggggaactgcaagaaagagaggtatttgaatttgcttttaatgacttatttatagtgtatagagactgtacaccgtatgcagcgtgtctgaaatgcatttcattttatgcaagagtaagagaattaagatattatagagattctgtgtatggagaaacattagaggctgaaaccaagacaccgttacatgagctgctgatacgctgttatagatgcctaaaacctctatgtccaacagacaaattaaagcatataactgaaaaaagaagattccataatatagctggaatatatacaggacagtgtcgtgggtgtcggacccgagcaagacacctaagacagcaacgacaagcgcgtagtgaaacactggtgtaaaacaatgcatggaccaaaagcaacactttgtgacattgttttagatttggaaccacacaattttgaggaagttgaccttgtgtgctacgagcaattacctgactccgactccgagaatgaaaaagatgaaccagatggagttaatcatcctttgctactagctagacgagctgaaccacagcgtcacaacattgtgtgtgtgtgttgtaagtgtaataatcaacttcagctagtagtagaaacctcgcaagacggattgcgagccttacagcagctgtttatggacgcactatcctttgtgtgtcctttgtgtgcagcaaaccagtaagaattc

SEQ ID NO: 53 - DNA, 836 bp, Human papillomavirus
aagcttatgtaatacgactcactatagggcgaattgggtaccatggattccatattcagcaatacacaggaacgtccacgaagcctgcaccatctgagcgaggtattacaaatacctttacttgatcttagattatcatgtgtatactgcaaaaaggaacttacaagtttagagttatataggtttgcatgtattgagttaaaactagtatatagaaacaattggccatatgcagtatgtagggtatgtttattgttttatagtaaggttagaaaatataggtactataaatattcagtgtatggggcaacattagaaagtataactaaaaaacagttatctgatttatcaataaggtgctaccgatgtcaatgtccgttaacaccggaggaaaaacaattgcactgtgaacataaaagacgatttcattatatagcatatgcatggaccgggtcatgtttgcagtgttggagacatacaagtagacaagctacagaatctacagtataaccatgcatggtaaagtaccaacgttgcaagaggttatattagaacttgcaccgcaaacggaaattgacctacaatgcaatgagcaattggacagctcagaggatgaggatgaggatgaaatagaccatttgctggagcggccacagcaagctagacaagctgaacaacataagtgttacctaattcacgtaccttgttgtaagtgtgagttggtggtgcagttggacattcagagtaccaaagaggagctacgtgtggtacaacagctgcttatgggtgcgttaacagtaacgtgcccactctgcgcatcatctaaataagaattc

SEQ ID NO: 54 - DNA, 809 bp, Human papillomavirus
ctgcagatgtaatacgactcactatagggcgaattgggtaccatgtttcaggacacagacgaaaaaccacgcaacttgcacgaattgtgtgaagctttggaaaccacggtgcatgaaataagtttgccctgcgtgcagtgcaaaaaaactttggacagaaacgaggtatatgactttttatttactgatttgaaaatagtatacagatgtgggaatccgtatggggtatgtaagcaatgcctaagactgttatcaaaagttagtgaatatagatattttaactattcagtatatggaaatactttagaagaaattgtgcataaaccattaaatgaaattacaataagatgtataacatgtcaaagaccattgtgtccacaagaaaagcaaaggcatgttgatcgcaaaaagcgatttcataatatttccaatcgttggacaggacggtgttcagtgtgttggagacctcaacgaacgcagacccaggtataatatttgaagccatgcgtggagacaaagctacattgcaagattatatactagatttaaaacccgaaacaactgacctatactgctatgagcaattgcatgacagctcagatgaggaggatgaagtaggcatggaccggccagatggacaagcaaaaccagatataaccaattaccatattgttactgtgtgtaacatctgtgagtgcactttgcgtttgtgcattcagagtacagcagcagacgtccgaacaatccagcagatgcttatgaacacactaggaatagtgtgtcccacctgtgcacaacaataagaattc

SEQ ID NO: 55 - DNA, 865 bp, Human papillomavirus
aagcttatgtaatacgactcactatagggcgaattgggtaccatggcgctatttcacaaccctgaggaacggccatacaaattgccagacctgtgcaggacattggacactacattgcatgacgttacaatagcctgtgtctattgcagaaggcaactacaacggacagaggtatatgaatttgcctttggcgacttaaatgtagtatatagggacggggtaccattagctgcatgccaatcatgtattaaattttatgctaaaatacgggaactacgatattactcagaatcggtgtatgccacaacattagaaaccataactaatacaaagttatatgatttatcaataaggtgcatgtgttgcttgaaaccattgagtccagctgaaaaactaaggcacctaaatgcaaaacgaagattccataaaatagcaggaaactttacaggacagtgtcgccactgctggaccagtaagcgagaggaccgcagacgcacacggcaagagacacaagtataaactaactatgcatggaccaaagcccaccgtgcaggaaattgtgttagagttatgtccatgcaatgaaatagagccggttgaccttgtatgtcacgagcaattaggagattcagacgatgaaatagatgaacccgaccatgcagttaatcaccaccaacatcaactactagccagacgggacgaacaacagcgtcacagaattcagtgtatgtgttgtaagtgtaacaacccactgcaactagtagtagaagcgtcgcgggagaacctgcggaagctacaactgctgtttatggactcactaaattttgtgtgtccgtggtgtgcaacggaaacccagtaactgcag

SEQ ID NO: 56 - DNA, 863 bp, Human papillomavirus
aagcttatgtaatacgactcactatagggcgaattgggtaccatggcgcgatttctcaatcctgcagaacggccatacaaattgcctgacctgtgcacggcgctggacactacattgcacgacattacaatagactgtgtctattgtaaaacacagctacagcaaacagaggtatatgaatttgcatttagtgatttatttatagtatatagaaacggggagccatatgctgcatgccaaaaatgtattaaatttcatgctaaagtaagggaactacggcattattcgaactcggtgtatgcaacaactttggaaagcataactaataccaagttatatgatttatcaataaggtgcatgagttgcctgaaaccattgtgtccagcagaaaaattaaggcatgttaataccaaaagaagatttcaccaaatagcaggaagctatacaggacagtgccgacactgctggaccagcaaccgggaggaccgcagacgtatacgaagagaaacacaagtataaatataaatatgcatggaccacggccgacattgcaagagattgttttagatttatatccatacaatgaaatacagccggtcgaccttgtatgtcacgagcaattagaagattcagacaatgaaacagatgaacccgaccatgtagttaatcaccaacaacaactactagccagacgggaagaaccacagcgtcacaaaatacagtgtatgtgttgtaagtgtaatactacactgcacttagtagtagaagcctcacaagagaacctgcgatctctactgcagctgtttatggagacactgtcatttgtgtgtccctggtgtgcatcgggaacccagtaagaattc

SEQ ID NO: 57 - DNA, 790 bp, Human papillomavirus
aagcttatgtaatacgactcactatagggcgaattgggtaccatgctgtttcccaattcagaagaacgaccatacaagctacaagcgttatgtgacgaagtgaatatttctatacatgatataaacctggactgtgtgttttgccaacgtggactgtacagatctgaggtatatgattttgcatttagtgatttgtgtattgtatatagaaaggataaaccatatggtgtatgtcaaccgtgtttaaaattttattctaaaattagagagtataggcgatatagacaatcagtatatggcactacgttagaaaatttaactaacaaacagttatgtaatattttaataaggtgcggaaaatgccaaaaaccattatgtccactggaaaagcaaaagcatgtagatgaaaaaaaacggtttcatcaaatagcagaacagtggaccggacgctgtacacggtgctggagaccatctgcaactgtggtgtaagatgcatggaaaaaaaacaaccttgcaggacattactttagacctgaaaccaacaaccgaaattgaccttacatgttacgagtcattggacaactcagaggatgaggatgaaacagacagccatctagacagacaagctgaacgagagtgttacagaatagttactgactgcacgaagtgtcagtgcacagtatgccttgccattgaaagcaacaaagctgatttaagagtgatagaagagttgcttatgggtacactaggtattgtgtgccccaactgttccagaaacctataagaattc

SEQ ID NO: 58 - DNA, 814 bp, Human papillomavirus
aagcttatgtaatacgactcactatagggcgaattgggtaccatgtttgaagacataagagaaagaccacgaacgctgcacgaattatgtgaagcctgcaatacgtctatgcacaatattcaggtattgtgtgtatattgtaaaaaggagttgtgtagagcagatgtgtataatgtagcatttacagaacttaggattgtatatagggacaatacgccatatgcagcatgcaaaaaatgcctaatgttttattctagaattagagaatacagaaggtatagtaggtctgtgtatggtgctacattagaggccattactaacaaaagtttatatgaattattaataaggtgtcatagatgtcagagaccacttgggcctgaagaaaagcaaaaggtggtggacgacaaaaaaaggtttcatgaaatagcgggacgctggacgggacagtgtgcaaattgcagaaaaccaccaagacaacgtagtgaaacccaggtgtaataacgccatgcgtggtaatgtaccacaattaaaggacatagtgttggagttaacaccacaacctgaaattgacttgcaatgctacgagcaatttgacagctcagacgaggaggatgaagtagataatatgcgtgaccagccagccagacaagctggacaggatacgtgttacagaattaaagtgcactgttgcaggtgttcgagtgttgtacagctcgcagtggaaagcagtggagacagccttcgcatatttcagcaaatgttactgggcgacctaagcctggtgtgcccgtggtgtgcgaccaactaagaattc

SEQ ID NO: 59 - DNA, 36 bp, Artificial Sequence
  Description of Artificial Sequence Synthetic oligonucleotide
atgtaatacgactcactatagggcgaattgggtacc

SEQ ID NO: 60 - DNA, 13 bp, Artificial Sequence
  Description of Artificial Sequence Synthetic probe
tccagatgtcttt

SEQ ID NO: 61 - DNA, 13 bp, Artificial Sequence
  Description of Artificial Sequence Synthetic probe
tccagatgtcttt

SEQ ID NO: 62 - DNA, 13 bp, Artificial Sequence
  Description of Artificial Sequence Synthetic probe
tccagatgtcttt

SEQ ID NO: 63 - DNA, 7904 bp, Human papillomavirus type 16
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacataatgtatatgtacataatgtaattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttcatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaa

SEQ ID NO: 64 - DNA, 7857 bp, Human papillomavirus type 18
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtcgatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaaccgctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcccacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcctgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttcccccccccccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 65 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 66 - DNA, 1267 bp, Homo sapiens
cgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcaaaaaaaaaaaaaa

SEQ ID NO: 67 - DNA, 14 bp, Artificial Sequence
  Description of Artificial Sequence Synthetic probe
ggaatctttgcttt

SEQ ID NO: 68 - DNA, 14 bp, Artificial Sequence
  Description of Artificial Sequence Synthetic probe
ggaatctttgcttt

SEQ ID NO: 69 - DNA, 14 bp, Artificial Sequence
  Description of Artificial Sequence Synthetic probe
ggaatctttgcttt